(19) United States
(12) Patent Application Publication
(10) Pub. No.: US 2023/0203029 A1
(43) Pub. Date: Jun. 29, 2023

(54) NAPHTHYRIDINE COMPOUNDS AS INHIBITORS OF MER TYROSINE KINASE AND AXL TYROSINE KINASE

(71) Applicant: Kinsensus Limited, Oxfordshire (GB)

(72) Inventors: Edward Richard Walker, Oxfordshire (GB); Timothy William Gorman, Oxfordshire (GB); Boris Aillard, Oxfordshire (GB); Clive McCarthy, Oxfordshire (GB)

(21) Appl. No.: 17/915,879

(22) PCT Filed: Apr. 1, 2021
(86) PCT No.: PCT/GB2021/050833

§ 371 Date: Sep. 29, 2022

(30) Foreign Application Priority Data
Apr. 3, 2020 (GB) 2004960.7

(51) Int. Cl. C07D 471/04

(52) U.S. Cl. CPC C07D 471/04

(57) ABSTRACT

The present invention relates to compounds of Formula (I) that function as inhibitors of MerTK activity, to processes for the preparation of such compounds, to pharmaceutical compositions comprising them and to their use in the treatment of proliferative disorders, such as cancer, as well as other diseases or conditions in which (MerTK) activity is implicated:

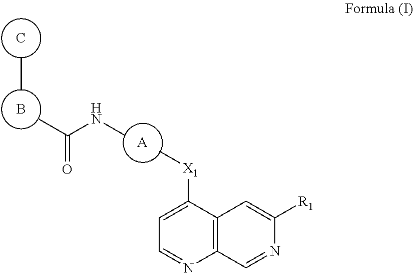

wherein R₁, X₁, Ring A, Ring B and Ring C are each as defined herein.

### INTRODUCTION

The present invention relates to certain compounds that function as inhibitors of protein kinase activity, in a particular the activity of Mer tyrosine kinase (MerTK) and AXL tyrosine kinase. The compounds of the present invention may be used to treat disease or conditions mediated, at least in part, by MerTK or AXL activity, for example hyperproliferative diseases, including cancer. The compounds of the present invention may also be used as anti-infective agents, immunostimulatory agents or immunomodulatory agents, anti-inflammatory agents, anti-thrombotic agents, anti-fibrotic agents and treatments for neurodegeneration. The invention also relates to processes for the preparation of these compounds, their use as pharmaceuticals, and to pharmaceutical compositions comprising them.

### BACKGROUND OF THE INVENTION

Protein phosphorylation is a key signalling pathway required for the maintenance of normal cell function and aberrant signalling occurs in pathophysiological settings such as cancer. Receptor tyrosine kinases (RTKs) relay signals from the cell surface to intracellular signalling pathways. One family of RTKs that plays a key role in cell signalling is the TAM family which consists of three kinases, MerTK, AXL and Tyro3. Members of this family have similar overall domain architecture consisting of an extracellular region containing Ig-like domains and FNIII domains, a transmembrane region and an intracellular kinase domain. The TAM family is activated by the ligands Gas6 and PROS1 leading to receptor dimerization and kinase domain cross-phosphorylation. Downstream signalling from the TAM enzymes is mediated in part by activation of the PI 3-kinase-Akt and Raf-MAPK pathways. The TAM family of enzymes are not frequently mutated oncogenic genes but are overexpressed in various cancers and also play a role in cancer maintenance and growth via modulation of immune cell function (reviewed in Graham et al. 2014).

MerTK is overexpressed in across a wide spectrum of cancer types including melanoma, gastric cancer, leukemia and lung cancer. RNAi knockdown affects the growth of multiple cancer cell types including mantle cell lymphoma (Shi et al. 2018) and non-small cell lung cancer (Cummings et al. 2015).

AXL is associated with the metastasis, invasion and migration of multiple cancers (Balaji et al. 2017). The epithelial to mesenchymal transition (EMT) is associated with tumour formation and growth. AXL has been shown to overexpressed in tumours that have gone through EMT and has been reported to be part of an EMT gene signature associated with resistance to targeted cancer therapeutics in non-small cell lung cancer (Byers et al. 2013). In addition, AXL overexpression is a mechanism for resistance to DNA damaging therapies through modulation of the normal DNA damage response (DDR) in cancer cells. AXL inhibition sensitises cancer cells to chemotherapy and radiation in cancers such as head and neck squamous cell carcinoma (Brand et al. 2015) and poly-ADP ribose polymerase (PARP) inhibition in multiple cancers (Balaji et al. 2017).

In addition to the direct control of cancer cell signalling, the TAM kinases exert their effects on tumour growth and maintenance via modulation of immune cell function. MerTK is expressed in tumour associated macrophages and activation of the kinase by apoptotic material creates an immunosuppressive microenvironment, a reduction of inflammatory cytokines such as IL-12 and interferon gamma and the creation of an autocrine activation loop via the increase of GAS6. Growth of syngeneic breast and melanoma tumours in mertk^{-/-} mice is impaired (Cook et al. 2013). MerTK is implicated in the control of efferocytosis, the mechanism by which apoptotic cells are cleared by phagocytic cells leading to a reduced tumour cell response by immune cells and increased likelihood of tumour residual disease (Werfel et al. 2019). In vivo validation of a role for MerTK in tumour efferocytosis has been established using anti-MerTK antibodies (Zhou et al. 2020). Activation of TAM kinases by PROS1 and GAS6 also suppresses the activation of dendritic cells and subsequent reduction in cytokines. The Pan-TAM kinase inhibitor BMS-777607 has been tested in combination with an anti-PD-1 monoclonal antibody in a model of triple-negative breast cancer (Kasikara et al. 2019). The combination of the two therapeutic agents significantly decreased tumour growth and lung metastasis. This was associated with an increase in the anti-tumour T lymphocytes. Roles of the TAM family in disease pathology in non-oncology indications are emerging. Liver diseases such as non-alcoholic steatohepatitis (NASH) are a rapidly growing global health burden. Gas6 levels are an indicative marker of liver dysfunction. In an experimental model of liver fibrosis caused by feeding mice with a methionine and choline deficient diet, inhibition of AXL by bencentinib was shown to decrease markers of fibrosis (Tutusaus et al. 2020).

Many of these studies have been performed using pharmacological tools that are either not selective for individual TAM kinases or have activity on other protein kinases. Hence, the specific functions of individual TAM kinases need to be verified with more selective therapeutic agents.

There is therefore a need for compounds that are potent inhibitors of MerTK and/or AXL.

The present invention was devised with the foregoing in mind.

References: Balaji K et al. (2017) Mol. Cancer Res. 15; 45. Brand T. M. et al. (2015) Clin. Cancer Res. 21; 2601. Byers L. A. et al. (2013) Clin. Cancer Res. 19; 279. Cook R. S. et al. (2013) J. Clin. Invest. 123; 3231. Cummings C. T. et al. (2015) Mol Cancer Ther. 14: 2014. Graham D. K. et al. (2014) Nature Reviews Cancer 14; 769. Kasikara C. et al. (2019) Cancer Research 79; 2669. Martinelli E. et al. (2015) Oncotarget 6; 23281. Shi C. et al. (2018) Journal of Hematology and Oncology 11; 43. Tutusaus A. et al. (2020) Cell. Mol. Gastroenterol. Hepatol. 9; 349. Werfel T. A et al. (2019) Cancer Res. 79; 171. Zhou Y. et al. (2020) Immunity 52; 357.

### SUMMARY OF THE INVENTION

According to a first aspect of the present invention, there is provided a compound, or a pharmaceutically acceptable salt or solvate thereof, as defined herein.

According to a further aspect of the present invention, there is provided a pharmaceutical composition comprising a compound as defined herein, or a pharmaceutically acceptable salt or solvate thereof, in admixture with a pharmaceutically acceptable diluent or carrier.

According to a further aspect of the present invention, there is provided a method of inhibiting MerTK and/or AXL activity, in vitro or in vivo, said method comprising contacting a cell with an effective amount of a compound, or a pharmaceutically acceptable salt or solvate thereof, as defined herein.

According to a further aspect of the present invention, there is provided a method of inhibiting cell proliferation, in vitro or in vivo, said method comprising contacting a cell with an effective amount of a compound, or a pharmaceutically acceptable salt or solvate thereof, as defined herein, or a pharmaceutical composition as defined herein.

According to a further aspect of the present invention, there is provided a method of treating a disease or disorder in which MerTK and/or AXL activity is implicated in a patient in need of such treatment, said method comprising administering to said patient a therapeutically effective amount of a compound, or a pharmaceutically acceptable salt or solvate thereof, as defined herein, or a pharmaceutical composition as defined herein.

Conditions in which MerTK and/or AXL activity is implicated include proliferative disorders, including cancer, as well as infections, immune modulation and/or stimulation, and disorders associated with platelet aggregation (e.g. thrombosis).

According to a further aspect of the present invention, there is provided a method of treating a proliferative disorder in a patient in need of such treatment, said method comprising administering to said patient a therapeutically effective amount of a compound or a pharmaceutically acceptable salt or solvate thereof as defined herein, or a pharmaceutical composition as defined herein.

According to a further aspect of the present invention, there is provided a method of treating cancer in a patient in need of such treatment, said method comprising administering to said patient a therapeutically effective amount of a compound or a pharmaceutically acceptable salt or solvate thereof as defined herein, or a pharmaceutical composition as defined herein.

According to a further aspect of the present invention, there is provided a compound, or a pharmaceutically acceptable salt or solvate thereof, or a pharmaceutical composition as defined herein for use in therapy.

According to a further aspect of the present invention, there is provided a compound or a pharmaceutically acceptable salt or solvate thereof as defined herein, or a pharmaceutical composition as defined herein, for use in the treatment of a proliferative condition.

According to a further aspect of the present invention, there is provided a compound, or a pharmaceutically acceptable salt or solvate thereof, or a pharmaceutical composition as defined herein for use in the treatment of cancer. In a particular embodiment, the cancer is human cancer.

According to a further aspect of the present invention, there is provided a compound, or a pharmaceutically acceptable salt or solvate thereof, as defined herein for use in the inhibition of MerTK and/or AXL activity.

According to a further aspect of the present invention, there is provided a compound, or a pharmaceutically acceptable salt or solvate thereof, as defined herein for use in the treatment of a disease or disorder in which MerTK and/or AXL activity is implicated.

According to a further aspect of the present invention, there is provided the use of a compound, or a pharmaceutically acceptable salt or solvate thereof, as defined herein in the manufacture of a medicament for the treatment of a proliferative condition.

Suitably, the proliferative disorder is cancer, suitably a human cancer (for example haematological cancers such as lymphomas (including diffuse large B-cell lymphoma (DLBCL), follicular lymphoma (FL), Burkitt lymphoma (BL) and angioimmunoblastic T-cell lymphoma (AITL)), leukaemias (including acute lymphoblastic leukaemia (ALL) and chronic myeloid leukaemia (CML)) and multiple myeloma, and solid tumours (including glioma, breast cancer, non-small cell lung cancer (NSCLC) and squamous cell carcinomas (SCC) (including SCC of the head and neck, oesophagus, lung and ovary)).

According to a further aspect of the present invention, there is provided the use of a compound, or a pharmaceutically acceptable salt or solvate thereof, as defined herein in the manufacture of a medicament for the treatment of cancer.

According to a further aspect of the present invention, there is provided a use of a compound, or a pharmaceutically acceptable salt or solvate thereof, as defined herein in the manufacture of a medicament for the inhibition of MerTK and/or AXL activity.

According to a further aspect of the present invention, there is provided a use of a compound, or a pharmaceutically acceptable salt or solvate thereof, as defined herein in the manufacture of a medicament for the treatment of a disease or disorder in which MerTK and/or AXL activity is implicated.

According to a further aspect of the present invention, there is provided a process for preparing a compound, or a pharmaceutically acceptable salt or solvate thereof, as defined herein.

According to a further aspect of the present invention, there is provided a compound, or a pharmaceutically acceptable salt or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

Features, including optional, suitable, and preferred features in relation to one aspect of the invention may also be features, including optional, suitable and preferred features in relation to any other aspect of the invention.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl.

An “alkylene” group is an alkyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene (—CH₂—), ethylene (—CH₂CH₂—), propylene (—CH₂CH₂CH₂—), 2-methylpropylene (—CH₂CH(CH₃)CH₂—), pentylene (—CH₂CH₂CH₂CH₂CH₂—), and the like.

The term “alkyenyl” refers to straight and branched chain alkyl groups comprising 2 or more carbon atoms, wherein at least one carbon-carbon double bond is present within the group. Examples of alkenyl groups include ethenyl, propenyl and but-2,3-enyl and includes all possible geometric (E/Z) isomers.

The term “alkynyl” refers to straight and branched chain alkyl groups comprising 2 or more carbon atoms, wherein at least one carbon-carbon triple bond is present within the group. Examples of alkynyl groups include acetylenyl and propynyl.

“(3-6C)cycloalkyl” means a hydrocarbon ring containing from 3 to 6 carbon atoms, for example, cyclopropyl, cyclobutyl, cyclopentyl, cyclohexyl.

The term “alkoxy” refers to O-linked straight and branched chain alkyl groups. Examples of alkoxy groups include methoxy, ethoxy and t-butoxy.

The term “haloalkyl” is used herein to refer to an alkyl group in which one or more hydrogen atoms have been replaced by halogen (e.g. fluorine) atoms. Examples of haloalkyl groups include —CH₂F, —CHF₂ and —CF₃.

The term “halo” or “halogeno” refers to fluoro, chloro, bromo and iodo, suitably fluoro, chloro and bromo, more suitably, fluoro and chloro.

The term “carbocyclyl”, “carbocyclic” or “carbocycle” means a non-aromatic saturated or partially saturated monocyclic, fused, bridged, or spiro bicyclic carbon-containing ring system(s). Monocyclic carbocyclic rings contain from about 3 to 12 (suitably from 3 to 7) ring atoms. Bicyclic carbocycles contain from 6 to 17 member atoms, suitably 7 to 12 member atoms, in the ring. Bicyclic carbocyclic(s) rings may be fused, spiro, or bridged ring systems. Examples of carbocyclic groups include cyclopropyl, cyclobutyl, cyclopentyl, cyclohexyl, cyclohexenyl and spiro[3.3]heptanyl.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. Heterocycles may comprise 1 or 2 oxo (═O) or thioxo (═S) substituents. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

Heterocyclyl rings may be C- or N-linked as appropriate and certain heteroaryl rings may also be C- or N-linked as appropriate, for example pyrazole rings can be N or C linked.

The following structures are examples of heterocyclyl groups, wherein [symbol] represents possible, non-limiting, points of attachment to the rest of the molecule, noting that the point of attachment of the heterocyclyl group to the rest of the molecule may also be via a ring nitrogen atom, as appropriate.

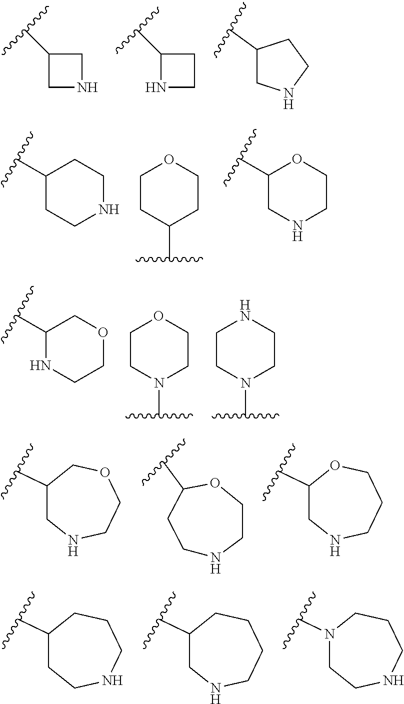

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bi-cyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:

a benzene ring fused to a 5- or 6-membered ring containing 1, 2 or 3 ring heteroatoms;

a pyridine ring fused to a 5- or 6-membered ring containing 1, 2 or 3 ring heteroatoms;

a pyrimidine ring fused to a 5- or 6-membered ring containing 1 or 2 ring heteroatoms;

a pyrrole ring fused to a 5- or 6-membered ring containing 1, 2 or 3 ring heteroatoms;

a pyrazole ring fused to a 5- or 6-membered ring containing 1 or 2 ring heteroatoms;

a pyrazine ring fused to a 5- or 6-membered ring containing 1 or 2 ring heteroatoms;

an imidazole ring fused to a 5- or 6-membered ring containing 1 or 2 ring heteroatoms;

an oxazole ring fused to a 5- or 6-membered ring containing 1 or 2 ring heteroatoms;

an isoxazole ring fused to a 5- or 6-membered ring containing 1 or 2 ring heteroatoms;

a thiazole ring fused to a 5- or 6-membered ring containing 1 or 2 ring heteroatoms;

an isothiazole ring fused to a 5- or 6-membered ring containing 1 or 2 ring heteroatoms;

a thiophene ring fused to a 5- or 6-membered ring containing 1, 2 or 3 ring heteroatoms;

a furan ring fused to a 5- or 6-membered ring containing 1, 2 or 3 ring heteroatoms;

a cyclohexyl ring fused to a 5- or 6-membered heteroaromatic ring containing 1, 2 or 3 ring heteroatoms; and

a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In a particular embodiment, an aryl is phenyl.

This specification also makes use of several composite terms to describe groups comprising more than one functionality. Such terms will be understood by a person skilled in the art. For example (3-6C)cycloalkyl(m-nC)alkyl comprises (m-nC)alkylene substituted by (3-6C)cycloalkyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In one aspect, the present invention relates to compounds, or pharmaceutically acceptable salts or solvates thereof, having the structural Formula (I), shown below:

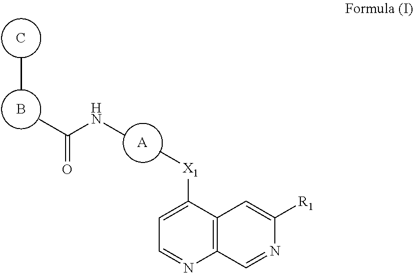

wherein:
R₁ is selected from:
(i) a cycloalkyl, aryl, heteroaryl or heterocyclyl ring,
   each of which is optionally substituted on an available carbon atom by one or more R_{100C} substituents;
   wherein each R_{100C} substituent present is independently selected from halo, hydroxy, cyano, Rₐ, NRₐR_{b}, ORₐ, C(O)Rₐ, C(O)ORₐ, OC(O)Rₐ, C(O)N(R_{b})Rₐ, C(O)N(R_{b})ORₐ, N(R_{b})C(O)Rₐ, S(O)_{y}Rₐ (wherein y is 0, 1 or 2), S(O)₂N(R_{b})Rₐ, N(R_{b})S(O)₂Rₐ, (CH₂)_{z}Rₐ or (CH₂)_{z}NRₐR_{b} (where z is 1, 2 or 3);
   wherein Rₐ is selected from:
   (a) (1-4C)alkyl which is optionally substituted by one or more substituents independently selected from halo, hydroxy, cyano, amino, (3-6C)cycloalkyl, (1-4C)alkylamino, di-(1-4C)alkylamino or (1-4C)alkoxy and wherein any alkyl or cycloalkyl moiety present in such substituent groups is optionally further substituted by halo, hydroxy and/or (1-2C)alkoxy; or
   (b) aryl, heteroaryl, cycloalkyl, heterocyclyl or heterocyclyl(1-2C)alkyl, each of which is optionally substituted on an available carbon atom by one or more substituents independently selected from halo, hydroxy, cyano, amino, (1-4C)alkyl, (1-4C)alkylamino, di-(1-4C)alkylamino, (1-4C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are each independently selected from hydrogen or (1-4C)alkyl, and wherein any alkyl moiety present in a substituent group is optionally further substituted by halo, hydroxy and/or (1-2C)alkoxy;
   and R_{b} is selected from hydrogen or (1-2C)alkyl;
   and, when R₁ or Rₐ is a heteroaryl, heterocyclyl or heterocyclyl(1-2C)alkyl, an available nitrogen atom (where valency permits) is optionally substituted by one or more R_{100N};
   wherein each R_{100N} is selected from:
   (a) (1-4C)alkyl which is optionally substituted by halo, oxo, hydroxy, cyano, amino and/or (1-4C)alkoxy (optionally substituted by halo and/or (1-2C)alkoxy); or
   (b) (3-6C)cycloalkyl, phenyl, heterocyclyl or heteroaryl, each of which is optionally further substituted on an available carbon atom by one or more substituents independently selected from halo, hydroxy, cyano, amino, (1-4C)alkyl, (1-4C)alkylamino, di-(1-4C)alkylamino, (1-4C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are as defined above, and wherein any alkyl moiety present in a substituent group is optionally further substituted by halo, hydroxy and/or (1-2C)alkoxy; and an available ring nitrogen atom (where valency permits) is optionally further substituted by (1-4C)alkyl optionally substituted by halo, oxo, hydroxy, cyano, amino and/or (1-4C)alkoxy (which is optionally further substituted by halo and/or (1-2C)alkoxy);
   (c) C(O)Rₐ₂, C(O)ORₐ₂, C(O)N(R_{b2})Rₐ₂ or C(O)N(R_{b2})ORₐ₂; wherein Rₐ₂ and R_{b2} are each independently selected from hydrogen or (1-4C)alkyl, and wherein any alkyl moiety present in a substituent group is optionally further substituted by halo, hydroxy and/or (1-2C)alkoxy;
   or
(ii) a group:

-L-R₂;
wherein:
L is a linking group selected from: —O—; —CO—; —COO—; —OCO—; —NR₁ₐ—; —CONR₁ₐ—; —CONR₁ₐ—O—; —NR₁ₐCO—; —NR₁ₐCOO—; or —NR₁ₐCONR_{1b}—;
R₁ₐ or R_{1b} are both independently selected from hydrogen or (1-4C)alkyl; and
R₂ is selected from:
   (a) hydrogen;
   (b) (1-4C)alkyl which is optionally substituted by halo, hydroxy, cyano, amino, (1-4C)alkylamino, di-(1-4C)alkylamino and/or (1-4C)alkoxy (which is optionally further substituted by halo and/or (1-4C)alkoxy); or
   (c) a cycloalkyl, aryl, heteroaryl, heterocyclyl, cycloalkyl(1-2C)alkyl, aryl(1-2C)alkyl, heteroaryl(1-2C)alkyl or heterocyclyl(1-2C)alkyl ring,
   wherein each ring system is optionally substituted on an available carbon atom by one or more R_{101C} substituents;
   wherein each R_{101C} substituent is independently selected from halo, hydroxy, cyano, R_{c}, NR_{d}R_{c}, OR_{c}, C(O)R_{c}, C(O)OR_{c}, OC(O)R_{c}, C(O)N(R_{d})R_{c}, C(O)N(R_{d})OR_{c}, N(R_{d})C(O)R_{c}, S(O)_{y}R_{c} (wherein y is 0, 1 or 2), S(O)₂N(R_{d})R_{c}, N(R_{d})S(O)₂R_{c}, (CH₂)_{z}R_{c} or (CH₂)_{z}NR_{c}R_{d} (where z is 1, 2 or 3);
and wherein R_{c} is selected from:
1. hydrogen;
2. (1-4C)alkyl which is optionally substituted by one or more substituents independently selected from halo, hydroxy, cyano, amino, (3-6C)cycloalkyl, (1-4C)alkylamino, di-(1-4C)alkylamino or (1-4C)alkoxy and wherein any alkyl or cycloalkyl moiety present in such substituent groups is optionally further substituted by halo, hydroxy and/or (1-2C)alkoxy; or
3. a cycloalkyl, aryl, heteroaryl, heterocyclyl or heterocyclyl(1-2C)alkyl ring,
   wherein each ring system is optionally substituted on an available carbon atom by one or more substituents independently selected from halo, hydroxy, cyano, amino, (1-4C)alkyl, (1-4C)alkylamino, di-(1-4C)alkylamino, (1-4C)alkoxy, C(O)R_{c1}, C(O)OR_{c1}, OC(O)R_{c1}, C(O)N(R_{d1})R_{c1}, C(O)N(R_{d1})OR_{c1} N(R_{d1})C(O)R_{c1}, S(O)_{y}R_{c1} (wherein y is 0, 1 or 2), S(O)₂N(R_{b1})R_{c1} or N(R_{d1})S(O)₂R_{c1}, wherein R_{c1} and R_{d1} are each independently selected from hydrogen or (1-4C)alkyl, and wherein any alkyl moiety present in a substituent group is optionally further substituted by halo, hydroxy and/or (1-2C)alkoxy;
and R_{d} is selected from hydrogen or (1-2C)alkyl;
and, when R₂ or R_{c} is a heteroaryl or heterocyclyl ring, an available nitrogen atom (where valency permits) is optionally further substituted by one or more R_{101N};
wherein each R_{101N} substituent is selected from:
   (a) (1-4C)alkyl which is optionally substituted by one or more substituents independently selected from halo, oxo, hydroxy, cyano, amino or (1-4C)alkoxy (which is optionally further substituted by halo and/or (1-2C)alkoxy);
   (b) (3-6C)cycloalkyl, phenyl, or a carbon-linked heterocyclyl or heteroaryl ring, each of which is optionally further substituted by one or more substituents independently selected from halo, hydroxy, cyano, amino, (1-4C)alkyl, (1-4C)alkylamino, di-(1-4C)alkylamino, (1-4C)alkoxy, C(O)R_{c1}, C(O)OR_{c1}, OC(O)R_{c1}, C(O)N(R_{d1})R_{c1}, C(O)N(R_{d1})OR_{c1} N(R_{d1})C(O)R_{c1}, S(O)_{y}R_{c1} (wherein y is 0, 1 or 2), S(O)₂N(R_{b1})R_{c1} or N(R_{d1})S(O)₂R_{c1}, wherein R_{c1} and R_{d1} are as defined above and wherein any alkyl moiety present in a substituent group is optionally further substituted by halo, hydroxy and/or (1-2C)alkoxy;
   (c) C(O)R_{c2}, C(O)OR_{c2}, OC(O)R_{c2}, C(O)N(R_{d2})R_{c2}, C(O)N(R_{d2})OR_{c2}; wherein R_{c2} and R_{d2} are each independently selected from hydrogen or (1-4C)alkyl, and wherein any alkyl moiety present in a substituent group is optionally further substituted by halo, hydroxy and/or (1-2C)alkoxy;
or, when L is a linking group selected from —NR₁ₐ—, —CONR₁ₐ—, or —NR₁ₐCO, R₁ₐ and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N};
or, when L is —NR₁ₐCONR_{1b}—, R_{1b} and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N};
X₁ is —NH— or —O—;
Ring A is selected from the following:

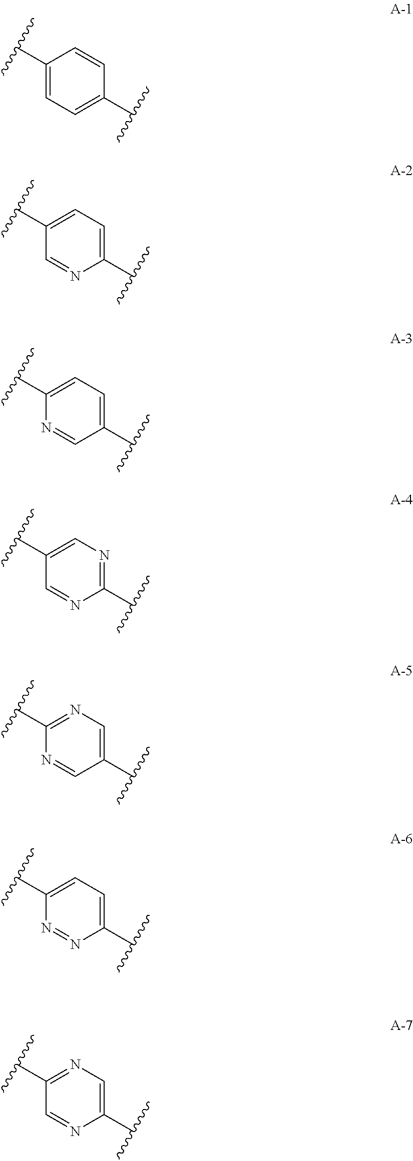

wherein [symbol] represents the respective points of attachment of Ring A to the rest of the compound of Formula (I) and wherein Ring A may be optionally substituted on an available carbon atom by one or more substituents independently selected from halo, (1-4C)alkyl and (1-4C)alkoxy;
Ring B is a 5- or 6-membered heteroaryl or a heterocyclic ring comprising one to three N atoms, linked via a carbon atom to the amide bond in the compound of Formula (I) and wherein:
(a) a heteroaryl ring is optionally substituted on an available carbon atom by one or more substituents independently selected from hydroxy, halo, cyano, (1-6C)alkyl or (1-6C)alkoxy; or on an available nitrogen atom by (1-6C)alkyl or O—; and wherein any alkyl moiety present in a substituent group is optionally further substituted by halo, hydroxy and/or (1-2C)alkoxy;
and
(b) a heterocyclyl ring is optionally substituted on an available carbon atom by one or more substituents independently selected from oxo, hydroxy, halo, cyano, (1-6C)alkyl, (1-6C)alkoxy; or on an available nitrogen atom by (1-6C)alkyl; and wherein any alkyl moiety present in a substituent group is optionally further substituted by halo, hydroxy and/or (1-2C)alkoxy;
Ring C is selected from the following:

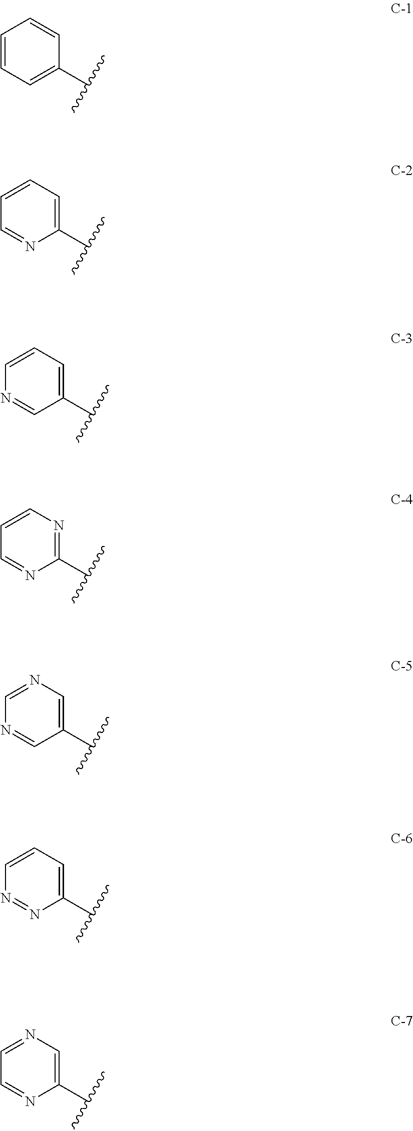

wherein [symbol] represents the point of attachment of Ring C to Ring B and wherein Ring C is optionally substituted on an available carbon atom by one or more substituents independently selected from hydroxy, halo, cyano, (1-6C)alkyl or (1-6C)alkoxy; or on an available nitrogen atom by (1-6C)alkyl; and wherein any alkyl moiety present in a substituent group is optionally further substituted by halo, hydroxy and/or (1-2C)alkoxy.
Particular compounds of the invention include, for example, compounds of the Formula (I), or pharmaceutically acceptable salts and/or solvates thereof, wherein, unless otherwise stated, each of R₁, X₁, Ring A, Ring B and Ring C (and any other associated variable definitions in compounds of Formula (I) or sub-formulae thereof) and any associated substituent groups has any of the meanings defined hereinbefore, or in any of paragraphs (1) to (24) hereinafter:
(1) R₁ is selected from:
(i) a cycloalkyl, aryl, heteroaryl or heterocyclyl ring,
   each of which is optionally substituted on an available carbon atom by one or more R_{100C} substituents;
   wherein each R_{100C} substituent is independently selected from halo, hydroxy, cyano, Rₐ, NRₐR_{b}, ORₐ, C(O)Rₐ, C(O)ORₐ, OC(O)Rₐ, C(O)N(R_{b})Rₐ, C(O)N(R_{b})ORₐ, N(R_{b})C(O)Rₐ, S(O)_{y}Rₐ (wherein y is 0, 1 or 2), S(O)₂N(R_{b})Rₐ, N(R_{b})S(O)₂Rₐ, (CH₂)_{z}Rₐ or (CH₂)_{z}NRₐR_{b} (where z is 1, 2 or 3);
   wherein Rₐ is selected from:
   (a) (1-2C)alkyl which is optionally substituted by one or more substituents independently selected from halo, hydroxy, cyano, amino, (3-4C)cycloalkyl, (1-2C)alkylamino, di-(1-2C)alkylamino or (1-2C)alkoxy and wherein any alkyl or cycloalkyl moiety present in such substituent groups is optionally further substituted by halo, hydroxy and/or (1-2C)alkoxy; or
   (b) aryl, heteroaryl, cycloalkyl, heterocyclyl or heterocyclyl(1-2C)alkyl, each of which is optionally substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b}, are each independently selected from hydrogen or (1-2C)alkyl, and wherein any alkyl moiety present in a substituent group is optionally further substituted by halo, hydroxy and/or (1-2C)alkoxy;
   and R_{b} is selected from hydrogen or (1-2C)alkyl;
   and, when R₁ or Rₐ is a heteroaryl, heterocyclyl or heterocyclyl(1-2C)alkyl, an available nitrogen atom (where valency permits) is optionally substituted by one or more R_{100N} substituents;
   wherein each R_{100N} substituent is selected from:
   (a) (1-4C)alkyl which is optionally substituted by halo, oxo, hydroxy, cyano, amino and/or (1-4C)alkoxy (optionally substituted by halo and/or (1-2C)alkoxy); or
   (b) (3-6C)cycloalkyl, phenyl, heterocyclyl or heteroaryl, each of which is optionally further substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are as defined above, and wherein any alkyl moiety present in a substituent group is optionally further substituted by halo, hydroxy and/or (1-2C)alkoxy;
      and an available ring nitrogen atom (where valency permits) is optionally further substituted by (1-4C)alkyl optionally substituted by halo, hydroxy, cyano, amino and/or (1-2C)alkoxy (which is optionally further substituted by halo and/or (1-2C)alkoxy);
   (c) C(O)Rₐ₂, C(O)ORₐ₂, C(O)N(R_{b2})Rₐ₂ or C(O)N(R_{b2})ORₐ₂; wherein Rₐ₂ and R_{b2} are each independently selected from hydrogen or (1-4C)alkyl, and wherein any alkyl moiety present in a substituent group is optionally further substituted by halo, hydroxy and/or (1-2C)alkoxy;
      or
(ii) a group:

-L-R₂;
wherein:
L is a linking group selected from: —O—; —CO—; —COO—; —OCO—; —NR₁ₐ—; —CONR₁ₐ—; —CONR₁ₐ—O—; —NR₁ₐCO—; —NR₁ₐCOO—; or NR₁ₐCONR_{1b}—;
R₁ₐ and R_{1b} are independently selected from hydrogen or (1-2C)alkyl; and
R₂ is selected from:
(a) hydrogen;
(b) (1-4C)alkyl which is optionally substituted by halo, hydroxy, cyano, amino, (1-2C)alkylamino, di-(1-2C)alkylamino and/or (1-4C)alkoxy (which is optionally further substituted by halo and/or (1-4C)alkoxy); or
(c) a cycloalkyl, aryl, heteroaryl, heterocyclyl, cycloalkyl(1-2C)alkyl, aryl(1-2C)alkyl, heteroaryl(1-2C)alkyl or heterocyclyl(1-2C)alkyl ring,
   wherein each ring system is optionally substituted on an available carbon atom by one or more R_{101C} substituents;
   wherein each R_{101C} substituent is independently selected from halo, hydroxy, cyano, R_{c}, NR_{d}R_{c}, OR_{c}, C(O)R_{c}, C(O)OR_{c}, OC(O)R_{c}, C(O)N(R_{d})R_{c}, C(O)N(R_{d})OR_{c}, N(R_{d})C(O)R_{c}, S(O)_{y}R_{c} (wherein y is 0, 1 or 2), S(O)₂N(R_{d})R_{c}, N(R_{d})S(O)₂R_{c}, (CH₂)_{z}R_{c}R_{d} or (CH₂)_{z}NR_{c}R_{d} (where z is 1, 2 or 3);
   and wherein R_{c} is selected from:
(a) hydrogen;
(b) (1-4C)alkyl which is optionally substituted by one or more substituents independently selected from halo, hydroxy, cyano, amino, (3-6C)cycloalkyl, (1-2C)alkylamino, di-(1-2C)alkylamino or (1-2C)alkoxy and wherein any alkyl or cycloalkyl moiety present in such substituent groups is optionally further substituted by halo, hydroxy and/or (1-2C)alkoxy; or
(c) a cycloalkyl, aryl, heteroaryl, heterocyclyl or heterocyclyl(1-2C)alkyl ring,
   wherein each ring system is optionally substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)R_{c1}, C(O)OR_{c1}, OC(O)R_{c1}, C(O)N(R_{d1})R_{c1}, C(O)N(R_{d1})OR_{c1} N(R_{d1})C(O)R_{c1}, S(O)_{y}R_{c1} (wherein y is 0, 1 or 2), S(O)₂N(R_{b1})R_{c1} or N(R_{d1})S(O)₂R_{c1}, wherein R_{c1} and R_{d1} are each independently selected from hydrogen or (1-2C)alkyl, and wherein any alkyl moiety present in a substituent group is optionally further substituted by halo, hydroxy and/or (1-2C)alkoxy;
   and R_{d} is selected from hydrogen or (1-2C)alkyl;
and, when R₂ or R_{c} is a heteroaryl or heterocyclyl ring, an available nitrogen atom (where valency permits) is optionally further substituted by one or more R_{101N} substituents;
wherein R_{101N} is selected from:
(a) (1-2C)alkyl which is optionally substituted by one or more substituents independently selected from halo, oxo, hydroxy, cyano, amino or (1-2C)alkoxy (which is optionally further substituted by halo and/or (1-2C)alkoxy);
(b) (3-6C)cycloalkyl, phenyl, or a carbon-linked heterocyclyl or heteroaryl ring, each of which is optionally further substituted by one or more substituents independently selected from halo, hydroxy, cyano, amino, (1-2C)alkyl, (1-2C)alkylamino, di-(1-2C)alkylamino, (1-2C)alkoxy, C(O)R_{c1}, C(O)OR_{c1}, OC(O)R_{c1}, C(O)N(R_{d1})R_{c1}, C(O)N(R_{d1})OR_{c1} N(R_{d1})C(O)R_{c1}, S(O)_{y}R_{c1} (wherein y is 0, 1 or 2), S(O)₂N(R_{b1})R_{c1} or N(R_{d1})S(O)₂R_{c1}, wherein R_{c1} and R_{d1} are as defined above and wherein any alkyl moiety present in a substituent group is optionally further substituted by halo, hydroxy and/or (1-2C)alkoxy;
(c) C(O)R_{c2}, C(O)OR_{c2}, C(O)N(R_{d2})R_{c2} or C(O)N(R_{d2})OR_{c2}; wherein R_{c2} and R_{d2} are each independently selected from hydrogen or (1-4C)alkyl, and wherein any alkyl moiety present in a substituent group is optionally further substituted by halo, hydroxy and/or (1-2C)alkoxy;
or, when L is a linking group selected from —NR₁ₐ—, —CONR₁ₐ—, or —NR₁ₐCO, R₁ₐ and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N};
or, when L is —NR₁ₐCONR_{1b}—, R_{1b} and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N}.
   (2) R₁ is selected from:
(i) a cycloalkyl, aryl, heteroaryl or heterocyclyl ring,
   each of which is optionally substituted on an available carbon atom by one or more R_{100C} substituents;
   wherein each R_{100C} substituent is independently selected from halo, hydroxy, cyano, Rₐ, NRₐR_{b}, ORₐ, C(O)Rₐ, C(O)ORₐ, OC(O)Rₐ, C(O)N(R_{b})Rₐ, C(O)N(R_{b})ORₐ, N(R_{b})C(O)Rₐ, S(O)_{y}Rₐ (wherein y is 0, 1 or 2), S(O)₂N(R_{b})Rₐ, N(R_{b})S(O)₂Rₐ, (CH₂)_{z}Rₐ or (CH₂)_{z}NRₐR_{b} (where z is 1, 2 or 3);
   wherein Rₐ is selected from:
   (a) (1-2C)alkyl which is optionally substituted by one or more substituents independently selected from halo, hydroxy, cyano, amino, (3-4C)cycloalkyl, (1-2C)alkylamino, di-(1-2C)alkylamino or (1-2C)alkoxy and wherein any alkyl or cycloalkyl moiety present in such substituent groups is optionally further substituted by halo, hydroxy and/or (1-2C)alkoxy; or
   (b) aryl, heteroaryl, heterocyclyl or heterocyclyl(1-2C)alkyl, each of which is optionally substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are each independently selected from hydrogen or (1-2C)alkyl, and wherein any alkyl moiety present in a substituent group is optionally further substituted by halo, hydroxy and/or (1-2C)alkoxy;
   and R_{b} is selected from hydrogen or (1-2C)alkyl;
   and, when R₁ or Rₐ is a heteroaryl, heterocyclyl or heterocyclyl(1-2C)alkyl, an available nitrogen atom (where valency permits) is optionally substituted by one or more R_{100N}:
   wherein R_{100N} is selected from:
   (a) (1-4C)alkyl which is optionally substituted by halo, hydroxy, cyano, amino and/or (1-1C)alkoxy (optionally substituted by halo and/or (1-2C)alkoxy); or
   (b) heterocyclyl or heteroaryl, each of which is optionally further substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are as defined above, and wherein any alkyl moiety present in a substituent group is optionally further substituted by halo, hydroxy and/or (1-2C)alkoxy;
   and an available ring nitrogen atom (where valency permits) is optionally further substituted by (1-4C)alkyl optionally substituted by halo, hydroxy, cyano, amino and/or (1-2C)alkoxy;
(c) C(O)Rₐ₂, C(O)ORₐ₂, C(O)N(R_{b2})Rₐ₂ or C(O)N(R_{b2})ORₐ₂; wherein Rₐ₂ and R_{b2} are each independently selected from hydrogen or (1-4C)alkyl, and wherein any alkyl moiety present in a substituent group is optionally further substituted by halo, hydroxy and/or (1-2C)alkoxy; or
(ii) a group:

-L-R₂;
wherein:
L is a linking group selected from: —O—; —CO—; —COO—; —OCO—; —NR₁ₐ—; —CONR₁ₐ—; —CONR₁ₐ—O—; —NR₁ₐCO—; —NR₁ₐCOO—; or NR₁ₐCONR_{1b}—;
R₁ₐ and R_{1b} are independently selected from hydrogen or (1-2C)alkyl; and
R₂ is selected from:
   (a) hydrogen;
   (b) (1-4C)alkyl which is optionally substituted by halo, hydroxy, cyano, amino, (1-2C)alkylamino, di-(1-2C)alkylamino and/or (1-2C)alkoxy (which is optionally further substituted by halo and/or (1-2C)alkoxy); or
   (c) a cycloalkyl, aryl, heteroaryl, heterocyclyl, or heterocyclyl(1-2C)alkyl ring,
      wherein each ring system is optionally substituted on an available carbon atom by one or more R_{101C} substituents;
      wherein each R_{101C} substituent is independently selected from halo, hydroxy, cyano, R_{c}, NR_{d}R_{c}, OR_{c}, C(O)R_{c}, C(O)OR_{c}, OC(O)R_{c}, C(O)N(R_{d})R_{c}, C(O)N(R_{d})OR_{c}, N(R_{d})C(O)R_{c}, S(O)_{y}R_{c} (wherein y is 0, 1 or 2), S(O)₂N(R_{d})R_{c}, N(R_{d})S(O)₂R_{c}, (CH₂)_{z}R_{c} or (CH₂)_{z}NR_{c}R_{d} (where z is 1, 2 or 3);
and wherein R_{c} is selected from:
1. hydrogen;
2. (1-4C)alkyl which is optionally substituted by one or more substituents independently selected from halo, hydroxy, cyano, amino, (3-6C)cycloalkyl, (1-2C)alkylamino, di-(1-2C)alkylamino or (1-2C)alkoxy and wherein any alkyl or cycloalkyl moiety present in such substituent groups is optionally further substituted by halo, hydroxy and/or (1-2C)alkoxy; or
3. aryl, heteroaryl, heterocyclyl or heterocyclyl(1-2C)alkyl ring,
   wherein each ring system is optionally substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)R_{c1}, C(O)OR_{c1}, OC(O)R_{c1}, C(O)N(R_{d1})R_{c1}, C(O)N(R_{d1})OR_{c1} N(R_{d1})C(O)R_{c1}, S(O)_{y}R_{c1} (wherein y is 0, 1 or 2), S(O)₂N(R_{b1})R_{c1} or N(R_{d1})S(O)₂R_{c1}, wherein R_{c1} and R_{d1} are each independently selected from hydrogen or (1-2C)alkyl, and wherein any alkyl moiety present in a substituent group is optionally further substituted by halo, hydroxy and/or (1-2C)alkoxy;
and R_{d} is selected from hydrogen or (1-2C)alkyl;
and, when R₂ or R_{c} is a heteroaryl or heterocyclyl ring, an available nitrogen atom (where valency permits) is optionally further substituted by one or more R_{101N} substituents, wherein R_{101N} is selected from:
a) a (1-4C)alkyl which is optionally substituted by one or more substituents independently selected from halo, oxo, hydroxy, cyano, amino or (1-2C)alkoxy; or
b) C(O)R_{c2}, C(O)OR_{c2}, C(O)N(R_{d2})R_{c2} or C(O)N(R_{c2})OR_{d2}; wherein R_{c2} and R_{d2} are each independently selected from hydrogen or (1-4C)alkyl, and wherein any alkyl moiety present in a substituent group is optionally further substituted by halo, hydroxy and/or (1-2C)alkoxy
   or, when L is —NR₁ₐCONR_{1b}—, R_{1b} and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N}.
(3) R₁ is selected from:
(i) a cycloalkyl, aryl, heteroaryl or heterocyclyl ring,
   each of which is optionally substituted on an available carbon atom by one or more R_{100C} substituents;
   wherein each R_{100C} substituent is independently selected from halo, hydroxy, cyano, Rₐ, NRₐR_{b}, ORₐ, C(O)Rₐ, C(O)ORₐ, OC(O)Rₐ, C(O)N(R_{b})Rₐ, C(O)N(R_{b})ORₐ, N(R_{b})C(O)Rₐ, S(O)_{y}Rₐ (wherein y is 0, 1 or 2), S(O)₂N(R_{b})Rₐ, N(R_{b})S(O)₂Rₐ, (CH₂)_{z}Rₐ or (CH₂)_{z}NRₐR_{b} (where z is 1, 2 or 3);
   wherein Rₐ is selected from:
   (a) (1-2C)alkyl which is optionally substituted by one or more substituents independently selected from halo, hydroxy, cyano, or amino; or
   (b) heteroaryl or heterocyclyl, each of which is optionally substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are each independently selected from hydrogen or (1-2C)alkyl, and wherein any alkyl moiety present in a substituent group is optionally further substituted by halo, hydroxy and/or (1-2C)alkoxy;
   and R_{b} is selected from hydrogen or (1-2C)alkyl;
   and, when R₁ or Rₐ is a heteroaryl, heterocyclyl or heterocyclyl(1-2C)alkyl, an available nitrogen atom (where valency permits) is optionally substituted by one or more R_{100N};
   wherein R_{100N} is selected from:
   (a) (1-4C)alkyl which is optionally substituted by halo, oxo, hydroxy, cyano, amino and/or (1-1C)alkoxy (optionally substituted by halo and/or (1-2C)alkoxy); or
   (b) heterocyclyl or heteroaryl, each of which is optionally further substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are as defined above, and wherein any alkyl moiety present in a substituent group is optionally further substituted by halo, hydroxy and/or (1-2C)alkoxy;
      and an available ring nitrogen atom (where valency permits) is optionally further substituted by (1-4C)alkyl optionally substituted by halo, hydroxy, cyano, amino and/or (1-2C)alkoxy;
   (c) C(O)Rₐ₂, C(O)ORₐ₂, C(O)N(R_{b2})Rₐ₂ or C(O)N(R_{b2})ORₐ₂; wherein Rₐ₂ and R_{b2} are each independently selected from hydrogen or (1-4C)alkyl, and wherein any alkyl moiety present in a substituent group is optionally further substituted by halo, hydroxy and/or (1-2C)alkoxy; or
(ii) a group:

-L-R₂;
wherein:
L is a linking group selected from: —O—; —CO—; —COO—; —OCO—; —NR₁ₐ—; —CONR₁ₐ—; —CONR₁ₐ—O—; —NR₁ₐCO—; or NR₁ₐCONR_{1b}—;
R₁ₐ and R_{1b} are independently selected from hydrogen or (1-2C)alkyl; and
R₂ is selected from:
(a) hydrogen;
(b) (1-4C)alkyl which is optionally substituted by halo, hydroxy, cyano, amino, (1-2C)alkylamino, di-(1-2C)alkylamino and/or (1-2C)alkoxy (which is optionally further substituted by halo and/or (1-2C)alkoxy); or
(c) a cycloalkyl, aryl, heteroaryl, heterocyclyl, or heterocyclyl(1-2C)alkyl ring,
   wherein each ring system is optionally substituted on an available carbon atom by one or more R_{101C} substituents;
   wherein each R_{101C} substituent is independently selected from halo, hydroxy, cyano, R_{c}, NR_{d}R_{c}, OR_{c}, C(O)R_{c}, C(O)OR_{c}, OC(O)R_{c}, C(O)N(R_{d})R_{c}, C(O)N(R_{d})OR_{c}, N(R_{d})C(O)R_{c}, S(O)_{y}R_{c} (wherein y is 0, 1 or 2), S(O)₂N(R_{d})R_{c}, N(R_{d})S(O)₂R_{c}, (CH₂)_{z}R_{c} or (CH₂)_{z}NR_{c}R_{d} (where z is 1, 2 or 3);
   and wherein R_{c} is selected from:
   (a) hydrogen;
   (b) (1-4C)alkyl which is optionally substituted by one or more substituents independently selected from halo, hydroxy, cyano, amino, (3-6C)cycloalkyl, (1-2C)alkylamino, di-(1-2C)alkylamino or (1-2C)alkoxy and wherein any alkyl or cycloalkyl moiety present in such substituent groups is optionally further substituted by halo, hydroxy and/or (1-2C)alkoxy; or
   (c) aryl, heteroaryl, heterocyclyl or heterocyclyl(1-2C)alkyl ring,
      wherein each ring system is optionally substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)R_{c1}, C(O)OR_{c1}, OC(O)R_{c1}, C(O)N(R_{d1})R_{c1}, C(O)N(R_{d1})OR_{c1} N(R_{d1})C(O)R_{c1}, S(O)_{y}R_{c1} (wherein y is 0, 1 or 2), S(O)₂N(R_{b1})R_{c1} or N(R_{d1})S(O)₂R_{c1}, wherein R_{c1} and R_{d1} are each independently selected from hydrogen or (1-2C)alkyl, and wherein any alkyl moiety present in a substituent group is optionally further substituted by halo, hydroxy and/or (1-2C)alkoxy;
   and R_{d} is selected from hydrogen or (1-2C)alkyl;
   and, when R₂ or R_{c} is a heteroaryl or heterocyclyl ring, an available nitrogen atom (where valency permits) is optionally further substituted by one or more R_{101N} substituents, wherein R_{101N} is selected from:
   a) (1-4C)alkyl which is optionally substituted by one or more substituents independently selected from halo, oxo, hydroxy, cyano, amino or (1-2C)alkoxy; or
   b) C(O)R_{c2}, C(O)OR_{c2}, C(O)N(R_{d2})R_{c2} or C(O)N(R_{d2})OR_{c2}; wherein R_{c2} and R_{d2} are each independently selected from hydrogen or (1-4C)alkyl, and wherein any alkyl moiety present in a substituent group is optionally further substituted by halo, hydroxy and/or (1-2C)alkoxy;
or, when L is —NR₁ₐCONR_{1b}—, R_{1b} and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N}.
(4) R₁ is selected from:
(i) an aryl, heteroaryl or heterocyclyl ring,
   each of which is optionally substituted on an available carbon atom by one or more R_{100C} substituents independently selected from hydroxy and Rₐ; wherein Rₐ is heterocyclyl;
   and, when R₁ or Rₐ is heteroaryl or heterocyclyl, an available nitrogen atom (where valency permits) is optionally substituted by one or more R_{100N}, wherein R_{100N} is selected from:
   (a) (1-4C)alkyl which is optionally substituted by hydroxy; or
   (b) heterocyclyl or heteroaryl, each of which is optionally substituted on an available ring nitrogen atom (where valency permits) by (1-4C)alkyl;
   (c) C(O)ORₐ₂; wherein Rₐ₂ is (1-4C)alkyl, which is optionally further substituted by halo, hydroxy and/or (1-2C)alkoxy; or
(ii) a group -L-R₂ wherein:
   L is a linking group selected from: —O—; —CO—; —COO—; —NR₁ₐ—; —CONR₁ₐ—; —CONR₁ₐ—O—; —NR₁ₐCO—; or NR₁ₐCONR_{1b}—;
   wherein R₁ₐ and R_{1b} are hydrogen; and
   R₂ is selected from:
   (a) hydrogen;
   (b) (1-4C)alkyl which is optionally substituted by hydroxy or di-(1-4C)alkylamino; or
   (c) a cycloalkyl, heteroaryl or heterocyclyl ring,
      wherein each ring system is optionally substituted on an available carbon atom by one or more R_{101C} substituents independently selected from hydroxy, R_{c}, NR_{d}R_{c}, OR_{c} and C(O)N(R_{d})OR_{c} wherein R_{c} is selected from:
      (a) (1-4C)alkyl which is optionally substituted by one or more substituents independently selected from halo, hydroxy, (3-6C)cycloalkyl or (1-4C)alkoxy; or
      (b) an aryl or heterocyclyl(1-2C)alkyl ring, wherein each ring system is optionally substituted on an available carbon atom by cyano;
      and R_{d} is selected from hydrogen or (1-2C)alkyl;
      and, when R₂ or R_{c} is a heterocyclyl ring, an available nitrogen atom (where valency permits) is optionally further substituted by one or more R_{101N} substituents, wherein R_{101N} is (1-4C)alkyl or C(O)OR₂; wherein R_{c2} is (1-4C)alkyl;
   or, when L is —NR₁ₐCONR_{1b}—, R_{1b} and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N}.
(5) R₁ is selected from:
(i) a heteroaryl or heterocyclyl ring,
   each of which is optionally substituted on an available carbon atom by one or more R_{100C}, wherein R_{100C} is hydroxy; and an available nitrogen atom (where valency permits) is optionally substituted by one or more R_{100N}, wherein R_{100N} is
      (1-4C)alkyl which is optionally substituted by hydroxy; or
      heterocyclyl which is optionally substituted on an available ring nitrogen atom (where valency permits) by (1-4C)alkyl;
      C(O)ORₐ₂; wherein Rₐ₂ is (1-4C)alkyl; or
(ii) a group -L-R₂ wherein:
   L is a linking group selected from: —CONR₁ₐ— or —CONR₁ₐ—O— or NR₁ₐCONR_{1b}—; wherein R₁ₐ and R_{1b} are hydrogen; and
   R₂ is selected from: hydrogen, (1-4C)alkyl (which is optionally substituted by hydroxy), a cycloalkyl, heteroaryl or heterocyclyl ring;
   wherein each ring system is optionally substituted on an available carbon atom by one or more R_{101C} substituents independently selected from hydroxy, R_{c}, OR_{c} and C(O)N(R_{d})OR_{c}, wherein R_{c} is selected from:
   (a) (1-4C)alkyl which is optionally substituted by one or more substituents independently selected from halo, hydroxy, (3-6C)cycloalkyl or (1-4C)alkoxy; or
   (b) an aryl or heterocyclyl(1-2C)alkyl ring, wherein each ring system is optionally substituted on an available carbon atom by cyano;
   and R_{d} is selected from hydrogen or (1-2C)alkyl;
   and, when R₂ is a heterocyclyl ring, an available nitrogen atom (where valency permits) is optionally further substituted by one or more R_{101N}, wherein R_{101N} is (1-4C)alkyl or C(O)OR₁₂; wherein R₁₂ is (1-4C)alkyl;
   or, when L is —NR₁ₐCONR_{1b}—, R_{1b} and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N}.
(6) R₁ is selected from: heteroaryl or heterocyclyl ring,
   each of which is optionally substituted on an available carbon atom by hydroxy; and
   an available nitrogen atom (where valency permits) is optionally substituted by:
      (1-4C)alkyl which is optionally substituted by hydroxy; or
      heterocyclyl which is optionally substituted on an available ring nitrogen atom (where valency permits) by (1-4C)alkyl.
(7) R₁ is a group -L-R₂ wherein:
   L is a linking group selected from: —CONR₁ₐ— or —CONR₁ₐ—O— or NR₁ₐCONR_{1b}—; wherein R₁ₐ and R_{1b} are hydrogen; and
   R₂ is selected from: hydrogen, (1-4C)alkyl (which is optionally substituted by hydroxy), a cycloalkyl, heteroaryl or heterocyclyl ring;
   wherein each ring system is optionally substituted on an available carbon atom by one or more substituents independently selected from hydroxy, R_{c}, OR_{c} and C(O)N(R_{d})OR_{c}, wherein R_{c} is selected from:
   (a) (1-4C)alkyl which is optionally substituted by one or more substituents independently selected from halo, hydroxy, (3-6C)cycloalkyl or (1-4C)alkoxy; or
   (b) an aryl or heterocyclyl(1-2C)alkyl ring, wherein each ring system is optionally substituted on an available carbon atom by cyano;
   and R_{d} is selected from hydrogen or (1-2C)alkyl;
   and, when R₂ is a heterocyclyl ring, an available nitrogen atom (where valency permits) is optionally further substituted by (1-4C)alkyl.
(7a) R₁ is a group -L-R₂ wherein:
   L is a linking group selected from: —CONR₁ₐ— or —CONR₁ₐ—O— or NR₁ₐCONR_{1b}—; wherein R₁ₐ and R_{1b} are hydrogen; and
   R₂ is selected from heteroaryl or heterocyclyl ring;
   wherein each ring system is optionally substituted on an available carbon atom by one or more substituents independently selected from hydroxy, R_{c}, OR_{c} and C(O)N(R_{d})OR_{c}, wherein R_{c} is selected from:
   (a) (1-4C)alkyl which is optionally substituted by one or more substituents independently selected from halo, hydroxy, (3-6C)cycloalkyl or (1-4C)alkoxy; or
   (b) an aryl or heterocyclyl(1-2C)alkyl ring, wherein each ring system is optionally substituted on an available carbon atom by cyano;
   and R_{d} is selected from hydrogen or (1-2C)alkyl;
   and, when R₂ is a heterocyclyl ring, an available nitrogen atom (where valency permits) is optionally further substituted by (1-4C)alkyl.
(7b) R₁ is selected from:
   i) a heterocyclyl ring, which may be optionally substituted on an available carbon atom by hydroxy;
   and may be optionally substituted on an available nitrogen atom (where valency permits) by (1-4C)alkyl which is optionally substituted by hydroxy.
   ii) a group -L-R₂
      wherein:
         L is a linking group selected from: —CONH— or —CONH—O— or NHCONH—; and
         R₂ is a heterocyclyl ring; which is optionally substituted on an available carbon atom by one or more substituents independently selected from hydroxy, R_{c}, OR_{c} and C(O)N(R_{d})OR_{c}; and may be optionally substituted on an available nitrogen atom (where valency permits) by (1-4C)alkyl which is optionally substituted by hydroxy;
         wherein R_{c} is selected from:
      a) (1-4C)alkyl which is optionally substituted by one or more substituents independently selected from halo, hydroxy, (3-6C)cycloalkyl or (1-4C)alkoxy; or
      b) an aryl or heterocyclyl(1-2C)alkyl ring, wherein each ring system is optionally substituted on an available carbon atom by cyano;
         and R_{d} is selected from hydrogen or (1-2C)alkyl;
         and, when R₂ is a heterocyclyl ring, an available nitrogen atom (where valency permits) is optionally further substituted by (1-4C)alkyl.
(8) X₁ is —NH—.
(9) X₁ is —O—.
(10) Ring A is selected from Ring A-1, i.e. phenyl, Ring A-2, Ring A-3, Ring A-4, Ring A-5 or Ring A-7, each optionally substituted by one or more substituents independently selected from halo, (1-4C)alkyl and (1-4C)alkoxy.
(11) Ring A is selected from Ring A-1, i.e. phenyl, Ring A-2, Ring A-3 or Ring A-7, each optionally substituted by one or more substituents independently selected from halo, (1-4C)alkyl and (1-4C)alkoxy.
(12) Ring A is Ring A-1, i.e. phenyl, optionally substituted by one or more substituents independently selected from halo, (1-4C)alkyl and (1-4C)alkoxy.
(13) Ring B is a 5- or 6-membered heteroaryl or a 5- or 6-membered heterocyclic ring, each of which comprises one to three N atoms and is linked via a carbon atom to the amide bond in the compound of Formula (I) and wherein:
   (a) a heteroaryl ring is optionally substituted on an available carbon atom by one or more substituents independently selected from hydroxy, halo, cyano, (1-4C)alkyl or (1-4C)alkoxy; or on an available nitrogen atom by (1-4C)alkyl; and wherein any alkyl moiety present in a substituent group is optionally further substituted by halo, hydroxy and/or (1-2C)alkoxy;
   and
   (b) a heterocyclyl ring is optionally substituted on an available carbon atom by one or more substituents independently selected from oxo, hydroxy, halo, cyano, (1-6C)alkyl, (1-6C)alkoxy; or on an available nitrogen atom by (1-6C)alkyl; and wherein any alkyl moiety present in a substituent group is optionally further substituted by halo, hydroxy and/or (1-2C)alkoxy;
(14) Ring B is a 5- or 6-membered heteroaryl or a 6-membered heterocyclic ring, each of which comprises one to three N atoms and is linked via a carbon atom to the amide bond in the compound of Formula (I) and wherein:
   (a) a heteroaryl ring is optionally substituted on an available carbon atom by one or more substituents independently selected from hydroxy, halo, cyano, (1-4C)alkyl or (1-4C)alkoxy; or on an available nitrogen atom by (1-4C)alkyl; and wherein any alkyl moiety present in a substituent group is optionally further substituted by halo, hydroxy and/or (1-2C)alkoxy;
   and
   (b) a heterocyclyl ring is optionally substituted on an available carbon atom by one or more substituents independently selected from oxo, hydroxy, halo, cyano, (1-6C)alkyl, (1-6C)alkoxy; or on an available nitrogen atom by (1-6C)alkyl; and wherein any alkyl moiety present in a substituent group is optionally further substituted by halo, hydroxy and/or (1-2C)alkoxy;
(15) Ring B is a 5- or 6-membered heteroaryl having the formula B-1 or B-2 shown below:

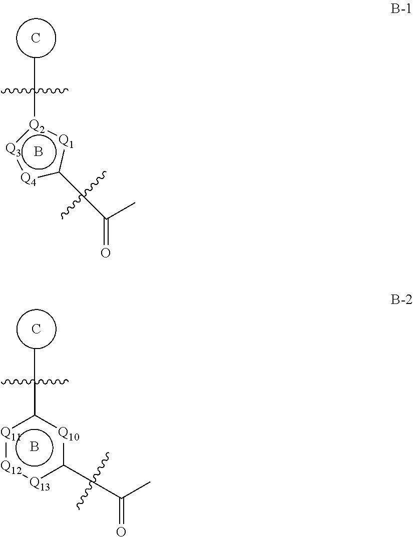

wherein
Q₁, Q₃ and Q₄ are CH, CR_{q1}, N or NRₙ₁ (where the valency permits);
Q₂ is C or N;
wherein up to three of Q₁, Q₂, Q₃ and Q₄ can be N;
R_{q1} is hydroxy, halo, cyano, (1-4C)alkyl or (1-4C)alkoxy;
Rₙ₁ is hydrogen or (1-4C)alkyl (where the valency permits);
and wherein any alkyl moiety present in a R_{q} or Rₙ₁ substituent group is optionally further substituted by halo, hydroxy and/or (1-2C)alkoxy;
Q₁₀, Q₁₁, Q₁₂ and Q₁₃ are CR_{q2} or N;
wherein up to three of Q₁₀, Q₁₁, Q₁₂ and Q₁₃ can be N and R_{q2} is hydrogen or a substituent selected from hydroxy, halo, cyano, (1-4C)alkyl or (1-4C)alkoxy; and wherein any alkyl moiety present in a R_{q2} substituent group is optionally further substituted by halo, hydroxy and/or (1-2C)alkoxy;
or Ring B is a 6-membered heterocyclic ring of the formula B-3 or B-4 shown below:

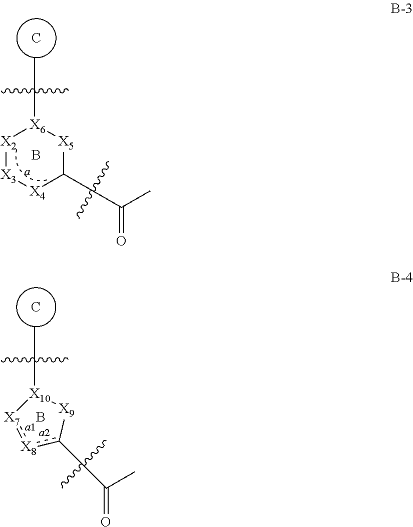

wherein
X₂, X₃ and X₄ are selected from C═O, CRₓ, CHRₓ or NR_{y};
X₅ is selected from C═O, CHRₓ or NR_{y};
X₆ is selected from CH or N;
wherein no more than three of X₂, X₃, X₄, X₅ and X₆ can be N and no more than three of X₂, X₃, X₄ and X₅ can be C═O;
Rₓ is hydrogen or a substituent selected from hydroxy, halo, cyano, (1-6C)alkyl, (1-6C)alkoxy;
R_{y} is hydrogen or (1-6C)alkyl;
and wherein any alkyl moiety present in a Rₓ or R_{y} substituent group is optionally further substituted by halo, hydroxy and/or (1-2C)alkoxy; and
[symbol] represents an optional double bond between X₂ and X₃ and/or between X₄ and the adjacent ring carbon atom linked to the amide bond;
X₇ and X₈ are selected from C═O, CRₓ CHRₓ or NR_{y};
X₉ is selected from C═O, CHRₓ or NR_{y};
X₁₀ is selected from CH or N;
wherein no more than two of X₇, X₈, X₉ and X₁₀ can be N and no more than two of X₇,
X₈ and X₉ can be C═O;
Rₓ and R_{y} are as defined above; and
both a1 and a2 are single bonds or one of a1 or a2 is a double bond;
or Ring B is a 6-membered heterocyclyl ring having the formula B-10 shown below:

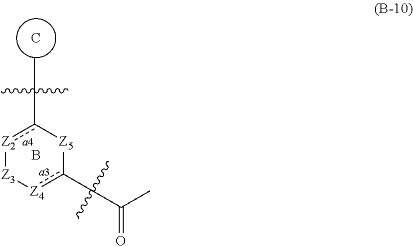

wherein
Z₂, Z₄ and Z₅ are independently selected from C═O, CRᵥ, CHRᵥ;
Z₃ is selected from C═O, CHRᵥ or NR_{w};
wherein no more than three of Z₂, Z₃, Z₄ and Z₅ can be C═O;
Rᵥ is hydrogen or a substituent selected from hydroxy, halo, cyano, (1-6C)alkyl, (1-6C)alkoxy;
R_{w} is hydrogen or (1-6C)alkyl;
and wherein any alkyl moiety present in a Rᵥ or R_{w} substituent group is optionally further substituted by halo, hydroxy and/or (1-2C)alkoxy; and
both a3 and a4 are single bonds, one of a3 and a4 is a double bond, or both of a3 and a4 are double bonds;
(16) Ring B is a 5- or 6-membered heteroaryl having the formula B-1 or B-2 shown below:

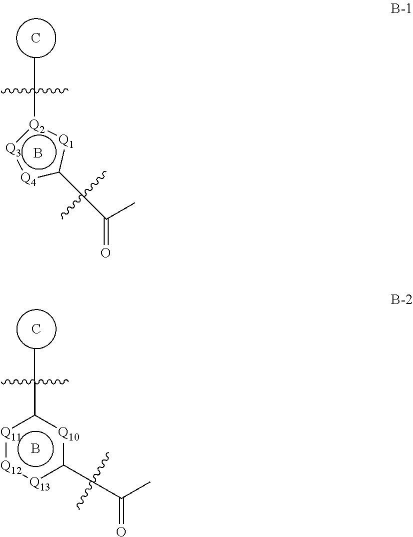

wherein
Q₁, Q₃ and Q₄ are CH, CR_{q1}, N or NRₙ₁ (where the valency permits);
Q₂ is C or N;
wherein up to three of Q₁, Q₂, Q₃ and Q₄ can be N;
R_{q1} is hydroxy, halo, cyano, (1-2C)alkyl or (1-2C)alkoxy;
Rₙ₁ is hydrogen or (1-2C)alkyl (where the valency permits);
Q₁₀, Q₁₁, Q₁₂ and Q₁₃ are CR_{q2} or N;
wherein up to two of Q₁₀, Q₁₁, Q₁₂ and Q₁₃ can be N and R_{q2} is hydrogen or a substituent selected from hydroxy, halo, cyano, (1-2C)alkyl or (1-2C)alkoxy;
   or Ring B is a 6-membered heterocyclic ring of the formula B-3 or B-4 shown below:

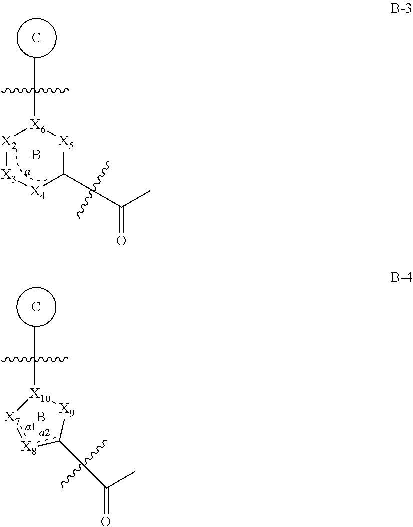

wherein
X₂, X₃ and X₄ are selected from C═O, CRₓ, CHRₓ or NR_{y};
X₅ is selected from C═O, CHRₓ or NR_{y};
X₆ is selected from CH or N;
wherein no more than three of X₂, X₃, X₄, X₅ and X₆ can be N and no more than two of X₂, X₃, X₄ and X₅ can be C═O;
Rₓ is hydrogen or a substituent selected from hydroxy, halo, cyano, (1-4C)alkyl, (1-4C)alkoxy;
R_{y} is hydrogen or (1-4C)alkyl;
and wherein any alkyl moiety present in a Rₓ or R_{y} substituent group is optionally further substituted by halo, hydroxy and/or (1-2C)alkoxy; and
[symbol] represents an optional double bond between X₂ and X₃ and/or between X₄ and the adjacent ring carbon atom linked to the amide bond;
X₇ and X₈ are selected from CRₓ CHRₓ or NR_{y};
X₉ is selected from C═O, CHRₓ or NR_{y};
X₁₀ is selected from CH or N;
wherein no more than two of X₇, X₈, X₉ and X₁₀ can be N;
Rₓ and R_{y} are as defined above; and
both a1 and a2 are single bonds or one of a1 or a2 is a double bond;
or Ring B is a 6-membered heterocyclyl ring having the formula B-10 shown below:

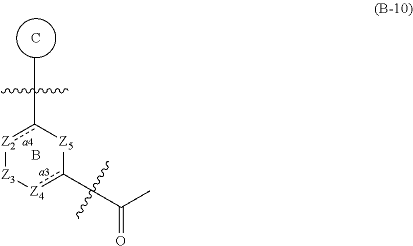

wherein
Z₂ and Z₄ are selected from C═O, CRᵥ, CHRᵥ;
Z₃ is selected from C═O, CHRᵥ or NR_{w};
Z₅ is C═O;
wherein no more than three of Z₂, Z₃, Z₄ and Z₅ can be C═O;
Rᵥ is hydrogen or a substituent selected from hydroxy, halo, cyano, (1-6C)alkyl, (1-6C)alkoxy;
R_{w} is hydrogen or (1-6C)alkyl;
and wherein any alkyl moiety present in a Rᵥ or R_{w} substituent group is optionally further substituted by halo, hydroxy and/or (1-2C)alkoxy; and
both a3 and a4 are single bonds, one of a3 and a4 is a double bond, or both of a3 and a4 are double bonds;
(17) Ring B is a 5- or 6-membered heteroaryl having the formula B-1 or B-2 shown below:

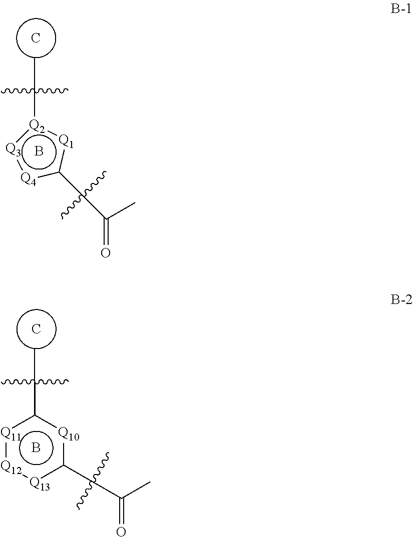

wherein
Q₁, Q₃ and Q₄ are CH or N;
Q₂ is C or N;
wherein up to two of Q₁, Q₂, Q₃ and Q₄ can be N;
Q₁₀, Q₁₁, Q₁₂ and Q₁₃ are CR_{q2} or N;
wherein up to two of Q₁₀, Q₁₁, Q₁₂ and Q₁₃ can be N and R_{q2} is hydrogen or a substituent selected from hydroxy, halo, cyano, (1-2C)alkyl or (1-2C)alkoxy;
or Ring B is a 6-membered heterocyclic ring of the formula B-3 or B-4 shown below:

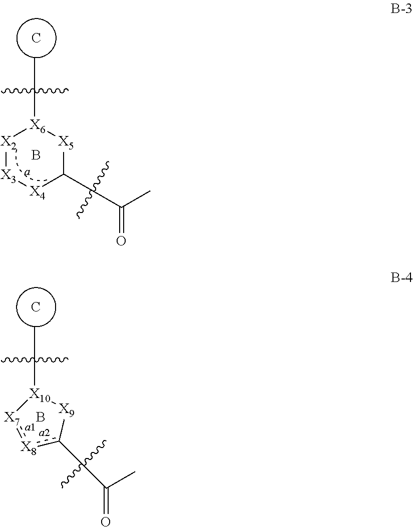

wherein
X₂, X₃ and X₄ are selected from C═O, CRₓ, CHRₓ or NR_{y};
X₅ is selected from C═O, CHRₓ or NR_{y};
X₆ is selected from CH or N;
wherein no more than three of X₂, X₃, X₄, X₅ and X₆ can be N and no more than two of X₂, X₃, X₄ and X₅ can be C═O;
Rₓ is hydrogen or a substituent selected from hydroxy, halo, cyano, (1-4C)alkyl, (1-4C)alkoxy;
R_{y} is hydrogen or (1-4C)alkyl;
and wherein any alkyl moiety present in a Rₓ or R_{y} substituent group is optionally further substituted by halo, hydroxy and/or (1-2C)alkoxy; and
[symbol] represents an optional double bond between X₂ and X₃ and/or between X₄ and the adjacent ring carbon atom linked to the amide bond;
X₇ and X₈ are selected from CRₓ CHRₓ or NR_{y};
X₉ is selected from C═O, CHRₓ or NR_{y};
X₁₀ is selected from CH or N;
wherein no more than two of X₇, X₈, X₉ and X₁₀ can be N;
Rₓ and R_{y} are as defined above; and
both a1 and a2 are single bonds or one of a1 or a2 is a double bond;
   or Ring B is a 6-membered heterocyclyl ring having the formula B-10 shown below:

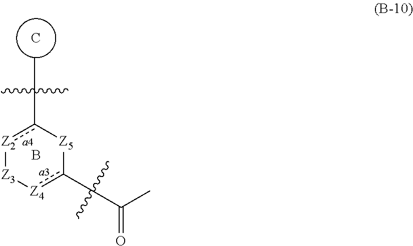

wherein
Z₂ and Z₄ are selected from C═O, CRᵥ, CHRᵥ;
Z₃ is selected from CHRᵥ or NR_{w};
Z₅ is C═O;
Rᵥ is hydrogen or a substituent selected from hydroxy, halo, cyano, (1-4C)alkyl, (1-4C)alkoxy;
R_{w} is hydrogen or (1-4C)alkyl;
and wherein any alkyl moiety present in a Rᵥ or R_{w} substituent group is optionally further substituted by halo, hydroxy and/or (1-2C)alkoxy; and
both a3 and a4 are single bonds, one of a3 and a4 is a double bond, or both of a3 and a4 are double bonds;
(18) Ring B is a 5- or 6-membered heteroaryl having the formula B-1 or B-2 shown below:

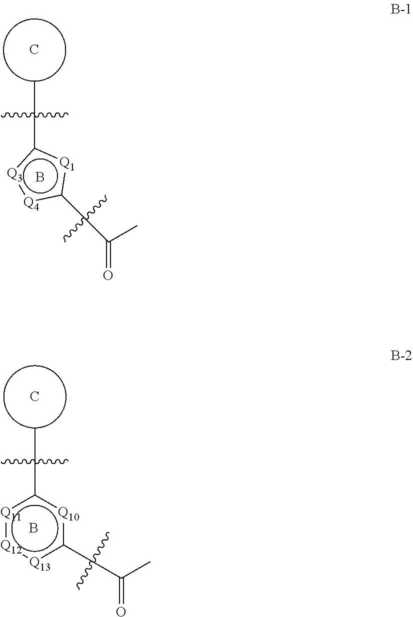

wherein
Q₁, Q₃ and Q₄ are CH or N;
wherein one of Q₁, Q₃ and Q₄ is N;
Q₁₀, Q₁₁, Q₁₂ and Q₁₃ are CR_{q2} or N;
wherein up to two of Q₁₀, Q₁₁, Q₁₂ and Q₁₃ can be N and R_{q2} is hydrogen or a substituent selected from hydroxy, halo, cyano, (1-2C)alkyl or (1-2C)alkoxy;
or Ring B is a 6-membered heterocyclic ring of the formula B-3 or B-4 shown below:

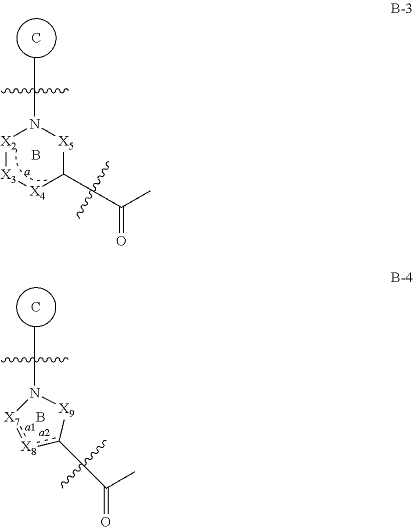

wherein
X₂, X₃ and X₄ are selected from C═O, CRₓ, CHRₓ or NR_{y};
X₅ is selected from C═O, CHRₓ or NR_{y};
wherein no more than two of X₂, X₃, X₄ and X₅ can be N and no more than two of X₂, X₃, X₄ and X₅ can be C═O;
Rₓ is hydrogen or a substituent selected from hydroxy, halo, cyano, (1-4C)alkyl, (1-4C)alkoxy;
R_{y} is hydrogen or (1-4C)alkyl;
and wherein any alkyl moiety present in a Rₓ or R_{y} substituent group is optionally further substituted by halo, hydroxy and/or (1-2C)alkoxy; and
[symbol] represents an optional double bond between X₂ and X₃ and/or between X₄ and the adjacent ring carbon atom linked to the amide bond;
X₇ and X₈ are selected from CRₓ CHRₓ or NR_{y};
X₉ is selected from C═O, CHRₓ or NR_{y};
wherein one of X₇, X₈ and X₉ can be N;
Rₓ and R_{y} are as defined above; and
both a1 and a2 are single bonds or one of a1 or a2 is a double bond;
or Ring B is a 6-membered heterocyclyl ring having the formula B-10 shown below:

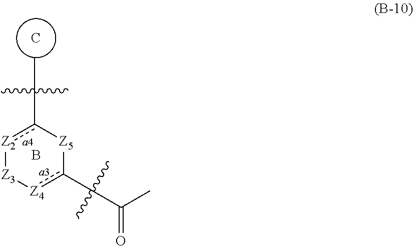

wherein
Z₂ and Z₄ are selected from C═O, CRᵥ, CHRᵥ;
Z₃ is NR_{w};
Z₅ is C═O;
R_{y} is hydrogen or a substituent selected from hydroxy, halo, cyano, (1-4C)alkyl, (1-4C)alkoxy;
R_{w} is hydrogen or (1-4C)alkyl;
and wherein any alkyl moiety present in a Rᵥ or R_{w} substituent group is optionally further substituted by halo, hydroxy and/or (1-2C)alkoxy; and
both a3 and a4 are single bonds, one of a3 and a4 is a double bond, or both of a3 and a4 are double bonds;
(19) Ring B is a 5- or 6-membered heteroaryl having the formula B-1 or B-2 shown below:

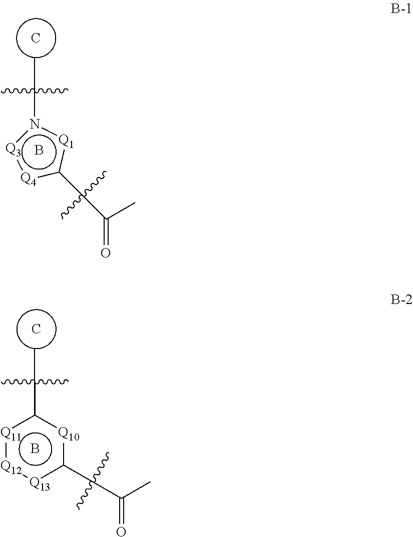

wherein
Q₁, Q₃ and Q₄ are CH or N;
wherein one of Q₁, Q₃ and Q₄ is N;
Q₁₀, Q₁₁, Q₁₂ and Q₁₃ are CR_{q2} or N;
wherein up to two of Q₁₀, Q₁₁, Q₁₂ and Q₁₃ can be N and R_{q2} is hydrogen or a substituent selected from hydroxy, halo, cyano, (1-2C)alkyl or (1-2C)alkoxy;
or Ring B is a 6-membered heterocyclic ring of the formula B-3 or B-4 shown below:

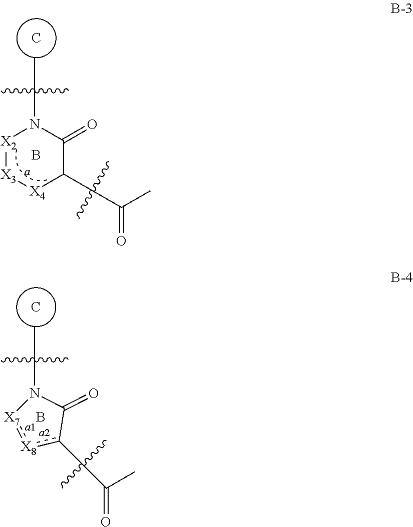

wherein
X₂, X₃ and X₄ are selected from C═O, CHRₓ or NR_{y};
wherein only one of X₂, X₃ and X₄ can be N and only one of X₂, X₃ and X₄ can be C═O;
Rₓ is hydrogen or a substituent selected from hydroxy, halo, cyano, (1-4C)alkyl, (1-4C)alkoxy;
R_{y} is hydrogen or (1-4C)alkyl;
and wherein any alkyl moiety present in a Rₓ or R_{y} substituent group is optionally further substituted by halo, hydroxy and/or (1-2C)alkoxy; and
[symbol] represents an optional double bond between X₂ and X₃ and/or between X₄ and the adjacent ring carbon atom linked to the amide bond;
X₇ is CHRₓ or NR_{y};
X₈ is CRₓ;
Rₓ and R_{y} are as defined above; and
both a1 and a2 are single bonds or a1 is a single bond and a2 is a double bond;
or Ring B is a 6-membered heterocyclyl ring having the formula B-10 shown below:

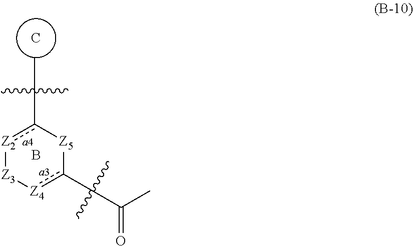

wherein
Z₂ and Z₄ are selected from C═O, CRᵥ, CHRᵥ;
Z₃ is NR_{w};
Z₅ is C═O;
Rᵥ is hydrogen or a substituent selected from hydroxy, halo, cyano, (1-4C)alkyl, (1-4C)alkoxy;
R_{w} is hydrogen or (1-4C)alkyl;
and wherein any alkyl moiety present in a Rᵥ or R_{w} substituent group is optionally further substituted by halo, hydroxy and/or (1-2C)alkoxy; and
one of a3 and a4 is a double bond or both of a3 and a4 are double bonds;
(20) Ring B is a group as depicted in (B-5), (B-6) or (B-7) below:

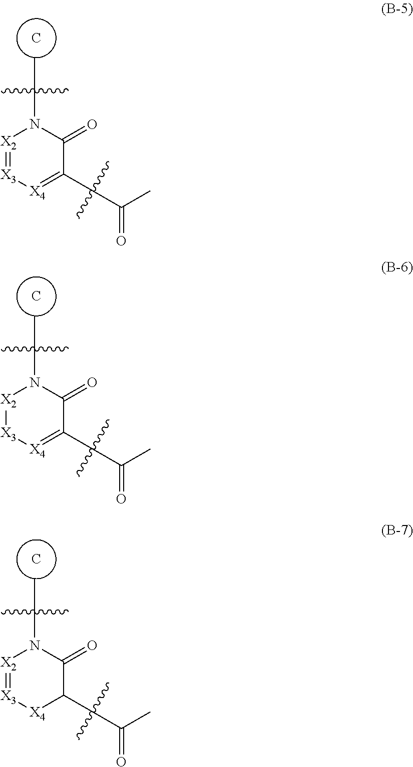

wherein
X₂ is N or CR₂ₓ wherein R₂ₓ is hydrogen, (1-4C)alkyl or (1-4C)alkoxy when there is a double bond between X₂ and X₃ or
X₂ is CO, CH₂, CHR₂ₓ or NR_{2y} wherein R_{2y} is hydrogen or (1-4C)alkyl when there is a single bond between X₂ and X₃;
X₃ is N or CR₃ₓ wherein R₃ₓ is hydrogen, (1-4C)alkyl or (1-4C)alkoxy when there is a double bond between X₂ and X₃ or
X₃ is CO, CH₂, CHR₃ₓ or NR_{3y} wherein R_{3y} is hydrogen or (1-4C)alkyl when there is a single bond between X₂ and X₃;
X₄ is N or CR₄ₓ wherein R₄ₓ is hydrogen, (1-4C)alkyl or (1-4C)alkoxy when there is a double bond between X₄ and the adjacent ring carbon atom linked to the amide bond or
X₄ is CO, CH₂, CHR₄ₓ or NR_{4y} wherein R_{4y} is hydrogen or (1-4C)alkyl when there is a single bond between X₄ and the adjacent ring carbon atom linked to the amide bond;
wherein any alkyl moiety present in a ring substituent group is optionally further substituted by halo, hydroxy or (1-2C)alkoxy;
and provided that:
   a. a maximum of one of X₂, X₃ and X₄ is N or NR_{2y}, NR_{3y} or R_{4y} respectively;
   b. a maximum of one of X₂, X₃ and X₄ is CO;
   c. when X₂ is N then X₃ is CO;
or Ring B is a 6-membered heterocyclyl ring having the formula B-11 shown below:

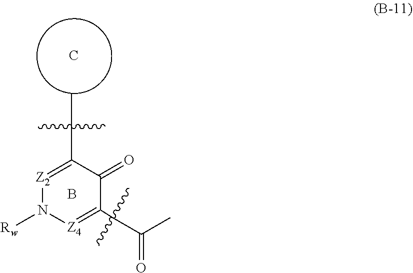

wherein
Z₂ and Z₄ are CRᵥ;
each R_{y} is independently selected from hydrogen or a substituent selected from hydroxy, halo, cyano, (1-4C)alkyl, (1-4C)alkoxy;
R_{w} is hydrogen or (1-4C)alkyl;
and wherein any alkyl moiety present in a Rᵥ or R_{w} substituent group is optionally further substituted by halo, hydroxy and/or (1-2C)alkoxy.
(21) Ring B is a group as depicted in (B-5) or (B-6) below:

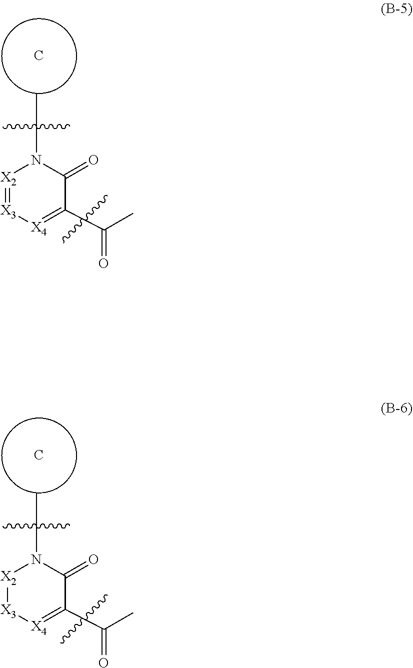

wherein
X₂ is N or CR₂ₓ wherein R₂ₓ is hydrogen, (1-4C)alkyl or (1-4C)alkoxy when there is a double bond between X₂ and X₃ or
X₂ is CO, CH₂, CHR₂ₓ or NR_{2y} wherein R_{2y} is hydrogen or (1-4C)alkyl when there is a single bond between X₂ and X₃;
X₃ is N or CR₃ wherein R₃ₓ is hydrogen, (1-4C)alkyl or (1-4C)alkoxy when there is a double bond between X₂ and X₃ or
X₃ is CO, CH₂, CHR₃ₓ or NR_{3y} wherein R_{3y} is hydrogen or (1-4C)alkyl when there is a single bond between X₂ and X₃;
X₄ is N or CR₄ₓ wherein R₄ₓ is hydrogen, (1-4C)alkyl or (1-4C)alkoxy when there is a double bond between X₄ and the adjacent ring carbon atom linked to the amide bond or
X₄ is CO, CH₂, CHR₄ₓ or NR_{4y} wherein R_{4y} is hydrogen or (1-4C)alkyl when there is a single bond between X₄ and the adjacent ring carbon atom linked to the amide bond;
wherein any alkyl moiety present in a ring substituent group is optionally further substituted by halo, hydroxy or (1-2C)alkoxy;
and provided that:
   a. a maximum of one of X₂, X₃ and X₄ is N or NR_{2y}, NR_{3y} or R_{4y} respectively;
   b. a maximum of one of X₂, X₃ and X₄ is CO;
   c. when X₂ is N then X₃ is CO;
   or Ring B is a 6-membered heterocyclyl ring having the formula B-11 shown below:

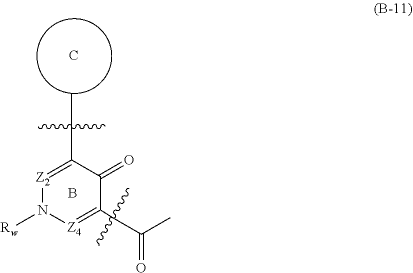

wherein
Z₂ and Z₄ are CRᵥ;
each Rᵥ is independently selected from hydrogen, (1-2C)alkyl or (1-2C)alkoxy;
R_{w} is hydrogen or (1-4C)alkyl; and
and wherein any alkyl moiety present in a Rᵥ or R_{w} substituent group is optionally further substituted by halo, hydroxy and/or (1-2C)alkoxy.
(22) Ring B is a group as depicted in (B-8), (B-9) or (B-12) below:

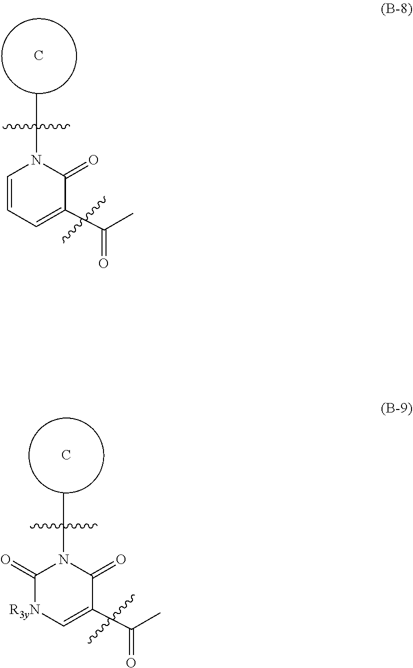

wherein R_{3y} is hydrogen or (1-4C)alkyl, in particular prop-2-yl (iso-propyl);
or Ring B is as depicted in (B-12) below:

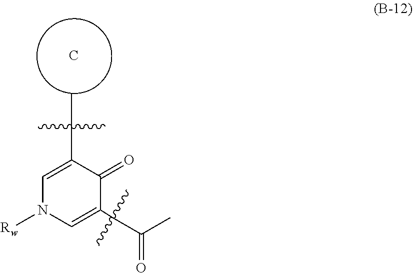

wherein R_{w} is hydrogen or (1-4C)alkyl (in particular prop-2-yl (iso-propyl)); wherein the (1-4C)alkyl is optionally further substituted by halo, hydroxy and/or (1-2C)alkoxy.
(23) Ring C is Ring C-1, i.e. phenyl, or Ring C-2, each optionally substituted by one or more halo.
(24) Ring C is Ring C-1, i.e. phenyl, optionally substituted by one or more halo.

Suitably, R₁ is as defined in any one of paragraphs (1) to (7), (7a) or (7b) above. More suitably, R₁ is as defined in paragraph (4) or (5), or (6) to (7b) above. Most suitably, R₁ is as defined in paragraph (6), (7), (7a) or (7b) above.

Suitably, X₁ is as defined in any one of paragraphs (8) or (9) above. Most suitably, X₁ is as defined in paragraph (8) above.

Suitably, Ring A is as defined in any one of paragraphs (10) to (12) above. Most suitably, Ring A is as defined in paragraph (12) above.

Suitably, Ring B is as defined in any one of paragraphs (13) to (22) above. More suitably, Ring B is as defined in any one of paragraphs (16) to (19) above. Most suitably, Ring B is as defined in paragraph (19), (20), (21) or (22) above.

Suitably, Ring C is as defined in any one of paragraphs (23) to (24) above. Most suitably, Ring C is as defined in paragraph (24) above.

In a particular group of compounds of the invention, X₁ is NH and R₁, Ring A, Ring B and Ring C are each as defined herein.

In a particular group of compounds of the invention, X₁ is O and R₁, Ring A, Ring B and Ring C are each as defined herein.

In a particular group of compounds of the invention, X₁ is NH and:
R₁ is as defined in any one of paragraphs (1) to (7), (7a) or (7b) above;
Ring A is as defined in any one of paragraphs (10) to (12) above;
Ring B is as defined in any one of paragraphs (13) to (22) above; and
Ring C is as defined in any one of paragraphs (23) to (24) above.

In a particular group of compounds of the invention, X₁ is NH and:
R₁ is as defined in paragraph (4) above;
Ring A is as defined in paragraph (11) above;
Ring B is as defined in paragraph (15) above; and
Ring C is as defined in paragraph (23) above.

In a particular group of compounds of the invention, X₁ is NH and:
R₁ is as defined in paragraph (5) above;
Ring A is as defined in paragraph (12) above;
Ring B is as defined in paragraph (16), (17), (18) or (19) above; and
Ring C is as defined in paragraph (24) above.

In a particular group of compounds of the invention, X₁ is O and:
R₁ is as defined in any one of paragraphs (1) to (7), (7a) or (7b) above;
Ring A is as defined in any one of paragraphs (10) to (12) above;
Ring B is as defined in any one of paragraphs (13) to (22) above; and
Ring C is as defined in any one of paragraphs (23) to (24) above.

In a particular group of compounds of the invention, X₁ is O and:
R₁ is as defined in paragraph (4) above;
Ring A is as defined in paragraph (11) above;
Ring B is as defined in paragraph (15) above; and
Ring C is as defined in paragraph (23) above.

In a particular group of compounds of the invention, X₁ is O and:
R₁ is as defined in paragraph (5) above;
Ring A is as defined in paragraph (12) above;
Ring B is as defined in paragraph (16), (17), (18) or (19) above; and
Ring C is as defined in paragraph (24) above.

In a particular group of compounds of the invention, X₁ is NH or O, Ring A is as defined in paragraph (11) above and:
R₁ is as defined in any one of paragraphs (1) to (7), (7a) or (7b) above;
Ring B is as defined in any one of paragraphs (13) to (22) above; and
Ring C is as defined in any one of paragraphs (23) to (24) above.

In a particular group of compounds of the invention, X₁ is NH or O, Ring A is as defined in paragraph (11) above and:
R₁ is as defined in paragraph (4) above;
Ring B is as defined in paragraph (15) above; and
Ring C is as defined in paragraph (23) above.

In a particular group of compounds of the invention, X₁ is NH or O, Ring A is as defined in paragraph (11) above and:
R₁ is as defined in paragraph (5) above;
Ring B is as defined in paragraph (16), (17), (18) or (19) above; and
Ring C is as defined in paragraph (24) above.

In a particular group of compounds of the invention, X₁ is NH, Ring A is as defined in paragraph (12) above and:
R₁ is as defined in any one of paragraphs (1) to (7), (7a) or (7b) above;
Ring B is as defined in any one of paragraphs (13) to (22) above; and
Ring C is as defined in any one of paragraphs (23) to (24) above.
   In a particular group of compounds of the invention, X₁ is NH, Ring A is as defined in paragraph (12) above and:
R₁ is as defined in paragraph (4) above;
Ring B is as defined in paragraph (15) above; and
Ring C is as defined in paragraph (23) above.

In a particular group of compounds of the invention, X₁ is NH, Ring A is as defined in paragraph (11) above and:
R₁ is as defined in paragraph (5) above;
Ring B is as defined in paragraph (16), (17), (18) or (19) above; and
Ring C is as defined in paragraph (24) above.

In a particular group of compounds of the invention, X₁ is NH or O, Ring A is as defined in paragraph (12) above, Ring B is as defined in paragraph (15) above and:
R₁ is as defined in any one of paragraphs (1) to (7), (7a) or (7b) above; and
Ring C is as defined in any one of paragraphs (23) to (24) above.

In a particular group of compounds of the invention, X₁ is NH or O, Ring A is as defined in paragraph (12) above, Ring B is as defined in paragraph (15) above and:
R₁ is as defined in paragraph (4) above; and
Ring C is as defined in paragraph (23) above.

In a particular group of compounds of the invention, X₁ is NH or O, Ring A is as defined in paragraph (12) above, Ring B is as defined in paragraph (15) above and:
R₁ is as defined in paragraph (5) above; and
Ring C is as defined in paragraph (24) above.

In a further group of compounds of the invention, the compounds have the structural Formula (Ia), or a pharmaceutically acceptable salt thereof, as shown below:

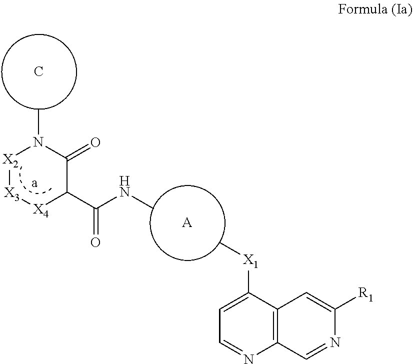

wherein R₁, X₁, Ring A and Ring B each have any one of the definitions set out herein;
[symbol] represents an optional double bond between X₂ and X₃ and/or between X₄ and the adjacent ring carbon atom linked to the amide bond;
X₂ is N or CR₂ₓ wherein R₂ₓ is hydrogen, (1-4C)alkyl or (1-4C)alkoxy when there is a double bond between X₂ and X₃ or
X₂ is CO, CH₂, CHR₂ₓ or NR_{2y} wherein R_{2y} is hydrogen or (1-4C)alkyl when there is a single bond between X₂ and X₃;
X₃ is N or CR₃ₓ wherein R₃ₓ is hydrogen, (1-4C)alkyl or (1-4C)alkoxy when there is a double bond between X₂ and X₃ or
X₃ is CO, CH₂, CHR₃ₓ or NR_{3y} wherein R_{3y} is hydrogen or (1-4C)alkyl when there is a single bond between X₂ and X₃;
X₄ is N or CR₄ₓ wherein R₄ₓ is hydrogen, (1-4C)alkyl or (1-4C)alkoxy when there is a double bond between X₄ and the adjacent ring carbon atom linked to the amide bond or
X₄ is CO, CH₂, CHR₄ₓ or NR_{4y} wherein R_{4y} is hydrogen or (1-4C)alkyl when there is a single bond between X₄ and the adjacent ring carbon atom linked to the amide bond;
wherein any alkyl moiety present in a ring substituent group is optionally further substituted by halo, hydroxy or (1-2C)alkoxy;
and provided that:
   a maximum of one of X₂, X₃ and X₄ is N or NR_{2y}, NR_{3y} or R_{4y} respectively;
   a maximum of one of X₂, X₃ and X₄ is CO; and
   when X₂ is N then X₃ is CO.

In a particular group of compounds of Formula Ia, X₁ is NH or O and:
R₁ is as defined in any one of paragraphs (1) to (7), (7a) or (7b) above;
Ring A is as defined in any one of paragraphs (10) to (12) above;
Ring C is as defined in any one of paragraphs (23) to (24) above;
X₂ is N or CR₂ₓ wherein R₂ₓ is hydrogen, (1-4C)alkyl or (1-4C)alkoxy when there is a double bond between X₂ and X₃ or
X₂ is CO, CH₂, CHR₂ₓ or NR_{2y} wherein R_{2y} is hydrogen or (1-4C)alkyl when there is a single bond between X₂ and X₃;
X₃ is N or CR₃ₓ wherein R₃ₓ is hydrogen, (1-4C)alkyl or (1-4C)alkoxy when there is a double bond between X₂ and X₃ or
X₃ is CO, CH₂, CHR₃ₓ or NR_{3y} wherein R_{3y} is hydrogen or (1-4C)alkyl when there is a single bond between X₂ and X₃;
X₄ is N or CR₄ₓ wherein R₄ₓ is hydrogen, (1-4C)alkyl or (1-4C)alkoxy when there is a double bond between X₄ and the adjacent ring carbon atom linked to the amide bond; or
X₄ is CO, CH₂, CHR₄ₓ or NR_{4y} wherein R_{4y} is hydrogen or (1-4C)alkyl when there is a single bond between X₄ and the adjacent ring carbon atom linked to the amide bond.

In a particular group of compounds of Formula Ia, X₁ is NH or O and:
R₁ is as defined in paragraph (4) above;
Ring A is as defined in paragraph (11) above;
Ring C is as defined in paragraph (23) above;
X₂ is N or CR₂ₓ wherein R₂ₓ is hydrogen or (1-2C)alkyl when there is a double bond between X₂ and X₃; or
X₂ is CO, CH₂, or CHR₂ₓ when there is a single bond between X₂ and X₃;
X₃ is CR₃ₓ wherein R₃ₓ is hydrogen or (1-2C)alkyl when there is a double bond between X₂ and X₃; or
X₃ is CH₂, CHR₃ₓ or NR_{3y} wherein R_{3y} is hydrogen or (1-4C)alkyl when there is a single bond between X₂ and X₃;
X₄ is N or CR₄ₓ wherein R₄ₓ is hydrogen, (1-4C)alkyl or (1-4C)alkoxy when there is a double bond between X₄ and the adjacent ring carbon atom linked to the amide bond; or
X₄ is CO, CH₂, CHR_{4X} or NR_{4y} wherein R_{4y} is hydrogen or (1-4C)alkyl when there is a single bond between X₄ and the adjacent ring carbon atom linked to the amide bond.

In a particular group of compounds of Formula Ia, X₁ is NH or O and:
R₁ is as defined in paragraph (5) above;
Ring A is as defined in paragraph (12) above; and
Ring C is as defined in paragraph (24) above;
X₂ is N or CR₂ₓ wherein R₂ₓ is hydrogen when there is a double bond between X₂ and X₃; or
X₂ is CO when there is a single bond between X₂ and X₃;
X₃ is CR₃ₓ wherein R₃ₓ is hydrogen when there is a double bond between X₂ and X₃; or
X₃ is NR_{3y} wherein R_{3y} is hydrogen or (1-4C)alkyl when there is a single bond between X₂ and X₃;
X₄ is N or CR₄ₓ wherein R₄ₓ is hydrogen, (1-4C)alkyl or (1-4C)alkoxy and there is a double bond between X₄ and the adjacent ring carbon atom linked to the amide bond.

In a particular group of compounds of Formula Ia, X₁ is NH or O and:
R₁ is as defined in any one of paragraphs (1) to (7), (7a) or (7b) above;
Ring A is as defined in any one of paragraphs (10) to (12) above;
Ring C is as defined in any one of paragraphs (23) to (24) above;
X₂ is N or CR₂ₓ wherein R₂ₓ is hydrogen, (1-4C)alkyl or (1-4C)alkoxy when there is a double bond between X₂ and X₃ or
X₂ is CO, CH₂, CHR₂ₓ or NR_{2y} wherein R_{2y} is hydrogen or (1-4C)alkyl when there is a single bond between X₂ and X₃;
X₃ is N or CR₃ₓ wherein R₃ₓ is hydrogen, (1-4C)alkyl or (1-4C)alkoxy when there is a double bond between X₂ and X₃ or
X₃ is CO, CH₂, CHR_{3X} or NR_{3y} wherein R_{3y} is hydrogen or (1-4C)alkyl when there is a single bond between X₂ and X₃;
X₄ is N or CR₄ₓ wherein R₄ₓ is hydrogen, (1-4C)alkyl or (1-4C)alkoxy when there is a double bond between X₄ and the adjacent ring carbon atom linked to the amide bond; or
X₄ is CO, CH₂, CHR₄ₓ or NR_{4y} wherein R_{4y} is hydrogen or (1-4C)alkyl when there is a single bond between X₄ and the adjacent ring carbon atom linked to the amide bond.

In a particular group of compounds of Formula Ia, X₁ is NH or O and:
R₁ is as defined in paragraph (4) above;
Ring A is as defined in paragraph (11) above;
Ring C is as defined in paragraph (23) above
X₂ is N or CR₂ₓ wherein R₂ₓ is hydrogen or (1-2C)alkyl when there is a double bond between X₂ and X₃; or
X₂ is CO, CH₂, or CHR₂ₓ when there is a single bond between X₂ and X₃;
X₃ is CR₃ₓ wherein R₃ₓ is hydrogen or (1-2C)alkyl when there is a double bond between X₂ and X₃; or
X₃ is CH₂, CHR₃ₓ or NR_{3y} wherein R_{3y} is hydrogen or (1-4C)alkyl when there is a single bond between X₂ and X₃;
X₄ is N or CR₄ₓ wherein R₄ₓ is hydrogen, (1-4C)alkyl or (1-4C)alkoxy when there is a double bond between X₄ and the adjacent ring carbon atom linked to the amide bond; or
X₄ is CO, CH₂, CHR₄ₓ or NR_{4y} wherein R_{4y} is hydrogen or (1-4C)alkyl when there is a single bond between X₄ and the adjacent ring carbon atom linked to the amide bond.

In a particular group of compounds of Formula Ia, X₁ is NH or O and:
R₁ is as defined in paragraph (5) above;
Ring A is as defined in paragraph (12) above;
Ring C is as defined in paragraph (24) above;
X₂ is N or CR₂ₓ wherein R₂ₓ is hydrogen when there is a double bond between X₂ and X₃; or
X₂ is CO when there is a single bond between X₂ and X₃;
X₃ is CR₃ₓ wherein R₃ₓ is hydrogen when there is a double bond between X₂ and X₃; or
X₃ is NR_{3y} wherein R_{3y} is hydrogen or (1-4C)alkyl when there is a single bond between X₂ and X₃;
X₄ is N or CR₄ₓ wherein R₄ₓ is hydrogen, (1-4C)alkyl or (1-4C)alkoxy and there is a double bond between X₄ and the adjacent ring carbon atom linked to the amide bond.

In a further group of compounds of the invention, the compounds have the structural formula Ib (a sub-definition of Formula (I)) shown below, or a pharmaceutically acceptable salt and/or solvate thereof:

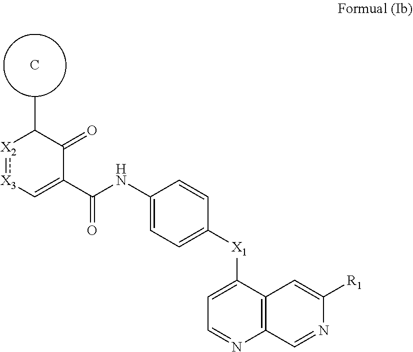

wherein R₁, X₁, X₂, X₃ and Ring C (and any associated substituent groups) have any of the meanings defined herein.

In a particular group of compounds of Formula Ib, X₁ is NH or O and:
R₁ is as defined in any one of paragraphs (1) to (7), (7a) or (7b) above;
Ring C is as defined in any one of paragraphs (23) to (24) above;
X₂ is N or CR₂ₓ wherein R₂ₓ is hydrogen, (1-4C)alkyl or (1-4C)alkoxy when there is a double bond between X₂ and X₃ or
X₂ is CO, CH₂, CHR₂ₓ or NR_{2y} wherein R_{2y} is hydrogen or (1-4C)alkyl when there is a single bond between X₂ and X₃;
X₃ is N or CR₃ₓ wherein R₃ₓ is hydrogen, (1-4C)alkyl or (1-4C)alkoxy when there is a double bond between X₂ and X₃ or
X₃ is CO, CH₂, CHR₃ₓ or NR_{3y} wherein R_{3y} is hydrogen or (1-4C)alkyl when there is a single bond between X₂ and X₃.

In a particular group of compounds of Formula Ib, X₁ is NH or O and:
R₁ is as defined in paragraph (4) above;
Ring C is as defined in paragraph (23) above;
X₂ is N or CR₂ₓ wherein R₂ₓ is hydrogen or (1-2C)alkyl when there is a double bond between X₂ and X₃; or
X₂ is CO, CH₂, or CHR₂ₓ when there is a single bond between X₂ and X₃;
X₃ is CR₃ₓ wherein R₃ₓ is hydrogen or (1-2C)alkyl when there is a double bond between X₂ and X₃; or
X₃ is CH₂, CHR₃ₓ or NR_{3y} wherein R_{3y} is hydrogen or (1-4C)alkyl when there is a single bond between X₂ and X₃.

In a particular group of compounds of Formula Ib, X₁ is NH or O and:
R₁ is as defined in paragraph (5) above;
Ring C is as defined in paragraph (24) above;
X₂ is N or CR₂ₓ wherein R₂ₓ is hydrogen when there is a double bond between X₂ and X₃; or
X₂ is CO when there is a single bond between X₂ and X₃;
X₃ is CR₃ₓ wherein R₃ₓ is hydrogen when there is a double bond between X₂ and X₃; or
X₃ is NR_{3y} wherein R_{3y} is hydrogen or (1-4C)alkyl when there is a single bond between X₂ and X₃.

In a particular group of compounds of Formula Ib, X₁ is NH or O and:
R₁ is as defined in any one of paragraphs (1) to (7), (7a) or (7b) above;
Ring C is as defined in any one of paragraphs (23) to (24) above;
X₂ is N or CR₂ₓ wherein R₂ₓ is hydrogen, (1-4C)alkyl or (1-4C)alkoxy when there is a double bond between X₂ and X₃ or
X₂ is CO, CH₂, CHR₂ₓ or NR_{2y} wherein R_{2y} is hydrogen or (1-4C)alkyl when there is a single bond between X₂ and X₃;
X₃ is N or CR₃ₓ wherein R₃ₓ is hydrogen, (1-4C)alkyl or (1-4C)alkoxy when there is a double bond between X₂ and X₃ or
X₃ is CO, CH₂, CHR_{3X} or NR_{3y} wherein R_{3y} is hydrogen or (1-4C)alkyl when there is a single bond between X₂ and X₃.

In a particular group of compounds of Formula Ib, X₁ is NH or O and:
R₁ is as defined in paragraph (4) above;
Ring C is as defined in paragraph (23) above;
X₂ is N or CR₂ₓ wherein R₂ₓ is hydrogen or (1-2C)alkyl when there is a double bond between X₂ and X₃; or
X₂ is CO, CH₂, or CHR₂ₓ when there is a single bond between X₂ and X₃;
X₃ is CR₃ₓ wherein R₃ₓ is hydrogen or (1-2C)alkyl when there is a double bond between X₂ and X₃; or
X₃ is CH₂, CHR₃ₓ or NR_{3y} wherein R_{3y} is hydrogen or (1-4C)alkyl when there is a single bond between X₂ and X₃.

In a particular group of compounds of Formula Ib, X₁ is NH or O and:
R₁ is as defined in paragraph (5) above;
Ring C is as defined in paragraph (24) above;
X₂ is N or CR₂ₓ wherein R₂ₓ is hydrogen when there is a double bond between X₂ and X₃; or
X₂ is CO when there is a single bond between X₂ and X₃;
X₃ is CR₃ wherein R₃ₓ is hydrogen when there is a double bond between X₂ and X₃; or X₃ is NR_{3y} wherein R_{3y} is hydrogen or (1-4C)alkyl when there is a single bond between X₂ and X₃.

In a particular group of compounds of Formula Ib, X₁ is NH or O and:
R₁ is as defined in paragraph (6) above;
Ring C is as defined in paragraph (24) above;
X₂ is N or CR₂ₓ wherein R₂ₓ is hydrogen when there is a double bond between X₂ and X₃; or
X₂ is CO when there is a single bond between X₂ and X₃;
X₃ is CR₃ₓ wherein R₃ₓ is hydrogen when there is a double bond between X₂ and X₃; or
X₃ is NR_{3y} wherein R_{3y} is hydrogen or (1-4C)alkyl when there is a single bond between X₂ and X₃.

In a particular group of compounds of Formula Ib, X₁ is NH or O and:
R₁ is as defined in paragraph (7), (7a) or (7b) above;
Ring C is as defined in paragraph (24) above;
X₂ is N or CR₂ₓ wherein R₂ₓ is hydrogen when there is a double bond between X₂ and X₃; or
X₂ is CO when there is a single bond between X₂ and X₃;
X₃ is CR₃ₓ wherein R₃ₓ is hydrogen when there is a double bond between X₂ and X₃; or
X₃ is NR_{3y} wherein R_{3y} is hydrogen or (1-4C)alkyl when there is a single bond between X₂ and X₃.

In a particular group of compounds of Formula Ia or Ib as defined herein, X₁ is —NH or —O—; X₂ is CH and X₃ is CH when there is a double bond between X₂ and X₃ or X₂ is CO and X₃ is NR_{3y} wherein R_{3y} is (1-4C)alkyl, in particular prop-2-yl (iso-propyl) when there is a single bond between X₂ and X₃; and Ring C is as defined in paragraph (23) or (24) above.

In a further particular group of compounds of the invention, the compounds have the structural formula Ic (a sub-definition of Formula (I)) shown below, or a pharmaceutically acceptable salt and/or solvate thereof:

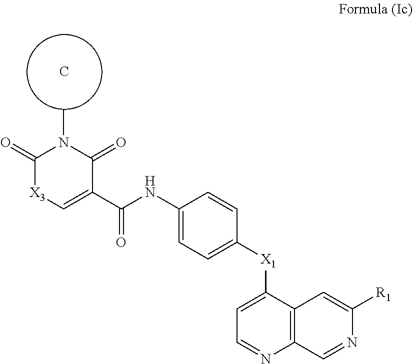

wherein R₁, Ring C and X₃ (and any associated substituent groups) have any of the meanings defined herein.

In a particular group of compounds of Formula Ic, X₁ is NH or O and:
R₁ is as defined in any one of paragraphs (1) to (7), (7a) or (7b) above;
Ring C is as defined in any one of paragraphs (23) to (24) above;
X₃ is CH₂ or NR_{3y} wherein R_{3y} is hydrogen or (1-4C)alkyl.

In a particular group of compounds of Formula Ic, X₁ is NH or O and:
R₁ is as defined in paragraph (4) above;
Ring C is as defined in paragraph (23) above;
X₃ is NR_{3y} wherein R_{3y} is hydrogen or (1-4C)alkyl.

In a particular group of compounds of Formula Ic, X₁ is NH or O and:
R₁ is as defined in paragraph (5) above;
Ring C is as defined in paragraph (24) above;
X₃ is NR_{3y} wherein R_{3y} is hydrogen or (1-4C)alkyl.

In a particular group of compounds of Formula Ic, X₁ is NH or O and:
R₁ is as defined in any one of paragraphs (1) to (7), (7a) or (7b) above;
Ring C is as defined in any one of paragraphs (23) to (24) above;
X₃ is CH₂ or NR_{3y} wherein R_{3y} is hydrogen or (1-4C)alkyl.

In a particular group of compounds of Formula Ic, X₁ is NH or O and:
R₁ is as defined in paragraph (4) above;
Ring C is as defined in paragraph (23) above
X₃ is NR_{3y} wherein R_{3y} is hydrogen or (1-4C)alkyl.

In a particular group of compounds of Formula Ic, X₁ is NH or O and:
R₁ is as defined in paragraph (5) above;
Ring C is as defined in paragraph (24) above
X₃ is NR_{3y} wherein R_{3y} is hydrogen or (1-4C)alkyl.

In a particular group of compounds of Formula Ic, X₁ is NH or O and:
R₁ is as defined in paragraph (7), (7a) or (7b) above;
Ring C is as defined in paragraph (24) above
X₃ is NR_{3y} wherein R_{3y} is hydrogen or (1-4C)alkyl.

In a particular group of compounds of Formula Ic, X₁ is —NH or —O—; X₃ is NR_{3y} wherein R_{3y} is (1-4C)alkyl, in particular prop-2-yl (iso-propyl); and Ring C is as defined in paragraph (23) or (24) above.

In a further particular group of compounds of the invention, the compounds have the structural formula Id (a sub-definition of Formula (I)) shown below, or a pharmaceutically acceptable salt and/or solvate thereof:

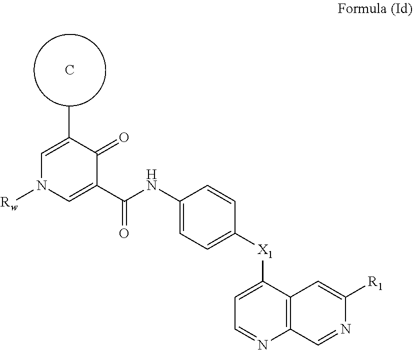

wherein R₁, Ring C and R_{w} (and any associated substituent groups) have any of the meanings defined herein.

In a particular group of compounds of Formula Id, X₁ is NH or O, and:
R₁ is as defined in any one of paragraphs (1) to (7), (7a) or (7b) above;
Ring C is as defined in any one of paragraphs (23) to (24) above;
R_{w} is as defined in any one of paragraphs (15) to (22) above.

In a particular group of compounds of Formula Id:
R₁ is as defined in paragraph (4) above;
Ring C is as defined in paragraph (23) above;
R_{w} is as defined in any one of paragraphs (17) to (22) above.

In a particular group of compounds of Formula Id, X₁ is NH or O and:
R₁ is as defined in paragraph (5) above;
Ring C is as defined in paragraph (24) above;
R_{w} is as defined in paragraph (22) above.

In a particular group of compounds of Formula Id, X₁ is NH or O and:
R₁ is as defined in paragraph (6) above;
Ring C is as defined in paragraph (24) above;
R_{w} is as defined in paragraph (22) above.

In a particular group of compounds of Formula Id, X₁ is NH or O and:
R₁ is as defined in paragraph (7), (7a) or (7b) above;
Ring C is as defined in paragraph (24) above;
R_{w} is as defined in paragraph (22) above.

In a further particular group of compounds of the invention, the compounds have the structural formula Ie (a sub-definition of Formula (I)) shown below, or a pharmaceutically acceptable salt and/or solvate thereof:

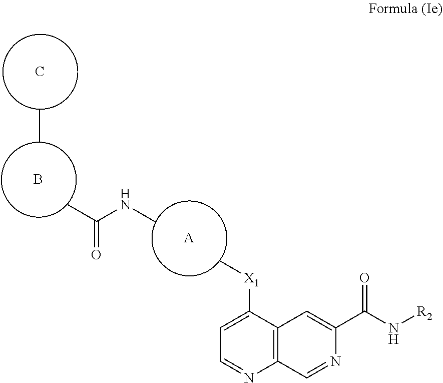

wherein R₂, Ring A, Ring B and Ring C (and any associated substituent groups) have any of the meanings defined herein.

In a particular group of compounds of Formula Ie:
X₁ is as defined in any one of paragraphs (8) or (9) above;
Ring A is as defined in any one of paragraphs (10) to (12) above;
Ring B is as defined in any one of paragraphs (13) to (22) above;
Ring C is as defined in paragraph (23) or (24) above;
R₂ is as defined in any one of paragraphs (1) to (7), (7a) or (7b) above.

In a particular group of compounds of Formula Ie:
X₁ is NH or O, and;
Ring A is as defined in paragraph (11) or (12) above;
Ring B is as defined in any one of paragraphs (16) to (19) above;
Ring C is as defined in paragraph (23) or (24) above;
R₂ is as defined in paragraph (6), (7), (7a) or (7b) above.

In a particular group of compounds of Formula Ie:
X₁ is O;
Ring A is as defined in paragraph (12) above;
Ring B is as defined in any one of paragraphs (19), (20), (21) or (22) above;
Ring C is as defined in paragraph (24) above;
R₂ is as defined in paragraph (7b) above.

Particular compounds of the present invention include any of the compounds exemplified in the present application, or a pharmaceutically acceptable salt or solvate thereof, and, in particular, any of the following:
2-oxo-1-phenyl-N-[4-[(6-piperazin-1-yl-1,7-naphthyridin-4-yl)amino]phenyl]pyridine-3-carboxamide;
1,5-dimethyl-3-oxo-2-phenyl-N-[4-[(6-piperazin-1-yl-1,7-naphthyridin-4-yl)amino]phenyl]pyrazole-4-carboxamide;
3-(4-fluorophenyl)-1-isopropyl-2,4-dioxo-N-[4-[(6-piperazin-1-yl-1,7-naphthyridin-4-yl)amino]phenyl]pyrimidine-5-carboxamide;
1-(4-fluorophenyl)-2-oxo-N-[4-[(6-piperazin-1-yl-1,7-naphthyridin-4-yl)amino]phenyl]pyridine-3-carboxamide;
1-isopropyl-2,4-dioxo-3-phenyl-N-[4-[(6-piperazin-1-yl-1,7-naphthyridin-4-yl)amino]phenyl]pyrimidine-5-carboxamide;
N-[4-[[6-(4-hydroxy-1-piperidyl)-1,7-naphthyridin-4-yl]amino]phenyl]-2-oxo-1-phenyl-pyridine-3-carboxamide;
N-[4-[(6-morpholino-1,7-naphthyridin-4-yl)amino]phenyl]-2-oxo-1-phenyl-pyridine-3-carboxamide;
N-[4-[[6-(1,4-diazepan-1-yl)-1,7-naphthyridin-4-yl]amino]phenyl]-2-oxo-1-phenyl-pyridine-3-carboxamide;
N-(1-methyl-4-piperidyl)-4-[4-[(2-oxo-1-phenyl-pyridine-3-carbonyl)amino]anilino]-1,7-naphthyridine-6-carboxamide;
N-[4-[[6-(1-methylpyrazol-4-yl)-1,7-naphthyridin-4-yl]amino]phenyl]-2-oxo-1-phenyl-pyridine-3-carboxamide;
N-[4-[[6-[1-(1-methyl-4-piperidyl)pyrazo]-4-yl]-1,7-naphthyridin-4-yl]amino]phenyl]-2-oxo-1-phenyl-pyridine-3-carboxamide;
N-(4-hydroxycyclohexyl)-4-[4-[(2-oxo-1-phenyl-pyridine-3-carbonyl)amino]anilino]-1,7-naphthyridine-6-carboxamide;
N-(2-hydroxyethoxy)-4-[4-[(2-oxo-1-phenyl-pyridine-3-carbonyl)amino]anilino]-1,7-naphthyridine-6-carboxamide;
4-[4-[(2-oxo-1-phenyl-pyridine-3-carbonyl)amino]anilino]-1,7-naphthyridine-6-carboxamide;
N-methyl-4-[4-[(2-oxo-1-phenyl-pyridine-3-carbonyl)amino]anilino]-1,7-naphthyridine-6-carboxamide;
N-(1-methyl-4-piperidyl)-4-[4-[(2-oxo-1-phenyl-pyridine-3-carbonyl)amino]phenoxy]-1,7-naphthyridine-6-carboxamide;
4-[4-[[3-(4-fluorophenyl)-1-isopropyl-2,4-dioxo-pyrimidine-5-carbonyl]amino]anilino]-N-(2-hydroxyethoxy)-1,7-naphthyridine-6-carboxamide;
N-[2-(dimethylamino)ethyl]-4-[4-[[3-(4-fluorophenyl)-1-isopropyl-2,4-dioxo-pyrimidine-5-carbonyl]amino]anilino]-1,7-naphthyridine-6-carboxamide;
4-[4-[(1,5-Dimethyl-3-oxo-2-phenyl-pyrazole-4-carbonyl)amino]phenoxy]-N-(1-methyl-4-piperidyl)-1,7-naphthyridine-6-carboxamide;
4-[4-[[1-(4-fluorophenyl)-2-oxo-pyridine-3-carbonyl]amino]phenoxy]-N-(1-methyl-4-piperidyl)-1,7-naphthyridine-6-carboxamide;
4-[4-[[3-(4-fluorophenyl)-1-isopropyl-2,4-dioxo-pyrimidine-5-carbonyl]amino]phenoxy]-N-(1-methyl-4-piperidyl)-1,7-naphthyridine-6-carboxamide;
4-[4-[[3-(5-Fluoro-2-pyridyl)-1-isopropyl-2,4-dioxo-pyrimidine-5-carbonyl]amino]phenoxy]-N-(1-methyl-4-piperidyl)-1,7-naphthyridine-6-carboxamide;
4-[4-[[3-(3,5-difluorophenyl)-1-isopropyl-2,4-dioxo-pyrimidine-5-carbonyl]amino]phenoxy]-N-(1-methyl-4-piperidyl)-1,7-naphthyridine-6-carboxamide;
N-[3-methyl-4-[(6-piperazin-1-yl-1,7-naphthyridin-4-yl)oxy]phenyl]-2-oxo-1-phenyl-pyridine-3-carboxamide;
2-oxo-1-phenyl-N-[5-[(6-piperazin-1-yl-1,7-naphthyridin-4-yl)oxy]-2-pyridyl]pyridine-3-carboxamide;
N-[3-fluoro-4-[(6-piperazin-1-yl-1,7-naphthyridin-4-yl)oxy]phenyl]-2-oxo-1-phenyl-pyridine-3-carboxamide;
1-(4-fluorophenyl)-2-oxo-N-[5-[(6-piperazin-1-yl-1,7-naphthyridin-4-yl)oxy]-2-pyridyl]pyridine-3-carboxamide;
1-(4-fluorophenyl)-N-[5-[[6-(4-methylpiperazin-1-yl)-1,7-naphthyridin-4-yl]oxy]-2-pyridyl]-2-oxo-pyridine-3-carboxamide;
1-(4-fluorophenyl)-N-[5-[[6-[4-(oxetan-3-yl)piperazin-1-yl]-1,7-naphthyridin-4-yl]oxy]-2-pyridyl]-2-oxo-pyridine-3-carboxamide;
4-[2-Methyl-4-[(2-oxo-1-phenyl-pyridine-3-carbonyl)amino]phenoxy]-N-(1-methyl-4-piperidyl)-1,7-naphthyridine-6-carboxamide;
4-[2-fluoro-4-[(2-oxo-1-phenyl-pyridine-3-carbonyl)amino]phenoxy]-N-(1-methyl-4-piperidyl)-1,7-naphthyridine-6-carboxamide;
4-[[6-[[3-(4-fluorophenyl)-1-isopropyl-2,4-dioxo-pyrimidine-5-carbonyl]amino]-3-pyridyl]oxy]-N-(1-methyl-4-piperidyl)-1,7-naphthyridine-6-carboxamide;
N-(1-methyl-4-piperidyl)-4-[[6-[(2-oxo-1-phenyl-pyridine-3-carbonyl)amino]-3-pyridyl]oxy]-1,7-naphthyridine-6-carboxamide;
4-[[6-[[1-(4-fluorophenyl)-2-oxo-pyridine-3-carbonyl]amino]-3-pyridyl]oxy]-N-(1-methyl-4-piperidyl)-1,7-naphthyridine-6-carboxamide;
2-Oxo-1-phenyl-N-[4-[(6-piperazin-1-yl-1,7-naphthyridin-4-yl)oxy]phenyl]pyridine-3-carboxamide;
2-oxo-N-[4-[(6-piperazin-1-yl-1,7-naphthyridin-4-yl)oxy]phenyl]-1-(2-pyridyl)pyridine-3-carboxamide;
1-(4-fluorophenyl)-2-oxo-N-[4-[(6-piperazin-1-yl-1,7-naphthyridin-4-yl)oxy]phenyl]pyridine-3-carboxamide;
1-isopropyl-2,4-dioxo-N-[4-[(6-piperazin-1-yl-1,7-naphthyridin-4-yl)oxy]phenyl]-3-(2-pyridyl)pyrimidine-5-carboxamide;
3-(5-fluoro-2-pyridyl)-1-isopropyl-2,4-dioxo-N-[4-[(6-piperazin-1-yl-1,7-naphthyridin-4-yl)oxy]phenyl]pyrimidine-5-carboxamide;
3-(4-fluorophenyl)-1-isopropyl-2,4-dioxo-N-[4-[(6-piperazin-1-yl-1,7-naphthyridin-4-yl)oxy]phenyl]pyrimidine-5-carboxamide;
6-(4-fluorophenyl)-1-oxido-N-[4-[(6-piperazin-1-yl-1,7-naphthyridin-4-yl)oxy]phenyl]pyridin-1-ium-2-carboxamide;
1-(4-fluorophenyl)-N-[4-[[6-(4-methylpiperazin-1-yl)-1,7-naphthyridin-4-yl]oxy]phenyl]-2-oxo-pyridine-3-carboxamide;
5-(4-fluorophenyl)-4-oxo-N-[4-[(6-piperazin-1-yl-1,7-naphthyridin-4-yl)oxy]phenyl]-1H-pyridine-3-carboxamide;
1,5-dimethyl-3-oxo-2-phenyl-N-[4-[(6-piperazin-1-yl-1,7-naphthyridin-4-yl)oxy]phenyl]pyrazole-4-carboxamide;
5-(4-Fluorophenyl)-1-isopropyl-4-oxo-N-[4-[(6-piperazin-1-yl-1,7-naphthyridin-4-yl)oxy]phenyl]pyridine-3-carboxamide;
5-(4-fluorophenyl)-1-methyl-4-oxo-N-[4-[(6-piperazin-1-yl-1,7-naphthyridin-4-yl)oxy]phenyl]pyridine-3-carboxamide;
4-[4-[[5-(4-fluorophenyl)-1-methyl-4-oxo-pyridine-3-carbonyl]amino]phenoxy]-N-(1-methyl-4-piperidyl)-1,7-naphthyridine-6-carboxamide;
4-[4-[[5-(4-fluorophenyl)-1-isopropyl-4-oxo-pyridine-3-carbonyl]amino]phenoxy]-N-(1-methyl-4-piperidyl)-1,7-naphthyridine-6-carboxamide;
4-[4-[[3-(4-Fluorophenyl)-2,4-dioxo-1-[(1R)-2-hydroxy-1-methyl-ethyl]pyrimidine-5-carbonyl]amino]phenoxy]-N-(1-methyl-4-piperidyl)-1,7-naphthyridine-6-carboxamide;
4-[4-[[1-Cyclopropyl-3-(4-fluorophenyl)-2,4-dioxo-pyrimidine-5-carbonyl]amino]phenoxy]-N-(1-methyl-4-piperidyl)-1,7-naphthyridine-6-carboxamide;
1-cyclopropyl-3-(4-fluorophenyl)-2,4-dioxo-N-[4-[(6-piperazin-1-yl-1,7-naphthyridin-4-yl)oxy]phenyl]pyrimidine-5-carboxamide;
1-(4-Fluorophenyl)-N-[5-[[6-(1-methyl-4-piperidyl)-1,7-naphthyridin-4-yl]oxy]-2-pyridyl]-2-oxo-pyridine-3-carboxamide;
1-(4-fluorophenyl)-N-[4-[[6-(1-methyl-4-piperidyl)-1,7-naphthyridin-4-yl]oxy]phenyl]-2-oxo-pyridine-3-carboxamide;
1-(4-Fluorophenyl)-N-[5-[[6-[(1-methylpyrrolidine-3-carbonyl)amino]-1,7-naphthyridin-4-yl]oxy]-2-pyridyl]-2-oxo-pyridine-3-carboxamide;
1-(4-fluorophenyl)-N-[5-[[6-[(1-methylpiperidine-4-carbonyl)amino]-1,7-naphthyridin-4-yl]oxy]-2-pyridyl]-2-oxo-pyridine-3-carboxamide;
4-[2-[[1-(4-Fluorophenyl)-2-oxo-pyridine-3-carbonyl]amino]pyrimidin-5-yl]oxy-N-(1-methyl-4-piperidyl)-1,7-naphthyridine-6-carboxamide.

Particular compounds of the present invention include any of the compounds exemplified in the present application, or a pharmaceutically acceptable salt or solvate thereof, and, in particular, any of the following:
2-oxo-1-phenyl-N-[4-[(6-piperazin-1-yl-1,7-naphthyridin-4-yl)amino]phenyl]pyridine-3-carboxamide
3-(4-fluorophenyl)-1-isopropyl-2,4-dioxo-N-[4-[(6-piperazin-1-yl-1,7-naphthyridin-4-yl)amino]phenyl]pyrimidine-5-carboxamide
1-(4-fluorophenyl)-2-oxo-N-[4-[(6-piperazin-1-yl-1,7-naphthyridin-4-yl)amino]phenyl]pyridine-3-carboxamide
1-isopropyl-2,4-dioxo-3-phenyl-N-[4-[(6-piperazin-1-yl-1,7-naphthyridin-4-yl)amino]phenyl]pyrimidine-5-carboxamide
1,5-dimethyl-3-oxo-2-phenyl-N-[4-[(6-piperazin-1-yl-1,7-naphthyridin-4-yl)amino]phenyl]pyrazole-4-carboxamide
N-[4-[[6-(4-hydroxy-1-piperidyl)-1,7-naphthyridin-4-yl]amino]phenyl]-2-oxo-1-phenyl-pyridine-3-carboxamide
N-[4-[(6-morpholino-1,7-naphthyridin-4-yl)amino]phenyl]-2-oxo-1-phenyl-pyridine-3-carboxamide
N-[4-[[6-(1,4-diazepan-1-yl)-1,7-naphthyridin-4-yl]amino]phenyl]-2-oxo-1-phenyl-pyridine-3-carboxamide
N-(1-methyl-4-piperidyl)-4-[4-[(2-oxo-1-phenyl-pyridine-3-carbonyl)amino]anilino]-1,7-naphthyridine-6-carboxamide
N-[4-[[6-(1-methylpyrazol-4-yl)-1,7-naphthyridin-4-yl]amino]phenyl]-2-oxo-1-phenyl-pyridine-3-carboxamide
N-[4-[[6-[1-(1-methyl-4-piperidyl)pyrazo]-4-yl]-1,7-naphthyridin-4-yl]amino]phenyl]-2-oxo-1-phenyl-pyridine-3-carboxamide
N-(4-hydroxycyclohexyl)-4-[4-[(2-oxo-1-phenyl-pyridine-3-carbonyl)amino]anilino]-1,7-naphthyridine-6-carboxamide
N-(2-hydroxyethoxy)-4-[4-[(2-oxo-1-phenyl-pyridine-3-carbonyl)amino]anilino]-1,7-naphthyridine-6-carboxamide
4-[4-[(2-oxo-1-phenyl-pyridine-3-carbonyl)amino]anilino]-1,7-naphthyridine-6-carboxamide
N-methyl-4-[4-[(2-oxo-1-phenyl-pyridine-3-carbonyl)amino]anilino]-1,7-naphthyridine-6-carboxamide
N-[4-[[6-(azetidine-1-carbonyl)-1,7-naphthyridin-4-yl]amino]phenyl]-2-oxo-1-phenyl-pyridine-3-carboxamide
N-(1-methyl-4-piperidyl)-4-[4-[(2-oxo-1-phenyl-pyridine-3-carbonyl)amino]phenoxy]-1,7-naphthyridine-6-carboxamide
4-[4-[[3-(4-fluorophenyl)-1-isopropyl-2,4-dioxo-pyrimidine-5-carbonyl]amino]anilino]-N-(2-hydroxyethoxy)-1,7-naphthyridine-6-carboxamide
N-[2-(dimethylamino)ethyl]-4-[4-[[3-(4-fluorophenyl)-1-isopropyl-2,4-dioxo-pyrimidine-5-carbonyl]amino]anilino]-1,7-naphthyridine-6-carboxamide
N-(1-methyl-4-piperidyl)-4-[4-[[2-oxo-1-(2-pyridyl)pyridine-3-carbonyl]amino]phenoxy]-1,7-naphthyridine-6-carboxamide
4-[4-[[1-(4-fluorophenyl)-2-oxo-pyridine-3-carbonyl]amino]phenoxy]-N-(1-methyl-4-piperidyl)-1,7-naphthyridine-6-carboxamide
4-[4-[[3-(4-fluorophenyl)-1-isopropyl-2,4-dioxo-pyrimidine-5-carbonyl]amino]phenoxy]-N-(1-methyl-4-piperidyl)-1,7-naphthyridine-6-carboxamide
4-[4-[(1,5-dimethyl-3-oxo-2-phenyl-pyrazole-4-carbonyl)amino]phenoxy]-N-(1-methyl-4-piperidyl)-1,7-naphthyridine-6-carboxamide
4-[4-[[1-(2-hydroxy-2-methyl-propyl)-5-methyl-3-oxo-2-phenyl-pyrazole-4-carbonyl]amino]phenoxy]-N-(1-methyl-4-piperidyl)-1,7-naphthyridine-6-carboxamide
4-[4-[[3-(5-fluoro-2-pyridyl)-1-isopropyl-2,4-dioxo-pyrimidine-5-carbonyl]amino]phenoxy]-N-(1-methyl-4-piperidyl)-1,7-naphthyridine-6-carboxamide
4-[4-[[3-(3,5-difluorophenyl)-1-isopropyl-2,4-dioxo-pyrimidine-5-carbonyl]amino]phenoxy]-N-(1-methyl-4-piperidyl)-1,7-naphthyridine-6-carboxamide
1-(4-fluorophenyl)-2-oxo-N-[5-[(6-piperazin-1-yl-1,7-naphthyridin-4-yl)oxy]-2-pyridyl]pyridine-3-carboxamide
1-(4-fluorophenyl)-N-[5-[[6-(4-methylpiperazin-1-yl)-1,7-naphthyridin-4-yl]oxy]-2-pyridyl]-2-oxo-pyridine-3-carboxamide
1-(4-fluorophenyl)-N-[5-[[6-[4-(oxetan-3-yl)piperazin-1-yl]-1,7-naphthyridin-4-yl]oxy]-2-pyridyl]-2-oxo-pyridine-3-carboxamide
4-[[6-[[1-(4-fluorophenyl)-2-oxo-pyridine-3-carbonyl]amino]-3-pyridyl]oxy]-N-(1-methyl-4-piperidyl)-1,7-naphthyridine-6-carboxamide
2-oxo-1-phenyl-N-[4-[(6-piperazin-1-yl-1,7-naphthyridin-4-yl)oxy]phenyl]pyridine-3-carboxamide
2-oxo-N-[4-[(6-piperazin-1-yl-1,7-naphthyridin-4-yl)oxy]phenyl]-1-(2-pyridyl)pyridine-3-carboxamide
1-(4-fluorophenyl)-2-oxo-N-[4-[(6-piperazin-1-yl-1,7-naphthyridin-4-yl)oxy]phenyl]pyridine-3-carboxamide
1-isopropyl-2,4-dioxo-N-[4-[(6-piperazin-1-yl-1,7-naphthyridin-4-yl)oxy]phenyl]-3-(2-pyridyl)pyrimidine-5-carboxamide
3-(5-fluoro-2-pyridyl)-1-isopropyl-2,4-dioxo-N-[4-[(6-piperazin-1-yl-1,7-naphthyridin-4-yl)oxy]phenyl]pyrimidine-5-carboxamide
3-(4-fluorophenyl)-1-isopropyl-2,4-dioxo-N-[4-[(6-piperazin-1-yl-1,7-naphthyridin-4-yl)oxy]phenyl]pyrimidine-5-carboxamide
6-(4-fluorophenyl)-1-oxido-N-[4-[(6-piperazin-1-yl-1,7-naphthyridin-4-yl)oxy]phenyl]pyridin-1-ium-2-carboxamide
5-(4-fluorophenyl)-4-oxo-N-[4-[(6-piperazin-1-yl-1,7-naphthyridin-4-yl)oxy]phenyl]-1H-pyridine-3-carboxamide
1,5-dimethyl-3-oxo-2-phenyl-N-[4-[(6-piperazin-1-yl-1,7-naphthyridin-4-yl)oxy]phenyl]pyrazole-4-carboxamide
5-(4-fluorophenyl)-1-isopropyl-4-oxo-N-[4-[(6-piperazin-1-yl-1,7-naphthyridin-4-yl)oxy]phenyl]pyridine-3-carboxamide
5-(4-fluorophenyl)-1-methyl-4-oxo-N-[4-[(6-piperazin-1-yl-1,7-naphthyridin-4-yl)oxy]phenyl]pyridine-3-carboxamide
4-[4-[[3-(4-fluorophenyl)-2,4-dioxo-1-[rac-(1R)-2-hydroxy-1-methyl-ethyl]pyrimidine-5-carbonyl]amino]phenoxy]-N-(1-methyl-4-piperidyl)-1,7-naphthyridine-6-carboxamide
4-[4-[[1-cyclopropyl-3-(4-fluorophenyl)-2,4-dioxo-pyrimidine-5-carbonyl]amino]phenoxy]-N-(1-methyl-4-piperidyl)-1,7-naphthyridine-6-carboxamide
1-(4-fluorophenyl)-N-[5-[[6-(1-methyl-4-piperidyl)-1,7-naphthyridin-4-yl]oxy]-2-pyridyl]-2-oxo-pyridine-3-carboxamide
tert-butyl 3-[4-[[6-[[1-(4-fluorophenyl)-2-oxo-pyridine-3-carbonyl]amino]-3-pyridyl]oxy]-1,7-naphthyridin-6-yl]pyrrolidine-1-carboxylate
1-(4-fluorophenyl)-N-[5-[[6-[1-(oxetan-3-yl)-3,6-dihydro-2H-pyridin-4-yl]-1,7-naphthyridin-4-yl]oxy]-2-pyridyl]-2-oxo-pyridine-3-carboxamide
1-(4-fluorophenyl)-N-[5-[[6-[1-(oxetan-3-yl)-4-piperidyl]-1,7-naphthyridin-4-yl]oxy]-2-pyridyl]-2-oxo-pyridine-3-carboxamide
tert-butyl 4-[4-[[6-[[1-(4-fluorophenyl)-2-oxo-pyridine-3-carbonyl]amino]-3-pyridyl]oxy]-1,7-naphthyridin-6-yl]piperidine-1-carboxylate
1-(4-fluorophenyl)-N-[4-[[6-(1-methyl-4-piperidyl)-1,7-naphthyridin-4-yl]oxy]phenyl]-2-oxo-pyridine-3-carboxamide
1-(4-fluorophenyl)-N-[5-[[6-[(1-methylpyrrolidine-3-carbonyl)amino]-1,7-naphthyridin-4-yl]oxy]-2-pyridyl]-2-oxo-pyridine-3-carboxamide
1-(4-fluorophenyl)-N-[5-[[6-[(1-methylpiperidine-4-carbonyl)amino]-1,7-naphthyridin-4-yl]oxy]-2-pyridyl]-2-oxo-pyridine-3-carboxamide
4-[2-[[1-(4-fluorophenyl)-2-oxo-pyridine-3-carbonyl]amino]pyrimidin-5-yl]oxy-N-(1-methyl-4-piperidyl)-1,7-naphthyridine-6-carboxamide
2-(4-fluorophenyl)-N-[5-[[6-[4-(oxetan-3-yl)piperazin-1-yl]-1,7-naphthyridin-4-yl]oxy]-2-pyridyl]-3-oxo-pyridazine-4-carboxamide
3-(4-fluorophenyl)-1-isopropyl-N-[5-[[6-[4-(oxetan-3-yl)piperazin-1-yl]-1,7-naphthyridin-4-yl]oxy]-2-pyridyl]-2,4-dioxo-pyrimidine-5-carboxamide
5-(4-fluorophenyl)-1-isopropyl-N-[5-[[6-[4-(oxetan-3-yl)piperazin-1-yl]-1,7-naphthyridin-4-yl]oxy]-2-pyridyl]-4-oxo-pyridine-3-carboxamide
1-(4-fluorophenyl)-2-oxo-N-[5-[(6-pyrrolidin-3-yl-1,7-naphthyridin-4-yl)oxy]-2-pyridyl]pyridine-3-carboxamide
1-(4-fluorophenyl)-2-oxo-N-[5-[[6-(4-piperidyl)-1,7-naphthyridin-4-yl]oxy]-2-pyridyl]pyridine-3-carboxamide
1-(4-fluorophenyl)-N-[5-[[6-[1-(2-methylpropanoyl)-4-piperidyl]-1,7-naphthyridin-4-yl]oxy]-2-pyridyl]-2-oxo-pyridine-3-carboxamide
5-(4-fluorophenyl)-1-isopropyl-N-[5-[[6-(1-methyl-4-piperidyl)-1,7-naphthyridin-4-yl]oxy]-2-pyridyl]-4-oxo-pyridine-3-carboxamide
3-(4-fluorophenyl)-1-isopropyl-N-[5-[[6-(1-methyl-4-piperidyl)-1,7-naphthyridin-4-yl]oxy]-2-pyridyl]-2,4-dioxo-pyrimidine-5-carboxamide
1-(4-fluorophenyl)-N-[5-[[6-[(1-methyl-4-piperidyl)oxy]-1,7-naphthyridin-4-yl]oxy]-2-pyridyl]-2-oxo-pyridine-3-carboxamide
4-[[6-[[1-(4-fluorophenyl)-2-oxo-pyridine-3-carbonyl]amino]-4-methyl-3-pyridyl]oxy]-N-(1-methyl-4-piperidyl)-1,7-naphthyridine-6-carboxamide
4-[[6-[[1-(4-fluorophenyl)-2-oxo-pyridine-3-carbonyl]amino]-2-methyl-3-pyridyl]oxy]-N-(1-methyl-4-piperidyl)-1,7-naphthyridine-6-carboxamide
4-[[6-[[3-(4-fluorophenyl)-1-isopropyl-2,4-dioxo-pyrimidine-5-carbonyl]amino]-3-pyridyl]oxy]-N-(4-piperidyl)-1,7-naphthyridine-6-carboxamide tert-butyl 4-[[4-[[6-[[3-(4-fluorophenyl)-1-isopropyl-2,4-dioxo-pyrimidine-5-carbonyl]amino]-3-pyridyl]oxy]-1,7-naphthyridine-6-carbonyl]amino]piperidine-1-carboxylate
4-[[6-[[3-(4-fluorophenyl)-1-isopropyl-2,4-dioxo-pyrimidine-5-carbonyl]amino]-3-pyridyl]oxy]-N-(8-methyl-8-azabicyclo[3.2.1]octan-3-yl)-1,7-naphthyridine-6-carboxamide
N-[2-(dimethylamino)ethyl]-4-[[6-[[3-(4-fluorophenyl)-1-isopropyl-2,4-dioxo-pyrimidine-5-carbonyl]amino]-3-pyridyl]oxy]-1,7-naphthyridine-6-carboxamide
4-[[6-[[3-(4-fluorophenyl)-1-isopropyl-2,4-dioxo-pyrimidine-5-carbonyl]amino]-3-pyridyl]oxy]-N-[rac-(3S)-1-methylpyrrolidin-3-yl]-1,7-naphthyridine-6-carboxamide
4-[[6-[[3-(4-fluorophenyl)-1-isopropyl-2,4-dioxo-pyrimidine-5-carbonyl]amino]-3-pyridyl]oxy]-N-(1-methyl-4-piperidyl)-1,7-naphthyridine-6-carboxamide
4-[[6-[[3-(4-fluorophenyl)-1-isopropyl-2,4-dioxo-pyrimidine-5-carbonyl]amino]-3-pyridyl]oxy]-N-[rac-(3R)-1-methylpyrrolidin-3-yl]-1,7-naphthyridine-6-carboxamide
4-[[6-[[3-(4-fluorophenyl)-1-isopropyl-2,4-dioxo-pyrimidine-5-carbonyl]amino]-3-pyridyl]oxy]-N-[(4-methylmorpholin-2-yl)methyl]-1,7-naphthyridine-6-carboxamide
N-[3-(dimethylamino)cyclobutyl]-4-[[6-[[3-(4-fluorophenyl)-1-isopropyl-2,4-dioxo-pyrimidine-5-carbonyl]amino]-3-pyridyl]oxy]-1,7-naphthyridine-6-carboxamide
4-[[6-[[3-(4-fluorophenyl)-1-isopropyl-2,4-dioxo-pyrimidine-5-carbonyl]amino]-3-pyridyl]oxy]-N-methyl-N-(1-methyl-4-piperidyl)-1,7-naphthyridine-6-carboxamide
4-[[6-[[5-(4-fluorophenyl)-1-isopropyl-4-oxo-pyridine-3-carbonyl]amino]-3-pyridyl]oxy]-N-(1-methyl-4-piperidyl)-1,7-naphthyridine-6-carboxamide
4-[[6-[[1-(4-fluorophenyl)-5-isopropyl-2-oxo-pyridine-3-carbonyl]amino]-3-pyridyl]oxy]-N-(1-methyl-4-piperidyl)-1,7-naphthyridine-6-carboxamide
N-(1-methyl-4-piperidyl)-4-[[6-[(2-oxo-1-phenyl-pyridine-3-carbonyl)amino]-3-pyridyl]oxy]-1,7-naphthyridine-6-carboxamide
4-[[6-[[5-(5-fluoro-2-pyridyl)-1-isopropyl-4-oxo-pyridine-3-carbonyl]amino]-3-pyridyl]oxy]-N-(1-methyl-4-piperidyl)-1,7-naphthyridine-6-carboxamide
N-[3-methyl-4-[(6-piperazin-1-yl-1,7-naphthyridin-4-yl)oxy]phenyl]-2-oxo-1-phenyl-pyridine-3-carboxamide
N-[3-fluoro-4-[(6-piperazin-1-yl-1,7-naphthyridin-4-yl)oxy]phenyl]-2-oxo-1-phenyl-pyridine-3-carboxamide
2-oxo-1-phenyl-N-[5-[(6-piperazin-1-yl-1,7-naphthyridin-4-yl)oxy]-2-pyridyl]pyridine-3-carboxamide
4-[2-fluoro-4-[(2-oxo-1-phenyl-pyridine-3-carbonyl)amino]phenoxy]-N-(1-methyl-4-piperidyl)-1,7-naphthyridine-6-carboxamide
4-[2-methyl-4-[(2-oxo-1-phenyl-pyridine-3-carbonyl)amino]phenoxy]-N-(1-methyl-4-piperidyl)-1,7-naphthyridine-6-carboxamide
1-(4-fluorophenyl)-N-[4-[[6-(4-methylpiperazin-1-yl)-1,7-naphthyridin-4-yl]oxy]phenyl]-2-oxo-pyridine-3-carboxamide
1-cyclopropyl-3-(4-fluorophenyl)-2,4-dioxo-N-[4-[(6-piperazin-1-yl-1,7-naphthyridin-4-yl)oxy]phenyl]pyrimidine-5-carboxamide
4-[4-[[5-(4-fluorophenyl)-1-methyl-4-oxo-pyridine-3-carbonyl]amino]phenoxy]-N-(1-methyl-4-piperidyl)-1,7-naphthyridine-6-carboxamide
4-[4-[[5-(4-fluorophenyl)-1-isopropyl-4-oxo-pyridine-3-carbonyl]amino]phenoxy]-N-(1-methyl-4-piperidyl)-1,7-naphthyridine-6-carboxamide.

The various functional groups and substituents making up the compounds of the Formula (I) are typically chosen such that the molecular weight of the compound of the Formula (I) does not exceed 1000. More usually, the molecular weight of the compound will be less than 900, for example less than 800, or less than 750, or less than 700, or less than 650. More preferably, the molecular weight is less than 600 and, for example, is 550 or less.

A suitable pharmaceutically acceptable salt of a compound of the invention is, for example, an acid addition salt of a compound of the invention which is sufficiently basic, for example, an acid addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris(2hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are nonsuperimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R and S sequencing rules of Cahn and Prelog, or by the manner in which the molecule rotates the plane of polarized light and designated as dextrorotatory or levorotatory (i.e., as (+) or ( ) isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R) or (S)stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E and Z isomers).

It is to be understood that the present invention encompasses all optical, diastereoisomers and geometric isomers and mixtures thereof that possess antiproliferative activity.

The present invention also encompasses compounds of the invention as defined herein which comprise one or more isotopic substitutions. For example, H may be in any isotopic form, including 1H, 2H(D), and 3H (T); C may be in any isotopic form, including 12C, 13C, and 14C; and O may be in any isotopic form, including 16O and 18O; and the like.

It is also to be understood that certain compounds of the Formula (I), or sub-formulae or sub-formulae Ia to Ic, may exist in solvated as well as unsolvated forms such as, for example, hydrated forms. It is to be understood that the invention encompasses all such solvated forms that possess antiproliferative activity.

It is also to be understood that certain compounds of the Formula (I), or sub-formulae Ia to Ic, may exhibit polymorphism, and that the invention encompasses all such forms that possess antiproliferative activity.

Compounds of the Formula (I), or sub-formulae Ia to Ic, may exist in a number of different tautomeric forms and references to compounds of the Formula (I), or sub-formulae Ia to Ic, include all such forms. For the avoidance of doubt, where a compound can exist in one of several tautomeric forms, and only one is specifically described or shown, all others are nevertheless embraced by Formula (I), or sub-formulae Ia to Ic. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

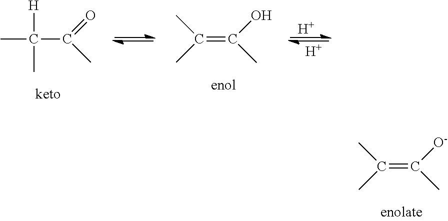

Compounds of the Formula (I), or sub-formulae Ia to Ic, containing an amine function may also form N-oxides. A reference herein to a compound of the Formula (I), or sub-formulae Ia to Ic, that contains an amine function also includes the N-oxide. Where a compound contains several amine functions, one or more than one nitrogen atom may be oxidised to form an N-oxide. Particular examples of N-oxides are the N-oxides of a tertiary amine, a nitrogen atom of a nitrogen-containing heterocycle, or a nitrogen atom of a nitrogen-containing heteroaryl (e.g. N-oxides of pyridine). N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of Formula (I), or sub-formulae Ia to Ic, may be administered in the form of a prodrug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula (I), or sub-formulae Ia to Ic, and in-vivo cleavable amide derivatives that may be formed at a carboxy group or an amino group in a compound of the Formula (I), or sub-formulae Ia to Ic.

Accordingly, the present invention includes those compounds of the Formula (I), or sub-formulae Ia to Ic, as defined hereinbefore, when made available by organic synthesis and when made available within the human or animal body by way of cleavage of a pro-drug thereof. Accordingly, the present invention includes those compounds of the Formula (I), or sub-formulae Ia to Ic, that are produced by organic synthetic means and also such compounds that are produced in the human or animal body by way of metabolism of a precursor compound, that is a compound of the Formula (I), or sub-formulae Ia to Ic, may be a synthetically-produced compound or a metabolically-produced compound.

A suitable pharmaceutically acceptable pro-drug of a compound of the Formula (I), or sub-formulae Ia to Ic, is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the Formula (I), or sub-formulae Ia to Ic, that possesses a carboxy group is, for example, an in vivo cleavable ester thereof. An in vivo cleavable ester of a compound of the Formula I, or sub-formulae Ia to Im, containing a carboxy group is, for example, a pharmaceutically acceptable ester which is cleaved in the human or animal body to produce the parent acid. Suitable pharmaceutically acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically acceptable pro-drug of a compound of the Formula (I), or sub-formulae Ia to Ic, that possesses a hydroxy group is, for example, an in vivo cleavable ester or ether thereof. An in vivo cleavable ester or ether of a compound of the Formula (I), or sub-formulae Ia to Ic, containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-(1-6C)₂carbamoyl, 2-dialkylaminoacetyl and 2-carboxyacetyl groups.

Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the Formula (I), or sub-formulae Ia to Ic, that possesses a carboxy group is, for example, an in vivo cleavable amide thereof, for example an amide formed with an amine such as ammonia, a (1-4C)alkylamine such as methylamine, a [(1-4C)alkyl]₂amine such as dimethylamine, N-ethylN-methylamine or diethylamine, a (1-4C)alkoxy(2-4C)alkylamine such as 2methoxyethylamine, a phenyl(1-4C)alkylamine such as benzylamine and amino acids such as glycine or an ester thereof.

A suitable pharmaceutically acceptable pro-drug of a compound of the Formula (I), or sub-formulae Ia to Ic, that possesses an amino group is, for example, an in vivo cleavable amide derivative thereof. Suitable pharmaceutically acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkyl)piperazin-1-ylmethyl.

The in vivo effects of a compound of the Formula (I), or sub-formulae Ia to Ic, may be exerted in part by one or more metabolites that are formed within the human or animal body after administration of a compound of the Formula (I), or sub-formulae Ia to Ic. As stated hereinbefore, the in vivo effects of a compound of the Formula (I), or sub-formulae Ia to Ic, may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladiumoncarbon.

A suitable protecting group for a carboxy group is, for example, an esterifying group, for example a methyl or an ethyl group which may be removed, for example, by hydrolysis with a base such as sodium hydroxide, or for example a tbutyl group which may be removed, for example, by treatment with an acid, for example an organic acid such as trifluoroacetic acid, or for example a benzyl group which may be removed, for example, by hydrogenation over a catalyst such as palladiumoncarbon.

Resins may also be used as a protecting group.

The methodology employed to synthesise a compound of Formula (I), or sub-formulae Ia to Ic, will vary depending on the nature of R₁, X₁, etc. . . . , and any substituent groups associated therewith. Suitable processes for their preparation are described further in the accompanying Examples.

Once a compound of Formula (I), or sub-formulae Ia to Ic, has been synthesised by any one of the processes defined herein, the processes may then further comprise the additional steps of:
(i) removing any protecting groups present;
(ii) converting the compound Formula (I) into another compound of Formula (I);
(iii) forming a pharmaceutically acceptable salt or solvate thereof; and/or
(iv) forming a prodrug thereof.

An example of (ii) above is when a compound of Formula (I) is synthesised and then one or more of the groups R₁, X₁, etc. . . . , may be further reacted to change the nature of the group and provide an alternative compound of Formula (I).

The resultant compounds of Formula (I), or sub-formulae Ia to Ic, can be isolated and purified using techniques well known in the art.

The compounds of Formula (I) may be synthesised by the general synthetic routes shown in the Schemes illustrated in the Examples section below, specific examples of which are described in more detail in the Examples.

### Biological Activity

The biological assays described in the Examples section herein may be used to measure the pharmacological effects of the compounds of the present invention.

Although the pharmacological properties of the compounds of Formula (I) vary with structural change, as expected, the compounds of the invention were found to be active in the MerTK and AXL in vitro assays described in the Examples section.

### Pharmaceutical Compositions

According to a further aspect of the invention there is provided a pharmaceutical composition which comprises a compound of the invention as defined hereinbefore, or a pharmaceutically acceptable salt or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that function as inhibitors of MerTK and/or AXL activity.

The present invention therefore provides a method of inhibiting MerTK and/or AXL activity in vitro or in vivo, said method comprising contacting a cell with an effective amount of a compound, or a pharmaceutically acceptable salt or solvate thereof, as defined herein.

The present invention also provides a method of treating a disease or disorder in which MerTK and/or AXL activity is implicated in a patient in need of such treatment, said method comprising administering to said patient a therapeutically effective amount of a compound, or a pharmaceutically acceptable salt or solvate thereof, or a pharmaceutical composition as defined herein.

Conditions in which MerTK and/or AXL activity is implicated include proliferative disorders, including cancer, as well as infections, immune modulation and/or stimulation, disorders associated with platelet aggregation (e.g. thrombosis) and liver diseases, including fibrosis and steatohepatitis (e.g. non-alcoholic steatohepatitis).

The present invention provides a method of inhibiting cell proliferation in vitro or in vivo, said method comprising contacting a cell with an effective amount of a compound, or a pharmaceutically acceptable salt or solvate thereof, as defined herein.

The present invention provides a method of treating a proliferative disorder in a patient in need of such treatment, said method comprising administering to said patient a therapeutically effective amount of a compound, or a pharmaceutically acceptable salt or solvate thereof, or a pharmaceutical composition as defined herein.

The present invention provides a method of treating cancer in a patient in need of such treatment, said method comprising administering to said patient a therapeutically effective amount of a compound, or a pharmaceutically acceptable salt or solvate thereof, or a pharmaceutical composition as defined herein.

The present invention provides a compound, or a pharmaceutically acceptable salt or solvate thereof, or a pharmaceutical composition as defined herein for use in therapy.

The present invention provides a compound, or a pharmaceutically acceptable salt or solvate thereof, or a pharmaceutical composition as defined herein for use in the treatment of a proliferative condition.

The present invention provides a compound, or a pharmaceutically acceptable salt or solvate thereof, or a pharmaceutical composition as defined herein for use in the treatment of cancer. In a particular embodiment, the cancer is human cancer.

The present invention provides a compound, or a pharmaceutically acceptable salt or solvate thereof, as defined herein, for use in the inhibition of MerTK and/or AXL activity.

The present invention provides a compound, or a pharmaceutically acceptable salt or solvate thereof, as defined herein for use in the treatment of a disease or disorder in which MerTK and/or AXL activity is implicated. Preferably, the present invention provides a compound, or a pharmaceutically acceptable salt or solvate thereof, as defined herein for use in the treatment of a disease or disorder in which MerTK and/or AXL activity is implicated.

The present invention provides a use of a compound, or a pharmaceutically acceptable salt or solvate thereof, as defined herein in the manufacture of a medicament for the treatment of a proliferative condition.

The present invention provides a use of a compound, or a pharmaceutically acceptable salt or solvate thereof, as defined herein in the manufacture of a medicament for the treatment of cancer. Suitably, the medicament is for use in the treatment of human cancers.

The present invention provides a use of a compound, or a pharmaceutically acceptable salt or solvate thereof, as defined herein in the manufacture of a medicament for the inhibition of MerTK and/or AXL activity.

The present invention provides a use of a compound, or a pharmaceutically acceptable salt or solvate thereof, as defined herein in the manufacture of a medicament for the treatment of a disease or disorder in which MerTK activity is implicated.

The term “proliferative disorder” and “proliferative condition” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers (including breast cancer (including triple negative breast cancer), non-small cell lung cancer (NSCLC) and squamous cell carcinomas (SCC) (including SCC of the head and neck, oesophagus, lung and ovary), leukemias (including acute lymphoblastic leukaemia (ALL) and chronic myeloid leukaemia (CML)), lymphomas (including mantle cell lymphoma), melanomas etc.), psoriasis, bone diseases, fibroproliferative disorders (e.g., of connective tissues), and atherosclerosis. Any type of cell may be treated, including but not limited to, lymphatic, blood, lung, colon, breast, ovarian, prostate, liver, pancreas, brain, and skin.

MerTK and AXL are associated with multiple cancer cell types. Particular cancers of note are:
mantle cell lymphoma (Shi et al. 2018);
non-small cell lung cancer (Cummings et al. 2015);
tumours that have undergone epithelial to mesenchymal transition (EMT), e.g. non-small cell lung cancer (Byers et al. 2013); and
head and neck squamous cell carcinoma (Brand et al. 2015).

The anti-cancer effect may arise through one or more mechanisms, including but not limited to, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures), tumour migration, the promotion of apoptosis (programmed cell death) and/or modulation of immune cell function.

Without wishing to be bound by any particular theory, there is evidence that, in addition to the direct control of cancer cell signalling, the TAM kinases can exert their effects on tumour growth and maintenance via modulation of immune cell function. MerTK is expressed in tumour associated macrophages and activation of the kinase by apoptotic material creates an immunosuppressive microenvironment, a reduction of inflammatory cytokines such as IL-12 and interferon gamma and the creation of an autocrine activation loop via the increase of GAS6. Growth of syngeneic breast and melanoma tumours in mertk^{-/-} mice is impaired (Cook et al. 2013). MerTK is implicated in the control of efferocytosis, the mechanism by which apoptotic cells are cleared by phagocytic cells leading to a reduced tumour cell response by immune cells and increased likelihood of tumour residual disease (Werfel et al. 2019). In vivo validation of a role for MerTK in tumour efferocytosis has been established using anti-MerTK antibodies (Zhou et al. 2020). Activation of TAM kinases by PROS1 and GAS6 also suppresses the activation of dendritic cells and subsequent reduction in cytokines. The Pan-TAM kinase inhibitor BMS-777607 has been tested in combination with an anti-PD-1 monoclonal antibody in a model of triple-negative breast cancer (Kasikara et al. 2019). The combination of the two therapeutic agents significantly decreased tumour growth and lung metastasis. This was associated with an increase in the anti-tumour T lymphocytes.

The compound of Formula (I), or a pharmaceutically acceptable salt thereof, being an inhibitor of MerTK and/or AXL, has potential therapeutic uses in a variety of MerTK and/or AXL-mediated disease states.

In particular, the compounds of the present invention may also be used as anti-infective agents, immunostimulatory agents or immunomodulatory agents, anti-inflammatory agents, anti-thrombotic agents, anti-fibrotic agents and treatments for neurodegeneration.

The present invention further provides a method of producing an immunomodulatory or immunostimulatory effect, or treating an infection, inflammation, a platelet aggregation disorder (e.g. thrombosis), liver disease (e.g. fibrosis and steatohepatitis, e.g. non-alcoholic steatohepatitis) or neurodegenration, in a patient in need of such treatment, said method comprising administering to said patient a therapeutically effective amount of a compound, or a pharmaceutically acceptable salt or solvate thereof, or a pharmaceutical composition as defined herein

The present invention further provides a compound, or a pharmaceutically acceptable salt or solvate thereof, or a pharmaceutical composition as defined herein for use in producing an immunomodulatory or immunostimulatory effect, or the treatment of an infection, inflammation, a platelet aggregation disorder (e.g. thrombosis), liver disease (e.g. fibrosis and steatohepatitis, e.g. non-alcoholic steatohepatitis) or neurodegeneration.

The present invention further provides the use of a compound, or a pharmaceutically acceptable salt or solvate thereof, or a pharmaceutical composition as defined herein, in the manufacture of a medicament for use in producing an immunomodulatory or immunostimulatory effect, or the treatment of an infection, inflammation, a platelet aggregation disorder (e.g. thrombosis), liver disease (e.g. fibrosis and steatohepatitis, e.g. non-alcoholic steatohepatitis) or neurodegeneration.

### Routes of Administration

The compounds of the invention or pharmaceutical compositions comprising these compounds may be administered to a subject by any convenient route of administration, whether systemically, peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The antiproliferative treatment defined hereinbefore may be applied as a sole therapy or may involve, in addition to the compound of the invention, conventional surgery or radiotherapy or chemotherapy. Such chemotherapy may include one or more of the following categories of anti-tumour agents:—
(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), steroid hormones, including progestogens (for example megestrol acetate) and corticosteroids (for example dexamethasone, prednisone and prednisolone), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt or solvate thereof, and another anti-tumour agent.

According to this aspect of the invention there is provided a combination for use in the treatment of a proliferative condition, such as cancer (for example a cancer involving a solid tumour), comprising a compound of the invention as defined hereinbefore, or a pharmaceutically acceptable salt or solvate thereof, and any one of the anti-tumour agents listed herein above.

In a further aspect of the invention there is provided a compound of the invention or a pharmaceutically acceptable salt or solvate thereof, for use in the treatment of cancer in combination with another anti-tumour agent, optionally selected from one listed herein above.

In a further aspect of the invention there is provided a compound of the invention or a pharmaceutically acceptable salt or solvate thereof, for use in the treatment of cancer in combination with a tyrosine kinase inhibitor, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt or solvate thereof, in combination with an anti-tumour agent (optionally selected from one listed herein above), in association with a pharmaceutically acceptable diluent or carrier.

Combination Therapy with Immune Checkpoint Inhibitors

Immune checkpoint proteins present on immune cells and/or cancer cells [e.g. CTLA4 (also known as cytotoxic T-lymphocyte-associated protein 4 and CD152), LAG3 (also known as lymphocyte-activation gene 3 and CD223), PD1 (also known as programmed cell death protein 1 and CD279) PD-L1 (also known as programmed death-ligand 1 and CD274), TIM-3 (also known as T-cell immunoglobulin mucin-3) and TIGIT (also known as T-cell Immunoreceptor with Ig and ITIM domains) are molecular targets that have been found to play an important role in regulating anti-tumour immune responses. Inhibitors of these immune checkpoint proteins (e.g. CTLA4, LAG3, PD1, PD-L1, TIM-3 and/or TIGIT inhibitors) promote an anti-tumour immune response that can be utilised to effectively treat certain forms of cancer.

In one aspect, the present invention relates to a combination comprising a compound as defined herein, or a pharmaceutically acceptable salt thereof, and an immune checkpoint inhibitor as defined herein, or a pharmaceutically acceptable salt thereof, for use in the treatment of a proliferative disorder.

In another aspect, the present invention relates to a use of a combination comprising a compound as defined herein, or a pharmaceutically acceptable salt thereof, and an immune checkpoint inhibitor as defined herein, or a pharmaceutically acceptable salt thereof, in the manufacture of a medicament for treating of a proliferative disorder.

In another aspect, the present invention relates to a method of treating of a proliferative disorder in a subject in need thereof comprising administering to said subject a combination comprising a compound as defined herein, or a pharmaceutically acceptable salt thereof, and an immune checkpoint inhibitor, or a pharmaceutically acceptable salt thereof, as defined herein.

In another aspect, the present invention relates to a compound as defined herein, or a pharmaceutically acceptable salt thereof, as defined herein for use in the treatment of a proliferative disorder, wherein the compound, or a pharmaceutically acceptable salt thereof, is for simultaneous, separate or sequential administeration with an immune checkpoint inhibitor, or a pharmaceutically acceptable salt thereof.

In another aspect, the present invention relates to an immune checkpoint inhibitor, or a pharmaceutically acceptable salt thereof, for use in the treatment of a proliferative disorder, wherein the immune checkpoint inhibitor is for simultaneous, separate or sequential administeration with a compound as defined herein, or a pharmaceutically acceptable salt thereof, as defined herein.

In another aspect, the present invention relates to a use of a compound as defined herein, or a pharmaceutically acceptable salt thereof, as defined herein in the manufacture of a medicament for treating a proliferative disorder, wherein the medicament is for simultaneous, separate or sequential administeration with an immune checkpoint inhibitor, or a pharmaceutically acceptable salt thereof.

In another aspect, the present invention relates to a use of an immune checkpoint inhibitor, or a pharmaceutically acceptable salt thereof, in the manufacture of a medicament for treating a proliferative disorder, wherein the medicament is for simultaneous, separate or sequential administeration with a compound as defined herein, or a pharmaceutically acceptable salt thereof.

In another aspect, the present invention relates to a method of treating a proliferative disorder comprising administering to a subject in need thereof a therapeutically effective amount of a compound as defined herein, or a pharmaceutically acceptable salt thereof, as defined herein and an immune checkpoint inhibitor, or a pharmaceutically acceptable salt thereof, either sequentially, separately or simultaneously

Any immune checkpoint inhibitor may be used in the combination therapy defined herein.

In one embodiment, the immune checkpoint inhibitor is selected from a PD1, a PD-L1 inhibitor, a LAG3 inhibitor, aCTLA-4 inhibitor, a TIM-3 inhibitor and/or a TIGIT inhibitor. In a particular embodiment, the immune checkpoint inhibitor is a PD1 or PD-L1 inhibitor.

PD-1 is a cell surface receptor protein present on T cells. PD-1 plays an important role in down-regulating the immune system and promoting self-tolerance by suppressing T cell inflammatory activity. The PD-1 protein is an immune checkpoint that guards against autoimmunity through a dual mechanism of promoting apoptosis (programmed cell death) in antigen specific T cells in lymph nodes, while simultaneously reducing apoptosis in regulatory T cells (anti-inflammatory suppressive T cells).

PD-1 therefore inhibits the immune system. This prevents autoimmune diseases, but it can also prevent the immune system from killing cancer cells.

PD1 binds two ligands, PD-L1 and PD-L2. PD-L1 is of particular interest as it is highly expressed in several cancers and hence the role of PD1 in cancer immune evasion is well established. Monoclonal antibodies targeting PD-1 that boost the immune system are being developed for the treatment of cancer. Many tumour cells express PD-L1, an immunosuppressive PD-1 ligand; inhibition of the interaction between PD-1 and PD-L1 can enhance T-cell responses in vitro and mediate preclinical antitumour activity. This is known as immune checkpoint blockade.

Examples of drugs that target PD-1 include pembrolizumab (Keytruda) and nivolumab (Opdivo). These drugs have been shown to be effective in treating several types of cancer, including melanoma of the skin, non-small cell lung cancer, kidney cancer, bladder cancer, head and neck cancers, and Hodgkin lymphoma. They are also being studied for use against many other types of cancer. Examples of drugs in development include BMS-936559 (Bristol Myers Squibb), MGA012 (MacroGenics) and MEDI-0680 (MedImmune).

Examples of drugs that inhibit PD-L1 include atezolizumab (Tecentriq), avelumab (Bavencio) and durvalumab (Imfinzi). These drugs have also been shown to be helpful in treating different types of cancer, including bladder cancer, non-small cell lung cancer, and Merkel cell skin cancer (Merkel cell carcinoma). They are also being studied for use against other types of cancer.

Examples of LAG3 inhibitors include BMS-986016/Relatlimab, TSR-033, REGN3767, MGD013 (bispecific DART binding PD-1 and LAG-3), GSK2831781 and LAG525.

Examples of CTLA-4 inhibitors include MDX-010/Ipilimumab, AGEN1884, and CP-675,206/Tremelimumab.

Examples of TIM-3 inhibitors include MBG453 (Novartis), TSR-022 (Tesaro), and LY3321367 (Lilly).

Examples of TIGIT inhibitors include Tiragolumab (MTIG7192A; RG6058; Genentech/Roche), AB154 (Arcus Bioscience), MK-7684 (Merck), BMS-986207 (Bristol-Myers Squibb), ASP8374 (Astellas Pharma; Potenza Therapeutics).

In one embodiment, the immune checkpoint inhibitor is selected from BMS-986016/Relatlimab, TSR-033, REGN3767, MGD013 (bispecific DART binding PD-1 and LAG-3), GSK2831781, LAG525, MDX-010/Ipilimumab, AGEN1884, and CP-675,206/Tremelimumab, pembrolizumab, nivolumab, atezolizumab, avelumab, durvalumab, MBG453, TSR-022, LY3321367, Tiragolumab (MTIG7192A; RG6058), AB154, MK-7684, BMS-986207, and/or ASP8374 or a pharmaceutically acceptable salt or solvate thereof.

### EXAMPLES

### General Conditions:

Mass spectra were run on LC-MS systems using electrospray ionization. These were run using either a Waters Acquity uPLC system with Waters PDA and ELS detectors or a Shimadzu LCMS-2010EV system. [M+H]+ refers to mono-isotopic molecular weights.

NMR spectra were run on either a Bruker Avance III HD 400 MHz NMR spectrometer or a Bruker Avance III HD 500 MHz. Spectra were recorded at 298K and were referenced using the solvent peak.

The following examples are intended to illustrate the invention and are not to be construed as being limitations thereon. Temperatures are given in degrees centigrade. If not mentioned otherwise, all evaporations are performed in vacuo, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, and NMR. Abbreviations used are those conventional in the art. If not defined, the terms have their generally accepted meanings.

### Abbreviations

app apparent
br broad
d doublet
dd doublet of doublets
DCM dichloromethane
DIAD diisopropyl azodicarboxylate
DIBAL Diisobutylaluminum hydride
DIEA diethylisopropylamine
DIPEA N,N-diisopropylethylamine
DMA N,N-dimethylacetamide
DMAP N,N-dimethylpyridin-4-amine
DMF N,N-dimethylformamide
EDC 1-(3-Dimethylaminopropyl)-3-ethylcarbodiimide hydrochloride
EtOAc ethyl acetate
HATU Hexafluorophosphate Azabenzotriazole Tetramethyl Uronium
HPLC high pressure liquid chromatography
IPA iso-propanol
LC-MS liquid chromatography and mass spectrometry
MeOH MeOH
MeCN acetonitrile
MS mass spectrometry
m multiplet
mins minute(s)
mL millilitre(s)
m/z mass to charge ratio
NMR nuclear magnetic resonance
ppm parts per million
rac- racemate
Rt retention time
s singlet
t triplet
TFA trifluoroacetic acid
THF tetrahydrofuran

Referring to the examples that follow, compounds of the preferred embodiments were synthesized using the methods described herein, or other methods, which are known in the art.

The various starting materials, intermediates, and compounds of the preferred embodiments may be isolated and purified, where appropriate, using conventional techniques such as precipitation, filtration, crystallization, evaporation, distillation, and chromatography. Unless otherwise stated, all starting materials are obtained from commercial suppliers and used without further purification. Salts may be prepared from compounds by known salt-forming procedures.

If not indicated otherwise, the analytical HPLC conditions are as follows:

Method 2A

| | |
|---|---|
| Column: | Kinetex Core-Shell C18 2.1 × 50 mm 5 μm |
| Column Temp | 40° C. |
| Eluents: | A: H20 + 0.1% formic acid, |
| | B: MeCN + 0.1% formic acid |
| Flow Rate: | 1.2 mL/min |
| Gradient: | 0-1.20 mins 5-100% B, 1.20-1.30 mins 100% B, |
| | 1.30-1.31 mins 100-5% B |

Method 1A

| | |
|---|---|
| Column: | Waters UPLC ® BEHTM C18 2.1 × 50 mm 1.7 μm |
| Column Temp | 40° C. |
| Eluents: | A: H20 + 0.1% formic acid, |
| | B: MeCN + 0.1% formic acid |
| Flow Rate: | 0.9 mL/min |
| Gradient: | 0-1.10 mins 5-100% B, 1.10-1.35 mins 100% B, |
| | 1.35-1.40 mins 100-5% B, 1.40-1.50mins 5% B |

Method 1B

| | |
|---|---|
| Column: | Waters BEH C18 30 × 2.1 mm, 1.7 μm |
| Column Temp | 40° C. |
| Eluents: | A: 2 mM ammonium bicarbonate, |
| | buffered to pH 10, B: MeCN |
| Flow Rate: | 1 mL/min |
| Gradient: | 0-0.75 mins 5-100% B, 0.75-0.85 mins 100% B, |
| | 0.85-0.90 mins 100-5% B, 0.90-1.0 mins 100% B |

Method 2.5B

| | |
|---|---|
| Column: | Phenomenex Gemini-NX C18 2 × 50 mm 3 μm |
| Column Temp | 40° C. |
| Eluents: | A: 2 mM ammonium bicarbonate, |
| | buffered to pH 10, B: MeCN |
| Flow Rate: | 1 mL/min |
| Gradient: | 0-1.80 mins 1-100% B, 1.80-2.10 mins 100% B, |
| | 2.10-2.30 mins 100-1% B |

Method 3A

| | |
|---|---|
| Column: | Kinetex Core-Shell C8 Part No. |
| | 00B-4608-AN 2.1 × 50 mm, 5 μm |
| Column Temp | 40° C. |
| Eluents: | A: H20 + 0.1% formic acid, |
| | B: MeCN + 0.1% formic acid |
| Flow Rate: | 1.2 mL/min |
| Gradient: | 0-1.83 mins 5-100% B, 1.83-2.25 mins 100% B, |
| | 2.25-2.26 mins 100-5% B |

Method 7A

| | |
|---|---|
| Column: | Phenomenex Kinetix-XB C18 2.1 × 100 mm, 1.7 μm |
| Column Temp | 40° C. |
| Eluents: | A: H20 0.1% formic acid, B: MeCN, 0.1% formic acid |
| Flow Rate: | 0.6 mL/min |
| Gradient: | 0-5.3 mins 5-100% B, 5.3-5.8 mins 100% B, |
| | 5.8-5.82 mins 100-5% B, 5.82-7.00 mins 5% B |

Method 7B

| | |
|---|---|
| Column: | Waters UPLC ® BEHTM C18, |
| | 2.1 mm × 100 mm, 1.7 μm column |
| Column Temp: | 40° C. |
| Eluents: | A: 2 mM amm. bicarbonate buffered to pH 10, B: MeCN |
| Flow Rate: | 0.6 mL/min |
| Gradient: | 0-5.3 mins 5-100% B, 5.3-5.8 mins 100% B, |
| | 5.8-5.82 mins 100-5% B, 5.82-7.00 mins 5% B |

### Preparative HPLC Using Acidic pH, Early Elution Method

Purifications by preparative HPLC (acidic pH, early elution method) were performed on a Gilson LC system using Waters Sunfire C18 columns (30 mm×100 mm, 10 μM; temperature: RT) and a gradient of 10-95% B (A=0.1% formic acid in water; B=0.1% formic acid in MeCN) over 14.44 mins then 95% B for 2.11 min, with an injection volume of 1500 μL and a flow rate of 40 mL/mins. UV spectra were recorded at 215 nm using a Gilson detector.

### Preparative HPLC Using Acidic pH, Standard Elution Method

Purifications by preparative HPLC (acidic pH, standard elution method) were performed on a Gilson LC system using Waters Sunfire C18 columns (30 mm×100 mm, 10 μM; temperature: RT) and a gradient of 30-95% B (A=0.1% formic acid in water; B=0.1% formic acid in MeCN) over 11 mins then 95% B for 2.11 min, with an injection volume of 1500 μL and a flow rate of 40 mL/mins. UV spectra were recorded at 215 nm using a Gilson detector.

### Preparative HPLC Using Basic pH, Early Elution Method

Purifications by preparative HPLC (basic pH, early elution method) were performed on a Gilson LC system using Waters Xbridge C18 columns (30 mm×100 mm, 10 μM; temperature: RT) and a gradient of 10-95% (A=0.2% ammonium hydroxide in water; B=0.2% ammonium hydroxide in MeCN) over 14.44 mins then 95% B for 2.11 min, with an injection volume of 1500 μL and a flow rate of 40 mL/mins. UV spectra were recorded at 215 nm using a Gilson detector.

### Preparative HPLC Using Basic pH, Standard Elution Method

Purifications by preparative HPLC (basic pH, standard elution method) were performed on a Gilson LC system using Waters Xbridge C18 columns (30 mm×100 mm, 10 μM; temperature: RT) and a gradient of 30-95% (A=0.2% ammonium hydroxide in water; B=0.2% ammonium hydroxide in MeCN) over 11 mins then 95% B for 2.11 min, with an injection volume of 1500 μL and a flow rate of 40 mL/mins. UV spectra were recorded at 215 nm using a Gilson detector.

### Example 1

### 2-oxo-1-phenyl-N-[4-[(6-piperazin-1-yl-1,7-naphthyridin-4-yl)amino]phenyl]pyridine-3-carboxamide

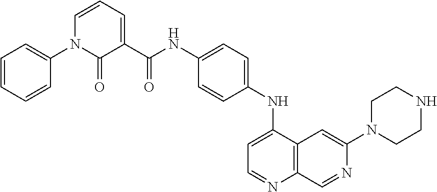 [SMILES: O=C(Nc1ccc(Nc2ccnc3cnc(N4CCNCC4)cc23)cc1)c1cccn(-c2ccccc2)c1=O]

### Step 1: 6-chloro-N-(4-nitrophenyl)-1,7-naphthyridin-4-amine

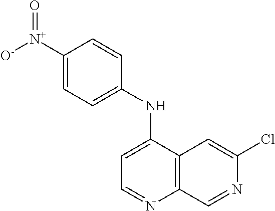 [SMILES: O=[N+]([O-])c1ccc(Nc2ccnc3cnc(Cl)cc23)cc1]

A suspension of 4,6-dichloro-1,7-naphthyridine (200 mg, 1.00 mmol) and 4-nitroaniline (153 mg, 1.11 mmol) in IPA (2 mL) was stirred at 60° C. for 18 h. The reaction was cooled to room temperature and the formed solid was collected by filtration, washed with IPA (3×3 ml) and dried in vacuo at 40° C. to afford 6-chloro-N-(4-nitrophenyl)-1,7-naphthyridin-4-amine (283 mg, 0.780 mmol, 78% Yield) as a yellow solid.

LC-MS (Method 2A): Rt 0.97 mins; MS m/z 300.9=[M+H]+

¹H NMR (400 MHz, DMSO-d6) δ 10.93-10.43 (m, 1H), 9.29 (d, J=8.0 Hz, 1H), 8.81 (d, J=6.0 Hz, 1H), 8.79-8.68 (m, 1H), 8.40-8.30 (m, 2H), 7.74-7.63 (m, 2H), 7.54-7.41 (m, 1H).

### Step 2: N-(4-nitrophenyl)-6-piperazin-1-yl-1,7-naphthyridin-4-amine

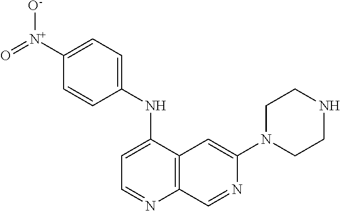 [SMILES: O=[N+]([O-])c1ccc(Nc2ccnc3cnc(N4CCNCC4)cc23)cc1]

6-chloro-N-(4-nitrophenyl)-1,7-naphthyridin-4-amine (Example 1, step 1) (282 mg, 0.938 mmol) and piperazine (1.00 g, 11.6 mmol) were stirred at 140° C. for 2.5 h, in a foil wrapped sealed reaction vial. The black reaction mixture was cooled to 80° C. and water (20 ml) was added. The resultant yellow/brown suspension was filtered with the collected solid washed with water (3 ml) and MeCN (1 ml) before being dried in vacuo at 40° C. to afford N-(4-nitrophenyl)-6-piperazin-1-yl-1,7-naphthyridin-4-amine (186 mg) as a brown solid.

LC-MS (Method 2A): Rt 0.72 mins; MS m/z 351.0=[M+H]+

¹H NMR (400 MHz, DMSO-d6) δ 9.40 (s, 1H), 8.99 (s, 1H), 8.46 (d, J=4.9 Hz, 1H), 8.23 (d, J=9.0 Hz, 2H), 7.47 (d, J=9.0 Hz, 2H), 7.37 (d, J=4.9 Hz, 1H), 7.18 (s, 1H), 3.60-3.46 (m, 4H), 2.90-2.80 (m, 4H)

### Step 3: tert-butyl 4-[4-(4-nitroanilino)-1,7-naphthyridin-6-yl]piperazine-1-carboxylate

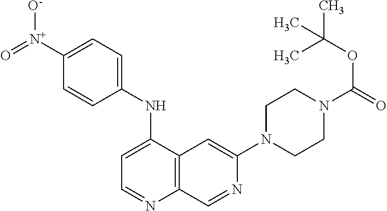 [SMILES: CC(C)(C)OC(=O)N1CCN(c2cc3c(Nc4ccc([N+](=O)[O-])cc4)ccnc3cn2)CC1]

A solution of N-(4-nitrophenyl)-6-piperazin-1-yl-1,7-naphthyridin-4-amine (Example 1, step 2) (184 mg, 0.525 mmol), tert-butoxycarbonyl tert-butyl carbonate (126 mg, 0.578 mmol) and DIPEA (0.14 mL, 0.788 mmol) in DCM (5 mL) was stirred at room temperature for 1 h. The reaction mixture was partitioned between DCM (10 mL) and saturated NaHCO₃ solution (10 mL). The organic layer was separated, washed with water (10 mL), dried over Na₂SO₄ and concentrated in vacuo. The crude material was purified by silica chromatography (Biotage KP-NH silica eluting with 0-100% EtOAc in heptane) to afford tert-butyl 4-[4-(4-nitroanilino)-1,7-naphthyridin-6-yl]piperazine-1-carboxylate (148 mg) as a yellow solid.

LC-MS (Method 2A): Rt 1.03 mins; MS m/z 451.7=[M+H]+

¹H NMR (500 MHz, DMSO-d6) δ 9.42 (s, 1H), 9.02 (s, 1H), 8.49 (d, J=4.9 Hz, 1H), 8.34-8.11 (m, 2H), 7.51-7.44 (m, 2H), 7.40 (d, J=4.9 Hz, 1H), 7.27 (s, 1H), 3.64-3.58 (m, 4H), 3.55-3.46 (m, 4H), 1.44 (s, 9H).

### Step 4: tert-butyl 4-[4-(4-aminoanilino)-1,7-naphthyridin-6-yl]piperazine-1-carboxylate

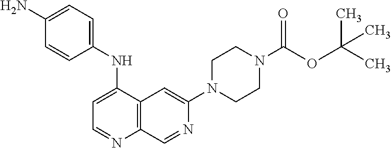 [SMILES: CC(C)(C)OC(=O)N1CCN(c2cc3c(Nc4ccc(N)cc4)ccnc3cn2)CC1]

To a solution of tert-butyl 4-[4-(4-nitroanilino)-1,7-naphthyridin-6-yl]piperazine-1-carboxylate (Example 1, step 3) (145 mg, 0.290 mmol) in Ethanol (1.5 mL) and water (0.5 mL) was added iron powder (49 mg, 0.869 mmol) and calcium chloride hydrate (70 mg, 0.319 mmol). The resulting mixture was stirred at 100° C. for 1 h. The reaction mixture was allowed to cool to room temperature before being filtered through a pad of Celite with the filter bed washed with MeOH (20 mL). The filtrate was partially concentrated in vacuo to remove the organic solvents with the remaining aqueous solution partitioned between EtOAc (10 mL) and saturated NaHCO₃ (10 mL). The organic layer was collected and concentrated in vacuo to afford tert-butyl 4-[4-(4-aminoanilino)-1,7-naphthyridin-6-yl]piperazine-1-carboxylate (100 mg) as a yellow solid.

LC-MS (Method 2A): Rt 0.97 mins; MS m/z 421.3=[M+H]+

¹H NMR (500 MHz, Methanol-d4) δ 8.89 (s, 1H), 8.16 (d, J=5.4 Hz, 1H), 7.30 (s, 1H), 7.10 (d, J=8.5 Hz, 2H), 6.82 (d, J=8.5 Hz, 2H), 6.60 (d, J=5.4 Hz, 1H), 3.80-3.46 (m, 8H), 1.50 (s, 9H).

### Step 5: 2-oxo-1-phenyl-N-[4-[(6-piperazin-1-yl-1,7-naphthyridin-4-yl)amino]phenyl]pyridine-3-carboxamide

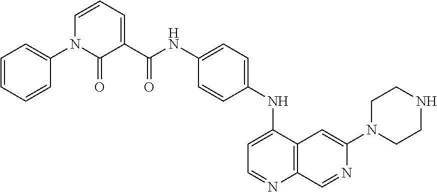 [SMILES: O=C(Nc1ccc(Nc2ccnc3cnc(N4CCNCC4)cc23)cc1)c1cccn(-c2ccccc2)c1=O]

A mixture of tert-butyl 4-[4-(4-aminoanilino)-1,7-naphthyridin-6-yl]piperazine-1-carboxylate (Example 1, step 4) (50 mg, 0.0951 mmol), 2-oxo-1-phenyl-pyridine-3-carboxylic acid (Intermediate A)(29 mg, 0.133 mmol), HATU (58 mg, 0.152 mmol) and DIPEA (0.042 mL, 0.238 mmol) in DMF (1 mL) was stirred at room temperature for 2 hours. The reaction mixture was concentrated in vacuo and the residue was partitioned between EtOAc (5 mL) and water (5 mL). The organic layer was collected, dried over Na₂SO₄ and concentrated in vacuo. The crude residue was purified by preparative HPLC (Acidic Early Elute Method). The desired fractions were combined and concentrated in vacuo to afford tert-butyl 4-[4-[4-[(2-oxo-1-phenyl-pyridine-3-carbonyl)amino]anilino]-1,7-naphthyridin-6-yl]piperazine-1-carboxylate. This material was dissolved in 1,4-Dioxane (1 mL) and 4 M HCl (4M in dioxane) (0.25 mL, 1.00 mmol) was added. The reaction mixture was stirred at room temperature for 1 h. The crude mixture was loaded onto a 1 g Isolute SCX-II column and the column was washed sequentially with MeOH (30 mL) followed by 3.5N NH3 in MeOH (10 mL). The methanolic ammonia fraction was concentrated in vacuo to afford 2-oxo-1-phenyl-N-[4-[(6-piperazin-1-yl-1,7-naphthyridin-4-yl)amino]phenyl]pyridine-3-carboxamide (24 mg) as a bright yellow solid.

LC-MS (Method 7A): Rt 1.35 mins; MS m/z 518.2=[M+H]+

¹H NMR (400 MHz, DMSO-d6) δ 11.99 (s, 1H), 8.91 (s, 1H), 8.77 (s, 1H), 8.59 (dd, J=7.3, 2.2 Hz, 1H), 8.27 (d, J=5.1 Hz, 1H), 8.11 (dd, J=6.6, 2.2 Hz, 1H), 7.83-7.70 (m, 2H), 7.64-7.49 (m, 5H), 7.40-7.27 (m, 3H), 6.84 (d, J=5.1 Hz, 1H), 6.73 (dd, J=7.1, 6.7 Hz, 1H), 3.65-3.55 (m, 4H), 3.03-2.94 (m, 4H).

The compounds of the following tabulated Examples (Table 1) were prepared analogously to Example 1 step 5 from tert-butyl 4-[4-(4-aminoanilino)-1,7-naphthyridin-6-yl]piperazine-1-carboxylate (Example 1, step 4) and the appropriate carboxylic acid.

TABLE 1

| Ex. | Structure and Name | Retention Time, [M + H]+, 1H NMR |
|---|---|---|
| 1.1 | 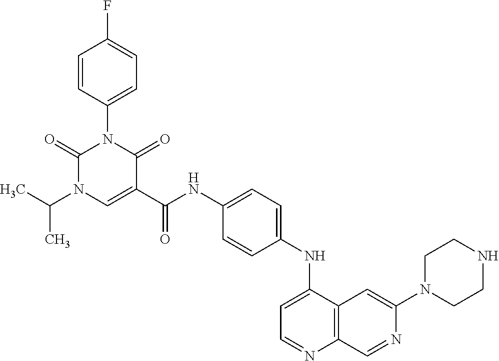 [SMILES: CC(C)n1cc(C(=O)Nc2ccc(Nc3ccnc4cnc(N5CCNCC5)cc34)cc2)c(=O)n(-c2ccc(F)cc2)c1=O] | LC-MS (Method 7A): Rt 1.53 mins; MS m/z 595.3 = [M + H]+ ¹H NMR (400 MHz, DMSO-d6) δ 10.85 (s, 1H), 8.89 (s, 1H), 8.76 (br s, 1H), 8.64 (s, 1H), 8.25 (d, J = 5.1 Hz, 1H), 7.75-7.67 (m, 2H), 7.47-7.39 (m, 2H), 7.38-7.29 (m, 5H), 6.83 (d, J = 5.1 Hz, 1H), 4.77 (hept, J = 6.6 Hz, 1H), 3.64-3.58 (m, 4H), 3.05-2.96 (m, 4H), 1.41 (d, J = 6.8 Hz, 6H) |
| | 3-(4-fluorophenyl)-1-isopropyl-2,4-dioxo-N-[4-[(6-piperazin-1-yl-1,7-naphthyridin- | |
| | 4-yl)amino]phenyl]pyrimidine-5-carboxamide | |
| 1.2 | 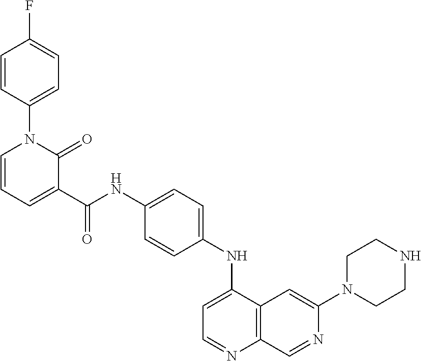 [SMILES: O=C(Nc1ccc(Nc2ccnc3cnc(N4CCNCC4)cc23)cc1)c1cccn(-c2ccc(F)cc2)c1=O] | LC-MS (Method 7A): Rt 1.37 mins; MS m/z 536.3 = [M + H]+ ¹H NMR (500 MHz, DMSO-d6) δ 11.94 (s, 1H), 8.88 (s, 1H), 8.75 (s, 1H), 8.58 (dd, J = 7.2, 2.2 Hz, 1H), 8.24 (d, J = 5.1 Hz, 1H), 8.10 (dd, J = 6.6, 2.2 Hz, 1H), 7.79-7.73 (m, 2H), 7.65-7.56 (m, 2H), 7.46-7.37 (m, 2H), 7.36-7.31 (m, 2H), 7.30 (s, 1H), 6.83 (d, J = 5.1 Hz, 1H), 6.72 (dd, J = 7.2, 6.6 Hz, 1H), 3.56-3.47 (m, 4H), 2.91-2.82 (m, 4H) |
| | 1-(4-fluorophenyl)-2-oxo-N-[4-[(6-piperazin-1-yl-1,7-naphthyridin-4- | |
| | yl)amino]phenyl]pyridine-3-carboxamide | |
| 1.3 | 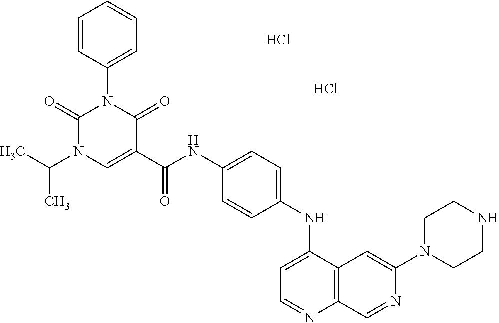 [SMILES: CC(C)n1cc(C(=O)Nc2ccc(Nc3ccnc4cnc(N5CCNCC5)cc34)cc2)c(=O)n(-c2ccccc2)c1=O ; Cl ; Cl] | LC-MS (Method 7A): Rt 1.58 mins; MS m/z 577.4 = [M + H]+ ¹H NMR (400 MHz, DMSO-d6) δ 14.55 (s, 1H), 11.04 (s, 2H), 9.24 (s, 2H), 9.12 (s, 1H), 8.67 (s, 1H), 8.40 (d, J = 6.7 Hz, 1H), 8.06 (s, 1H), 7.90-7.84 (m, 2H), 7.56-7.50 (m, 2H), 7.49-7.44 (m, 3H), 7.41-7.33 (m, 2H), 6.78 (d, J = 6.7 Hz, 1H), 4.79 (h, J = 6.7 Hz, 1H), 4.01-3.91 (m, 4H), 3.28-3.21 (m, 4H), 1.43 (d, J = 6.8 Hz, 6H) |
| | 1-isopropyl-2,4-dioxo-3-phenyl-N-[4-[(6-piperazin-1-yl-1,7-naphthyridin-4- | |
| | yl)amino]phenyl]pyrimidine-5-carboxamide (bis HCl salt) | |
| 1.4 | 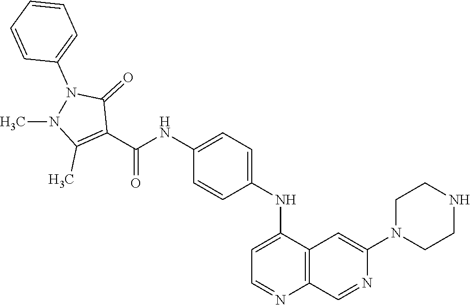 [SMILES: Cc1c(C(=O)Nc2ccc(Nc3ccnc4cnc(N5CCNCC5)cc34)cc2)c(=O)n(-c2ccccc2)n1C] | LC-MS (Method 7A): Rt 1.30 mins; MS m/z 535.5 = [M + H]+ ¹H NMR (400 MHz, DMSO-d6) δ 10.77 (s, 1H), 8.88 (s, 1H), 8.72 (br s, 1H), 8.24 (d, J = 5.1 Hz, 1H), 7.70- 7.65 (m, 2H), 7.63-7.57 (m, 2H), 7.54- 7.49 (m, 1H), 7.47-7.40 (m, 2H), 7.35- 7.27 (m, 3H), 6.79 (d, J = 5.1 Hz, 1H), 3.55-3.51 (m, 4H), 3.36 (s, 3H), 2.91- 2.87 (m, 4H), 2.72 (s, 3H) |
| | 1,5-dimethyl-3-oxo-2-phenyl-N-[4-[(6-piperazin-1-yl-1,7-naphthyridin-4- | |
| | yl)amino]phenyl]pyrazole-4-carboxamide | |

### Example 2

### N-[4-[[6-(4-hydroxy-1-piperidyl)-1,7-naphthyridin-4-yl]amino]phenyl]-2-oxo-1-phenyl-pyridine-3-carboxamide

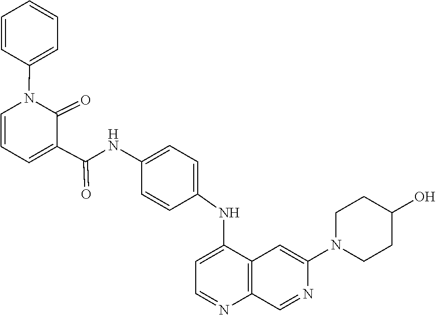 [SMILES: O=C(Nc1ccc(Nc2ccnc3cnc(N4CCC(O)CC4)cc23)cc1)c1cccn(-c2ccccc2)c1=O]

### Step 1: tert-butyl N-[4-[(6-chloro-1,7-naphthyridin-4-yl)amino]phenyl]carbamate

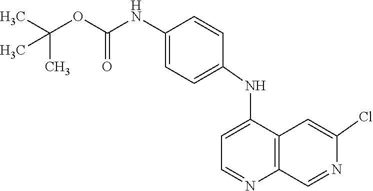 [SMILES: CC(C)(C)OC(=O)Nc1ccc(Nc2ccnc3cnc(Cl)cc23)cc1]

A suspension of 4,6-dichloro-1,7-naphthyridine (250 mg, 1.26 mmol) and tert-butyl (4-aminophenyl)carbamate (288 mg, 1.38 mmol) in IPA (5 mL) was stirred at 60° C. for 18 h. The reaction mixture was filtered and the collected solid was washed with IPA (3×3 mL) to afford tert-butyl N-[4-[(6-chloro-1,7-naphthyridin-4-yl)amino]phenyl]carbamate (390 mg) as a yellow solid.

LC-MS (Method 2A): Rt 0.95 mins; MS m/z 371.0=[M+H]+

¹H NMR (400 MHz, DMSO-d6) δ 11.08 (s, 1H), 9.62 (s, 1H), 9.28 (s, 1H), 8.90 (s, 1H), 8.60 (d, J=6.9 Hz, 1H), 7.73-7.53 (m, 2H), 7.38-7.26 (m, 2H), 6.88 (d, J=6.9 Hz, 1H), 1.50 (s, 9H)

### Step 2: N1-(6-chloro-1,7-naphthyridin-4-yl)benzene-1,4-diamine

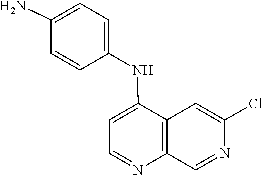 [SMILES: Nc1ccc(Nc2ccnc3cnc(Cl)cc23)cc1]

Trifluoroacetic acid (0.78 mL, 10.5 mmol) was added to suspension of tert-butyl N-[4-[(6-chloro-1,7-naphthyridin-4-yl)amino]phenyl]carbamate (Example 2, step 1) (390 mg, 1.05 mmol) in DCM (10 mL) and the resulting solution was stirred for 3 h. The reaction mixture was concentrated in vacuo before the crude mixture was loaded onto a 5 g Isolute SCX cartridge and the column was washed sequentially with MeOH (30 ml) and 7M NH3 in MeOH (30 ml). The methanolic ammonia fraction was concentrated in vacuo to afford N1-(6-chloro-1,7-naphthyridin-4-yl)benzene-1,4-diamine (255 mg) as a yellow solid.

LC-MS (Method 2A): Rt 0.59 mins; MS m/z 270.9=[M+H]+

¹H NMR (400 MHz, DMSO-d6) δ 9.00 (d, J=0.4 Hz, 1H), 8.93 (s, 1H), 8.49-8.42 (m, 2H), 7.05-6.97 (m, 2H), 6.70-6.61 (m, 3H), 5.16 (s, 2H)

### Step 3: N-[4-[(6-chloro-1,7-naphthyridin-4-yl)amino]phenyl]-2-oxo-1-phenyl-pyridine-3-carboxamide

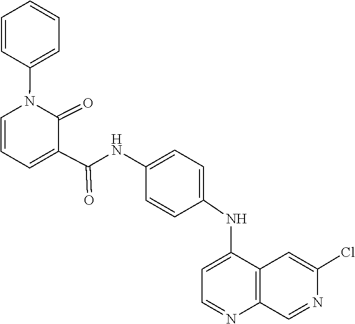 [SMILES: O=C(Nc1ccc(Nc2ccnc3cnc(Cl)cc23)cc1)c1cccn(-c2ccccc2)c1=O]

A mixture of N1-(6-chloro-1,7-naphthyridin-4-yl)benzene-1,4-diamine (Example 2, step 2) (255 mg, 0.942 mmol), 2-oxo-1-phenyl-pyridine-3-carboxylic acid (Intermediate A)(223 mg, 1.04 mmol), HATU (537 mg, 1.41 mmol) and DIPEA (0.41 mL, 2.35 mmol) in DMF (10 mL) was stirred at room temperature for 2 h during which time a solid precipitated from the reaction mixture. The solid was collected by filtration, was washed with DMF (2×5 mL) and water (3×5 mL) and dried to afford N-[4-[(6-chloro-1,7-naphthyridin-4-yl)amino]phenyl]-2-oxo-1-phenyl-pyridine-3-carboxamide (370 mg) as a yellow solid.

LC-MS (Method 1A): Rt 0.65 mins; MS m/z 468.2=[M+H]+

¹H NMR (400 MHz, DMSO-d6) δ 12.00 (s, 1H), 9.21 (s, 1H), 9.06 (s, 1H), 8.59 (dd, J=7.3, 2.2 Hz, 1H), 8.57 (d, J=5.3 Hz, 1H), 8.50 (s, 1H), 8.11 (dd, J=6.6, 2.2 Hz, 1H), 7.83-7.75 (m, 2H), 7.64-7.50 (m, 5H), 7.41-7.33 (m, 2H), 7.02 (d, J=5.3 Hz, 1H), 6.73 (dd, J=7.1, 6.7 Hz, 1H)

### Step 4: N-[4-[[6-(4-hydroxy-1-piperidyl)-1,7-naphthyridin-4-yl]amino]phenyl]-2-oxo-1-phenyl-pyridine-3-carboxamide

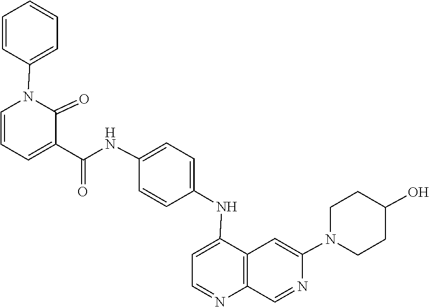 [SMILES: O=C(Nc1ccc(Nc2ccnc3cnc(N4CCC(O)CC4)cc23)cc1)c1cccn(-c2ccccc2)c1=O]

A mixture of N-[4-[(6-chloro-1,7-naphthyridin-4-yl)amino]phenyl]-2-oxo-1-phenyl-pyridine-3-carboxamide (Example 2, step 3) (35 mg, 0.0748 mmol) and piperidin-4-ol (0.81 mL, 8.12 mmol) was heated to 140° C. and was stirred for 18 h. The cooled reaction mixture was partitioned between water (10 mL) and EtOAc (10 mL), the mixture was filtered to remove the formed solids and the filtrate layers were separated. The aqueous layer was further extracted with EtOAc (2×10 mL), the combined organic layers were washed with brine (10 ml) and dried over MgSO₄ before being concentrated in vacuo to afford an orange oil. The oil was combined with the filtered solids and the crude residue purified by preparative HPLC (basic early elute method) to afford N-[4-[[6-(4-hydroxy-1-piperidyl)-1,7-naphthyridin-4-yl]amino]phenyl]-2-oxo-1-phenyl-pyridine-3-carboxamide (8.1 mg) as a yellow solid.

LC-MS (Method 7A): Rt 1.99 mins; MS m/z 533.2=[M+H]+

¹H NMR (500 MHz, DMSO-d6) δ 11.99 (s, 1H), 8.88 (s, 1H), 8.74 (s, 1H), 8.60 (dd, J=7.2, 2.2 Hz, 1H), 8.24 (d, J=5.1 Hz, 1H), 8.12 (dd, J=6.7, 2.2 Hz, 1H), 7.80-7.74 (m, 2H), 7.63-7.50 (m, 5H), 7.37-7.32 (m, 3H), 6.83 (d, J=5.1 Hz, 1H), 6.73 (dd, J=7.2, 6.7 Hz, 1H), 4.71 (d, J=4.3 Hz, 1H), 4.18-4.10 (m, 2H), 3.78-3.70 (m, 1H), 3.22-3.14 (m, 2H), 1.89-1.82 (m, 2H), 1.49-1.41 (m, 2H)

The compounds of the following tabulated Examples (Table 2) were prepared analogously to Example 2 step 4 from N-[4-[(6-chloro-1,7-naphthyridin-4-yl)amino]phenyl]-2-oxo-1-phenyl-pyridine-3-carboxamide (Example 2, step 3) and the appropriate amine.

TABLE 2

| Ex. | Structure and Name | Retention Time, [M + H]+, 1H NMR |
|---|---|---|
| 2.1 | 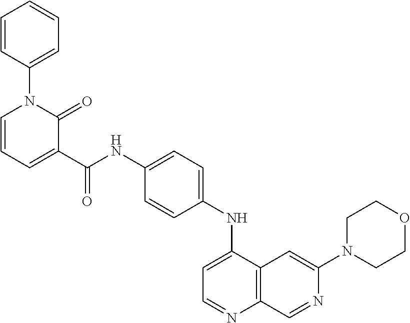 [SMILES: O=C(Nc1ccc(Nc2ccnc3cnc(N4CCOCC4)cc23)cc1)c1cccn(-c2ccccc2)c1=O] | LC-MS (Method 7A): Rt 2.09 mins; MS m/z 519.1 = [M + H]+ ¹H NMR (500 MHz, DMSO-d6) δ 11.99 (s, 1H), 8.92 (s, 1H), 8.79 (s, 1H), 8.59 (dd, J = 7.3, 2.1 Hz, 1H), 8.28 (d, J = 5.1 Hz, 1H), 8.12 (dd, J = 6.6, 2.1 Hz, 1H), 7.81-7.74 (m, 2H), 7.63-7.57 (m, 2H), 7.57-7.52 (m, 3H), 7.38 (s, 1H), 7.36-7.30 (m, 2H), 6.85 (d, J = 5.1 Hz, 1H), 6.73 (dd, J = 6.9 Hz, 1H), 3.82-3.77 (m, 4H), 3.57-3.52 (m, 4H). |
| | N-[4-[(6-morpholino-1,7-naphthyridin-4-yl)amino]phenyl]-2-oxo- | |
| | 1-phenyl-pyridine-3-carboxamide | |
| 2.2 | 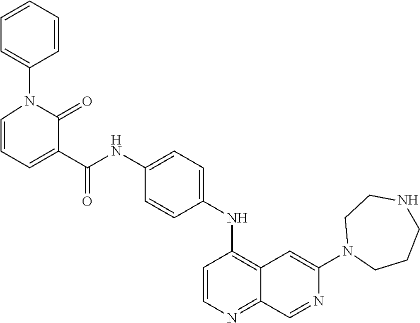 [SMILES: O=C(Nc1ccc(Nc2ccnc3cnc(N4CCCNCC4)cc23)cc1)c1cccn(-c2ccccc2)c1=O] | LC-MS (Method 7A): Rt 1.44 mins; MS m/z 532.3 = [M + H]+ ¹H NMR (500 MHz, Chloroform-d) δ 11.94 (s, 1H), 9.07 (s, 1H), 8.76 (dd, J = 7.2, 2.2 Hz, 1H), 8.32 (d, J = 5.0 Hz, 1H), 7.84-7.75 (m, 2H), 7.65 (dd, J = 6.6, 2.2 Hz, 1H), 7.63-7.57 (m, 2H), 7.56-7.52 (m, 1H), 7.45-7.40 (m, 2H), 7.26-7.23 (m, 2H), 6.88 (d, J = 5.0 Hz, 1H), 6.61 (dd, J = 7.1 Hz, 1H), 6.43 (s, 1H), 6.25 (s, 1H), 3.95- 3.80 (m, 4H), 3.14-3.07 (m, 2H), 2.93-2.86 (m, 2H), 1.99 (p, J = 5.9 Hz, 2H) |
| | N-[4-[[6-(1,4-diazepan-1-yl)-1,7-naphthyridin-4-yl]amino]phenyl]-2- | |
| | oxo-1-phenyl-pyridine-3-carboxamide | |

### Example 3

### N-(1-methyl-4-piperidyl)-4-[4-[(2-oxo-1-phenyl-pyridine-3-carbonyl)amino]anilino]-1,7-naphthyridine-6-carboxamide

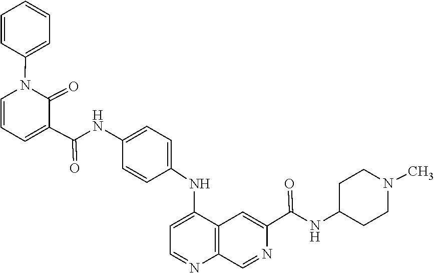 [SMILES: CN1CCC(NC(=O)c2cc3c(Nc4ccc(NC(=O)c5cccn(-c6ccccc6)c5=O)cc4)ccnc3cn2)CC1]

XantPhos Pd-G3 (8.1 mg, 8.55 μmol) and sodium carbonate (36 mg, 0.342 mmol) were added to chamber A of a COware apparatus. The apparatus was flushed with nitrogen before the addition of a nitrogen flushed solution of 1-methylpiperidin-4-amine (29 mg, 0.256 mmol) and N-[4-[(6-chloro-1,7-naphthyridin-4-yl)amino]phenyl]-2-oxo-1-phenyl-pyridine-3-carboxamide (Example 2, step 3) (40 mg, 0.0855 mmol) in anhydrous DMA (1 mL). A solution of formic acid (12 mg, 0.256 mmol) and methanesulfonyl chloride (29 mg, 0.256 mmol) in nitrogen flushed anhydrous Toluene (1 mL) was added to Chamber B before the addition of triethylamine (0.071 mL, 0.513 mmol) to chamber B. The reaction mixture was heated to 100° C. and was stirred for 18 h. The chamber A reaction mixture was partitioned between DCM (5 mL) and water (5 mL). The aqueous layer was extracted with DCM (2×5 mL) and the combined organic fractions were passed through a hydrophobic frit before being concentrated in vacuo. The crude residue was purified by preparative HPLC (Basic Early Elute Method) to afford N-(1-methyl-4-piperidyl)-4-[4-[(2-oxo-1-phenyl-pyridine-3-carbonyl)amino]anilino]-1,7-naphthyridine-6-carboxamide (15.5 mg) as a yellow solid.

LC-MS (Method 7A): Rt 1.39 mins; MS m/z 574.3=[M+H]+

¹H NMR (500 MHz, Chloroform-d) δ 11.99 (s, 1H), 9.28 (s, 1H), 8.77 (dd, J=7.3, 2.2 Hz, 1H), 8.73 (s, 1H), 8.65 (d, J=5.4 Hz, 1H), 8.16 (d, J=8.4 Hz, 1H), 7.87-7.80 (m, 2H), 7.65 (dd, J=6.6, 2.2 Hz, 1H), 7.63-7.51 (m, 3H), 7.45-7.40 (m, 2H), 7.32-7.27 (m, 2H), 7.08 (s, 1H), 7.04 (d, J=5.4 Hz, 1H), 6.62 (dd, J=7.0 Hz, 1H), 4.11-3.98 (m, 1H), 2.90-2.79 (m, 2H), 2.33 (s, 3H), 2.27-2.15 (m, 2H), 2.11-2.03 (m, 2H), 1.76-1.66 (m, 2H)

### Example 4

### N-[4-[[6-(1-methylpyrazol-4-yl)-1,7-naphthyridin-4-yl]amino]phenyl]-2-oxo-1-phenyl-pyridine-3-carboxamide

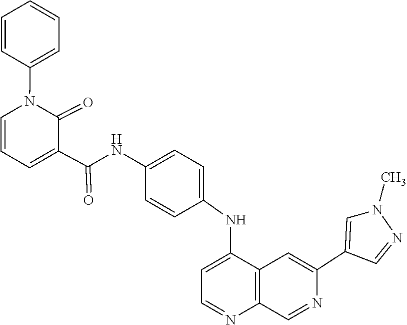 [SMILES: Cn1cc(-c2cc3c(Nc4ccc(NC(=O)c5cccn(-c6ccccc6)c5=O)cc4)ccnc3cn2)cn1]

A suspension of N-[4-[(6-chloro-1,7-naphthyridin-4-yl)amino]phenyl]-2-oxo-1-phenyl-pyridine-3-carboxamide (Example 2, step 3) (50 mg, 0.107 mmol), cesium carbonate (87 mg, 0.267 mmol) and 1-methyl-4-(4,4,5,5-tetramethyl-1,3,2-dioxaborolan-2-yl)pyrazole (33 mg, 0.160 mmol) in DMF (2 mL) was flushed with nitrogen for 5 minutes before the addition of Pd(dppf)Cl₂ (8.7 mg, 0.0107 mmol). The reaction mixture was warmed to 100° C. and was stirred for 18 h. The cooled reaction mixture was partitioned between DCM (5 mL) and water (5 mL) and the aqueous layer was extracted with DCM (2×5 mL). The combined organic fractions were concentrated in vacuo and purified by preparative HPLC (Basic Early Elute Method). The product containing fractions were concentrated in vacuo to afford a yellow solid which was purified further by silica chromatography (Biotage KP-NH silica eluting with a gradient of 0-100% EtOAc in heptane). The pure fractions were combined and the material loaded onto a 1 g Isolute SCX cartridge, the column was washed sequentially with MeCN (10 ml), MeOH (10 ml) and 7M NH₃ in MeOH (10 ml). The methanolic ammonia fraction was concentrated in vacuo to afford N-[4-[[6-(1-methylpyrazol-4-yl)-1,7-naphthyridin-4-yl]amino]phenyl]-2-oxo-1-phenyl-pyridine-3-carboxamide (3.8 mg) as a yellow solid.

LC-MS (Method 7A): Rt 2.04 mins; MS m/z 514.4=[M+H]+

¹H NMR (500 MHz, DMSO-d6) δ 12.04 (s, 1H), 9.44 (s, 1H), 9.19 (s, 1H), 8.60 (dd, J=7.3, 2.2 Hz, 1H), 8.57 (s, 1H), 8.49 (d, J=5.6 Hz, 1H), 8.29 (s, 1H), 8.12 (dd, J=6.6, 2.2 Hz, 1H), 8.06 (s, 1H), 7.84 (d, J=8.7 Hz, 2H), 7.71-7.51 (m, 5H), 7.42 (d, J=8.7 Hz, 2H), 6.94 (d, J=5.6 Hz, 1H), 6.73 (dd, J=7.3, 6.9 Hz, 1H), 3.93 (s, 3H)

The compound of the following tabulated Example (Table 3) was prepared analogously to Example 4 from N-[4-[(6-chloro-1,7-naphthyridin-4-yl)amino]phenyl]-2-oxo-1-phenyl-pyridine-3-carboxamide (Example 2, step 3) and the appropriate boronate ester.

TABLE 3

| Ex. | Structure and Name | Retention Time, [M + H]+, 1H NMR |
|---|---|---|
| 4.1 | 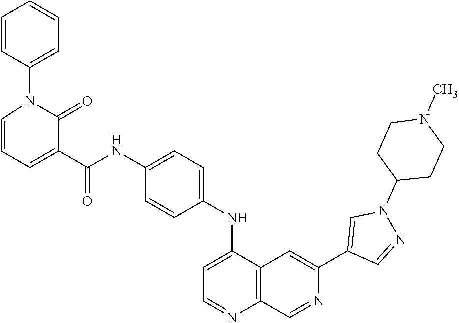 [SMILES: CN1CCC(n2cc(-c3cc4c(Nc5ccc(NC(=O)c6cccn(-c7ccccc7)c6=O)cc5)ccnc4cn3)cn2)CC1] | LC-MS (Method 7A): Rt 1.48 mins; MS m/z 597.4 = [M + H]+ ¹H NMR (500 MHz, DMSO-d6) δ 12.02 (s, 1H), 9.15 (d, J = 0.6 Hz, 1H), 9.02 (s, 1H), 8.60 (dd, J = 7.3, 2.2 Hz, 1H), 8.52 (s, 1H), 8.46 (d, J = 5.3 Hz, 1H), 8.34 (s, 1H), 8.19 (s, 1H), 8.12 (dd, J = 6.6, 2.2 Hz, 1H), 8.10 (d, J = 0.5 Hz, 1H), 7.85-7.79 (m, 2H), 7.63-7.50 (m, 5H), 7.44-7.34 (m, 2H), 6.95 (d, J = 5.3 Hz, 1H), 6.73 (dd, J = 7.3, 6.7 Hz, 1H), 4.25-4.14 (m, 1H), 2.92-2.83 (m, 2H), 2.22 (s, 3H), 2.15-1.98 (m, 6H) |
| | N-[4-[[6-[1-(1-methyl-4-piperidyl)pyrazol-4-yl]-1,7-naphthyridin-4- | |
| | yl]amino]phenyl]-2-oxo-1-phenyl-pyridine-3-carboxamide (mono | |
| | formate salt) | |

### Example 5

### N-(4-hydroxycyclohexyl)-4-[4-[(2-oxo-1-phenyl-pyridine-3-carbonyl)amino]anilino]-1,7-naphthyridine-6-carboxamide

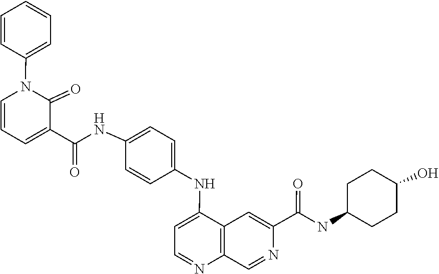

### Step 1: 4-[4-[(2-oxo-1-phenyl-pyridine-3-carbonyl)amino]anilino]-1,7-naphthyridine-6-carboxylic acid

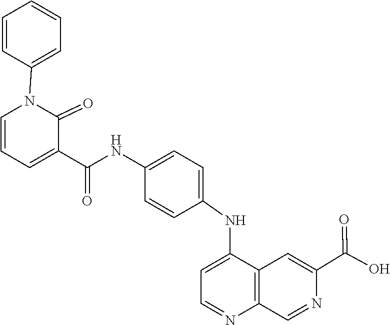 [SMILES: O=C(O)c1cc2c(Nc3ccc(NC(=O)c4cccn(-c5ccccc5)c4=O)cc3)ccnc2cn1]

A mixture of N-[4-[(6-chloro-1,7-naphthyridin-4-yl)amino]phenyl]-2-oxo-1-phenyl-pyridine-3-carboxamide (Example 2, step 3) (250 mg, 0.534 mmol), XantPhos Pd-G3 (51 mg, 0.0534 mmol), sodium carbonate (170 mg, 1.60 mmol), formic acid (81 μL, 2.14 mmol) and methanesulfonyl chloride (124 μL, 1.60 mmol) in anhydrous DMA (5 mL) was degassed with nitrogen. The reaction vessel was sealed and then triethylamine (0.45 mL, 3.21 mmol) was added. The reaction mixture was heated to 120° C. and stirred for 16 h. DCM (10 mL) and water (10 mL) were added to the reaction mixture at which point a precipitate formed which was collected by filtration and dried in a vacuum oven at 40° C. to afford 4-[4-[(2-oxo-1-phenyl-pyridine-3-carbonyl)amino]anilino]-1,7-naphthyridine-6-carboxylic acid (264 mg) as a green solid. The crude material could be further purified by recrystallisation from DMSO/MeCN/water.

LC-MS (Method 7B): Rt 1.82 mins; MS m/z 478.3=[M+H]+

¹H NMR (500 MHz, DMSO-d6) δ 11.99 (s, 1H), 9.56 (s, 1H), 9.31-9.15 (m, 1H), 9.04 (s, 1H), 8.63-8.51 (m, 2H), 8.11 (dd, J=6.6, 2.1 Hz, 1H), 7.76 (d, J=8.8 Hz, 2H), 7.63-7.49 (m, 6H), 7.41 (d, J=8.8 Hz, 2H), 7.04 (d, J=5.3 Hz, 1H), 6.73 (t, J=6.9 Hz, 1H).

### Step 2: N-(4-hydroxycyclohexyl)-4-[4-[(2-oxo-1-phenyl-pyridine-3-carbonyl)amino]anilino]-1,7-naphthyridine-6-carboxamide

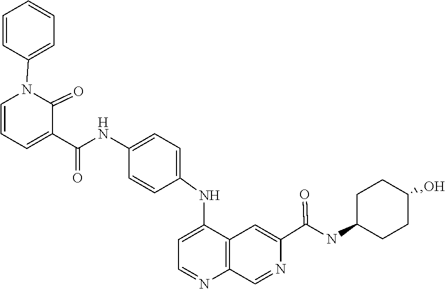

A mixture of 4-[4-[(2-oxo-1-phenyl-pyridine-3-carbonyl)amino]anilino]-1,7-naphthyridine-6-carboxylic acid (Example 5, step 1) (38 mg, 0.0796 mmol), trans-4-aminocyclohexanol hydrochloride (0.031 mL, 0.159 mmol), HATU (45 mg, 0.119 mmol) and DIPEA (0.063 mL, 0.358 mmol) in DMF (1 mL) was stirred at room temperature for 18 h. The reaction mixture was partitioned between DCM (10 mL) and water (10 mL), the aqueous was extracted with DCM (2×10 mL), the combined organic layers were dried by passage through a hydrophobic frit and were concentrated in vacuo. The crude residue was purified by low pH reverse phase chromatography (eluting with 10-100% MeCN (0.1% formic acid) in water (0.1% formic acid)) to afford N-(4-hydroxycyclohexyl)-4-[4-[(2-oxo-1-phenyl-pyridine-3-carbonyl)amino]anilino]-1,7-naphthyridine-6-carboxamide (3.8 mg) as a yellow solid.

LC-MS (Method 7A): Rt 1.89 mins; MS m/z 575.3=[M+H]+

¹H NMR (400 MHz, Chloroform-d) δ 11.99 (s, 1H), 9.26 (s, 1H), 8.77 (dd, J=7.3, 2.2 Hz, 1H), 8.74 (s, 1H), 8.64 (d, J=5.4 Hz, 1H), 8.10 (d, J=8.5 Hz, 1H), 7.88-7.79 (m, 2H), 7.65 (dd, J=6.6, 2.2 Hz, 1H), 7.63-7.50 (m, 3H), 7.47-7.39 (m, 2H), 7.33-7.27 (m, 2H), 7.08 (s, 1H), 7.03 (d, J=5.4 Hz, 1H), 6.62 (dd, J=7.0 Hz, 1H), 4.07-3.95 (m, 1H), 3.77-3.65 (m, 1H), 2.20-2.11 (m, 2H), 2.10-2.01 (m, 2H), 1.54-1.37 (m, 4H)

The compounds of the following tabulated Examples (Table 4) were prepared analogously to Example 5 step 2 from 4-[4-[(2-oxo-1-phenyl-pyridine-3-carbonyl)amino]anilino]-1,7-naphthyridine-6-carboxylic acid (Example 5, step 1) and the appropriate amine.

TABLE 4

| Ex. | Structure and Name | Retention Time, [M + H]+, 1H NMR |
|---|---|---|
| 5.1 | 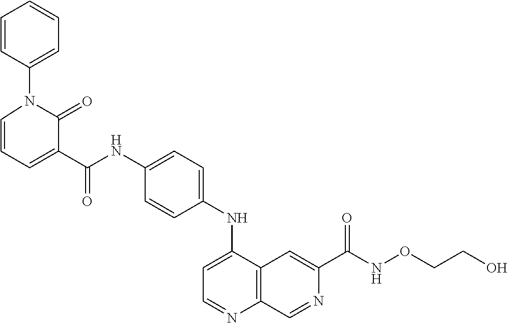 [SMILES: O=C(NOCCO)c1cc2c(Nc3ccc(NC(=O)c4cccn(-c5ccccc5)c4=O)cc3)ccnc2cn1] | LC-MS (Method 7A): 1.80 mins; MS m/z 537.4 = [M + H]+ ¹H NMR (400 MHz, DMSO-d6) δ 12.12 (s, 1H), 12.07 (s, 1H), 9.24 (s, 1H), 9.18 (s, 1H), 8.64 (d, J = 6.1 Hz, 1H), 8.61 (dd, J = 7.2, 2.2 Hz, 1H), 8.13 (dd, J = 6.7, 2.2 Hz, 1H), 7.91- 7.80 (m, 2H), 7.66-7.51 (m, 5H), 7.47-7.40 (m, 2H), 7.07 (d, J = 6.1 Hz, 1H), 6.74 (dd, J = 7.2, 6.7 Hz, 1H), 3.99 (t, J = 5.0 Hz, 2H), 3.65 (t, J = 5.0 Hz, 2H) |
| | N-(2-hydroxyethoxy)-4-[4-[(2-oxo-1-phenyl-pyridine-3-carbonyl)amino]anilino]-1,7- | |
| | naphthyridine-6-carboxamide | |
| 5.2 | 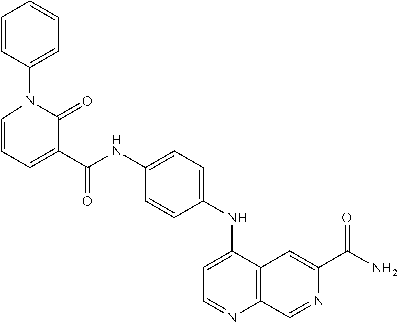 [SMILES: NC(=O)c1cc2c(Nc3ccc(NC(=O)c4cccn(-c5ccccc5)c4=O)cc3)ccnc2cn1] | LC-MS (Method 7A): Rt 1.75 mins; MS m/z 477.3 = [M + H]+ ¹H NMR (400 MHz, DMSO-d6) δ 11.99 (s, 1H), 9.60 (s, 1H), 9.18 (s, 1H), 9.10 (s, 1H), 8.66-8.55 (m, 2H), 8.24 (s, 1H), 8.11 (dd, J = 6.6, 2.2 Hz, 1H), 7.82-7.74 (m, 2H), 7.71 (s, 1H), 7.62-7.52 (m, 5H), 7.46-7.30 (m, 2H), 7.12-6.98 (m, 1H), 6.73 (dd, J = 7.0, 6.6 Hz, 1H) |
| | 4-[4-[(2-oxo-1-phenyl-pyridine-3-carbonyl)amino]anilino]-1,7- | |
| | naphthyridine-6-carboxamide | |
| 5.3 | 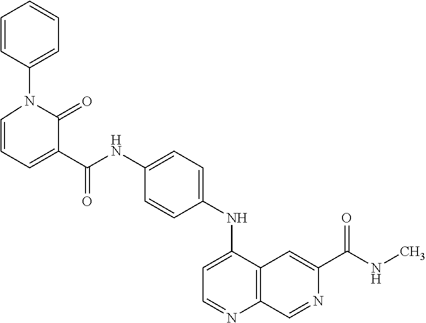 [SMILES: CNC(=O)c1cc2c(Nc3ccc(NC(=O)c4cccn(-c5ccccc5)c4=O)cc3)ccnc2cn1] | LC-MS (Method 7A): Rt 1.85 mins; MS m/z 491.3 = [M + H]+ ¹H NMR (500 MHz, Chloroform-d) δ 11.99 (s, 1H), 9.27 (s, 1H), 8.81-8.73 (m, 2H), 8.64 (d, J = 5.4 Hz, 1H), 8.23 (q, J = 5.0 Hz, 1H), 7.85-7.80 (m, 2H), 7.65 (dd, J = 6.6, 2.2 Hz, 1H), 7.62- 7.57 (m, 2H), 7.57-7.52 (m, 1H), 7.45- 7.40 (m, 2H), 7.32-7.27 (m, 2H), 7.15 (s, 1H), 7.03 (d, J = 5.4 Hz, 1H), 6.62 (dd, J = 7.0 Hz, 1H), 3.08 (d, J = 5.1 Hz, 3H) |
| | N-methyl-4-[4-[(2-oxo-1-phenyl-pyridine-3-carbonyl)amino]anilino]- | |
| | 1,7-naphthyridine-6-carboxamide | |
| 5.4 | 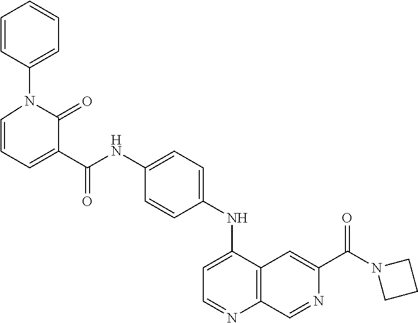 [SMILES: O=C(Nc1ccc(Nc2ccnc3cnc(C(=O)N4CCC4)cc23)cc1)c1cccn(-c2ccccc2)c1=O] | LC-MS (Method 7A): Rt 1.93 mins; MS m/z 517.3 = [M + H]+ 1H NMR (500 MHz, Chloroform-d) δ 11.97 (s, 1H), 9.27 (s, 1H), 8.90 (s, 1H), 8.81-8.74 (m, 1H), 8.62 (d, J = 5.4 Hz, 1H), 7.86-7.78 (m, 2H), 7.67- 7.48 (m, 5H), 7.45-7.38 (m, 2H), 7.30-7.26 (m, 2H), 6.99 (d, J = 5.4 Hz, 1H), 6.64-6.58 (m, 1H), 4.83 (t, J = 7.8 Hz, 2H), 4.24-4.14 (m, 2H), 2.35 (p, J = 7.8 Hz, 2H). |
| | N-[4-[[6-(azetidine-1-carbonyl)-1,7-naphthyridin-4-yl]amino]phenyl]- | |
| | 2-oxo-1-phenyl-pyridine-3-carboxamide | |

### Example 6

### N-(1-methyl-4-piperidyl)-4-[4-[(2-oxo-1-phenyl-pyridine-3-carbonyl)amino]phenoxy]-1,7-naphthyridine-6-carboxamide

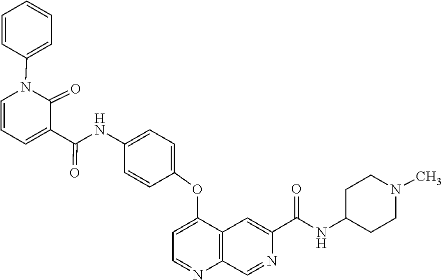 [SMILES: CN1CCC(NC(=O)c2cc3c(Oc4ccc(NC(=O)c5cccn(-c6ccccc6)c5=O)cc4)ccnc3cn2)CC1]

### Step 1: tert-butyl N-[4-[(6-chloro-1,7-naphthyridin-4-yl)oxy]phenyl]carbamate

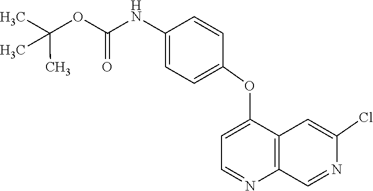 [SMILES: CC(C)(C)OC(=O)Nc1ccc(Oc2ccnc3cnc(Cl)cc23)cc1]

A suspension of 4,6-dichloro-1,7-naphthyridine (175 mg, 0.879 mmol), potassium hydroxide (99 mg, 1.76 mmol) and tert-butyl (4-hydroxyphenyl)carbamate (552 mg, 2.64 mmol) in IPA (8.75 mL) was stirred at 60° C. for 18 h. The cooled reaction mixture was concentrated in vacuo, the resultant solid was partitioned between DCM (5 mL) and water (5 mL), the aqueous layer was extracted with DCM (2×5 mL) and the combined organic layers were concentrated in vacuo. The crude residue was purified by silica chromatography (eluting with a gradient of 20-100% EtOAc in heptane) to afford tert-butyl N-[4-[(6-chloro-1,7-naphthyridin-4-yl)oxy]phenyl]carbamate (235 mg) as a white solid.

LC-MS (Method 2A): Rt 1.37 mins; MS m/z 372.2=[M+H]+

¹H NMR (500 MHz, DMSO-d6) δ 9.54 (s, 1H), 9.28 (d, J=0.8 Hz, 1H), 8.83 (d, J=5.2 Hz, 1H), 8.25 (d, J=0.8 Hz, 1H), 7.66-7.58 (m, 2H), 7.29-7.21 (m, 2H), 6.81 (d, J=5.2 Hz, 1H), 1.49 (s, 9H)

### Step 2: 4-[(6-chloro-1,7-naphthyridin-4-yl)oxy]aniline

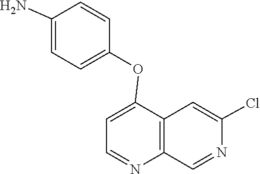 [SMILES: Nc1ccc(Oc2ccnc3cnc(Cl)cc23)cc1]

A solution of tert-butyl N-[4-[(6-chloro-1,7-naphthyridin-4-yl)oxy]phenyl]carbamate (Example 6, step 1)(235 mg, 0.632 mmol) in 4 M hydrogen chloride in dioxane (15 mL, 60.0 mmol) was stirred at room temperature for 72 h. The reaction mixture was concentrated in vacuo to afford 4-[(6-chloro-1,7-naphthyridin-4-yl)oxy]aniline (215 mg) as a white solid.

LC-MS (Method 2A): Rt 1.02 mins; MS m/z 272.0=[M+H]+

¹H NMR (500 MHz, DMSO-d6) δ 9.31 (s, 1H), 8.87 (d, J=5.0 Hz, 1H), 8.24 (s, 1H), 7.45-7.25 (m, 4H), 6.92-6.88 (m, 1H)

### Step 3: N-[4-[(6-chloro-1,7-naphthyridin-4-yl)oxy]phenyl]-2-oxo-1-phenyl-pyridine-3-carboxamide

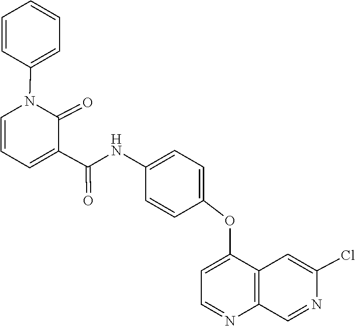 [SMILES: O=C(Nc1ccc(Oc2ccnc3cnc(Cl)cc23)cc1)c1cccn(-c2ccccc2)c1=O]

A mixture of 4-[(6-chloro-1,7-naphthyridin-4-yl)oxy]aniline dihydrochloride (Example 6, step 2) (100 mg, 0.291 mmol), 2-oxo-1-phenyl-pyridine-3-carboxylic acid (Intermediate A)(63 mg, 0.291 mmol), HATU (177 mg, 0.466 mmol) and DIPEA (0.13 mL, 0.728 mmol) in DMF (2 mL) was stirred at room temperature for 2 h. Water (10 mL) was added slowly with stirring to the reaction mixture and the resulting precipitate was collected by filtration, washed with water (3×2 mL) and dried to afford N-[4-[(6-chloro-1,7-naphthyridin-4-yl)oxy]phenyl]-2-oxo-1-phenyl-pyridine-3-carboxamide (140 mg) as a yellow solid.

LC-MS (Method 2A): Rt 1.37 mins; MS m/z 469.3=[M+H]+

¹H NMR (500 MHz, DMSO-d6) δ 12.08 (s, 1H), 9.29 (s, 1H), 8.85 (d, J=5.1 Hz, 1H), 8.60 (dd, J=7.3, 2.1 Hz, 1H), 8.25 (s, 1H), 8.13 (dd, J=6.6, 2.1 Hz, 1H), 7.91-7.83 (m, 2H), 7.64-7.52 (m, 5H), 7.39-7.29 (m, 2H), 6.89 (d, J=5.1 Hz, 1H), 6.73 (dd, J=6.9 Hz, 1H)

### Step 4: N-(1-methyl-4-piperidyl)-4-[4-[(2-oxo-1-phenyl-pyridine-3-carbonyl)amino]phenoxy]-1,7-naphthyridine-6-carboxamide

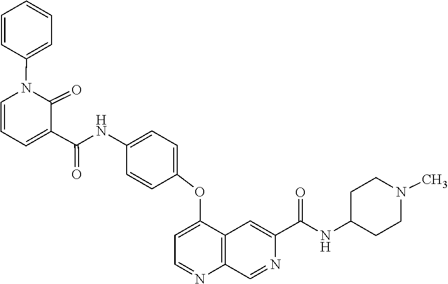 [SMILES: CN1CCC(NC(=O)c2cc3c(Oc4ccc(NC(=O)c5cccn(-c6ccccc6)c5=O)cc4)ccnc3cn2)CC1]

XantPhos Pd-G3 (10 mg, 0.0107 mmol) and sodium carbonate (45 mg, 0.427 mmol) were added to chamber A of a COware apparatus. The apparatus was flushed with nitrogen before the addition of a nitrogen flushed solution of 1-methylpiperidin-4-amine (37 mg, 0.320 mmol) and N-[4-[(6-chloro-1,7-naphthyridin-4-yl)oxy]phenyl]-2-oxo-1-phenyl-pyridine-3-carboxamide (Example 6, step 3)(50 mg, 0.107 mmol) in anhydrous DMA (1 mL). A solution of formic acid (15 mg, 0.320 mmol) and methanesulfonyl chloride (37 mg, 0.320 mmol) in nitrogen flushed anhydrous toluene (1 mL) was added to Chamber B, the system was sealed and triethylamine (0.089 mL, 0.640 mmol) was added to chamber B. The reaction mixture was heated to 100° C. and stirred for 18 h. The cooled reaction mixture was partitioned between DCM (5 mL) and water (5 mL), the aqueous layer was extracted with DCM (2×5 mL) and the combined organics were passed through a hydrophobic frit before being concentrated in vacuo. The residue was purified by preparative HPLC (early elute basic method) to afford N-(1-methyl-4-piperidyl)-4-[4-[(2-oxo-1-phenyl-pyridine-3-carbonyl)amino]phenoxy]-1,7-naphthyridine-6-carboxamide (19.3 mg) as a white solid.

LC-MS (Method 7A): Rt 1.99 mins; MS m/z 575.3=[M+H]+

¹H NMR (500 MHz, DMSO-d6) δ 12.08 (s, 1H), 9.43 (d, J=0.8 Hz, 1H), 8.91 (d, J=5.2 Hz, 1H), 8.79 (d, J=0.8 Hz, 1H), 8.76 (d, J=8.4 Hz, 1H), 8.61 (dd, J=7.2, 2.2 Hz, 1H), 8.13 (dd, J=6.6, 2.2 Hz, 1H), 7.92-7.86 (m, 2H), 7.61-7.56 (m, 2H), 7.56-7.51 (m, 3H), 7.40-7.35 (m, 2H), 6.93 (d, J=5.2 Hz, 1H), 6.73 (dd, J=7.2, 6.6 Hz, 1H), 3.89-3.78 (m, 1H), 2.80-2.72 (m, 2H), 2.17 (s, 3H), 2.04-1.95 (m, 2H), 1.83-1.68 (m, 4H)

### Example 7

### 4-[4-[[3-(4-fluorophenyl)-1-isopropyl-2,4-dioxo-pyrimidine-5-carbonyl]amino]anilino]-N-(2-hydroxyethoxy)-1,7-naphthyridine-6-carboxamide

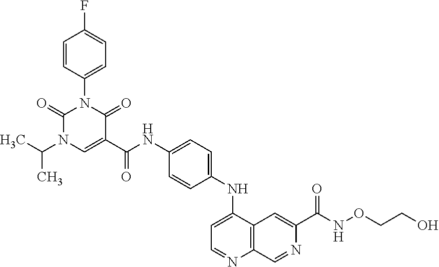 [SMILES: CC(C)n1cc(C(=O)Nc2ccc(Nc3ccnc4cnc(C(=O)NOCCO)cc34)cc2)c(=O)n(-c2ccc(F)cc2)c1=O]

### Step 1: N-[4-[(6-chloro-1,7-naphthyridin-4-yl)amino]phenyl]-3-(4-fluorophenyl)-1-isopropyl-2,4-dioxo-pyrimidine-5-carboxamide

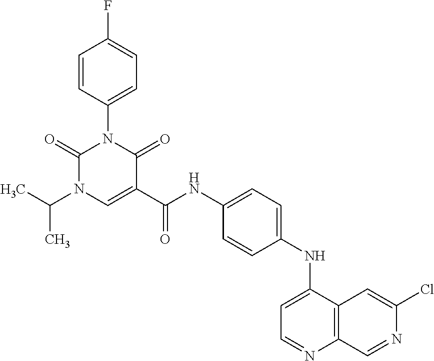 [SMILES: CC(C)n1cc(C(=O)Nc2ccc(Nc3ccnc4cnc(Cl)cc34)cc2)c(=O)n(-c2ccc(F)cc2)c1=O]

A mixture of N1-(6-chloro-1,7-naphthyridin-4-yl)benzene-1,4-diamine (Example 2, step 2)(100 mg, 0.369 mmol), 3-(4-fluorophenyl)-1-isopropyl-2,4-dioxo-pyrimidine-5-carboxylic acid (Intermediate C)(119 mg, 0.406 mmol), HATU (211 mg, 0.554 mmol) and DIPEA (0.16 mL, 0.923 mmol) in DMF (4 mL) was stirred at room temperature for 2.5 h. The reaction mixture was partitioned between water (15 mL) and EtOAc (15 mL), the organic fraction separated and aqueous layer further extracted with EtOAc (2×10 mL). The organic fractions were combined, washed with brine (2×20 mL), dried over MgSO₄ and concentrated in vacuo to afford N-[4-[(6-chloro-1,7-naphthyridin-4-yl)amino]phenyl]-3-(4-fluorophenyl)-1-isopropyl-2,4-dioxo-pyrimidine-5-carboxamide (200 mg) as a yellow solid.

LC-MS (Method 2A): Rt 1.08 mins; MS m/z 545.3=[M+H]+

¹H NMR (500 MHz, DMSO-d6) δ 10.91 (s, 1H), 9.21 (s, 1H), 9.07 (s, 1H), 8.67 (s, 1H), 8.57 (d, J=5.3 Hz, 1H), 8.50 (s, 1H), 7.79-7.71 (m, 2H), 7.48-7.40 (m, 2H), 7.40-7.31 (m, 4H), 7.03 (d, J=5.3 Hz, 1H), 4.79 (hept, J=6.9 Hz, 1H), 1.43 (d, J=6.8 Hz, 6H)

### Step 2: 4-[4-[[3-(4-fluorophenyl)-1-isopropyl-2,4-dioxo-pyrimidine-5-carbonyl]amino]anilino]-N-(2-hydroxyethoxy)-1,7-naphthyridine-6-carboxamide

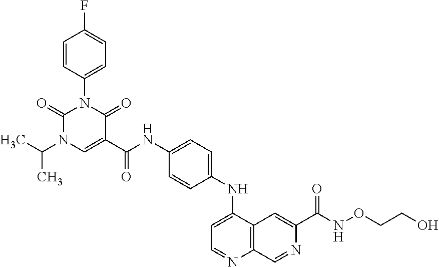 [SMILES: CC(C)n1cc(C(=O)Nc2ccc(Nc3ccnc4cnc(C(=O)NOCCO)cc34)cc2)c(=O)n(-c2ccc(F)cc2)c1=O]

Methanesulfonyl chloride (21 μL, 0.275 mmol) and formic acid (21 μL, 0.550 mmol) were added to a degassed suspension of XantPhos Pd-G3 (8.7 mg, 9.17 μmol), sodium carbonate (39 mg, 0.367 mmol), triethylamine (0.077 mL, 0.550 mmol) and N-[4-[(6-chloro-1,7-naphthyridin-4-yl)amino]phenyl]-3-(4-fluorophenyl)-1-isopropyl-2,4-dioxo-pyrimidine-5-carboxamide (Example 7, step 1)(50 mg, 0.0917 mmol) in anhydrous DMA (1 mL). The reaction mixture was heated in a sealed tube at 100° C. for 18 h. The cooled reaction mixture was loaded onto a 2 g Isolute PE-AX cartridge and the column was washed sequentially with MeOH (10 ml) and 4M HCl in dioxane (2×5 ml). The two fractions were combined and concentrated in vacuo. The residue was purified by reverse phase silica chromatography (eluting with a gradient of 10-100% MeCN (0.1% formic acid) in water (0.1% formic acid)) to afford the intermediate carboxylic acid as a yellow solid. A mixture of the isolated solid, 2-(aminooxy)ethanol (1.4 mg, 0.0180 mmol), HATU (8.2 mg, 0.0216 mmol) and DIPEA (0.0078 mL, 0.0451 mmol) in DMF (1 mL) was stirred at room temperature for 72 h. The reaction mixture was partitioned between DCM (5 mL) and water (5 mL), the aqueous layer was extracted with DCM (2×5 mL) and the combined organic layers were concentrated in vacuo to afford a yellow oil. The oil was purified by low pH reverse phase chromatography (eluting with a gradient of 10-100% MeCN (0.1% formic acid) in water (0.1% formic acid)) to afford 4-[4-[[3-(4-fluorophenyl)-1-isopropyl-2,4-dioxo-pyrimidine-5-carbonyl]amino]anilino]-N-(2-hydroxyethoxy)-1,7-naphthyridine-6-carboxamide (2.8 mg) as a yellow solid.

LC-MS (Method 7A): Rt 1.97 mins; MS m/z 614.2 [M+H]+

¹H NMR (400 MHz, DMSO-d6) δ 12.05 (s, 1H), 10.90 (s, 1H), 9.61 (s, 1H), 9.17 (s, 1H), 9.06 (s, 1H), 8.66 (s, 1H), 8.61 (d, J=5.4 Hz, 1H), 7.82-7.68 (m, 2H), 7.52-7.31 (m, 6H), 7.07 (d, J=5.4 Hz, 1H), 4.89-4.71 (m, 2H), 3.97 (t, J=5.0 Hz, 2H), 3.67-3.58 (m, 2H), 1.43 (d, J=6.8 Hz, 6H)

The compound of the following tabulated Example (Table 5) was prepared analogously to Example 7 step 2 from N-[4-[(6-chloro-1,7-naphthyridin-4-yl)amino]phenyl]-3-(4-fluorophenyl)-1-isopropyl-2,4-dioxo-pyrimidine-5-carboxamide (Example 7, step 1) and the appropriate amine.

TABLE 5

| Ex. | Structure and Name | Retention Time, [M + H]+, 1H NMR |
|---|---|---|
| 7.1 | 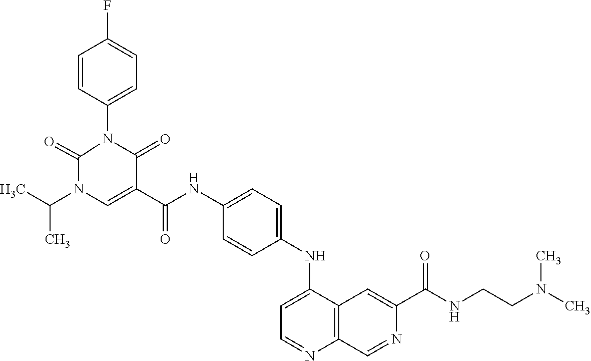 [SMILES: CC(C)n1cc(C(=O)Nc2ccc(Nc3ccnc4cnc(C(=O)NCCN(C)C)cc34)cc2)c(=O)n(-c2ccc(F)cc2)c1=O] | LC-MS (Method 7A): Rt 1.57 mins; MS m/z 625.3 = [M + H]+ ¹H NMR (400 MHz, Methanol-d4) δ 9.21 (s, 1H), 9.02 (s, 1H), 8.72 (s, 1H), 8.56 (d, J = 5.6 Hz, 1H), 7.80- 7.71 (m, 2H), 7.44-7.32 (m, 4H), 7.32-7.21 (m, 2H), 7.07 (d, J = 5.6 Hz, 1H), 4.97-4.89 (m, 1H), 3.86 (t, J = 5.9 Hz, 2H), 3.38 (t, J = 5.9 Hz, 2H), 2.95 (s, 6H), 1.50 (d, J = 6.8 Hz, 6H). |
| | N-[2-(dimethylamino)ethyl]-4-[4-[[3-(4-fluorophenyl)-1-isopropyl-2,4-dioxo-pyrimidine-5- | |
| | carbonyl]amino]anilino]-1,7-naphthyridine-6-carboxamide | |

### Example 8

### N-(1-methyl-4-piperidyl)-4-[4-[[2-oxo-1-(2-pyridyl)pyridine-3-carbonyl]amino]phenoxy]-1,7-naphthyridine-6-carboxamide

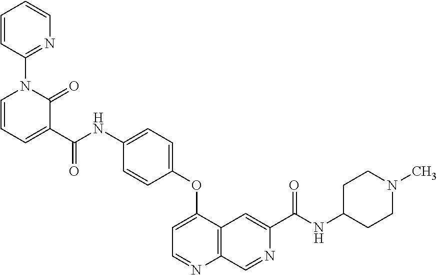 [SMILES: CN1CCC(NC(=O)c2cc3c(Oc4ccc(NC(=O)c5cccn(-c6ccccn6)c5=O)cc4)ccnc3cn2)CC1]

### Step 1: Tert-butyl N-[4-[[6-[(1-methyl-4-piperidyl)carbamoyl]-1,7-naphthyridin-4-yl]oxy]phenyl]carbamate

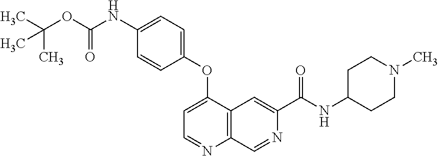 [SMILES: CN1CCC(NC(=O)c2cc3c(Oc4ccc(NC(=O)OC(C)(C)C)cc4)ccnc3cn2)CC1]

Tert-butyl N-[4-[(6-chloro-1,7-naphthyridin-4-yl)oxy]phenyl]carbamate (Example 6, step 1) (1.00 g, 2.69 mmol), 1-methylpiperidin-4-amine (921 mg, 8.07 mmol), XantPhos-Pd-G3 (255 mg, 0.269 mmol) and sodium carbonate (855 mg, 8.07 mmol) in DMA (30 mL) were added to chamber A of a 400 mL COware apparatus. The suspension was flushed with nitrogen. A solution of methanesulfonyl chloride (924 mg, 8.07 mmol) and formic acid (0.30 mL, 8.07 mmol) in nitrogen flushed toluene (5 mL) was added to chamber B, the vessel was sealed, triethylamine (2.2 mL, 16.1 mmol) was added to chamber B and the reaction mixture was stirred at 100° C. for 18 h. The cooled reaction mixture was filtered and the collected solids were partitioned between EtOAc (10 mL) and water (10 mL). The filtrate was diluted with EtOAc (70 mL) and water (50 mL), the phases were separated and the aqueous layer was extracted with EtOAc (30 mL). The combined organic fractions were then washed with water (30 mL) and brine (2×30 mL). A significant amount of solid was present in both phases so the mixture was filtered and the collected solids washed with EtOAc (10 mL) and water (10 mL) to afford a white solid. The organic phase of the filtrate was concentrated in vacuo and the resulting oil was triturated with EtOAc (2×5 mL) and filtered to afford a white solid. The two batches of solid material were combined to afford tert-butyl N-[4-[[6-[(1-methyl-4-piperidyl)carbamoyl]-1,7-naphthyridin-4-yl]oxy]phenyl]carbamate (612 mg) as a white solid.

LC-MS (Method 2A): Rt 0.65 min; MS m/z 478.3=[M+H]+

1H NMR (500 MHz, DMSO-d6) δ 9.55 (s, 1H), 9.42 (d, J=0.8 Hz, 1H), 8.90 (d, J=5.2 Hz, 1H), 8.78 (d, J=0.8 Hz, 1H), 8.75 (d, J=8.4 Hz, 1H), 7.62 (d, J=8.9 Hz, 2H), 7.31-7.24 (m, 2H), 6.86 (d, J=5.2 Hz, ¹H), 3.89-3.79 (m, 1H), 2.81-2.72 (m, 2H), 2.17 (s, 3H), 2.05-1.93 (m, 2H), 1.82-1.68 (m, 4H), 1.50 (s, 9H).

### Step 2: 4-(4-Aminophenoxy)-N-(1-methyl-4-piperidyl)-1,7-naphthyridine-6-carboxamide

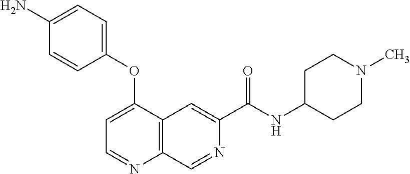 [SMILES: CN1CCC(NC(=O)c2cc3c(Oc4ccc(N)cc4)ccnc3cn2)CC1]

A solution of tert-butyl N-[4-[[6-[(1-methyl-4-piperidyl)carbamoyl]-1,7-naphthyridin-4-yl]oxy]phenyl]carbamate (Example 8, step 1)(765 mg, 1.60 mmol) and trifluoracetic acid (5.0 mL, 64.9 mmol) in DCM (20 mL) was stirred at room temperature for 18 h. The reaction mixture was concentrated in vacuo and the resulting viscous oil was loaded onto a 10 g SCX cartridge. The column was washed sequentially with MeOH (3×10 mL) and 7 M NH₃ in MeOH (3×10 mL). The methanolic ammonia fraction was concentrated in vacuo to afford 4-(4-aminophenoxy)-N-(1-methyl-4-piperidyl)-1,7-naphthyridine-6-carboxamide (565 mg) as a yellow solid.

LC-MS (Method 2A) 0.73 min; MS m/z 378.2=[M+H]+

¹H NMR (500 MHz, DMSO-d6) δ 9.39 (s, 1H), 8.88 (d, J=5.2 Hz, 1H), 8.77 (d, J=0.7 Hz, 1H), 8.74 (d, J=8.4 Hz, 1H), 7.03-6.97 (m, 2H), 6.83 (d, J=5.2 Hz, 1H), 6.73-6.66 (m, 2H), 5.24 (s, 2H), 3.89-3.77 (m, 1H), 2.79-2.72 (m, 2H), 2.17 (s, 3H), 2.05-1.95 (m, 2H), 1.81-1.68 (m, 4H).

### Step 3: 4-[4-[(1,5-Dimethyl-3-oxo-2-phenyl-pyrazole-4-carbonyl)amino]phenoxy]-N-(1-methyl-4-piperidyl)-1,7-naphthyridine-6-carboxamide

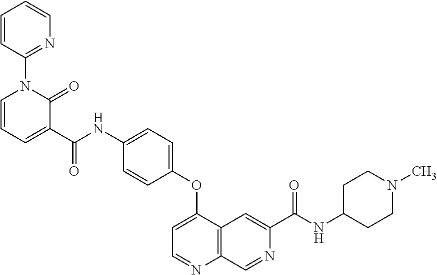 [SMILES: CN1CCC(NC(=O)c2cc3c(Oc4ccc(NC(=O)c5cccn(-c6ccccn6)c5=O)cc4)ccnc3cn2)CC1]

2-oxo-1-(2-pyridyl)pyridine-3-carboxylic acid (31 mg, 0.141 mmol) was added to a solution of 4-(4-aminophenoxy)-N-(1-methyl-4-piperidyl)-1,7-naphthyridine-6-carboxamide (Example 8 step 2)(50 mg, 0.128 mmol), HATU (64 mg, 0.167 mmol) and DIPEA (0.056 mL, 0.321 mmol) in DMF (2 mL). The solution was stirred for 2 h at room temperature. Water (10 mL) was added to the reaction mixture and the solution was stirred for 15 minutes after which time a precipitate had formed. The precipitate was collected by filtration, washed with water (3×5 mL) and dried to afford 4-[4-[(1,5-Dimethyl-3-oxo-2-phenyl-pyrazole-4-carbonyl)amino]phenoxy]-N-(1-methyl-4-piperidyl)-1,7-naphthyridine-6-carboxamide (22.1 mg) as a brown solid.

LC-MS (Method 7A): 1.75 min; MS m/z 576.3=[M+H]+

¹H NMR (500 MHz, DMSO-d6) δ 11.94 (s, 1H), 9.45 (s, 1H), 9.16-9.02 (m, 1H), 8.93 (d, J=5.2 Hz, 1H), 8.86-8.75 (m, 1H), 8.71-8.65 (m, 1H), 8.62 (dd, J=7.2, 2.2 Hz, 1H), 8.27 (dd, J=6.7, 2.2 Hz, 1H), 8.13-8.06 (m, 1H), 7.93-7.87 (m, 2H), 7.85 (d, J=8.1 Hz, 1H), 7.62 (ddd, J=7.5, 4.9, 0.9 Hz, 1H), 7.42-7.31 (m, 2H), 6.96 (d, J=5.2 Hz, 1H), 6.78 (dd, J=7.0 Hz, 1H), 4.22-4.00 (m, 1H), 3.45-3.36 (m, 2H), 3.16-2.95 (m, 2H), 2.73 (s, 3H), 2.08-1.87 (m, 4H).

The compounds of the following tabulated examples (Table 6) were prepared analogously to Example 8 step 3 from 4-(4-aminophenoxy)-N-(1-methyl-4-piperidyl)-1,7-naphthyridine-6-carboxamide (Example 8, step 2) and the appropriate carboxylic acid.

TABLE 6

| Ex. | Structure and Name | Retention Time, [M + H]+, 1H NMR |
|---|---|---|
| 8.1 | 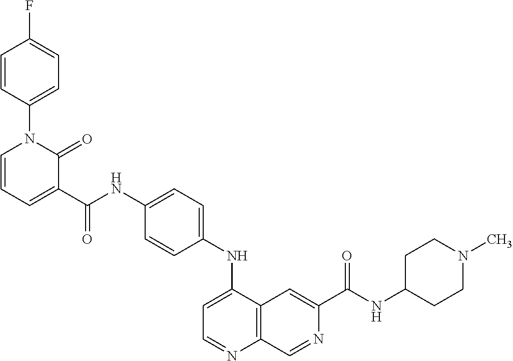 [SMILES: CN1CCC(NC(=O)c2cc3c(Nc4ccc(NC(=O)c5cccn(-c6ccc(F)cc6)c5=O)cc4)ccnc3cn2)CC1] | LC-MS (Method 7A): 2.05 min; MS m/z 593.3 = [M + H]+ ¹H NMR (500 MHz, DMSO-d6) δ 12.05 (s, 1H), 9.43 (d, J = 0.8 Hz, 1H), 8.92 (d, J = 5.2 Hz, 1H), 8.81 (d, J = 8.4 Hz, 1H), 8.79 (d, J = 0.8 Hz, 1H), 8.60 (dd, J = 7.3, 2.2 Hz, 1H), 8.12 (dd, J = 6.6, 2.2 Hz, 1H), 7.92-7.85 (m, 2H), 7.62 (ddt, J = 8.4, 5.7, 2.8 Hz, 2H), 7.48-7.40 (m, 2H), 7.39-7.33 (m, 2H), 6.93 (d, J = 5.2 Hz, 1H), 6.73 (dd, 1H), 3.94-3.83 (m, 1H), 2.92-2.80 (m, 2H), 2.27 (s, 3H), 2.17 (s, 2H), 1.86-1.71 (m, 4H). |
| | 4-[4-[[1-(4-fluorophenyl)-2-oxo-pyridine-3-carbonyl]amino]phenoxy]-N-(1-methyl-4- | |
| | piperidyl)-1,7-naphthyridine-6-carboxamide | |
| 8.2 | 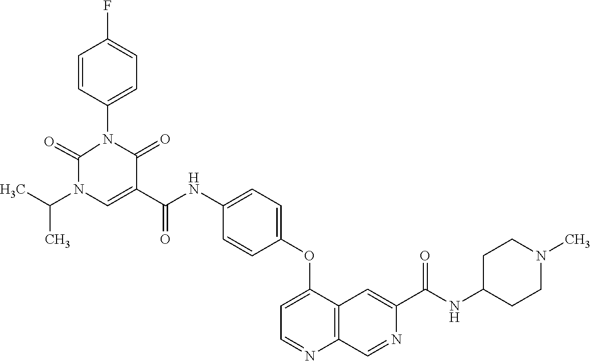 [SMILES: CC(C)n1cc(C(=O)Nc2ccc(Oc3ccnc4cnc(C(=O)NC5CCN(C)CC5)cc34)cc2)c(=O)n(-c2ccc(F)cc2)c1=O] | LC-MS (Method 7A): 2.22 min; MS m/z 652.3 = [M + H]+ 1H NMR (500 MHz, DMSO-d6) δ 10.98 (s, 1H), 9.43 (d, J = 0.8 Hz, 1H), 8.91 (d, J = 5.2 Hz, 1H), 8.78 (d, J = 0.8 Hz, 1H), 8.76 (d, J = 8.4 Hz, 1H), 8.68 (s, 1H), 7.89-7.82 (m, 2H), 7.47-7.40 (m, 2H), 7.40-7.32 (m, 4H), 6.93 (d, J = 5.2 Hz, 1H), 4.78 (hept, J = 6.9 Hz, 1H), 3.89-3.79 (m, 1H), 2.82-2.72 (m, 2H), 2.18 (s, 3H), 2.07-1.95 (m, 2H), 1.83-1.68 (m, 4H), 1.43 (d, J = 6.8 Hz, 6H). |
| | 4-[4-[[3-(4-fluorophenyl)-1-isopropyl-2,4-dioxo-pyrimidine-5-carbonyl]amino]phenoxy]-N-(1- | |
| | methyl-4-piperidyl)-1,7-naphthyridine-6-carboxamide | |
| 8.3 | 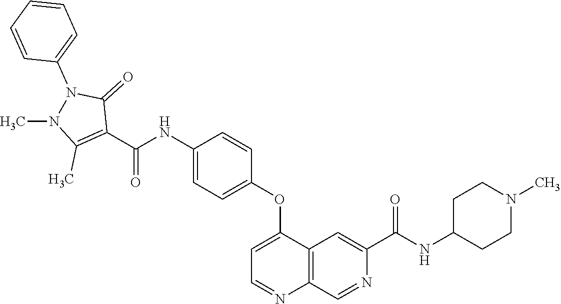 [SMILES: Cc1c(C(=O)Nc2ccc(Oc3ccnc4cnc(C(=O)NC5CCN(C)CC5)cc34)cc2)c(=O)n(-c2ccccc2)n1C] | LC-MS (Method 7B): 2.96 min; MS m/z 592.4 = [M + H]+ ¹H NMR (400 MHz, DMSO-d6) δ 10.89 (s, 1H), 9.43 (d, J = 0.8 Hz, 1H), 8.92 (d, J = 5.2 Hz, 1H), 8.80 (d, J = 0.8 Hz, 1H), 8.75 (d, J = 8.4 Hz, 1H), 7.83-7.73 (m, 2H), 7.66-7.48 (m, 3H), 7.48-7.41 (m, 2H), 7.39-7.28 (m, 2H), 6.93 (d, J = 5.2 Hz, 1H), 3.94-3.77 (m, 1H), 3.38 (s, 3H), 2.81 -2.73 (m, 2H), 2.73 (s, 3H), 2.19 (s, 3H), 2.07-1.94 (m, 2H), 1.83-1.66 (m, 4H). |
| | 4-[4-[(1,5-Dimethyl-3-oxo-2-phenyl-pyrazole-4-carbonyl)amino]phenoxy]-N-(1-methyl-4- | |
| | piperidyl)-1,7-naphthyridine-6-carboxamide | |
| 8.4 | 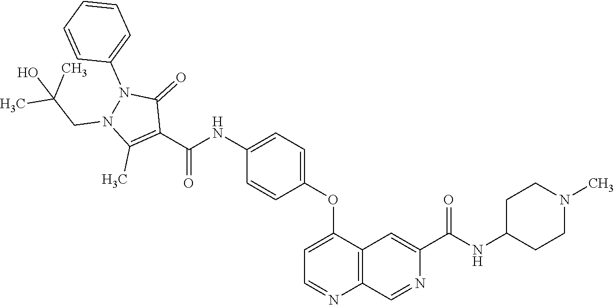 [SMILES: Cc1c(C(=O)Nc2ccc(Oc3ccnc4cnc(C(=O)NC5CCN(C)CC5)cc34)cc2)c(=O)n(-c2ccccc2)n1CC(C)(C)O] | LC-MS (Method 7A): 1.86 min; MS m/z 650.4 = [M + H]+ 1H NMR (400 MHz, DMSO-d6) δ 10.87 (s, 1H), 9.43 (d, J = 0.7 Hz, 1H), 8.91 (d, J = 5.2 Hz, 1H), 8.84-8.71 (m, 2H), 7.82-7.73 (m, 2H), 7.61- 7.53 (m, 2H), 7.50-7.42 (m, 1H), 7.40-7.28 (m, 4H), 6.92 (d, J = 5.2 Hz, 1H), 4.83 (s, 1H), 3.86 (s, 3H), 2.89-2.74 (m, 5H), 2.21 (s, 3H), 2.16- 1.97 (m, 2H), 1.88-1.67 (m, 4H), 0.97 (s, 6H). |
| | 4-[4-[[1-(2-hydroxy-2-methyl-propyl)-5-methyl-3-oxo-2-phenyl-pyrazole-4-carbonyl]amino]phenoxy]- | |
| | N-(1-methyl-4-piperidyl)-1,7-naphthyridine-6-carboxamide | |

### Example 9

### 4-[4-[[3-(5-Fluoro-2-pyridyl)-1-isopropyl-2,4-dioxo-pyrimidine-5-carbonyl]amino]phenoxy]-N-(1-methyl-4-piperidyl)-1,7-naphthyridine-6-carboxamide

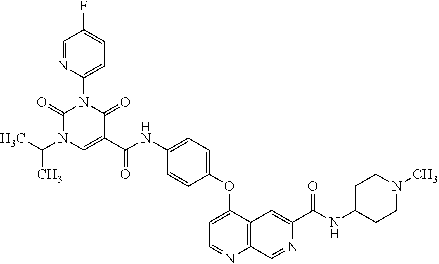 [SMILES: CC(C)n1cc(C(=O)Nc2ccc(Oc3ccnc4cnc(C(=O)NC5CCN(C)CC5)cc34)cc2)c(=O)n(-c2ccc(F)cn2)c1=O]

### 4-[4-[[3-(5-Fluoro-2-pyridyl)-1-isopropyl-2,4-dioxo-pyrimidine-5-carbonyl]amino]phenoxy]-N-(1-methyl-4-piperidyl)-1,7-naphthyridine-6-carboxamide

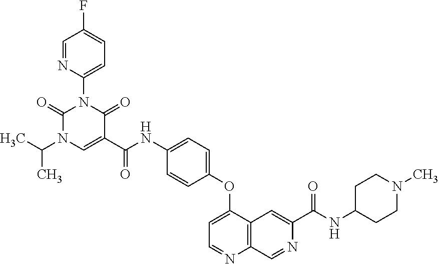 [SMILES: CC(C)n1cc(C(=O)Nc2ccc(Oc3ccnc4cnc(C(=O)NC5CCN(C)CC5)cc34)cc2)c(=O)n(-c2ccc(F)cn2)c1=O]

HATU (64 mg, 0.167 mmol) was added to a solution of 4-(4-aminophenoxy)-N-(1-methyl-4-piperidyl)-1,7-naphthyridine-6-carboxamide (Example 8, step 2)(50 mg, 0.128 mmol), 3-(5-fluoro-2-pyridyl)-1-isopropyl-2,4-dioxo-pyrimidine-5-carboxylic acid (Intermediate E)(38 mg, 0.128 mmol) and DIPEA (56 μL, 0.321 mmol) in DMF (2 mL). The solution was stirred at room temperature for 18 h. The formed precipitate was collected by filtration, washed with water (2×1 mL) and dried in vacuo at 40° C. to give 4-[4-[[3-(5-Fluoro-2-pyridyl)-1-isopropyl-2,4-dioxo-pyrimidine-5-carbonyl]amino]phenoxy]-N-(1-methyl-4-piperidyl)-1,7-naphthyridine-6-carboxamide (37 mg) as a white solid.

LC-MS (Method 7A): 1.97 min; MS m/z 653.3=[M+H]+

¹H NMR (400 MHz, DMSO-d6) δ 10.81 (s, 1H), 9.43 (d, J=0.8 Hz, 1H), 8.91 (d, J=5.2 Hz, 1H), 8.78 (d, J=0.8 Hz, 1H), 8.75 (d, J=8.4 Hz, 1H), 8.71 (s, 1H), 8.66 (d, J=3.1 Hz, 1H), 8.02 (td, J=8.4, 3.1 Hz, 1H), 7.91-7.82 (m, 2H), 7.68 (dd, J=8.8, 4.1 Hz, 1H), 7.40-7.31 (m, 2H), 6.93 (d, J=5.2 Hz, 1H), 4.77 (hept, J=6.7 Hz, 1H), 3.90-3.78 (m, 1H), 2.76 (m, 2H), 2.18 (s, 3H), 2.06-1.96 (m, 2H), 1.82-1.67 (m, 4H), 1.44 (d, J=6.8 Hz, 6H).

The compound of the following tabulated example (Table 7) was prepared analogously to Example 9 from 4-(4-aminophenoxy)-N-(1-methyl-4-piperidyl)-1,7-naphthyridine-6-carboxamide (Example 8, step 2) and the appropriate carboxylic acid.

TABLE 7

| Ex. | Structure and Name | Retention Time, [M + H]+, 1H NMR |
|---|---|---|
| 9.1 | 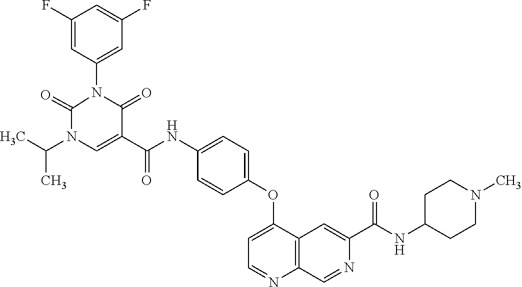 [SMILES: CC(C)n1cc(C(=O)Nc2ccc(Oc3ccnc4cnc(C(=O)NC5CCN(C)CC5)cc34)cc2)c(=O)n(-c2cc(F)cc(F)c2)c1=O] | LC-MS (Method 7A): 2.32 min; MS m/z 670.3 = [M + H]+ ¹H NMR (500 MHz, DMSO-d6) δ 10.88 (s, 1H), 9.43 (d, J = 0.8 Hz, 1H), 8.92 (d, J = 5.2 Hz, 1H), 8.78 (d, J = 0.8 Hz, 1H), 8.76 (d, J = 8.4 Hz, 1H), 8.70 (s, 1H), 7.92-7.81 (m, 2H), 7.48-7.39 (m, 1H), 7.40-7.33 (m, 2H), 7.33-7.22 (m, 2H), 6.94 (d, J = 5.2 Hz, 1H), 4.87-4.68 (m, 1H), 3.90-3.76 (m, 1H), 2.80-2.73 (m, 2H), 2.18 (s, 3H), 2.07-1.94 (m, 2H), 1.84-1.68 (m, 4H), 1.43 (d, J = 6.8 Hz, 6H). |
| | 4-[4-[[3-(3,5-difluorophenyl)-1-isopropyl-2,4-dioxo-pyrimidine-5-carbonyl]amino] | |
| | phenoxy]-N-(1-methyl-4-piperidyl)-1,7-naphthyridine-6-carboxamide | |

### Example 10

### 1-(4-fluorophenyl)-2-oxo-N-[5-[(6-piperazin-1-yl-1,7-naphthyridin-4-yl)oxy]-2-pyridyl]pyridine-3-carboxamide

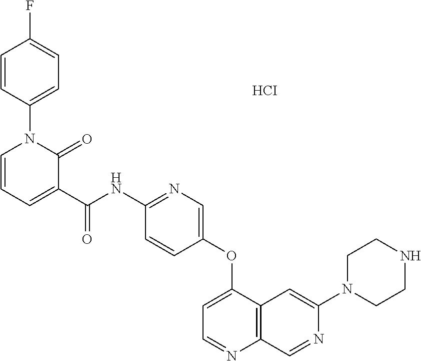 [SMILES: O=C(Nc1ccc(Oc2ccnc3cnc(N4CCNCC4)cc23)cn1)c1cccn(-c2ccc(F)cc2)c1=O ; "HCI"]

### Step 1: 6-chloro-4-[(6-nitro-3-pyridyl)oxy]-1,7-naphthyridine

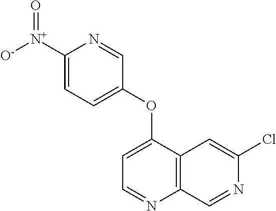 [SMILES: O=[N+]([O-])c1ccc(Oc2ccnc3cnc(Cl)cc23)cn1]

4,6-dichloro-1,7-naphthyridine (2.40 g, 12.1 mmol) and 6-nitropyridin-3-ol (2.04 g, 14.6 mmol) were added to a dry round bottom flask and dissolved in chlorobenzene (30 mL) before DIPEA (4.2 mL, 24.2 mmol) was added. The reaction mixture was then stirred and warmed to 150° C. for 20 h. On cooling to room temperature, water (10 mL) was added and biphasic mixture was stirred and then filtered. The collected solid was washed with water (20 mL) and diethyl ether (20 mL) and then dried in vacuo to afford 6-chloro-4-[(6-nitro-3-pyridyl)oxy]-1,7-naphthyridine (2.71 g) as a light grey solid.

LC-MS (Method 2A): 1.06 min; MS m/z 303.0=[M+H]+

¹H NMR (400 MHz, DMSO) δ 9.37 (d, J=0.7 Hz, 1H), 8.98 (d, J=5.1 Hz, 1H), 8.76 (d, J=2.7 Hz, 1H), 8.50 (d, J=8.9 Hz, 1H), 8.29 (d, J=0.7 Hz, 1H), 8.20 (dd, J=8.9, 2.7 Hz, 1H), 7.34 (d, J=5.1 Hz, 1H).

### Step 2: 5-[(6-chloro-1,7-naphthyridin-4-yl)oxy]pyridin-2-amine

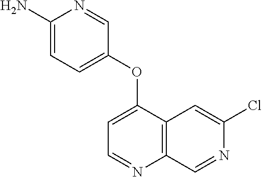 [SMILES: Nc1ccc(Oc2ccnc3cnc(Cl)cc23)cn1]

6-Chloro-4-[(6-nitro-3-pyridyl)oxy]-1,7-naphthyridine (Example 10, step 1) (5.29 g, 17.5 mmol), iron powder (4.88 g, 87.4 mmol) and ammonium chloride (4.67 g, 87.4 mmol) in methanol (100 mL) and water (30 mL) was heated to 75° C. and stirred for 1 h. The reaction mixture was cooled to room temperature, filtered through celite and the filter bed washed with MeOH (3×30 mL) and CHCl₃/IPA (2:1, 3×30 mL). The combined filtrates were concentrated in vacuo. The residue was partitioned between CHCl₃/IPA (2:1, 50 mL) and water (50 mL), the organic separated and the aqueous layer further extracted with CHCl3/IPA (2:1, 2×50 mL). The combined organics were passed through a hydrophobic frit before being concentrated in vacuo to afford 5-[(6-chloro-1,7-naphthyridin-4-yl)oxy]pyridin-2-amine (4.6 g) as a brown solid.

LC-MS (Method 2A): 0.83 min; MS m/z=273.3 [M+H]+

¹H NMR (400 MHz, DMSO) δ 9.27 (s, 1H), 8.83 (d, J=5.1 Hz, 1H), 8.23 (s, 1H), 7.95 (d, J=2.8 Hz, 1H), 7.43 (dd, J=8.9, 2.8 Hz, 1H), 6.86 (d, J=5.1 Hz, 1H), 6.59 (d, J=8.9 Hz, 1H), 6.18 (s, 2H).

### Step 3: N-[5-[(6-chloro-1,7-naphthyridin-4-yl)oxy]-2-pyridyl]-1-(4-fluorophenyl)-2-oxo-pyridine-3-carboxamide

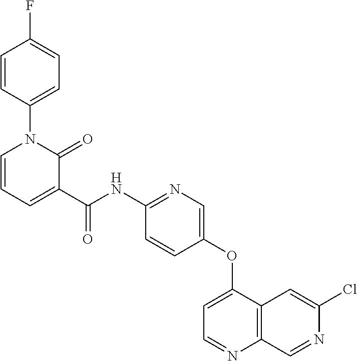 [SMILES: O=C(Nc1ccc(Oc2ccnc3cnc(Cl)cc23)cn1)c1cccn(-c2ccc(F)cc2)c1=O]

HATU (179 mg, 0.472 mmol) was added to a solution of 5-[(6-chloro-1,7-naphthyridin-4-yl)oxy]pyridin-2-amine (Example 10, step 2) (99 mg, 0.363 mmol), 1-(4-fluorophenyl)-2-oxo-pyridine-3-carboxylic acid (Intermediate B)(85 mg, 0.363 mmol) and N,N-Diisopropylethylamine (0.16 mL, 0.908 mmol) in DMF (2 mL). The solution was allowed to stir at room temperature for 60 h. Water (5 mL) was added to the reaction mixture and the formed precipitate was collected by filtration, washed with water (3×5 mL) and dried to afford N-[5-[(6-chloro-1,7-naphthyridin-4-yl)oxy]-2-pyridyl]-1-(4-fluorophenyl)-2-oxo-pyridine-3-carboxamide (137 mg) as a light brown solid.

LC-MS (Method 2A): 1.26 min; MS m/z=488.1 [M+H]+

¹H NMR (400 MHz, DMSO-d6) δ 12.47 (s, 1H), 9.32 (d, J=0.7 Hz, 1H), 8.88 (d, J=5.2 Hz, 1H), 8.65 (dd, J=7.3, 2.2 Hz, 1H), 8.51-8.40 (m, 2H), 8.29 (d, J=0.7 Hz, 1H), 8.17 (dd, J=6.6, 2.2 Hz, 1H), 7.95 (dd, J=9.0, 2.9 Hz, 1H), 7.68-7.57 (m, 2H), 7.49-7.37 (m, 2H), 7.01 (d, J=5.2 Hz, 1H), 6.80-6.69 (m, 1H).

### Step 4: tert-butyl 4-[4-[[6-[[1-(4-fluorophenyl)-2-oxo-pyridine-3-carbonyl]amino]-3-pyridyl]oxy]-1,7-naphthyridin-6-yl]piperazine-1-carboxylate

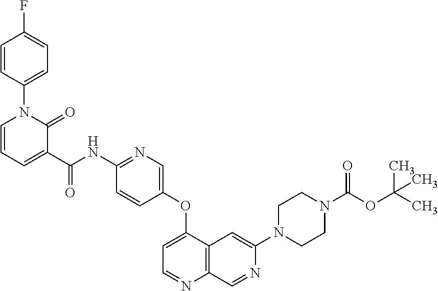 [SMILES: CC(C)(C)OC(=O)N1CCN(c2cc3c(Oc4ccc(NC(=O)c5cccn(-c6ccc(F)cc6)c5=O)nc4)ccnc3cn2)CC1]

A mixture of N-[5-[(6-chloro-1,7-naphthyridin-4-yl)oxy]-2-pyridyl]-1-(4-fluorophenyl)-2-oxo-pyridine-3-carboxamide (Example 10, step 3)(68 mg, 0.139 mmol), tert-butyl piperazine-1-carboxylate (39 mg, 0.209 mmol), cesium carbonate (27 mg, 0.139 mmol) and RuPhos Pd G3 (12 mg, 0.0139 mmol) in dry DMA (5 mL) was degassed with nitrogen for 5 mins before being heated to 90° C. and stirred for 1 h.

The reaction was allowed to cool to room temperature before water (10 mL) was added, the resultant suspension was extracted with DCM (3×10 mL) and the combined organics were passed through a hydrophobic frit and concentrated in vacuo. The crude material was purified by acidic reverse phase chromatography (eluting with 10-100% MeCN (0.1% formic acid) in water (0.1% formic acid)) to afford tert-butyl 4-[4-[[6-[[1-(4-fluorophenyl)-2-oxo-pyridine-3-carbonyl]amino]-3-pyridyl]oxy]-1,7-naphthyridin-6-yl]piperazine-1-carboxylate (49 mg) as a pale yellow solid.

LC-MS (Method 2A): 1.36 min; MS m/z=638.3 [M+H]+

¹H NMR (400 MHz, DMSO-d6) δ 12.44 (s, 1H), 9.10 (d, J=0.7 Hz, 1H), 8.64 (dd, J=7.3, 2.2 Hz, 1H), 8.48 (d, J=5.0 Hz, 1H), 8.44 (d, J=9.4 Hz, 1H), 8.38 (d, J=2.9 Hz, 1H), 8.16 (dd, J=6.6, 2.2 Hz, 1H), 7.88 (dd, J=9.0, 2.9 Hz, 1H), 7.67-7.57 (m, 2H), 7.49-7.37 (m, 2H), 7.19 (s, 1H), 6.79-6.69 (m, 2H), 3.67-3.56 (m, 4H), 3.56-3.44 (m, 4H), 1.44 (s, 9H).

### Step 5: 1-(4-fluorophenyl)-2-oxo-N-[5-[(6-piperazin-1-yl-1,7-naphthyridin-4-yl)oxy]-2-pyridyl]pyridine-3-carboxamide

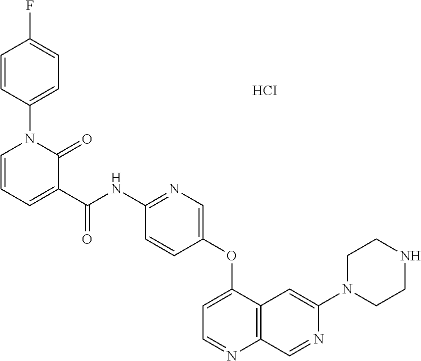 [SMILES: O=C(Nc1ccc(Oc2ccnc3cnc(N4CCNCC4)cc23)cn1)c1cccn(-c2ccc(F)cc2)c1=O ; "HCI"]

4M HCl in dioxane (2.0 mL, 8.00 mmol) was added to a solution of tert-butyl 4-[4-[[6-[[1-(4-fluorophenyl)-2-oxo-pyridine-3-carbonyl]amino]-3-pyridyl]oxy]-1,7-naphthyridin-6-yl]piperazine-1-carboxylate (Example 10, step 4) (49 mg, 0.0768 mmol) in 1,4-dioxane (2 mL) and the reaction mixture was stirred at room temperature for 1 h. The formed suspension was filtered and the collected solid was dried in vacuo and then freeze dried to afford 1-(4-fluorophenyl)-2-oxo-N-[5-[(6-piperazin-1-yl-1,7-naphthyridin-4-yl)oxy]-2-pyridyl]pyridine-3-carboxamide hydrochloride (28.5 mg) as a yellow solid.

LC-MS (Method 7A): 1.80 min; MS m/z=538.2 [M+H]+

1H NMR (500 MHz, DMSO-d6) δ 12.46 (s, 1H), 9.15 (d, J=0.7 Hz, 1H), 8.87 (s, 2H), 8.64 (dd, J=7.3, 2.2 Hz, 1H), 8.55 (d, J=5.0 Hz, 1H), 8.46 (d, J=9.0 Hz, 1H), 8.39 (d, J=2.9 Hz, 1H), 8.17 (dd, J=6.6, 2.2 Hz, 1H), 7.89 (dd, J=9.0, 2.9 Hz, 1H), 7.67-7.59 (m, 2H), 7.48-7.40 (m, 2H), 7.33 (s, 1H), 6.79 (d, J=5.0 Hz, 1H), 6.77-6.73 (m, 1H), 3.89-3.84 (m, 4H), 3.31-3.21 (m, 4H).

The compounds of the following tabulated examples (Table 8) were prepared analogously to Example 10, step 4 from N-[5-[(6-chloro-1,7-naphthyridin-4-yl)oxy]-2-pyridyl]-1-(4-fluorophenyl)-2-oxo-pyridine-3-carboxamide (Example 10, step 3) and the appropriate substituted piperazine.

TABLE 8

| Ex. | Structure and Name | Retention Time, [M + H]+, 1H NMR |
|---|---|---|
| 10.1 | 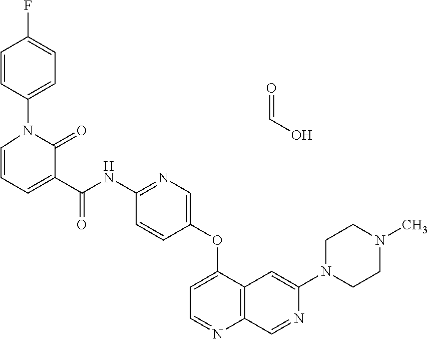 [SMILES: CN1CCN(c2cc3c(Oc4ccc(NC(=O)c5cccn(-c6ccc(F)cc6)c5=O)nc4)ccnc3cn2)CC1 ; O=CO] | LC-MS (Method 7A): 1.84 min; MS m/z 552.2 = [M + H]+ ¹H NMR (500 MHz, DMSO-d₆) δ 12.44 (s, 1H), 9.08 (d, J = 0.7 Hz, 1H), 8.63 (dd, J = 7.3, 2.2 Hz, 1H), 8.47 (d, J = 5.0 Hz, 1H), 8.45-8.41 (m, 1H), 8.39- 8.37 (m, 1H), 8.16 (dd, J = 6.6, 2.2 Hz, 1H), 8.14 (s, 1H), 7.88 (dd, J = 9.0, 2.9 Hz, 1H), 7.64-7.59 (m, 2H), 7.46- 7.39 (m, 2H), 7.16 (s, 1H), 6.76- 6.72 (m, 1H), 6.71 (d, J = 5.0 Hz, 1H), 3.66-3.58 (m, 4H), 2.52-2.51 (m, 4H), 2.28 (s, 3H). |
| | 1-(4-fluorophenyl)-N-[5-[[6-(4-methylpiperazin-1-yl)-1,7-naphthyridin- | |
| | 4-yl]oxy]-2-pyridyl]-2-oxo-pyridine-3-carboxamide formate salt | |
| 10.2 | 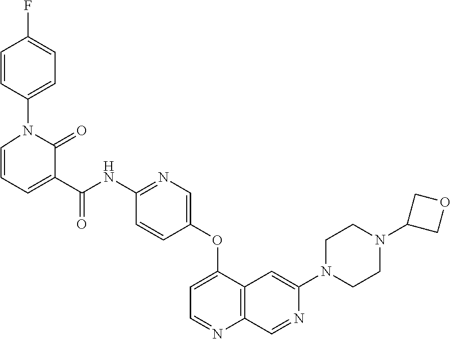 [SMILES: O=C(Nc1ccc(Oc2ccnc3cnc(N4CCN(C5COC5)CC4)cc23)cn1)c1cccn(-c2ccc(F)cc2)c1=O] | LC-MS (Method 7A): 1.93 min; MS m/z 594.2 = [M + H]+ ¹H NMR (500 MHz, DMSO-d₆) δ 12.44 (s, 1H), 9.12-9.06 (m, 1H), 8.64 (dd, J = 7.3, 2.2 Hz, 1H), 8.47 (d, J = 5.0 Hz, 1H), 8.46-8.41 (m, 1H), 8.40-8.35 (m, 1H), 8.16 (dd, J = 6.6, 2.2 Hz, 1H), 7.89 (dd, J = 9.0, 2.9 Hz, 1H), 7.66- 7.58 (m, 2H), 7.47-7.40 (m, 2H), 7.16 (s, 1H), 6.77-6.73 (m, 1H), 6.72 (d, J = 5.0 Hz, 1H), 4.58 (t, J = 6.5 Hz, 2H), 4.50 (t, J = 6.1 Hz, 2H), 3.68-3.61 (m, 4H), 3.49-3.43 (m, 1H), 2.45-2.39 (m, 4H). |
| | 1-(4-fluorophenyl)-N-[5-[[6-[4-(oxetan-3-yl)piperazin-1-yl]-1,7- | |
| | naphthyridin-4-yl]oxy]-2-pyridyl]-2-oxo-pyridine-3-carboxamide | |

### Example 11

### 4-[[6-[[1-(4-fluorophenyl)-2-oxo-pyridine-3-carbonyl]amino]-3-pyridyl]oxy]-N-(1-methyl-4-piperidyl)-1,7-naphthyridine-6-carboxamide

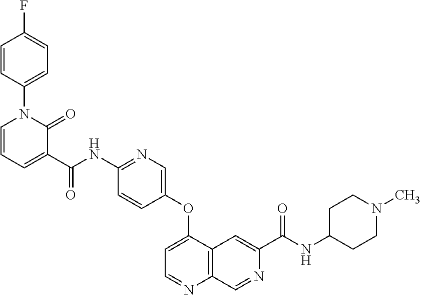 [SMILES: CN1CCC(NC(=O)c2cc3c(Oc4ccc(NC(=O)c5cccn(-c6ccc(F)cc6)c5=O)nc4)ccnc3cn2)CC1]

### 4-[[6-[[1-(4-fluorophenyl)-2-oxo-pyridine-3-carbonyl]amino]-3-pyridyl]oxy]-N-(1-methyl-4-piperidyl)-1,7-naphthyridine-6-carboxamide

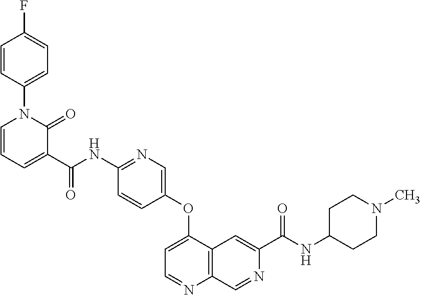 [SMILES: CN1CCC(NC(=O)c2cc3c(Oc4ccc(NC(=O)c5cccn(-c6ccc(F)cc6)c5=O)nc4)ccnc3cn2)CC1]

1-methylpiperidin-4-amine (70 mg, 0.615 mmol), N-[5-[(6-chloro-1,7-naphthyridin-4-yl)oxy]-2-pyridyl]-1-(4-fluorophenyl)-2-oxo-pyridine-3-carboxamide (Example 10, step 3)(100 mg, 0.205 mmol), [1,1-bis(diphenylphosphino)ferrocene]dichloropalladium(II) (15 mg, 0.0205 mmol) and sodium carbonate (65 mg, 0.615 mmol) in dry DMA (4 mL) were added to chamber A of a 20 mL COware apparatus. The suspension was flushed with nitrogen. Formic acid (23 uL, 0.615 mmol) and methanesulfonyl chloride (48 uL, 0.615 mmol) in nitrogen flushed dry toluene (2 mL) was added to chamber B of the apparatus. Triethylamine (0.17 mL, 1.23 mmol) was added to chamber B before the reaction mixture was heated to 105° C. and stirred for 18 h. The reaction mixture was allowed to cool to room temperature and was degassed with nitrogen for 5 mins. Water (10 mL) was added and the suspension was extracted with DCM (3×10 mL). The organic fractions were combined before being passed through a hydrophobic frit and concentrated in vacuo. The crude residue was purified by low pH reverse phase chromatography (eluting with 10-100% MeCN (0.1% formic acid) in water (0.1% formic acid)) with product containing fractions concentrated in vacuo and freeze dried to afford 4-[[6-[[1-(4-fluorophenyl)-2-oxo-pyridine-3-carbonyl]amino]-3-pyridyl]oxy]-N-(1-methyl-4-piperidyl)-1,7-naphthyridine-6-carboxamide (33.7 mg) as a brown solid.

LC-MS (Method 7A): 1.91 min; MS m/z=594.3 [M+H]+

¹H NMR (400 MHz, DMSO-d6) δ 12.46 (s, 1H), 9.45 (d, J=0.8 Hz, 1H), 8.93 (d, J=5.2 Hz, 1H), 8.80 (d, J=0.8 Hz, 1H), 8.79-8.73 (m, 1H), 8.64 (dd, J=7.3, 2.2 Hz, 1H), 8.50-8.42 (m, 2H), 8.16 (dd, J=6.6, 2.2 Hz, 1H), 7.96 (dd, J=8.9, 3.0 Hz, 1H), 7.66-7.59 (m, 2H), 7.47-7.38 (m, 2H), 7.04 (d, J=5.2 Hz, 1H), 6.79-6.68 (m, 1H), 3.91-3.78 (m, 1H), 2.82-2.72 (m, 2H), 2.18 (s, 3H), 2.06-1.96 (m, 2H), 1.82-1.68 (m, 4H).

### Example 12

### 2-Oxo-1-phenyl-N-[4-[(6-piperazin-1-yl-1,7-naphthyridin-4-yl)oxy]phenyl]pyridine-3-carboxamide

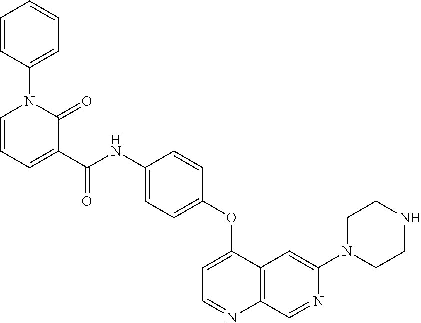 [SMILES: O=C(Nc1ccc(Oc2ccnc3cnc(N4CCNCC4)cc23)cc1)c1cccn(-c2ccccc2)c1=O]

### Step 1: 6-Chloro-4-(4-nitrophenoxy)-1,7-naphthyridine

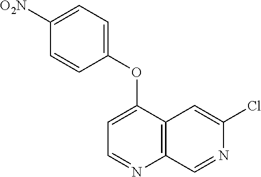 [SMILES: O=[N+]([O-])c1ccc(Oc2ccnc3cnc(Cl)cc23)cc1]

4,6-Dichloro-1,7-naphthyridine (4.00 g, 20.1 mmol), DIPEA (7.0 mL, 40.3 mmol) and 4-nitrophenol (3.36 g, 24.2 mmol) were suspended in chlorobenzene (60 mL). The reaction mixture was stirred at 150° C. for 20 h. The reaction mixture was cooled to room temperature and a solid formed. The solid was collected by filtration, washed with diethyl ether (3×5 mL) and dried to afford 6-Chloro-4-(4-nitrophenoxy)-1,7-naphthyridine (4.34 g) as a beige solid.

LC-MS (Method 2A): 1.21 min; MS m/z 301.8=[M+H]+

¹H NMR (400 MHz, DMSO-d6) δ 9.34 (m, 1H), 8.95 (d, J=5.1 Hz, 1H), 8.45-8.36 (m, 2H), 8.25-8.20 (m, 1H), 7.64-7.56 (m, 2H), 7.20 (d, J=5.1 Hz, 1H).

### Step 2: Tert-butyl 4-[4-(4-nitrophenoxy)-1,7-naphthyridin-6-yl]piperazine-1-carboxylate

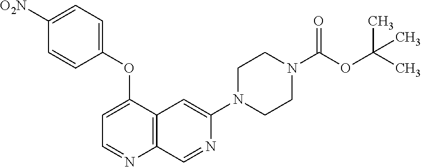 [SMILES: CC(C)(C)OC(=O)N1CCN(c2cc3c(Oc4ccc([N+](=O)[O-])cc4)ccnc3cn2)CC1]

6-Chloro-4-(4-nitrophenoxy)-1,7-naphthyridine (Example 12, step 1)(2.00 g, 6.63 mmol), tert-butyl piperazine-1-carboxylate (1.86 g, 9.98 mmol), BINAP (418 mg, 0.671 mmol) and KO^{t}Bu (2.24 g, 20.0 mmol) were suspended in toluene (30 mL). The reaction mixture was degassed for 10 mins before addition of Pd₂dba₃ (608 mg, 0.664 mmol). The resulting mixture was degassed for a further 10 mins before being stirred at 90° C. for 16 h. The reaction was cooled to room temperature and water (30 mL) was added followed by DCM (30 mL) and brine (30 mL). The organic fraction was separated and the aqueous layer was further extracted with DCM (3×35 mL). The combined organic extracts were filtered through a hydrophobic frit and concentrated in vacuo to afford the crude material which was purified by silica chromatography (eluting with a gradient of 0-75% EtOAc in heptane) to afford tert-butyl 4-[4-(4-nitrophenoxy)-1,7-naphthyridin-6-yl]piperazine-1-carboxylate (503 mg) as a yellow solid.

LC-MS (Method 2A): 1.33 min; MS m/z 452.1=[M+H]+

¹H NMR (400 MHz, DMSO-d6) δ 9.14 (m, 1H), 8.58 (d, J=4.9 Hz, 1H), 8.41-8.32 (m, 2H), 7.54-7.45 (m, 2H), 7.05 (m, 1H), 7.01 (d, J=4.9 Hz, 1H), 3.61-3.57 (m, 4H), 3.52-3.44 (m, 4H), 1.43 (s, 9H).

### Step 3: Tert-butyl 4-[4-(4-aminophenoxy)-1,7-naphthyridin-6-yl]piperazine-1-carboxylate

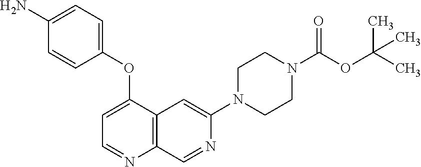 [SMILES: CC(C)(C)OC(=O)N1CCN(c2cc3c(Oc4ccc(N)cc4)ccnc3cn2)CC1]

Tert-butyl 4-[4-(4-nitrophenoxy)-1,7-naphthyridin-6-yl]piperazine-1-carboxylate (Example 12, step 2)(880 mg, 1.95 mmol), iron (545 mg, 9.76 mmol) and ammonium chloride (526 mg, 9.83 mmol) were suspended in a mixture of MeOH (15 mL) and water (4 mL). The reaction mixture was stirred at 75° C. for 1 h. The reaction mixture was cooled to room temperature then filtered through a pad of celite. The filter bed was washed with MeOH (3×15 mL) and the filtrate concentrated in vacuo. DCM (20 mL) and water (20 mL) were added and the organic phase was separated by passage through a hydrophobic frit and concentrated in vacuo. The crude solid was suspended in hot diethyl ether (20 mL), filtered and the filtrate concentrated in vacuo to afford tert-butyl 4-[4-(4-aminophenoxy)-1,7-naphthyridin-6-yl]piperazine-1-carboxylate (740 mg) as a yellow solid.

LC-MS (Method 2A): 1.09 min; MS m/z 422.2=[M+H]+

¹H NMR (400 MHz, DMSO-d6) δ 9.09-9.03 (m, 1H), 8.44 (d, J=5.0 Hz, 1H), 7.17 (s, 1H), 6.98-6.89 (m, 2H), 6.72-6.64 (m, 2H), 6.52 (d, J=5.0 Hz, 1H), 5.19 (s, 2H), 3.63-3.57 (m, 4H), 3.52-3.47 (m, 4H), 1.44 (s, 9H).

### Step 4: 2-Oxo-1-phenyl-N-[4-[(6-piperazin-1-yl-1,7-naphthyridin-4-yl)oxy]phenyl]pyridine-3-carboxamide

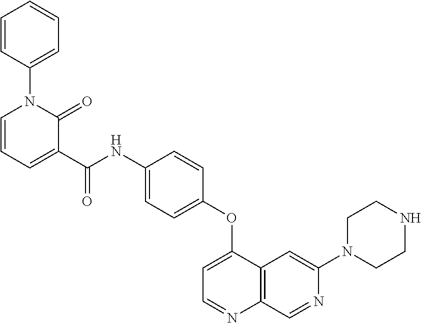 [SMILES: O=C(Nc1ccc(Oc2ccnc3cnc(N4CCNCC4)cc23)cc1)c1cccn(-c2ccccc2)c1=O]

HATU (59 mg, 0.154 mmol) was added to a solution of tert-butyl 4-[4-(4-aminophenoxy)-1,7-naphthyridin-6-yl]piperazine-1-carboxylate (Example 12, step 3)(50 mg, 0.119 mmol), 2-oxo-1-phenyl-pyridine-3-carboxylic acid (Intermediate A)(26 mg, 0.119 mmol) and DIPEA (52 μL, 0.297 mmol) in DMF (2 mL) and the mixture was stirred at room temperature for 16 h. Water (5 mL) was added to the mixture and the resulting suspension was filtered. The solid was washed with water (3×5 mL) and dried in vacuo at 40° C. to afford the protected coupled intermediate. This material was suspended in 4 M HCl in dioxane (2.0 mL, 8.00 mmol) and stirred for 1 h. The suspension was filtered under vacuum and the resulting solid was freeze dried to afford 2-oxo-1-phenyl-N-[4-[(6-piperazin-1-yl-1,7-naphthyridin-4-yl)oxy]phenyl]pyridine-3-carboxamide hydrochloride (48.9 mg) as a yellow solid.

LC-MS (Method 7A): 1.86 min; MS m/z 519.2=[M+H]+

¹H NMR (500 MHz, DMSO-d6) δ 12.08 (s, 1H), 9.16 (s, 1H), 9.04-8.97 (m, 2H), 8.61 (dd, J=7.3, 2.2 Hz, 1H), 8.56 (d, J=5.1 Hz, 1H), 8.14 (dd, J=6.6, 2.2 Hz, 1H), 7.92-7.83 (m, 2H), 7.64-7.51 (m, 5H), 7.35-7.27 (m, 3H), 6.78-6.68 (m, 2H), 3.92-3.84 (m, 4H), 3.28-3.23 (m, 4H).

The compounds of the following tabulated examples (Table 9) were prepared analogously to Example 12, step 4 from tert-butyl 4-[4-(4-aminophenoxy)-1,7-naphthyridin-6-yl]piperazine-1-carboxylate (Example 12, step 3) and the appropriate carboxylic acid.

TABLE 9

| Ex. | Structure and Name | Retention Time, [M + H]+, 1H NMR |
|---|---|---|
| 12.1 | 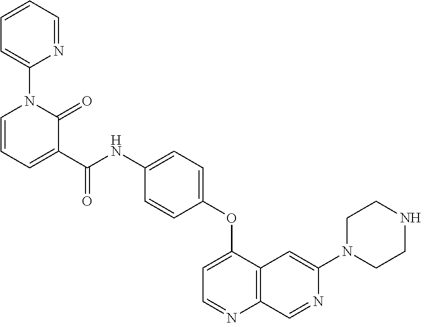 [SMILES: O=C(Nc1ccc(Oc2ccnc3cnc(N4CCNCC4)cc23)cc1)c1cccn(-c2ccccn2)c1=O] | LC-MS (Method 7A): 1.63 min; MS m/z = 520.2 [M + H]+ ¹H NMR (400 MHz, DMSO-d6) δ 11.83 (s, 1H), 9.00 (s, 1H), 8.61 (d, J = 3.8 Hz, 1H), 8.55 (dd, J = 7.2, 2.2 Hz, 1H), 8.38 (d, J = 5.0 Hz, 1H), 8.19 (dd, J = 6.7, 2.2 Hz, 1H), 8.05-8.00 (m, 1H), 7.82-7.76 (m, 3H), 7.54 (dd, J = 7.0, 5.4 Hz, 1H), 7.24-7.20 (m, 2H), 7.05- 7.00 (m, 1H), 6.75-6.66 (m, 1H), 6.56 (d, J = 5.0 Hz, 1H), 3.70-3.64 (m, 1H), 3.49-3.42 (m, 4H), 2.83-2.74 (m, 4H). |
| | 2-oxo-N-[4-[(6-piperazin-1-yl-1,7-naphthyridin-4-yl)oxy]phenyl]-1-(2- | |
| | pyridyl)pyridine-3-carboxamide | |
| 12.2 | 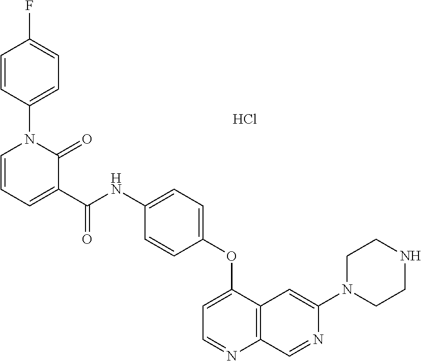 [SMILES: O=C(Nc1ccc(Oc2ccnc3cnc(N4CCNCC4)cc23)cc1)c1cccn(-c2ccc(F)cc2)c1=O ; Cl] | LC-MS (Method 7A): 1.88 min; MS m/z = 537.3 [M+H]+ ¹H NMR (500 MHz, DMSO-d6) δ 12.03 (s, 1H), 9.15 (s, 1H), 9.01 (s, 2H), 8.59 (dd, J = 7.3, 2.2 Hz, 1H), 8.55 (d, J = 5.1 Hz, 1H), 8.13 (dd, J = 6.6, 2.2 Hz, 1H), 7.96-7.80 (m, 2H), 7.65-7.58 (m, 2H), 7.47-7.40 (m, 2H), 7.33- 7.28 (m, 3H), 6.76-6.71 (m, 1H), 6.70 (d, J = 5.1 Hz, 1H), 3.92-3.82 (m, 4H), 3.28-3.21 (m, 4H). |
| | 1-(4-fluorophenyl)-2-oxo-N-[4-[(6-piperazin-1-yl-1,7-naphthyridin-4- | |
| | yl)oxy]phenyl]pyridine-3-carboxamide hydrochloride | |
| 12.3 | 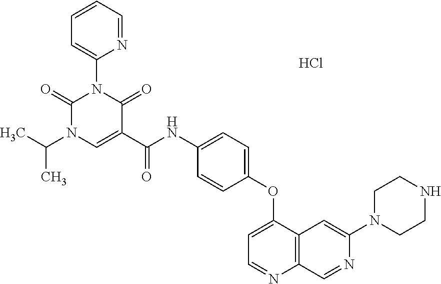 [SMILES: CC(C)n1cc(C(=O)Nc2ccc(Oc3ccnc4cnc(N5CCNCC5)cc34)cc2)c(=O)n(-c2ccccn2)c1=O ; Cl] | LC-MS (Method 7A): 1.68 min; MS m/z = 579.3 [M + H]+ ¹H NMR (400 MHz, DMSO-d6) δ 10.82 (s, 1H), 9.08 (d, J = 0.6 Hz, 1H), 8.71 (s, 1H), 8.68-8.61 (m, 1H), 8.46 (d, J = 5.0 Hz, 1H), 8.24 (s, 1H), 8.07 (td, J = 7.7, 1.9 Hz, 1H), 7.86-7.79 (m, 2H), 7.61-7.52 (m, 2H), 7.33-7.25 (m, 2H), 7.13 (s, 1H), 6.63 (d, J = 5.0 Hz, 1H), 4.84-4.71 (m, 1H), 3.62-3.55 (m, 4H), 2.97-2.87 (m, 4H), 1.45 (d, J = 6.8 Hz, 6H). |
| | 1-isopropyl-2,4-dioxo-N-[4-[(6-piperazin-1-yl-1,7-naphthyridin-4- | |
| | yl)oxy]phenyl]-3-(2-pyridyl)pyrimidine-5-carboxamide hydrochloride | |
| 12.4 | 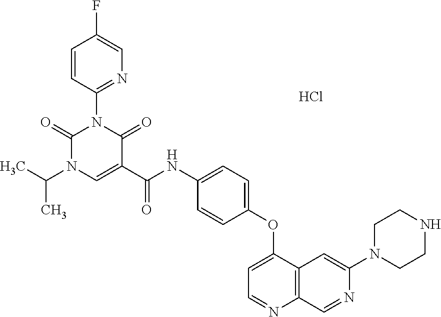 [SMILES: CC(C)n1cc(C(=O)Nc2ccc(Oc3ccnc4cnc(N5CCNCC5)cc34)cc2)c(=O)n(-c2ccc(F)cn2)c1=O ; Cl] | LC-MS (Method 7A): 1.87 min; MS m/z = 597.3 [M + H]+ ¹H NMR (500 MHz, DMSO-d₆) δ 10.80 (s, 1H), 9.16 (s, 1H), 9.09 (s, 2H), 8.70 (s, 1H), 8.68-8.64 (m, 1H), 8.59- 8.53 (m, 1H), 8.07-7.98 (m, 1H), 7.88- 7.80 (m, 2H), 7.68 (dd, J = 8.8, 4.1 Hz, 1H), 7.33-7.25 (m, 3H), 6.76- 6.66 (m, 1H), 4.83-4.70 (m, 1H), 3.92- 3.85 (m, 4H), 3.26-3.21 (m, 4H), 1.44 (d, J = 6.8 Hz, 6H). |
| | 3-(5-fluoro-2-pyridyl)-1-isopropyl-2,4-dioxo-N-[4-[(6-piperazin-1-yl-1,7- | |
| | naphthyridin-4-yl)oxy]phenyl]pyrimidine-5-carboxamide hydrochloride | |
| 12.5 | 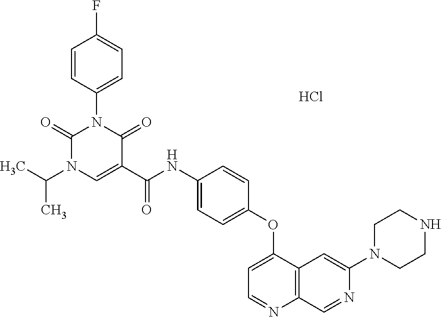 [SMILES: CC(C)n1cc(C(=O)Nc2ccc(Oc3ccnc4cnc(N5CCNCC5)cc34)cc2)c(=O)n(-c2ccc(F)cc2)c1=O ; Cl] | LC-MS (Method 7A): 2.11 min; MS m/z = 596.2 [M + H]+ ¹H NMR (400 MHz, DMSO-d6) δ 10.96 (s, 1H), 9.26-9.05 (m, 3H), 8.66 (s, 1H), 8.56 (d, J = 5.2 Hz, 1H), 7.90- 7.78 (m, 2H), 7.47-7.40 (m, 2H), 7.39- 7.33 (m, 2H), 7.33-7.27 (m, 3H), 6.71 (d, J = 5.2 Hz, 1H), 4.84-4.72 (m, 1H), 3.91-3.85 (m, 4H), 3.28-3.20 (m, 4H), 1.43 (d, J = 6.8 Hz, 6H). |
| | 3-(4-fluorophenyl)-1-isopropyl-2,4-dioxo-N-[4-[(6-piperazin-1-yl-1,7- | |
| | naphthyridin-4-yl)oxy]phenyl]pyrimidine-5-carboxamide hydrochloride | |
| 12.6 | 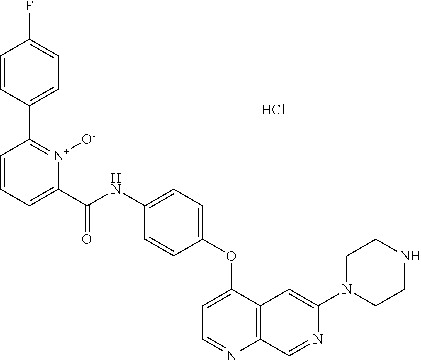 [SMILES: O=C(Nc1ccc(Oc2ccnc3cnc(N4CCNCC4)cc23)cc1)c1cccc(-c2ccc(F)cc2)[n+]1[O-] ; Cl] | LC-MS (Method 7A): 2.03 min; MS m/z = 537.2 [M + H]+ ¹H NMR (500 MHz, DMSO-d6) δ 13.46 (s, 1H), 9.15 (s, 1H), 9.07-8.94 (m, 2H), 8.55 (d, J = 5.1 Hz, 1H), 8.32 (dd, J = 8.0, 2.2 Hz, 1H), 7.97-7.84 (m, 5H), 7.76-7.71 (m, 1H), 7.42-7.31 (m, 5H), 6.72 (d, J = 5.1 Hz, 1H), 3.90- 3.84 (m, 4H), 3.30-3.22 (m, 4H). |
| | 6-(4-fluorophenyl)-1-oxido-N-[4-[(6-piperazin-1-yl-1,7-naphthyridin-4- | |
| | yl)oxy]phenyl]pyridin-1-ium-2-carboxamide hydrochloride | |
| 12.7 | 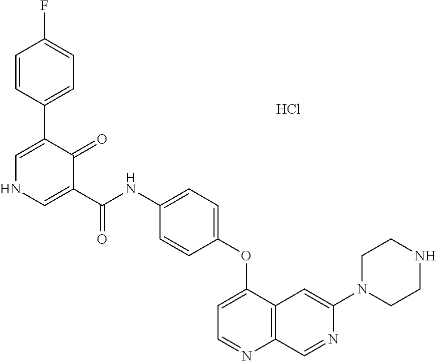 [SMILES: O=C(Nc1ccc(Oc2ccnc3cnc(N4CCNCC4)cc23)cc1)c1c[nH]cc(-c2ccc(F)cc2)c1=O ; Cl] | LC-MS (Method 7A): 1.87 min; MS m/z = 537.2 [M + H]+ ¹H NMR (400 MHz, DMSO-d₆) δ 13.10 (s, 1H), 12.83-12.69 (m, 1H), 9.19 (s, 1H), 9.15-9.01 (m, 2H), 8.66-8.61 (m, 1H), 8.60 (d, J = 5.3 Hz, 1H), 8.10 (dd, J = 5.5, 1.6 Hz, 1H), 7.94-7.83 (m, 2H), 7.76-7.66 (m, 2H), 7.42- 7.19 (m, 5H), 6.76 (d, J = 5.3 Hz, 1H), 3.94-3.86 (m, 4H), 3.27-3.19 (m, 4H). |
| | 5-(4-fluorophenyl)-4-oxo-N-[4-[(6-piperazin-1-yl-1,7-naphthyridin-4- | |
| | yl)oxy]phenyl]-1H-pyridine-3-carboxamide hydrochloride | |
| 12.8 | 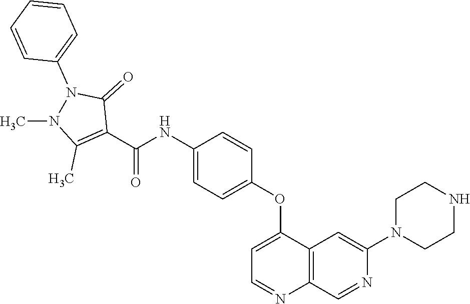 [SMILES: Cc1c(C(=O)Nc2ccc(Oc3ccnc4cnc(N5CCNCC5)cc34)cc2)c(=O)n(-c2ccccc2)n1C] | LC-MS (Method 7A): 1.69 min; MS m/z = 536.2 [M + H]+ ¹H NMR (500 MHz, DMSO-d₆) δ 10.88 (s, 1H), 9.17 (s, 1H), 9.08-8.97 (m, 2H), 8.57 (d, J = 5.2 Hz, 1H), 7.81- 7.74 (m, 2H), 7.63-7.57 (m, 2H), 7.55- 7.50 (m, 1H), 7.47-7.42 (m, 2H), 7.34 (s, 1H), 7.30-7.24 (m, 2H), 6.71 (d, J = 5.2 Hz, 1H), 3.92-3.84 (m, 4H), 3.38 (s, 3H), 3.30-3.23 (m, 4H), 2.72 (s, 3H). |
| | 1,5-dimethyl-3-oxo-2-phenyl-N-[4-[(6-piperazin-1-yl-1,7-naphthyridin- | |
| | 4-yl)oxy]phenyl]pyrazole-4-carboxamide hydrochloride | |

### Example 13

### 5-(4-Fluorophenyl)-1-isopropyl-4-oxo-N-[4-[(6-piperazin-1-yl-1,7-naphthyridin-4-yl)oxy]phenyl]pyridine-3-carboxamide hydrochloride

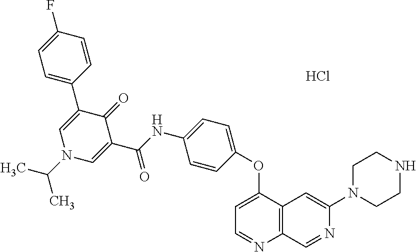 [SMILES: CC(C)n1cc(C(=O)Nc2ccc(Oc3ccnc4cnc(N5CCNCC5)cc34)cc2)c(=O)c(-c2ccc(F)cc2)c1 ; Cl]

### 5-(4-Fluorophenyl)-1-isopropyl-4-oxo-N-[4-[(6-piperazin-1-yl-1,7-naphthyridin-4-yl)oxy]phenyl]pyridine-3-carboxamide hydrochloride

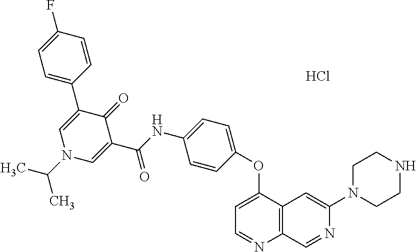 [SMILES: CC(C)n1cc(C(=O)Nc2ccc(Oc3ccnc4cnc(N5CCNCC5)cc34)cc2)c(=O)c(-c2ccc(F)cc2)c1 ; Cl]

5-(4-Fluorophenyl)-1-isopropyl-4-oxo-pyridine-3-carboxylic acid (Intermediate H)(60 mg, 0.218 mmol) and HATU (108 mg, 0.284 mmol) were suspended in DMF (1.5 mL). A suspension of tert-butyl 4-[4-(4-aminophenoxy)-1,7-naphthyridin-6-yl]piperazine-1-carboxylate (Example 12, step 3)(60 mg, 0.142 mmol) and N-methyl-morpholine (48 μL, 0.437 mmol) in DMF (1.5 mL) was added to the carboxylic acid/HATU mixture and the reaction was stirred at room temperature for 16 h.

Water (5 mL) was added and a precipitate formed which was collected by filtration, re-dissolved in DCM (10 mL) and concentrated in vacuo. The crude material was purified by silica chromatography (eluting with a gradient of 5% to 100% heptane/EtOAc) to afford the protected coupled intermediate as a yellow solid. This material was suspended in a mixture of DCM/MeOH (1:1, 2 mL). 4 M HCl in dioxane (2.0 mL, 8.00 mmol) was added and the reaction was stirred at room temperature for 1 h.

The reaction mixture was evaporated and the residue was suspended in diethyl ether (3 mL) with the solid collected by filtration to afford 5-(4-fluorophenyl)-1-isopropyl-4-oxo-N-[4-[(6-piperazin-1-yl-1,7-naphthyridin-4-yl)oxy]phenyl]pyridine-3-carboxamide hydrochloride (51 mg) as a yellow solid.

LC-MS (Method 7A): 2.16 min; MS m/z=579.2 [M+H]+

¹H NMR (400 MHz, DMSO-d₆) δ 13.11 (s, 1H), 9.16 (s, 1H), 9.03-8.97 (m, 2H), 8.80-8.74 (m, 1H), 8.57 (d, J=5.1 Hz, 1H), 8.31-8.26 (m, 1H), 7.93-7.86 (m, 2H), 7.79-7.70 (m, 2H), 7.36-7.25 (m, 4H), 6.73 (d, J=5.1 Hz, 1H), 4.70-4.61 (m, 1H), 3.92-3.84 (m, 4H), 3.30-3.22 (m, 4H), 1.53 (d, J=6.7 Hz, 6H).

The compound in the following tabulated example (Table 10) was prepared analogously to Example 13 from tert-butyl 4-[4-(4-aminophenoxy)-1,7-naphthyridin-6-yl]piperazine-1-carboxylate and the appropriate acid.

TABLE 10

| Ex. | Structure and Name | Retention Time, [M + H]+, 1H NMR |
|---|---|---|
| 13.1 | 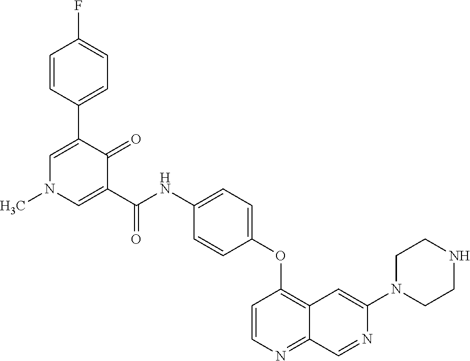 [SMILES: Cn1cc(C(=O)Nc2ccc(Oc3ccnc4cnc(N5CCNCC5)cc34)cc2)c(=O)c(-c2ccc(F)cc2)c1] | LC-MS (Method 7A): 1.95 min; MS m/z = 551.2 [M + H]+ ¹H NMR (500 MHz, DMSO-d6) δ 13.13 (s, 1H), 9.29-9.04 (m, 3H), 8.70 (d, J = 2.3 Hz, 1H), 8.59 (s, 1H), 8.17 (d, J = 2.3 Hz, 1H), 7.89 (d, J = 8.9 Hz, 2H), 7.79-7.67 (m, 2H), 7.41-7.24 (m, 5H), 6.74 (s, 1H), 3.93 (s, 3H), 3.89 (m, 4H), 3.25 (m, 4H). |
| | 5-(4-fluorophenyl)-1-methyl-4-oxo- | |
| | N-[4-[(6-piperazin-1-yl-1,7-naphthyridin-4- | |
| | yl)oxy]phenyl]pyridine-3-carboxamide hydrochloride | |

### Example 14

### 4-[4-[[3-(4-Fluorophenyl)-2,4-dioxo-1-[(1R)-2-hydroxy-1-methyl-ethyl]pyrimidine-5-carbonyl]amino]phenoxy]-N-(1-methyl-4-piperidyl)-1,7-naphthyridine-6-carboxamide

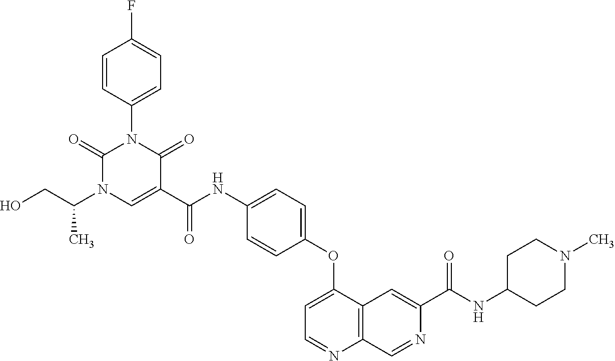 [SMILES: C[C@H](CO)n1cc(C(=O)Nc2ccc(Oc3ccnc4cnc(C(=O)NC5CCN(C)CC5)cc34)cc2)c(=O)n(-c2ccc(F)cc2)c1=O]

### 4-[4-[[3-(4-Fluorophenyl)-2,4-dioxo-1-[(1R)-2-hydroxy-1-methyl-ethyl]pyrimidine-5-carbonyl]amino]phenoxy]-N-(1-methyl-4-piperidyl)-1,7-naphthyridine-6-carboxamide

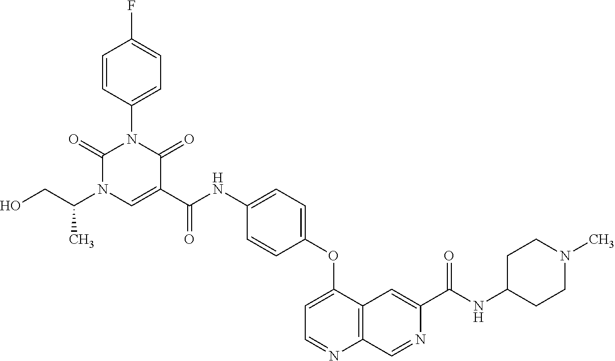 [SMILES: C[C@H](CO)n1cc(C(=O)Nc2ccc(Oc3ccnc4cnc(C(=O)NC5CCN(C)CC5)cc34)cc2)c(=O)n(-c2ccc(F)cc2)c1=O]

HATU (35 mg, 0.0928 mmol) was added to a solution of 4-(4-aminophenoxy)-N-(1-methyl-4-piperidyl)-1,7-naphthyridine-6-carboxamide (Example 8, step 2)(27 mg, 0.0714 mmol), 3-(4-fluorophenyl)-2,4-dioxo-1-[(1R)-2-hydroxy-1-methyl-ethyl]pyrimidine-5-carboxylic acid (Intermediate 1)(22 mg, 0.0714 mmol) and DIPEA (0.031 mL, 0.178 mmol) in DMF (1 mL). The solution was stirred at room temperature for 2 h. The resulting suspension was filtered and the collected solid washed with water (3×5 mL) and dried in vacuo at 40° C. to afford 4-[4-[[3-(4-fluorophenyl)-2,4-dioxo-1-[(1R)-2-hydroxy-1-methyl-ethyl]pyrimidine-5-carbonyl]amino]phenoxy]-N-(1-methyl-4-piperidyl)-1,7-naphthyridine-6-carboxamide (20.3 mg) as a white solid.

LC-MS (Method 7A): 1.96 min; MS m/z 668.4=[M+H]+

¹H NMR (500 MHz, DMSO-d₆) δ 10.96 (s, 1H), 9.43 (d, J=0.7 Hz, 1H), 8.91 (d, J=5.2 Hz, 1H), 8.81 (d, J=8.0 Hz, 1H), 8.78 (d, J=0.7 Hz, 1H), 8.70 (s, 1H), 7.88-7.82 (m, 2H), 7.45-7.39 (m, 2H), 7.39-7.30 (m, 4H), 6.93 (d, J=5.2 Hz, 1H), 5.18 (t, J=5.6 Hz, 1H), 4.74-4.64 (m, 1H), 3.95-3.82 (m, 1H), 3.78-3.60 (m, 2H), 2.94-2.79 (m, 2H), 2.32-2.08 (m, 5H), 1.86-1.70 (m, 4H), 1.39 (d, J=7.0 Hz, 3H).

### Example 15

### 4-[4-[[1-Cyclopropyl-3-(4-fluorophenyl)-2,4-dioxo-pyrimidine-5-carbonyl]amino]phenoxy]-N-(1-methyl-4-piperidyl)-1,7-naphthyridine-6-carboxamide

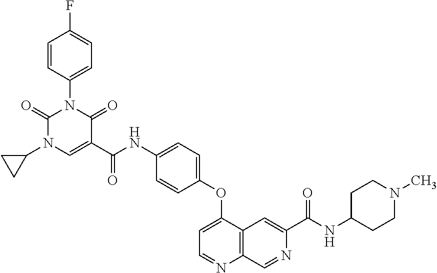 [SMILES: CN1CCC(NC(=O)c2cc3c(Oc4ccc(NC(=O)c5cn(C6CC6)c(=O)n(-c6ccc(F)cc6)c5=O)cc4)ccnc3cn2)CC1]

### 4-[4-[[1-Cyclopropyl-3-(4-fluorophenyl)-2,4-dioxo-pyrimidine-5-carbonyl]amino]phenoxy]-N-(1-methyl-4-piperidyl)-1,7-naphthyridine-6-carboxamide

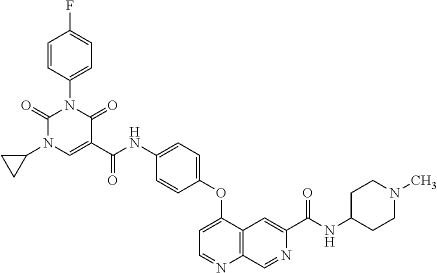 [SMILES: CN1CCC(NC(=O)c2cc3c(Oc4ccc(NC(=O)c5cn(C6CC6)c(=O)n(-c6ccc(F)cc6)c5=O)cc4)ccnc3cn2)CC1]

A mixture of 1-cyclopropyl-3-(4-fluorophenyl)-2,4-dioxo-pyrimidine-5-carboxylic acid (Intermediate J)(62 mg, 0.212 mmol), DIPEA (0.084 mL, 0.482 mmol) and HATU (95 mg, 0.251 mmol) in DMF (2 mL) was stirred for 10 mins before the addition of 4-(4-aminophenoxy)-N-(1-methyl-4-piperidyl)-1,7-naphthyridine-6-carboxamide (Example 8, step 2) (75 mg, 0.193 mmol). The reaction mixture was stirred for 30 mins at room temperature. The resulting suspension was diluted with DMF (4 mL) and the formed solid collected by filtration, washed with water (3×5 mL) and dried to afford 4-[4-[[1-cyclopropyl-3-(4-fluorophenyl)-2,4-dioxo-pyrimidine-5-carbonyl]amino]phenoxy]-N-(1-methyl-4-piperidyl)-1,7-naphthyridine-6-carboxamide (68.8 mg) as a white solid.

LC-MS (Method 7A): 2.08 min; MS m/z 650.3=[M+H]+

¹H NMR (500 MHz, Methanol-d4) δ 9.40 (d, J=0.8 Hz, 1H), 8.96 (d, J=0.8 Hz, 1H), 8.85 (d, J=5.3 Hz, 1H), 8.66 (s, 1H), 7.88-7.78 (m, 2H), 7.40-7.33 (m, 2H), 7.33-7.24 (m, 4H), 6.93 (d, J=5.3 Hz, 1H), 4.12-3.96 (m, 1H), 3.38-3.33 (m, 1H), 3.08-2.95 (m, 2H), 2.51-2.33 (m, 5H), 2.10-2.02 (m, 2H), 1.90-1.76 (m, 2H), 1.19-1.09 (m, 2H), 1.09-1.01 (m, 2H).

### Example 16

### 1-(4-Fluorophenyl)-N-[5-[[6-(1-methyl-4-piperidyl)-1,7-naphthyridin-4-yl]oxy]-2-pyridyl]-2-oxo-pyridine-3-carboxamide

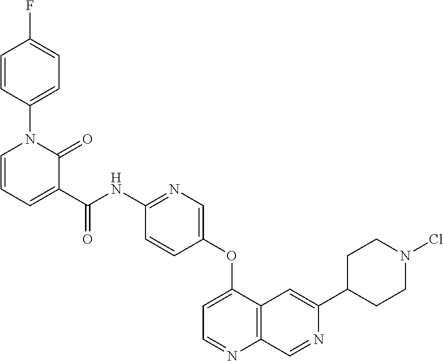 [SMILES: O=C(Nc1ccc(Oc2ccnc3cnc(C4CCN(Cl)CC4)cc23)cn1)c1cccn(-c2ccc(F)cc2)c1=O]

### Step 1: 1-(4-Fluorophenyl)-N-[5-[[6-(1-methyl-3,6-dihydro-2H-pyridin-4-yl)-1,7-naphthyridin-4-yl]oxy]-2-pyridyl]-2-oxo-pyridine-3-carboxamide

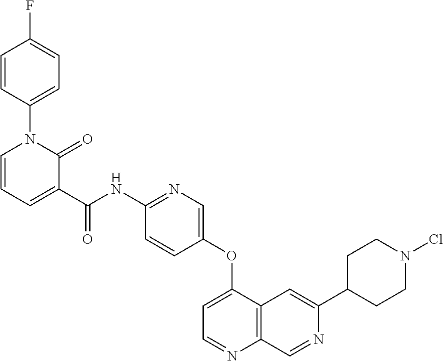 [SMILES: O=C(Nc1ccc(Oc2ccnc3cnc(C4CCN(Cl)CC4)cc23)cn1)c1cccn(-c2ccc(F)cc2)c1=O]

Pd(PPh₃)₄ (12 ing, 0.0103 mmol), 1-methyl-4-(4,4,5,5-tetramethyl-1,3,2-dioxaborolan-2-yl)-3,6-dihydro-2H-pyridine (27 ing, 0.123 mol) and potassium carbonate (27 ing, 0.205 mmol) were suspended in 1,4-dioxane (2 mL) and water (0.50 mL). The mixture was degassed with nitrogen for 10 mins. N-[5-[(6-chloro-1,7-naphthyridin-4-yl)oxy]-2-pyridyl]-1-(4-fluorophenyl)-2-oxo-pyridine-3-carboxamide (Example 10, step 3)(50 mg, 0.103 mmol) was added and the reaction mixture was degassed for a further 10 min. The reaction mixture was stirred at 95° C. for 1.5 h in a sealed vessel. After cooling to room temperature, water (5 mL) was added to the reaction mixture. The resulting suspension was extracted with DCM (3×5 mL) and the combined organics were passed through a hydrophobic frit and concentrated in vacuo. The crude residue was purified by low pH reverse phase chromatography (eluting with a gradient of 10-100% MeCN (0.1% formic acid) in water (0.1% formic acid)) to afford 1-(4-fluorophenyl)-N-[5-[[6-(1-methyl-3,6-dihydro-2H-pyridin-4-yl)-1,7-naphthyridin-4-yl]oxy]-2-pyridyl]-2-oxo-pyridine-3-carboxamide (21 mg) as a brown solid.

LC-MS (Method 2A): 1.02 min; MS m/z 549.2=[M+H]+

¹H NMR (500 MHz, DMSO-d6) δ 12.46 (s, 1H), 9.38 (d, J=0.7 Hz, 1H), 8.77 (d, J=5.2 Hz, 1H), 8.65 (dd, J=7.3, 2.2 Hz, 1H), 8.49-8.39 (m, 2H), 8.19-8.12 (m, 2H), 8.06 (s, 1H), 7.94 (dd, J=9.0, 2.9 Hz, 1H), 7.66-7.58 (m, 2H), 7.48-7.39 (m, 2H), 7.04 (d, J=3.6 Hz, 1H), 6.92 (d, J=5.1 Hz, 1H), 6.79-6.69 (m, 1H), 3.21-3.12 (m, 2H), 2.74-2.66 (m, 4H), 2.35 (s, 3H).

### Step 2: 1-(4-Fluorophenyl)-N-[5-[[6-(1-methyl-4-piperidyl)-1,7-naphthyridin-4-yl]oxy]-2-pyridyl]-2-oxo-pyridine-3-carboxamide

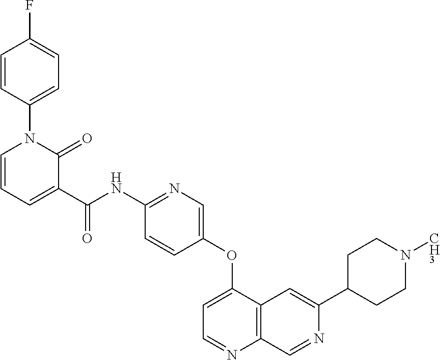 [SMILES: CN1CCC(c2cc3c(Oc4ccc(NC(=O)c5cccn(-c6ccc(F)cc6)c5=O)nc4)ccnc3cn2)CC1]

1-(4-Fluorophenyl)-N-[5-[[6-(1-methyl-3,6-dihydro-2H-pyridin-4-yl)-1,7-naphthyridin-4-yl]oxy]-2-pyridyl]-2-oxo-pyridine-3-carboxamide (Example 16, step 1)(55 mg, 0.100 mmol) was suspended in 1,4-dioxane (5 mL) and the mixture was purged with nitrogen. 10% Pd/C (21 mg, 0.0201 mmol) was added and the reaction was purged with nitrogen before charging with hydrogen. The reaction was stirred for 30 h at room temperature after which time the mixture was filtered through celite and the filtrate concentrated in vacuo. The crude product was purified by basic preparative HPLC (early elute method) to afford 1-(4-fluorophenyl)-N-[5-[[6-(1-methyl-4-piperidyl)-1,7-naphthyridin-4-yl]oxy]-2-pyridyl]-2-oxo-pyridine-3-carboxamide (7.1 mg) as a white solid.

LC-MS (Method 7B): 3.25 min; MS m/z 551.3=[M+H]+

¹H NMR (500 MHz, DMSO-d6) δ 12.46 (s, 1H), 9.38 (d, J=0.7 Hz, 1H), 8.78 (d, J=5.1 Hz, 1H), 8.65 (dd, J=7.3, 2.2 Hz, 1H), 8.50-8.44 (m, 1H), 8.43 (d, J=2.9 Hz, 1H), 8.16 (dd, J=6.6, 2.2 Hz, 1H), 7.98 (s, 1H), 7.93 (dd, J=9.0, 2.9 Hz, 1H), 7.66-7.59 (m, 2H), 7.48-7.40 (m, 2H), 6.91 (d, J=5.1 Hz, 1H), 6.78-6.71 (m, 1H), 2.94-2.88 (m, 2H), 2.88-2.82 (m, 1H), 2.22 (s, 3H), 2.07-1.98 (m, 2H), 1.98-1.81 (m, 4H).

The compounds in the following tabulated examples (Table 11) were prepared analogously to Example 16 from N-[5-[(6-chloro-1,7-naphthyridin-4-yl)oxy]-2-pyridyl]-1-(4-fluorophenyl)-2-oxo-pyridine-3-carboxamide (Example 10, step 3) and the appropriate boronate ester.

TABLE 11

| Ex. | Structure and Name | Retention Time, [M + H]+, 1H NMR |
|---|---|---|
| 16.1 | 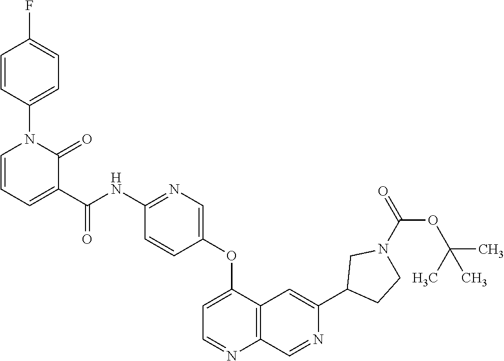 [SMILES: CC(C)(C)OC(=O)N1CCC(c2cc3c(Oc4ccc(NC(=O)c5cccn(-c6ccc(F)cc6)c5=O)nc4)ccnc3cn2)C1] | LC-MS (Method 7B): 3.98 min; MS m/z = 623.5 [M + H]+ ¹H NMR (400 MHz, DMSO) δ 12.46 (s, 1H), 9.41 (s, 1H), 8.81 (d, J = 5.1 Hz, 1H), 8.65 (dd, J = 7.3, 2.2 Hz, 1H), 8.46 (d, J = 9.2 Hz, 1H), 8.43 (d, J = 2.9 Hz, 1H), 8.16 (dd, J = 6.6, 2.2 Hz, 1H), 8.08 (s, 1H), 7.93 (dd, J = 9.0, 2.9 Hz, 1H), 7.67-7.58 (m, 2H), 7.48-7.40 (m, 2H), 6.93 (d, J = 5.1 Hz, 1H), 6.79- 6.71 (m, 1H), 3.88-3.74 (m, 2H), 3.60-3.50 (m, 2H), 2.29-2.14 (m, 1H), 1.45-1.40 (m, 11H). |
| | tert-butyl 3-[4-[[6-[[1-(4-fluorophenyl)-2-oxo-pyridine-3- | |
| | carbonyl]amino]-3-pyridyl]oxy]-1,7- | |
| | naphthyridin-6-yl]pyrrolidine-1-carboxylate | |
| 16.2 | 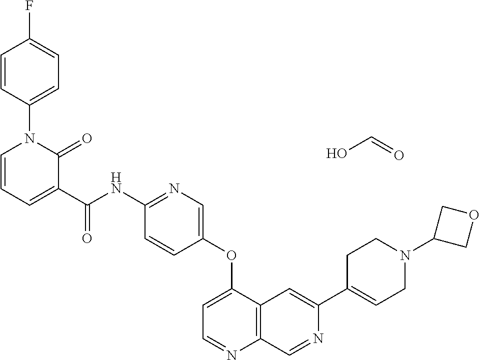 [SMILES: O=C(Nc1ccc(Oc2ccnc3cnc(C4=CCN(C5COC5)CC4)cc23)cn1)c1cccn(-c2ccc(F)cc2)c1=O ; O=CO] | LC-MS (Method 7A): 1.84 min; MS m/z = 591.3 [M + H]+ ¹H NMR (500 MHz, CDCl3) δ 12.44 (s, 1H), 9.45 (s, 1H), 8.77-8.68 (m, 2H), 8.52 (d, J = 9.0 Hz, 1H), 8.27 (d, J = 2.8 Hz, 1H), 8.14 (s, 1H), 8.05 (s, 1H), 7.66 (dd, J = 6.6, 2.1 Hz, 1H), 7.58 (dd, J = 9.0, 2.8 Hz, 1H), 7.46-7.36 (m, 2H), 7.23 (d, J = 8.6 Hz, 2H), 7.03 (s, 1H), 6.69 (d, J = 5.2 Hz, 1H), 6.61 (t, J = 7.0 Hz, 1H), 4.78 (dt, J = 21.1, 6.5 Hz, 4H), 3.81 (q, J = 6.5 Hz, 1H), 3.31-3.26 (m, 2H), 2.85 (s, 2H), 2.77 (t, J = 5.6 Hz, 2H). |
| | 1-(4-fluorophenyl)-N-[5-[[6-[1-(oxetan-3-yl)-3,6-dihydro-2H- | |
| | pyridin-4-yl]-1,7-naphthyridin-4-yl]oxy]-2-pyridyl]-2- | |
| | oxo-pyridine-3-carboxamide formate salt | |
| 16.3 | 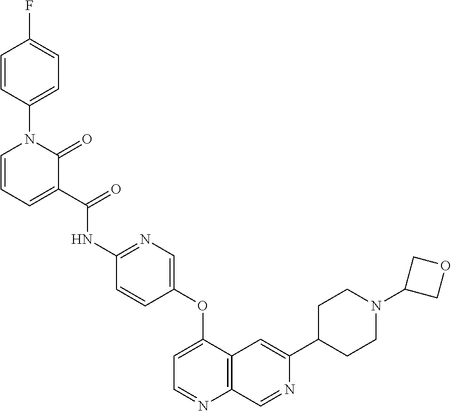 [SMILES: O=C(Nc1ccc(Oc2ccnc3cnc(C4CCN(C5COC5)CC4)cc23)cn1)c1cccn(-c2ccc(F)cc2)c1=O] | LC-MS (Method 7B): 3.07 min; MS m/z = 593.5 [M + H]+ ¹H NMR (400 MHz, DMSO) δ 12.46 (s, 1H), 9.39 (s, 1H), 8.79 (d, J = 5.1 Hz, 1H), 8.65 (dd, J = 7.3, 2.1 Hz, 1H), 8.50- 8.38 (m, 2H), 8.16 (dd, J = 6.6, 2.1 Hz, 1H), 7.99 (s, 1H), 7.93 (dd, J = 9.0, 2.9 Hz, 1H), 7.63 (dd, J = 8.9, 4.9 Hz, 2H), 7.44 (t, J = 8.8 Hz, 2H), 6.92 (d, J = 5.1 Hz, 1H), 6.75 (t, J = 7.0 Hz, 1H), 4.57 (t, J = 6.5 Hz, 2H), 4.47 (t, J = 6.1 Hz, 2H), 3.48-3.43 (m, 1H), 2.98- 2.88 (m, 1H), 2.88-2.80 (m, 2H), 2.03- 1.77 (m, 6H). |
| | 1-(4-fluorophenyl)-N-[5-[[6-[1-(oxetan-3-yl)-4-piperidyl]-1,7- | |
| | naphthyridin-4-yl]oxy]-2-pyridyl]-2-oxo-pyridine-3-carboxamide | |
| 16.4 | 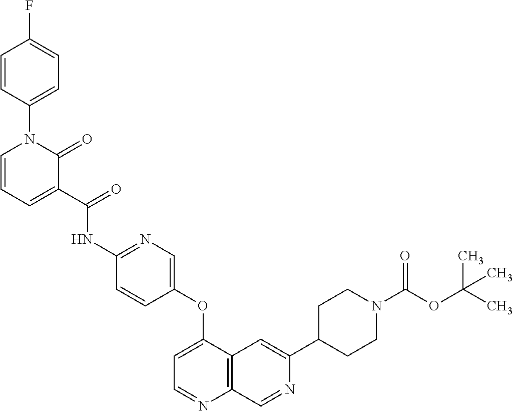 [SMILES: CC(C)(C)OC(=O)N1CCC(c2cc3c(Oc4ccc(NC(=O)c5cccn(-c6ccc(F)cc6)c5=O)nc4)ccnc3cn2)CC1] | LC-MS (Method 7B): 4.13 min; MS m/z = 637.6 [M + H]+ ¹H NMR (400 MHz, DMSO) δ 12.46 (s, 1H), 9.39 (s, 1H), 8.79 (d, J = 5.1 Hz, 1H), 8.65 (dd, J = 7.3, 2.2 Hz, 1H), 8.50- 8.41 (m, 2H), 8.16 (dd, J = 6.6, 2.2 Hz, 1H), 8.00 (s, 1H), 7.93 (dd, J = 9.0, 2.9 Hz, 1H), 7.67-7.58 (m, 2H), 7.50- 7.38 (m, 2H), 6.92 (d, J = 5.1 Hz, 1H), 6.79-6.71 (m, 1H), 4.12 (d, J = 10.7 Hz, 2H), 3.19-3.10 (m, 1H), 2.90 (m, 2H), 1.96 (d, J = 12.2 Hz, 2H), 1.72 (tt, J = 12.6, 6.3 Hz, 2H), 1.43 (s, 9H). |
| | tert-butyl 4-[4-[[6-[[1-(4-fluorophenyl)-2-oxo-pyridine-3- | |
| | carbonyl]amino]-3-pyridyl]oxy]-1,7- | |
| | naphthyridin-6-yl]piperidine-1-carboxylate | |

### Example 17

### 1-(4-fluorophenyl)-N-[4-[[6-(1-methyl-4-piperidyl)-1,7-naphthyridin-4-yl]oxy]phenyl]-2-oxo-pyridine-3-carboxamide

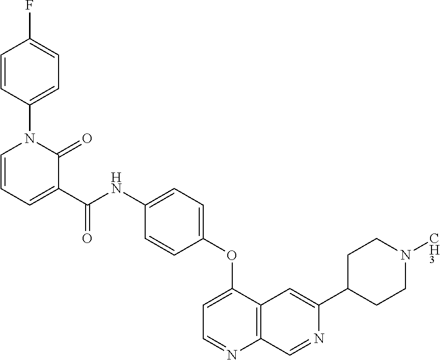 [SMILES: CN1CCC(c2cc3c(Oc4ccc(NC(=O)c5cccn(-c6ccc(F)cc6)c5=O)cc4)ccnc3cn2)CC1]

### Step 1: 6-(1-Methyl-3,6-dihydro-2H-pyridin-4-yl)-4-(4-nitrophenoxy)-1,7-naphthyridine

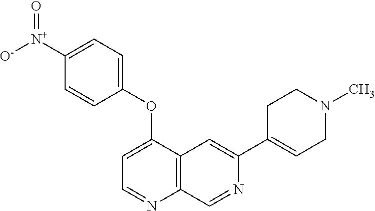 [SMILES: CN1CC=C(c2cc3c(Oc4ccc([N+](=O)[O-])cc4)ccnc3cn2)CC1]

Pd(PPh₃)₄ (77 mg, 0.0666 mmol), 1-methyl-4-(4,4,5,5-tetramethyl-1,3,2-dioxaborolan-2-yl)-3,6-dihydro-2H-pyridine (178 mg, 0.798 mmol) and potassium carbonate (175 mg, 1.32 mmol) were suspended in 1,4-dioxane (3 mL) and water (1 mL). The mixture was degassed with nitrogen for 10 mins. 6-chloro-4-(4-nitrophenoxy)-1,7-naphthyridine (200 mg, 0.663 mmol) was added and the reaction mixture was degassed for a further 10 mins before stirring at 90° C. for 16 h.

The reaction was cooled to room temperature, water (8 mL) was added and the resultant mixture was extracted with DCM (3×10 mL). The organic layer was filtered through a hydrophobic frit and concentrated in vacuo. The crude material was purified by silica chromatography (eluting with a gradient of heptane/EtOAc 0-100% then EtOAc/MeOH 0-45%) to afford 6-(1-methyl-3,6-dihydro-2H-pyridin-4-yl)-4-(4-nitrophenoxy)-1,7-naphthyridine (140 mg) as a yellow solid.

LC-MS (Method 2A): 0.85 min; MS m/z 362.9=[M+H]+

¹H NMR (500 MHz, DMSO-d6) δ 9.41 (d, J=0.8 Hz, 1H), 8.86 (d, J=5.1 Hz, 1H), 8.43-8.36 (m, 2H), 7.95 (s, 1H), 7.62-7.55 (m, 2H), 7.14 (d, J=5.1 Hz, 1H), 7.05-7.02 (m, 1H), 3.14-3.10 (m, 2H), 2.67-2.58 (m, 4H), 2.31 (s, 3H).

### Step 2: 4-[[6-(1-Methyl-4-piperidyl)-1,7-naphthyridin-4-yl]oxy]aniline

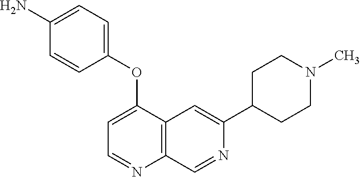 [SMILES: CN1CCC(c2cc3c(Oc4ccc(N)cc4)ccnc3cn2)CC1]

6-(1-Methyl-3,6-dihydro-2H-pyridin-4-yl)-4-(4-nitrophenoxy)-1,7-naphthyridine (Example 17, step 1)(140 mg, 0.386 mmol) was suspended in 1,4-dioxane (8 mL). The flask was flushed with nitrogen before 10% Pd/C (410 mg, 0.387 mmol) was added and the flask was flushed with nitrogen before charging with hydrogen and stirring for 18 h at room temperature. The reaction mixture was filtered through a pad of celite and the filter bed was washed with MeOH (3×25 mL). The combined filtrates were concentrated in vacuo and the crude material purified by Prep HPLC (early elute-high pH method) with the product containing fractions combined, concentrated in vacuo and freeze-dried to afford 4-[[6-(1-methyl-4-piperidyl)-1,7-naphthyridin-4-yl]oxy]aniline (16 mg) as a white solid.

LC-MS (Method 2.5B): 1.43 min; MS m/z 335.3=[M+H]+

¹H NMR (500 MHz, DMSO-d₆) δ 9.33 (d, J=0.7 Hz, 1H), 8.72 (d, J=5.1 Hz, 1H), 7.93 (s, 1H), 7.01-6.94 (m, 2H), 6.72-6.65 (m, 3H), 5.22 (s, 2H), 2.95-2.88 (m, 2H), 2.90-2.80 (m, 1H), 2.22 (s, 3H), 2.07-1.98 (m, 2H), 1.96-1.91 (m, 2H), 1.92-1.83 (m, 2H).

### Step 3: 1-(4-Fluorophenyl)-N-[4-[[6-(1-methyl-4-piperidyl)-1,7-naphthyridin-4-yl]oxy]phenyl]-2-oxo-pyridine-3-carboxamide

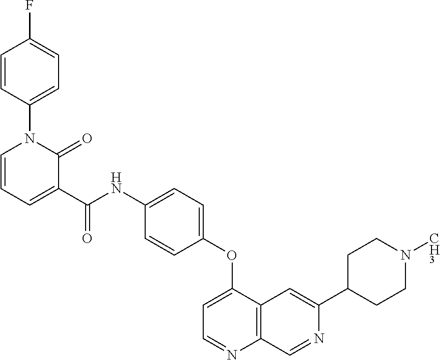 [SMILES: CN1CCC(c2cc3c(Oc4ccc(NC(=O)c5cccn(-c6ccc(F)cc6)c5=O)cc4)ccnc3cn2)CC1]

1-(4-fluorophenyl)-2-oxo-pyridine-3-carboxylic acid (Intermediate B)(17 mg, 0.0708 mmol) and HATU (36 mg, 0.0947 mmol) were suspended in DMF (0.75 mL). 4-[[6-(1-Methyl-4-piperidyl)-1,7-naphthyridin-4-yl]oxy]aniline (Example 17, step 2)(16 mg, 0.0469 mmol) and N-methyl morpholine (15 μL, 0.141 mmol) were separately suspended in DMF (0.75 mL) and added to the activated acid solution at room temperature, The mixture was stirred for 1 h before water (10 mL) was added and the product was extracted with CHCl₃/IPA (2:1, 3×15 mL). The combined organic extracts were filtered through a hydrophobic frit and concentrated in vacuo. The crude material was purified by high pH reverse phase chromatography (eluting with a gradient of 10-100% MeCN (0.1% ammonium hydroxide) in water (0.1% ammonium hydroxide)) to afford a yellow solid. The solid was suspended in diethyl ether (2 mL), collected by filtration and dried to afford 1-(4-Fluorophenyl)-N-[4-[[6-(1-methyl-4-piperidyl)-1,7-naphthyridin-4-yl]oxy]phenyl]-2-oxo-pyridine-3-carboxamide (4.7 mg) as a beige solid.

LC-MS (Method 7A): 2.01 min; MS m/z 550.2=[M+H]+

¹H NMR (400 MHz, DMSO-d6) δ 11.96 (s, 1H), 9.30 (s, 1H), 8.70 (d, J=5.1 Hz, 1H), 8.53 (dd, J=7.3, 2.2 Hz, 1H), 8.05 (dd, J=6.6, 2.2 Hz, 1H), 7.89 (s, 1H), 7.84-7.77 (m, 2H), 7.59-7.51 (m, 2H), 7.41-7.31 (m, 2H), 7.30-7.22 (m, 2H), 6.74 (d, J=5.1 Hz, 1H), 6.71-6.62 (m, 1H), 2.98-2.90 (m, 2H), 2.89-2.80 (m, 1H), 2.25 (s, 3H), 2.18-2.05 (m, 2H), 1.95-1.77 (m, 4H).

### Example 18

### 1-(4-Fluorophenyl)-N-[5-[[6-[(1-methylpyrrolidine-3-carbonyl)amino]-1,7-naphthyridin-4-yl]oxy]-2-pyridyl]-2-oxo-pyridine-3-carboxamide formate salt

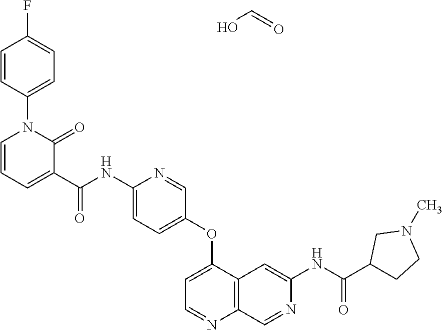 [SMILES: CN1CCC(C(=O)Nc2cc3c(Oc4ccc(NC(=O)c5cccn(-c6ccc(F)cc6)c5=O)nc4)ccnc3cn2)C1 ; O=CO]

XantPhos-Pd-G3 (15 mg, 0.0154 mmol) was added to a nitrogen flushed suspension of N-[5-[(6-chloro-1,7-naphthyridin-4-yl)oxy]-2-pyridyl]-1-(4-fluorophenyl)-2-oxo-pyridine-3-carboxamide (Example 10, step 3)(75 mg, 0.154 mmol), 1-methylpyrrolidine-3-carboxamide (Intermediate K)(20 mg, 0.154 mmol) and cesium carbonate (150 mg, 0.461 mmol) in 1,4-dioxane (2 mL). The reaction mixture was stirred at 100° C. for 2 h. The reaction mixture was cooled to room temperature and partitioned between water (10 mL) and DCM (10 mL). The organic fraction was separated and the aqueous layer was further extracted with DCM (2×10 mL). The combined organic fractions were passed through a hydrophobic frit before being concentrated in vacuo. The oil was purified by low pH reverse phase chromatography (eluting with a gradient of 10-100% MeCN (0.1% formic acid) in water (0.1% formic acid)) to afford 1-(4-fluorophenyl)-N-[5-[[6-[(1-methylpyrrolidine-3-carbonyl)amino]-1,7-naphthyridin-4-yl]oxy]-2-pyridyl]-2-oxo-pyridine-3-carboxamide formate salt (44 mg) as a brown solid.

LC-MS (Method 7A): 1.79 mm; MS m/z 580.3=[M+H]+

¹H NMR (400 MHz, DMSO-d6) δ 12.45 (s, 1H), 10.85 (s, 1H), 9.23 (d, J=0.8 Hz, 1H), 8.91-8.85 (m, 1H), 8.70 (d, J=5.1 Hz, 1H), 8.65 (dd, J=7.3, 2.2 Hz, 1H), 8.46 (d, J=9.1 Hz, 1H), 8.42 (d, J=2.8 Hz, 1H), 8.20 (s, 1H), 8.16 (dd, J=6.6, 2.2 Hz, 1H), 7.92 (dd, J=9.0, 2.9 Hz, 1H), 7.68)-7.58 (m, 2H), 7.48-7.39 (m, 2H), 6.87 (d, J=5.1 Hz, 1H), 6.80-6.71 (m, 1H), 3.34-3.25 (m, 1H), 2.93-2.85 (m, 1H), 2.71-2.58 (i, 3H), 2.32 (s, 3H), 2.12-1.99 (in, 2H).

The compound in the following tabulated example (Table 12) was prepared analogously to Example 18 from N-[5-[(6-chloro-1,7-naphthyridin-4-yl)oxy]-2-pyridyl]-1-(4-fluorophenyl)-2-oxo-pyridine-3-carboxamide (Example 10, step 3) and the appropriate amide.

TABLE 12

| Ex. | Structure and Name | Retention Time, [M + H]+, 1H NMR |
|---|---|---|
| 18.1 | 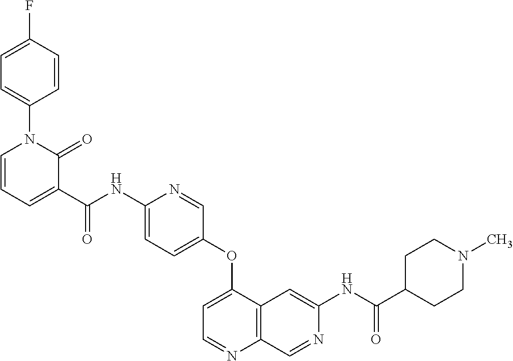 [SMILES: CN1CCC(C(=O)Nc2cc3c(Oc4ccc(NC(=O)c5cccn(-c6ccc(F)cc6)c5=O)nc4)ccnc3cn2)CC1] | LC-MS (Method 7A): 2.95 min; MS m/z 594.3 = [M + H]+ ¹H NMR (400 MHz, DMSO-d₆) δ 12.45 (s, 1H), 10.82 (s, 1H), 9.22 (d, J = 0.8 Hz, 1H), 8.87 (d, J = 0.7 Hz, 1H), 8.69 (d, J = 5.1 Hz, 1H), 8.64 (dd, J = 7.3, 2.2 Hz, 1H), 8.45 (d, J = 9.1 Hz, 1H), 8.41 (d, J = 2.8 Hz, 1H), 8.22-8.11 (m, 3H), 7.91 (dd, J = 9.0, 2.9 Hz, 1H), 7.67-7.59 (m, 2H), 7.49-7.36 (m, 2H), 6.86 (d, J = 5.1 Hz, 1H), 6.78-6.70 (m, 1H), 2.91-2.85 (m, 2H), 2.61-2.54 (m, 1H), 2.23 (s, 3H), 2.08-1.93 (m, 2H), 1.86-1.76 (m, 2H), 1.76-1.60 (m, 2H). |
| | 1-(4-fluorophenyl)-N-[5-[[6-[(1-methylpiperidine-4- | |
| | carbonyl)amino]-1,7-naphthyridin-4-yl]oxy]-2- | |
| | pyridyl]-2-oxo-pyridine-3-carboxamide;formic acid | |

### Example 19

### 4-[2-[[1-(4-Fluorophenyl)-2-oxo-pyridine-3-carbonyl]amino]pyrimidin-5-yl]oxy-N-(1-methyl-4-piperidyl)-1,7-naphthyridine-6-carboxamide formate salt

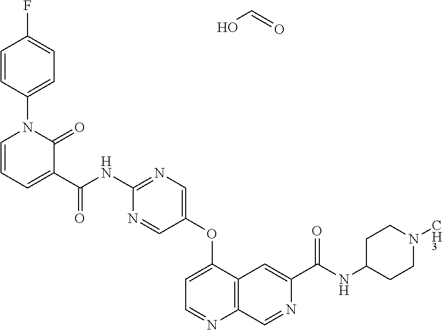 [SMILES: CN1CCC(NC(=O)c2cc3c(Oc4cnc(NC(=O)c5cccn(-c6ccc(F)cc6)c5=O)nc4)ccnc3cn2)CC1 ; O=CO]

### Step 1: 2-Chloro-5-(methoxymethoxy)pyrimidine

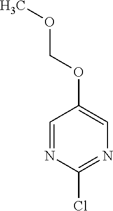

Chloro(methoxy)methane (0.51 mL, 2.30 mmol) was added to an ice cooled stirring suspension of 2-chloropyrimidin-5-ol (250 mg, 1.92 mmol) and potassium carbonate (529 mg, 3.83 mmol) in DMF (10 mL). The reaction mixture was allowed to warm to room temperature and was stirred for 18 h.

The reaction mixture was partitioned between water (20 mL) and EtOAc (20 mL), the organic fraction separated and the aqueous layer was further extracted with EtOAc (10 mL). The combined organic fractions were washed with water (20 mL) and brine (3×10 mL) before being dried over MgSO4 and concentrated in vacuo. The crude product was purified by silica chromatography (eluting with a gradient of 0-100% EtOAc in heptane) to afford 2-chloro-5-(methoxymethoxy)pyrimidine (210 mg) as a colourless liquid.

LC-MS (Method 2A): 0.91 min; MS m/z 175.1=[M+H]+

¹H NMR (500 MHz, CDCl₃) δ 8.42 (s, 2H), 5.22 (s, 2H), 3.50 (s, 3H).

### Step 2: 1-(4-Fluorophenyl)-N-[5-(methoxymethoxy)pyrimidin-2-yl]-2-oxo-pyridine-3-carboxamide

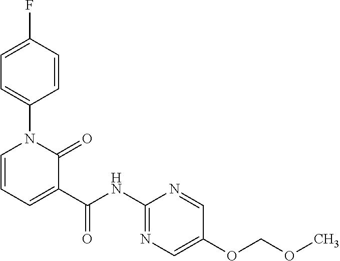 [SMILES: COCOc1cnc(NC(=O)c2cccn(-c3ccc(F)cc3)c2=O)nc1]

XantPhos-Pd-G3 (103 mg, 0.109 mmol) was added to a nitrogen flushed suspension of 1-(4-fluorophenyl)-2-oxo-pyridine-3-carboxamide (Intermediate L)(253 mg, 1.09 mmol), 2-chloro-5-(methoxymethoxy)pyrimidine (Example 19, step 1)(190 mg, 1.09 mmol) and cesium carbonate (1.06 mg, 3.26 mmol) in 1,4-dioxane (5 mL) and the reaction mixture was stirred at 100° C. for 2 h.

The reaction mixture was cooled to room temperature and partitioned between water (20 mL) and DCM (20 mL). The organic fraction was separated and the aqueous layer was further extracted with DCM (2×20 mL). The combined organic fractions were passed through a hydrophobic frit before being concentrated in vacuo. The crude product was suspended in MeOH (5 mL) and the solid collected by filtration, washed with MeOH (2×1 mL) and dried to afford 1-(4-fluorophenyl)-N-[5-(methoxymethoxy)pyrimidin-2-yl]-2-oxo-pyridine-3-carboxamide (170 mg) as an off-white solid.

LC-MS (Method 2A): 1.04 min; MS m/z 370.9=[M+H]+

¹H NMR (500 MHz, DMSO-d6) δ 12.33 (s, 1H), 8.57 (dd, J=7.3, 2.2 Hz, 1H), 8.49 (s, 2H), 8.12 (dd, J=6.6, 2.2 Hz, 1H), 7.65-7.57 (m, 2H), 7.49-7.36 (m, 2H), 6.71 (dd, J=7.2, 6.7 Hz, 1H), 5.28 (s, 2H), 3.42 (s, 3H).

### Step 3: 1-(4-Fluorophenyl)-N-(5-hydroxypyrimidin-2-yl)-2-oxo-pyridine-3-carboxamide

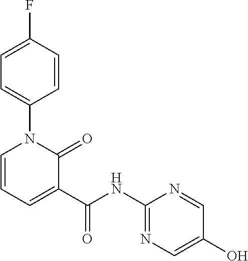 [SMILES: O=C(Nc1ncc(O)cn1)c1cccn(-c2ccc(F)cc2)c1=O]

1-(4-Fluorophenyl)-N-[5-(methoxymethoxy)pyrimidin-2-yl]-2-oxo-pyridine-3-carboxamide (Example 19, step 2)(170 mg, 0.459 mmol) was suspended in 1,4-dioxane (1 mL) and 4 M hydrochloric acid in dioxane (1.0 mL, 4.00 mmol) was added. The reaction mixture was stirred at room temperature for 1 h after which time the solid was collected by filtration, washed with diethyl ether (3×2 mL) and dried to afford 1-(4-fluorophenyl)-N-(5-hydroxypyrimidin-2-yl)-2-oxo-pyridine-3-carboxamide (150 mg) as an off-white solid.

LC-MS (Method 2A): 0.95 min; MS m/z 327.0=[M+H]+

¹H NMR (500 MHz, DMSO-d6) δ 12.20 (s, 1H), 10.26 (s, 1H), 8.55 (dd, J=7.3, 2.2 Hz, 1H), 8.25 (s, 2H), 8.10 (dd, J=6.6, 2.2 Hz, 1H), 7.65-7.54 (m, 2H), 7.49-7.35 (m, 2H), 6.70 (dd, J=7.2, 6.7 Hz, 1H).

### Step 4: N-[5-[(6-chloro-1,7-naphthyridin-4-yl)oxy]pyrimidin-2-yl]-1-(4-fluorophenyl)-2-oxo-pyridine-3-carboxamide

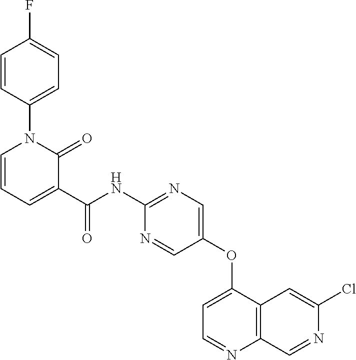 [SMILES: O=C(Nc1ncc(Oc2ccnc3cnc(Cl)cc23)cn1)c1cccn(-c2ccc(F)cc2)c1=O]

A solution of 4,6-dichloro-1,7-naphthyridine (92 mg, 0.460 mmol), 1-(4-fluorophenyl)-N-(5-hydroxypyrimidin-2-yl)-2-oxo-pyridine-3-carboxamide (Example 19, step 3) (150 mg, 0.460 mmol) and DIPEA (0.16 mL, 0.919 mmol) in chlorobenzene (20 mL) was stirred at 150° C. for 48 h.

The reaction mixture was cooled to room temperature and partitioned between DCM (15 mL) and water (15 mL). The organic fraction was separated and the aqueous layer further extracted with DCM (2×10 mL). The combined organic fractions were passed through a hydrophobic frit and concentrated in vacuo. The crude solid was suspended in diethyl ether (5 mL), the solid collected by filtration, washed with diethyl ether (2×2 mL) and dried to afford N-[5-[(6-chloro-1,7-naphthyridin-4-yl)oxy]pyrimidin-2-yl]-1-(4-fluorophenyl)-2-oxo-pyridine-3-carboxamide (75 mg) as a brown solid.

LC-MS (Method 2A): 1.13 min; MS m/z 489.0=[M+H]+

¹H NMR (500 MHz, DMSO-d6) δ 12.65 (s, 1H), 9.33 (d, J=0.8 Hz, 1H), 8.89 (d, J=5.2 Hz, 1H), 8.86 (s, 2H), 8.61 (dd, J=7.3, 2.2 Hz, 1H), 8.30 (d, J=0.7 Hz, 1H), 8.16 (dd, J=6.6, 2.2 Hz, 1H), 7.66-7.58 (m, 2H), 7.45-7.41 (m, 2H), 7.19 (d, J=5.2 Hz, 1H), 6.77-6.71 (m, 1H).

### Step 5: 4-[2-[[1-(4-Fluorophenyl)-2-oxo-pyridine-3-carbonyl]amino]pyrimidin-5-yl]oxy-N-(1-methyl-4-piperidyl)-1,7-naphthyridine-6-carboxamide formate salt

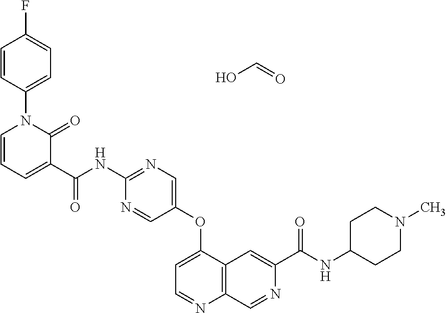 [SMILES: CN1CCC(NC(=O)c2cc3c(Oc4cnc(NC(=O)c5cccn(-c6ccc(F)cc6)c5=O)nc4)ccnc3cn2)CC1 ; O=CO]

N-[5-[(6-chloro-1,7-naphthyridin-4-yl)oxy]pyrimidin-2-yl]-1-(4-fluorophenyl)-2-oxo-pyridine-3-carboxamide (Example 19, step 4) (75 mg, 0.153 mmol), 1-methylpiperidin-4-amine (53 mg, 0.460 mmol), XantPhos-Pd-G3 (15 mg, 0.0153 mmol) and sodium carbonate (49 mg, 0.460 mmol) in DMA (30 mL) were added to chamber A of a 400 mL COware apparatus and the suspension was flushed with nitrogen. A solution of methanesulfonyl chloride (36 μL, 0.460 mmol) and formic acid (17 μL, 0.460 mmol) in nitrogen flushed toluene (5 mL) was added to chamber B and the vessel was sealed. Triethylamine (128 μL, 0.921 mmol) was added to chamber B and the reaction mixture was stirred at 100° C. for 5 h.

The reaction mixture was cooled to room temperature and partitioned between water (10 mL) and DCM (10 mL). The organic fraction was separated and the aqueous layer was further extracted with DCM (2×10 mL). The combined organic fractions were passed through a hydrophobic frit before being concentrated in vacuo. The crude product was purified by acidic pH reverse phase chromatography (eluting with a gradient of 10-100% MeCN (0.1% formic acid) in water (0.1% formic acid)) to afford 4-[2-[[1-(4-fluorophenyl)-2-oxo-pyridine-3-carbonyl]amino]pyrimidin-5-yl]oxy-N-(1-methyl-4-piperidyl)-1,7-naphthyridine-6-carboxamide formate salt (20.9 mg) as an orange solid.

LC-MS (Method 7A): 1.57 min; MS m/z 595.3=[M+H]+

¹H NMR (500 MHz, DMSO-d6) δ 12.66 (s, 1H), 9.46 (d, J=0.8 Hz, 1H), 8.95 (d, J=5.2 Hz, 1H), 8.89 (s, 2H), 8.86-8.77 (m, 2H), 8.61 (dd, J=7.3, 2.2 Hz, 1H), 8.18 (s, 1H), 8.16 (dd, J=6.6, 2.2 Hz, 1H), 7.67-7.56 (m, 2H), 7.48-7.36 (m, 2H), 7.22 (d, J=5.2 Hz, 1H), 6.79-6.68 (m, 1H), 3.91-3.83 (m, 1H), 2.85-2.77 (m, 2H), 2.22 (s, 3H), 2.14-2.02 (m, 2H), 1.87-1.70 (m, 4H).

### Example 20

### 2-(4-fluorophenyl)-N-[5-[[6-[4-(oxetan-3-yl)piperazin-1-yl]-1,7-naphthyridin-4-yl]oxy]-2-pyridyl]-3-oxo-pyridazine-4-carboxamide

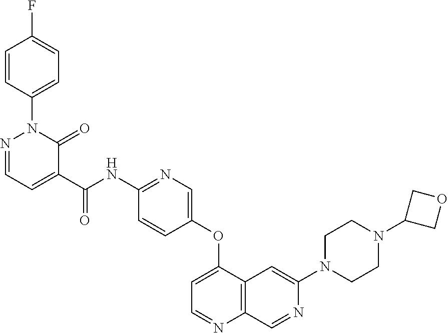 [SMILES: O=C(Nc1ccc(Oc2ccnc3cnc(N4CCN(C5COC5)CC4)cc23)cn1)c1ccnn(-c2ccc(F)cc2)c1=O]

### Step 1: 4-[(6-nitro-3-pyridyl)oxy]-6-[4-(oxetan-3-yl)piperazin-1-yl]-1,7-naphthyridine

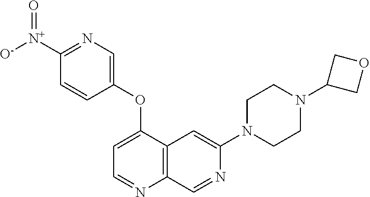 [SMILES: O=[N+]([O-])c1ccc(Oc2ccnc3cnc(N4CCN(C5COC5)CC4)cc23)cn1]

A mixture of 6-chloro-4-[(6-nitro-3-pyridyl)oxy]-1,7-naphthyridine (Example 10, step 1)(500 mg, 1.65 mmol), 1-(oxetan-3-yl)piperazine (352 mg, 2.48 mmol), cesium carbonate (1076 mg, 3.30 mmol), and RuPhos Pd-G3 (138 mg, 0.165 mmol) in dry DMA (20 mL) was degassed with nitrogen for 5 mins before being heated to 90° C. and allowed to stir for 2 h. The reaction mixture was partitioned between water (50 mL) and DCM (50 mL). The organic fraction was separated and the aqueous layer was further extracted with DCM (2×50 mL). The combined organic fractions were passed through a hydrophobic frit before being concentrated in vacuo. MeOH (20 ml) was added and the resultant solid was collected by filtration, washed with MeOH and dried to afford 4-[(6-nitro-3-pyridyl)oxy]-6-[4-(oxetan-3-yl)piperazin-1-yl]-1,7-naphthyridine (350 mg) as an orange solid.

LC-MS (Method 2A): 0.81 min; MS m/z 409.1=[M+H]+

¹H NMR (400 MHz, DMSO) δ 9.14 (s, 1H), 8.71 (d, J=2.7 Hz, 1H), 8.58 (d, J=4.9 Hz, 1H), 8.45 (d, J=8.9 Hz, 1H), 8.07 (dd, J=8.9, 2.8 Hz, 1H), 7.09 (d, J=4.9 Hz, 1H), 7.07 (s, 1H), 4.59-4.55 (m, 2H), 4.52-4.47 (m, 2H), 3.66-3.60 (m, 4H), 3.48-3.44 (m, 1H), 2.43-2.40 (m, 4H).

### Step 2: 5-[[6-[4-(oxetan-3-yl)piperazin-1-yl]-1,7-naphthyridin-4-yl]oxy]pyridin-2-amine

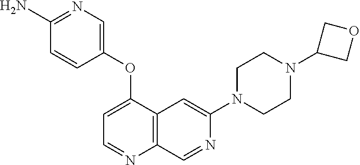 [SMILES: Nc1ccc(Oc2ccnc3cnc(N4CCN(C5COC5)CC4)cc23)cn1]

A suspension of 4-[(6-nitro-3-pyridyl)oxy]-6-[4-(oxetan-3-yl)piperazin-1-yl]-1,7-naphthyridine (Example 20, step 1)(350 mg, 0.857 mmol), iron powder (239 mg, 4.28 mmol) and ammonium chloride (229 mg, 4.28 mmol) in methanol (15 mL) and water (5 mL) was heated to 75° C. in a sealed tube for 1 h. The reaction mixture was cooled to room temperature before being filtered through glass fibre paper. The solids were washed with MeOH (2×10 mL) and CHCl₃/IPA (2:1, 2×10 mL) and the combined filtrates were concentrated in vacuo. The residue was partitioned between CHCl₃/iIPA (2:1) (3×30 mL) and water (30 mL) and the combined organics were passed through a hydrophobic frit, dried over MgSO4 and concentrated in vacuo. The crude product was purified by high pH reverse phase chromatography (eluting with a gradient of 10-100% MeCN (0.1% ammonium hydroxide) in water (0.1% ammonium hydroxide)) to afford 5-[[6-[4-(oxetan-3-yl)piperazin-1-yl]-1,7-naphthyridin-4-yl]oxy]pyridin-2-amine (170 mg) as a yellow solid.

LC-MS (Method 2A): 0.48 min; MS m/z 379.3=[M+H]+

¹H NMR (400 MHz, DMSO) δ 9.06 (s, 1H), 8.45 (d, J=5.0 Hz, 1H), 7.90 (d, J=2.8 Hz, 1H), 7.38 (dd, J=8.9, 2.9 Hz, 1H), 7.15 (s, 1H), 6.57 (dd, J=6.8, 1.8 Hz, 2H), 6.08 (s, 2H), 4.59 (t, J=6.5 Hz, 2H), 4.50 (t, J=6.1 Hz, 2H), 3.67-3.60 (m, 4H), 3.51-3.43 (m, 1H), 2.44-2.42 (m, 4H).

### Step 3: 2-(4-fluorophenyl)-N-[5-[[6-[4-(oxetan-3-yl)piperazin-1-yl]-1,7-naphthyridin-4-yl]oxy]-2-pyridyl]-3-oxo-pyridazine-4-carboxamide

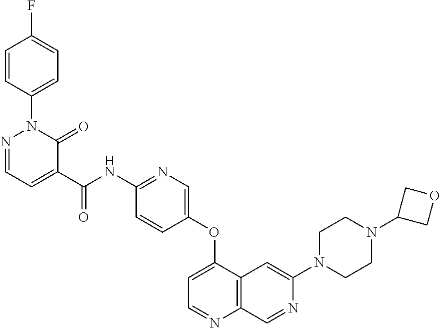 [SMILES: O=C(Nc1ccc(Oc2ccnc3cnc(N4CCN(C5COC5)CC4)cc23)cn1)c1ccnn(-c2ccc(F)cc2)c1=O]

5-[[6-[4-(oxetan-3-yl)piperazin-1-yl]-1,7-naphthyridin-4-yl]oxy]pyridin-2-amine (Example 20, step 2)(40 mg, 0.106 mmol) was added to a solution of 2-(4-fluorophenyl)-3-oxo-pyridazine-4-carboxylic acid (25 mg, 0.106 mmol), HATU (0.060 g, 0.159 mmol) and DIPEA (0.055 mL, 0.317 mmol) in DMF (1 mL). The reaction mixture was stirred at room temperature for 88 h and the crude mixture purified by prep HPLC (early elute high pH method) to afford 2-(4-fluorophenyl)-N-[5-[[6-[4-(oxetan-3-yl)piperazin-1-yl]-1,7-naphthyridin-4-yl]oxy]-2-pyridyl]-3-oxo-pyridazine-4-carboxamide (22 mg) as a brown solid.

LC-MS (Method 7B): 3.32 min; MS m/z=595.5 [M+H]+

1H NMR (400 MHz, DMSO) δ 12.11 (s, 1H), 9.10 (s, 1H), 8.48 (d, J=5.0 Hz, 1H), 8.46-8.38 (m, 3H), 8.34 (d, J=4.2 Hz, 1H), 7.93 (dd, J=9.0, 2.9 Hz, 1H), 7.74-7.66 (m, 2H), 7.48-7.38 (m, 2H), 7.17 (s, 1H), 6.74 (d, J=5.0 Hz, 1H), 4.59 (t, J=6.5 Hz, 2H), 4.50 (t, J=6.0 Hz, 2H), 3.69-3.61 (m, 4H), 3.49-3.43 (m, 1H), 2.44-2.42 (m, 4H).

The compounds of the following tabulated examples (Table 13) were prepared analogously to Example 20 step 3 from 5-[[6-[4-(oxetan-3-yl)piperazin-1-yl]-1,7-naphthyridin-4-yl]oxy]pyridin-2-amine (Example 20, step 2) and the appropriate carboxylic acid.

TABLE 13

| Ex. | Structure and Name | Retention Time, [M + H]+, 1H NMR |
|---|---|---|
| 20.1 | 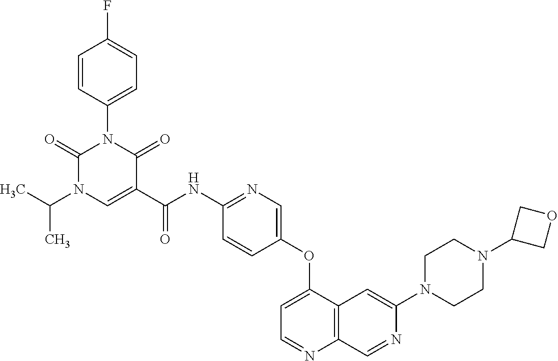 [SMILES: CC(C)n1cc(C(=O)Nc2ccc(Oc3ccnc4cnc(N5CCN(C6COC6)CC5)cc34)cn2)c(=O)n(-c2ccc(F)cc2)c1=O] | LC-MS (Method 7B): 3.35 min; MS m/z = 653.6 [M + H]+ ¹H NMR (400 MHz, DMSO) δ 11.47 (s, 1H), 9.15 (d, J = 0.7 Hz, 1H), 8.79 (s, 1H), 8.53 (d, J = 5.0 Hz, 1H), 8.44 (dd, J = 6.0, 2.6 Hz, 2H), 7.95 (dd, J = 9.1, 2.8 Hz, 1H), 7.49 (ddd, J = 8.1, 5.3, 2.7 Hz, 2H), 7.46-7.37 (m, 2H), 7.21 (s, 1H), 6.78 (d, J = 5.0 Hz, 1H), 4.90- 4.78 (m, 1H), 4.64 (t, J = 6.5 Hz, 2H), 4.56 (t, J = 6.1 Hz, 2H), 3.76-3.62 (m, 4H), 3.57-3.47 (m, 1H), 2.50-2.47 (m, 4H), 1.50 (d, J = 6.8 Hz, 6H). |
| | 3-(4-fluorophenyl)-1-isopropyl-N-[5-[[6-[4-(oxetan-3-yl)piperazin-1-yl]- | |
| | 1,7-naphthyridin-4-yl]oxy]-2-pyridyl]-2,4-dioxo-pyrimidine-5-carboxamide | |
| 20.2 | 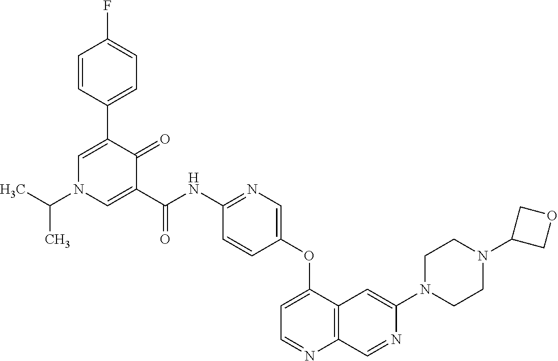 [SMILES: CC(C)n1cc(C(=O)Nc2ccc(Oc3ccnc4cnc(N5CCN(C6COC6)CC5)cc34)cn2)c(=O)c(-c2ccc(F)cc2)c1] | LC-MS (Method 7B): 3.43 min; MS m/z = 636.6 [M + H]+ ¹H NMR (500 MHz, DMSO) δ 13.44 (s, 1H), 9.10 (s, 1H), 8.80 (d, J = 2.4 Hz, 1H), 8.48 (d, J = 5.0 Hz, 1H), 8.44 (d, J = 9.0 Hz, 1H), 8.40 (d, J = 2.9 Hz, 1H), 8.28 (d, J = 2.4 Hz, 1H), 7.88 (dd, J = 9.0, 2.9 Hz, 1H), 7.79-7.72 (m, 2H), 7.33-7.26 (m, 2H), 7.18 (s, 1H), 6.73 (d, J = 5.0 Hz, 1H), 4.69-4.62 (m, 1H), 4.59 (t, J = 6.5 Hz, 2H), 4.50 (t, J = 6.1 Hz, 2H), 3.68-3.62 (m, 4H), 3.51- 3.42 (m, 1H), 2.46-2.42 (m, 4H), 1.53 (d, J = 6.7 Hz, 6H). |
| | 5-(4-fluorophenyl)-1-isopropyl-N-[5-[[6-[4-(oxetan-3-yl)piperazin-1-yl]- | |
| | 1,7-naphthyridin-4-yl]oxy]-2-pyridyl]-4-oxo-pyridine-3-carboxamide | |

### Example 21: 1-(4-fluorophenyl)-2-oxo-N-[5-[(6-pyrrolidin-3-yl-1,7-naphthyridin-4-yl)oxy]-2-pyridyl]pyridine-3-carboxamide

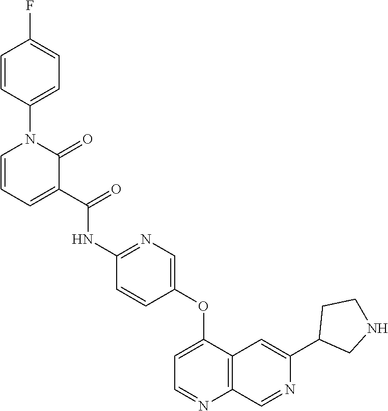 [SMILES: O=C(Nc1ccc(Oc2ccnc3cnc(C4CCNC4)cc23)cn1)c1cccn(-c2ccc(F)cc2)c1=O]

Trifluoroacetic acid (12 μL, 0.161 mmol) was added dropwise at 0° C. to a solution of tert-butyl 3-[4-[[6-[[1-(4-fluorophenyl)-2-oxo-pyridine-3-carbonyl]amino]-3-pyridyl]oxy]-1,7-naphthyridin-6-yl]pyrrolidine-1-carboxylate (Example 16.1)(10 mg, 0.0161 mmol) in DCM (1 mL) and stirred at room temperature for 4 h. The material was as concentrated in vacuo and loaded in minimal MeCN onto a 1 g SCX cartridge, before the column was washed sequentially with MeCN (3×5 mL) and 7M NH₃/MeOH (3×5 mL). The methanolic ammonia fraction was concentrated in vacuo to afford 1-(4-fluorophenyl)-2-oxo-N-[5-[(6-pyrrolidin-3-yl-1,7-naphthyridin-4-yl)oxy]-2-pyridyl]pyridine-3-carboxamide (7.0 mg) as a white solid.

LC-MS (Method 7A): 1.96 min; MS m/z=523.3 [M+H]+

¹H NMR (400 MHz, DMSO) δ 12.46 (s, 1H), 9.39 (s, 1H), 8.78 (d, J=5.1 Hz, 1H), 8.65 (dd, J=7.3, 2.1 Hz, 1H), 8.49-8.39 (m, 2H), 8.16 (dd, J=6.6, 2.2 Hz, 1H), 8.06 (s, 1H), 7.93 (dd, J=9.0, 2.9 Hz, 1H), 7.66-7.58 (m, 2H), 7.48-7.39 (m, 2H), 6.91 (d, J=5.1 Hz, 1H), 6.78-6.71 (m, 1H), 3.67-3.59 (m, 1H), 3.15-3.07 (m, 2H), 3.05-2.96 (m, 2H), 2.26-2.20 (m, 1H), 2.08-1.96 (m, 1H).

The compound of the following tabulated example (Table 14) was prepared analogously to Example 21 using Example 16.4.

TABLE 14

| Ex. | Structure and Name | Retention Time, [M + H]+, 1H NMR |
|---|---|---|
| 21.1 | 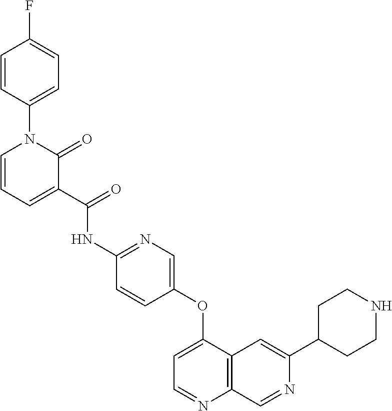 [SMILES: O=C(Nc1ccc(Oc2ccnc3cnc(C4CCNCC4)cc23)cn1)c1cccn(-c2ccc(F)cc2)c1=O] | LC-MS (Method 7B): 2.87 min; MS m/z = 537.5 [M + H]+ ¹H NMR (500 MHz, DMSO) δ 12.46 (s, 1H), 9.38 (s, 1H), 8.78 (d, J = 5.1 Hz, 1H), 8.65 (dd, J = 7.3, 2.2 Hz, 1H), 8.46 (d, J = 9.3 Hz, 1H), 8.43 (d, J = 2.9 Hz, 1H), 8.17 (dd, J = 6.6, 2.2 Hz, 1H), 7.97-7.91 (m, 2H), 7.66-7.59 (m, 2H), 7.48-7.40 (m, 2H), 6.91 (d, J = 5.1 Hz, 1H), 6.78-6.72 (m, 1H), 3.10 (d, J = 11.8 Hz, 2H), 3.02 (t, J = 11.9 Hz, 1H), 2.68 (t, J = 11.2 Hz, 2H), 1.91 (d, J = 12.4 Hz, 2H), 1.81-1.68 (m, 2H). |
| | 1-(4-fluorophenyl)-2-oxo-N-[5-[[6-(4-piperidyl)-1,7-naphthyridin- | |
| | 4-yl]oxy]-2-pyridyl]pyridine-3-carboxamide | |

### Example 22: 1-(4-fluorophenyl)-N-[5-[[6-[1-(2-methylpropanoyl)-4-piperidyl]-1,7-naphthyridin-4-yl]oxy]-2-pyridyl]-2-oxo-pyridine-3-carboxamide

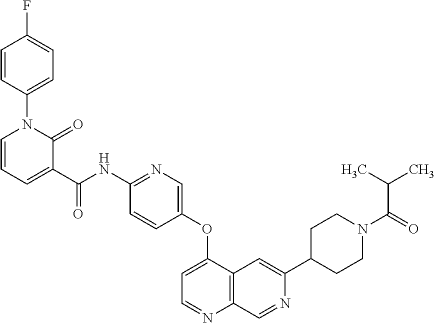 [SMILES: CC(C)C(=O)N1CCC(c2cc3c(Oc4ccc(NC(=O)c5cccn(-c6ccc(F)cc6)c5=O)nc4)ccnc3cn2)CC1]

1-(4-fluorophenyl)-2-oxo-N-[5-[[6-(4-piperidyl)-1,7-naphthyridin-4-yl]oxy]-2-pyridyl]pyridine-3-carboxamide (Example 21.1)(25 mg, 0.0466 mmol) was added to a solution of 2-methylpropanoic acid (4.9 mg, 0.0559 mmol), HATU (0.027 g, 0.0699 mmol) and DIPEA (24 μL, 0.140 mmol) in DMF (1 mL). The reaction mixture was stirred at room temperature for 17 h and crude mixture purified by prep HPLC (early elute-high pH method) to afford 1-(4-fluorophenyl)-N-[5-[[6-[1-(2-methylpropanoyl)-4-piperidyl]-1,7-naphthyridin-4-yl]oxy]-2-pyridyl]-2-oxo-pyridine-3-carboxamide (16 mg) as a white solid.

LC-MS (Method 7A): 3.27 min; MS m/z=607.3 [M+H]+

¹H NMR (400 MHz, DMSO) δ 12.45 (s, 1H), 9.38 (s, 1H), 8.78 (d, J=5.1 Hz, 1H), 8.64 (dd, J=7.3, 2.2 Hz, 1H), 8.45 (d, J=9.2 Hz, 1H), 8.42 (d, J=2.9 Hz, 1H), 8.16 (dd, J=6.6, 2.2 Hz, 1H), 8.01 (s, 1H), 7.93 (dd, J=9.0, 2.9 Hz, 1H), 7.66-7.58 (m, 2H), 7.47-7.39 (m, 2H), 6.91 (d, J=5.1 Hz, 1H), 6.78-6.71 (m, 1H), 4.67-4.52 (m, 1H), 4.18-4.05 (m, 1H), 3.24-3.13 (m, 2H), 2.98-2.86 (m, 1H), 2.07-1.94 (m, 2H), 1.86-1.71 (m, 1H), 1.71-1.57 (m, 1H), 1.05-0.97 (m, 6H).

### Example 23: 5-(4-fluorophenyl)-1-isopropyl-N-[5-[[6-(1-methyl-4-piperidyl)-1,7-naphthyridin-4-yl]oxy]-2-pyridyl]-4-oxo-pyridine-3-carboxamide

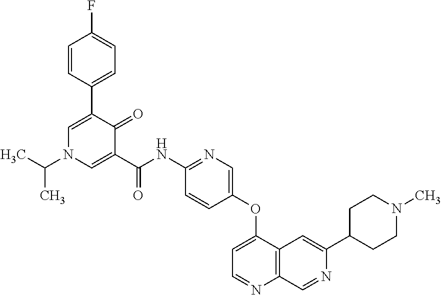 [SMILES: CC(C)n1cc(C(=O)Nc2ccc(Oc3ccnc4cnc(C5CCN(C)CC5)cc34)cn2)c(=O)c(-c2ccc(F)cc2)c1]

### Step 1: tert-butyl N-tert-butoxycarbonyl-N-[5-[(6-chloro-1,7-naphthyridin-4-yl)oxy]-2-pyridyl]carbamate

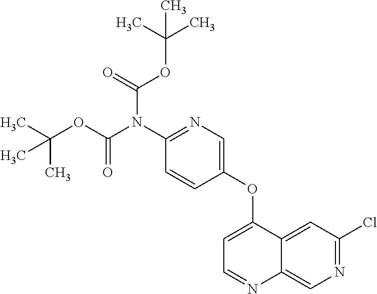 [SMILES: CC(C)(C)OC(=O)N(C(=O)OC(C)(C)C)c1ccc(Oc2ccnc3cnc(Cl)cc23)cn1]

5-[(6-chloro-1,7-naphthyridin-4-yl)oxy]pyridin-2-amine (Example 10, step 2)(0.40 g, 1.47 mmol), triethylamine (0.49 mL, 3.53 mmol) and DMAP (18 mg, 0.147 mmol) were suspended in THF (20 mL). Di-tert-butyl dicarbonate (707 mg, 3.24 mmol) was added portion wise and the reaction was stirred at 50° C. for 18 h. Further di-tert-butyl dicarbonate (175 mg, 0.80 mmol) and triethylamine (0.125 mL, 0.88 mmol) were added and the reaction heated for a further 4 h. The reaction mixture was cooled to room temperature and concentrated in vacuo. The crude residue was redissolved in DCM (30 mL), washed with water (2×20 mL), dried over MgSO4 and concentrated in vacuo. The crude product was purified by silica chromatography (eluting with 0-50% EtOAc in heptanes) to afford tert-butyl N-tert-butoxycarbonyl-N-[5-[(6-chloro-1,7-naphthyridin-4-yl)oxy]-2-pyridyl]carbamate (0.52 g) as a white solid.

LC-MS (Method 2A): 1.32 min; MS m/z=473.2 [M+H]+

¹H NMR (400 MHz, DMSO-d6) δ 9.33 (d, J=0.8 Hz, 1H), 8.92 (d, J=5.1 Hz, 1H), 8.57-8.53 (m, 1H), 8.29 (d, J=0.7 Hz, 1H), 7.96 (dd, J=8.7, 2.9 Hz, 1H), 7.63-7.58 (m, 1H), 6.89 (d, J=5.1 Hz, 1H), 1.43 (s, 18H).

### Step 2: tert-butyl N-tert-butoxycarbonyl-N-[5-[[6-(1-methyl-3,6-dihydro-2H-pyridin-4-yl)-1,7-naphthyridin-4-yl]oxy]-2-pyridyl]carbamate

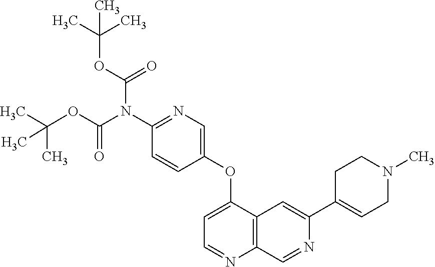 [SMILES: CN1CC=C(c2cc3c(Oc4ccc(N(C(=O)OC(C)(C)C)C(=O)OC(C)(C)C)nc4)ccnc3cn2)CC1]

tert-butyl N-tert-butoxycarbonyl-N-[5-[(6-chloro-1,7-naphthyridin-4-yl)oxy]-2-pyridyl]carbamate (Example 23, step 1)(470 mg, 0.994 mmol), 1-methyl-4-(4,4,5,5-tetramethyl-1,3,2-dioxaborolan-2-yl)-3,6-dihydro-2H-pyridine (333 mg, 1.49 mmol) and tripotassium phosphate (422 mg, 1.99 mmol) were suspended in 1,4-dioxane (4 mL) and water (0.5 mL). The mixture was degassed with nitrogen, Pd(dppf)Cl₂ (81 mg, 0.0994 mmol) was added and the mixture heated at 90° C. for 6 h. After cooling to room temperature, the mixture was partitioned between DCM (10 mL) and water (10 mL), the phases were separated by passing the mixture through a hydrophobic frit and the organic phase concentration in vacuo. The crude product was purified by reverse phase chromatography (eluting with a gradient of 10-100% MeCN (0.1% formic acid) in water (0.1% formic acid)) to afford tert-butyl N-tert-butoxycarbonyl-N-[5-[[6-(1-methyl-3,6-dihydro-2H-pyridin-4-yl)-1,7-naphthyridin-4-yl]oxy]-2-pyridyl]carbamate (350 mg) as a yellow foam.

LC-MS (Method 2A): 1.00 min; MS m/z=534.3 [M+H]+

¹H NMR (500 MHz, DMSO) δ 9.39 (d, J=0.8 Hz, 1H), 8.83 (d, J=5.1 Hz, 1H), 8.58-8.53 (m, 1H), 8.04 (s, 1H), 8.00-7.93 (m, 1H), 7.63-7.57 (m, 1H), 7.05 (s, 1H), 6.81 (d, J=5.1 Hz, 1H), 3.14 (d, J=3.0 Hz, 2H), 2.71-2.62 (m, 4H), 2.32 (s, 3H), 1.43 (s, 18H).

### Step 3: tert-butyl N-(5-{[6-(1-methylpiperidin-4-yl)-1,7-naphthyridin-4-yl]oxy}pyridin-2-yl)carbamate

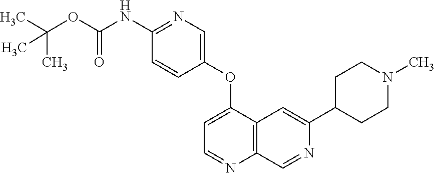 [SMILES: CN1CCC(c2cc3c(Oc4ccc(NC(=O)OC(C)(C)C)nc4)ccnc3cn2)CC1]

To a solution of tert-butyl N-tert-butoxycarbonyl-N-[5-[[6-(1-methyl-3,6-dihydro-2H-pyridin-4-yl)-1,7-naphthyridin-4-yl]oxy]-2-pyridyl]carbamate (Example 23, step 2)(250 mg, 0.468 mmol) and acetic acid (0.054 mL, 0.937 mmol) in ethanol (10 mL) was added 10% w/w Pd/C (50 mg, 0.0468 mmol) and the reaction was stirred under a 1 atm pressure of hydrogen for 16 h. The mixture was filtered through a pad of celite and concentrated in vacuo. The crude product was purified by reverse phase chromatography (eluting with a gradient of 10-100% MeCN (0.1% formic acid) in water (0.1% formic acid)) to afford tert-butyl N-(5-{[6-(1-methylpiperidin-4-yl)-1,7-naphthyridin-4-yl]oxy}pyridin-2-yl)carbamate (200 mg) as an orange foam.

LC-MS (Method 2A): 0.92 min; MS m/z=436.3 [M+H]+

¹H NMR (500 MHz, MeOD) δ 9.43-9.36 (m, 1H), 8.84-8.78 (m, 1H), 8.31-8.28 (m, 1H), 8.26-8.23 (m, 1H), 8.19-8.15 (m, 1H), 8.08-8.04 (m, 1H), 7.92-7.68 (m, 2H), 6.93-6.87 (m, 1H), 3.70-3.64 (m, 2H), 3.27-3.21 (m, 2H), 2.96 (s, 3H), 2.35-2.22 (m, 4H), 1.59-1.45 (m, 9H).

### Step 4: 5-[[6-(1-methyl-4-piperidyl)-1,7-naphthyridin-4-yl]oxy]pyridin-2-amine

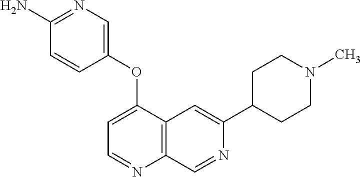 [SMILES: CN1CCC(c2cc3c(Oc4ccc(N)nc4)ccnc3cn2)CC1]

To a solution of tert-butyl N-(5-{[6-(1-methylpiperidin-4-yl)-1,7-naphthyridin-4-yl]oxy}pyridin-2-yl)carbamate (Example 23, step 3)(200 mg, 0.459 mmol) in DCM (1 mL) at 0° C. was added trifluoroacetic acid (0.58 mL, 7.47 mmol) and the reaction was stirred whilst slowly warming up to room temperature over 5 h. The mixture was concentrated in vacuo, saturated NaHCO₃ solution (10 mL) was added and the solution extracted with CHCl₃/IPA (3:1) (20 mL×3). The combined organics were passed through a hydrophobic frit and concentrated in vacuo to afford 5-[[6-(1-methyl-4-piperidyl)-1,7-naphthyridin-4-yl]oxy]pyridin-2-amine (120 mg) as an orange foam.

LC-MS (Method 7B): 2.12 min; MS m/z=336.4 [M+H]+

¹H NMR (500 MHz, MeOD) δ 9.21 (d, J=0.8 Hz, 1H), 8.63 (d, J=5.2 Hz, 1H), 7.99 (s, 1H), 7.83-7.79 (m, 1H), 7.35 (dd, J=9.0, 2.9 Hz, 1H), 6.74 (d, J=5.2 Hz, 1H), 6.63 (dd, J=9.0, 0.6 Hz, 1H), 2.98 (d, J=11.7 Hz, 2H), 2.90-2.81 (m, 1H), 2.27 (s, 3H), 2.20-2.11 (m, 2H), 2.04-1.96 (m, 2H), 1.94-1.83 (m, 2H).

### Step 5: 5-(4-fluorophenyl)-1-isopropyl-N-[5-[[6-(1-methyl-4-piperidyl)-1,7-naphthyridin-4-yl]oxy]-2-pyridyl]-4-oxo-pyridine-3-carboxamide

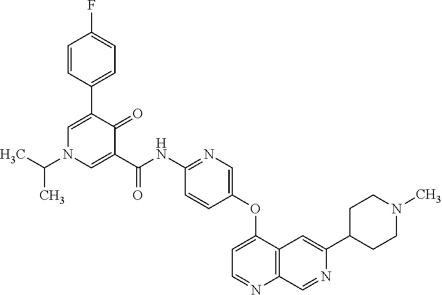 [SMILES: CC(C)n1cc(C(=O)Nc2ccc(Oc3ccnc4cnc(C5CCN(C)CC5)cc34)cn2)c(=O)c(-c2ccc(F)cc2)c1]

5-[[6-(1-methyl-4-piperidyl)-1,7-naphthyridin-4-yl]oxy]pyridin-2-amine (Example 23, step 4) (50 mg, 0.149 mmol) was added to a solution of 5-(4-fluorophenyl)-1-isopropyl-4-oxo-pyridine-3-carboxylic acid (Intermediate H)(41 mg, 0.149 mmol), HATU (0.085 g, 0.224 mmol) and DIPEA (0.078 mL, 0.447 mmol) in DMF (0.75 mL) and the reaction mixture was stirred at room temperature for 65 h. The mixture was diluted with water (15 mL) and stirred for 5 minutes before the formed solid was collected by filtration and washed with water (2×5 mL). The crude solid was purified by prep HPLC (standard high pH method) to afford 5-(4-fluorophenyl)-1-isopropyl-N-[5-[[6-(1-methyl-4-piperidyl)-1,7-naphthyridin-4-yl]oxy]-2-pyridyl]-4-oxo-pyridine-3-carboxamide (39 mg) as an off-white solid.

LC-MS (Method 7B): 3.67 min; MS m/z=593.7 [M+H]+

1H NMR (500 MHz, DMSO) δ 13.45 (s, 1H), 9.39 (d, J=0.7 Hz, 1H), 8.81 (d, J=2.4 Hz, 1H), 8.78 (d, J=5.1 Hz, 1H), 8.48-8.42 (m, 2H), 8.29 (d, J=2.4 Hz, 1H), 7.99 (s, 1H), 7.93 (dd, J=9.0, 2.9 Hz, 1H), 7.81-7.70 (m, 2H), 7.35-7.24 (m, 2H), 6.92 (d, J=5.1 Hz, 1H), 4.66 (p, J=6.7 Hz, 1H), 2.96-2.82 (m, 3H), 2.22 (s, 3H), 2.07-1.98 (m, 2H), 1.99-1.83 (m, 4H), 1.53 (d, J=6.7 Hz, 6H).

The compound of the following tabulated example (Table 15) was prepared analogously to Example 23, step 5 using 5-[[6-(1-methyl-4-piperidyl)-1,7-naphthyridin-4-yl]oxy]pyridin-2-amine (Example 23, step 4) and Intermediate C.

TABLE 15

| Ex. | Structure and Name | Retention Time, [M + H]+, 1H NMR |
|---|---|---|
| 23.1 | 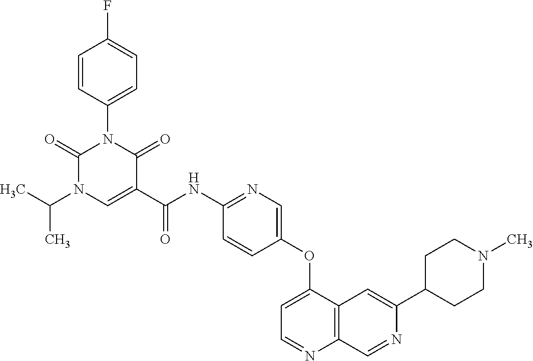 [SMILES: CC(C)n1cc(C(=O)Nc2ccc(Oc3ccnc4cnc(C5CCN(C)CC5)cc34)cn2)c(=O)n(-c2ccc(F)cc2)c1=O] | LC-MS (Method 7A): 2.03 min; MS m/z = 610.3 [M + H]+ ¹H NMR (500 MHz, DMSO) δ 11.44 (s, 1H), 9.41 (s, 1H), 8.81 (d, J = 5.1 Hz, 1H), 8.74 (s, 1H), 8.44-8.39 (m, 2H), 8.03 (s, 1H), 7.95 (dd, J = 9.0, 2.9 Hz, 1H), 7.48-7.40 (m, 2H), 71.40-7.33 (m, 2H), 6.94 (d, J = 5.1 Hz, 1H), 4.85-4.75 (m, 1H), 3.41 (s, 2H), 3.21- 3.13 (m, 1H), 2.74-2.67 (m, 2H), 2.18- 2.04 (m, 4H), 1.44 (d, J = 6.8 Hz, 6H) |
| | 3-(4-fluorophenyl)-1-isopropyl-N-[5-[[6-(1-methyl-4-piperidyl)-1,7- | |
| | naphthyridin-4-yl]oxy]-2-pyridyl]-2,4-dioxo-pyrimidine-5-carboxamide | |

### Example 24: 1-(4-fluorophenyl)-N-[5-[[6-[(1-methyl-4-piperidyl)oxy]-1,7-naphthyridin-4-yl]oxy]-2-pyridyl]-2-oxo-pyridine-3-carboxamide

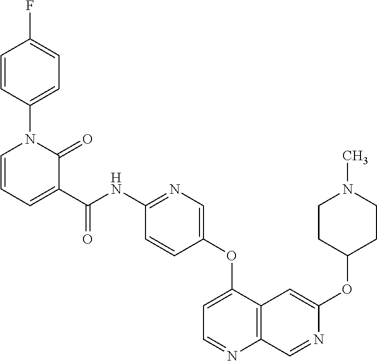 [SMILES: CN1CCC(Oc2cc3c(Oc4ccc(NC(=O)c5cccn(-c6ccc(F)cc6)c5=O)nc4)ccnc3cn2)CC1]

### Step 1: 4-[(6-nitro-3-pyridyl)oxy]-1,7-naphthyridin-6-ol

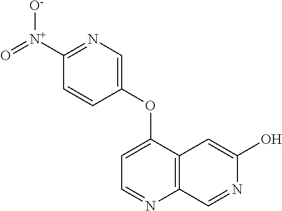 [SMILES: O=[N+]([O-])c1ccc(Oc2ccnc3cnc(O)cc23)cn1]

A mixture of 6-chloro-4-[(6-nitro-3-pyridyl)oxy]-1,7-naphthyridine (Example 10, step 1)(250 mg, 0.826 mmol), tBuXPhos-Pd-G3 (66 mg, 0.0826 mmol) and sodium tert-butoxide (159 mg, 1.65 mmol) in 1,4-dioxane (1 mL) was degassed with nitrogen for 5 mins before being stirred at room temperature for 6 h. The reaction mixture was partitioned between DCM (30 mL) and water (30 mL), the organic fraction separated and the aqueous layer further extracted with DCM (3×15 mL). The combined organic fractions were passed through a hydrophobic frit and concentrated in vacuo. The crude solid was suspended in MeCN:DMSO (5:1, 5 mL), the solid collected by filtration, washed with MeCN (2×1 mL) and dried to afford 4-[(6-nitro-3-pyridyl)oxy]-1,7-naphthyridin-6-ol (90 mg) as a yellow solid.

LC-MS (Method 2A): 0.83 min; MS m/z=285.0 [M+H]+

¹H NM R (500 MHz, DMSO) δ 11.28 (s, 1H), 9.10 (s, 1H), 8.71 (d, J=2.8 Hz, 1H), 8.67 (d, J=4.9 Hz, 1H), 8.45 (d, J=8.9 Hz, 1H), 8.09 (dd, J=8.9, 2.8 Hz, 1H), 7.15 (d, J=4.9 Hz, 1H), 7.07 (s, 1H).

### Step 2: 6-[(1-methyl-4-piperidyl)oxy]-4-[(6-nitro-3-pyridyl)oxy]-1,7-naphthyridine

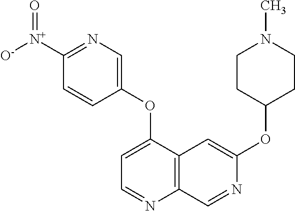 [SMILES: CN1CCC(Oc2cc3c(Oc4ccc([N+](=O)[O-])nc4)ccnc3cn2)CC1]

A solution of DIAD (77 uL, 0.394 mmol) and triphenylphosphine (103 mg, 0.394 mmol) in THF (2 mL) was stirred at 0° C. under nitrogen for 20 minutes before being added dropwise to a solution of 4-[(6-nitro-3-pyridyl)oxy]-1,7-naphthyridin-6-ol (Example 24, step 1)(70 mg, 0.246 mmol) and 1-methylpiperidin-4-ol (43 mg, 0.372 mmol) in THF (3 mL) at 0° C. The reaction mixture was allowed to warm to room temperature and was stirred for 1 h. The reaction mixture was partitioned between EtOAc (10 mL) and water (10 mL), the organic fraction separated and the aqueous layer further extracted with EtOAc (10 mL). The combined organic fractions were washed with brine (20 mL), dried over MgSO₄ and concentrated in vacuo. The crude residue was loaded onto an SCX cartridge (2 g column) which was sequentially washed with MeOH (3×5 mL) and 7M NH₃ in MeOH (2×5 mL). The methanolic ammonia fraction was concentrated in vacuo to afford 6-[(1-methyl-4-piperidyl)oxy]-4-[(6-nitro-3-pyridyl)oxy]-1,7-naphthyridine (66 mg) as an orange solid.

LC-MS (Method 2A): 0.85 min; MS m/z=382.1 [M+H]+

¹H NMR (500 MHz, DMSO) δ 9.22 (d, J=0.7 Hz, 1H), 8.75 (d, J=4.9 Hz, 1H), 8.71 (d, J=2.7 Hz, 1H), 8.45 (d, J=8.9 Hz, 1H), 8.10 (dd, J=8.9, 2.8 Hz, 1H), 7.31 (d, J=0.7 Hz, 1H), 7.20 (d, J=4.9 Hz, 1H), 5.16-5.06 (m, 1H), 2.71-2.63 (m, 2H), 2.19 (m, 5H), 1.99 (m, 2H), 1.79-1.68 (m, 2H).

### Step 3: 5-[[6-[(1-methyl-4-piperidyl)oxy]-1,7-naphthyridin-4-yl]oxy]pyridin-2-amine

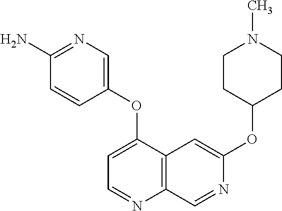 [SMILES: CN1CCC(Oc2cc3c(Oc4ccc(N)nc4)ccnc3cn2)CC1]

A suspension of 6-[(1-methyl-4-piperidyl)oxy]-4-[(6-nitro-3-pyridyl)oxy]-1,7-naphthyridine (Example 24, step 2) (100 mg, 0.223 mmol), iron powder (62 mg, 1.11 mmol) and ammonium chloride (60 mg, 1.11 mmol) in methanol (3 mL) and water (1 mL) was heated to 75° C. and stirred for 1 h. The mixture was filtered through a pad of celite and the filter bed washed with MeOH (20 mL) and CHCl₃/IPA (2:1, 20 mL). The combined filtrates were loaded onto an SCX cartridge (5 g column) and the column was sequentially washed with MeOH (2×20 mL) and 7M NH₃ in MeOH (2×20 mL). The methanolic ammonia fraction was concentrated in vacuo to afford 5-[[6-[(1-methyl-4-piperidyl)oxy]-1,7-naphthyridin-4-yl]oxy]pyridin-2-amine (35 mg) as an orange solid.

LC-MS (Method 2A): 0.50 min; MS m/z=352.3 [M+H]+

¹H NMR (500 MHz, DMSO) δ 9.12 (d, J=0.8 Hz, 1H), 8.60 (d, J=5.0 Hz, 1H), 7.91 (d, J=2.9 Hz, 1H), 7.39 (dd, J=8.9, 3.0 Hz, 1H), 7.37-7.33 (m, 1H), 6.66 (d, J=5.0 Hz, 1H), 6.62-6.56 (m, 1H), 6.10 (s, 2H), 5.14-5.06 (m, 1H), 2.75-2.66 (m, 2H), 2.33-2.19 (m, 5H), 2.07-1.95 (m, 2H), 1.81-1.70 (m, 2H).

### Step 4: 1-(4-fluorophenyl)-N-[5-[[6-[(1-methyl-4-piperidyl)oxy]-1,7-naphthyridin-4-yl]oxy]-2-pyridyl]-2-oxo-pyridine-3-carboxamide

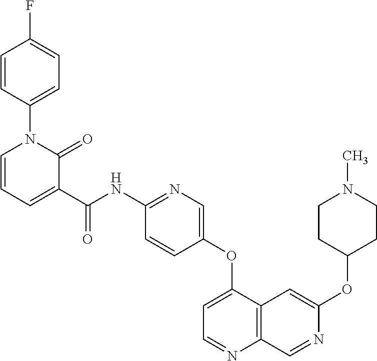 [SMILES: CN1CCC(Oc2cc3c(Oc4ccc(NC(=O)c5cccn(-c6ccc(F)cc6)c5=O)nc4)ccnc3cn2)CC1]

5-[[6-[(1-methyl-4-piperidyl)oxy]-1,7-naphthyridin-4-yl]oxy]pyridin-2-amine (Example 24, step 3) (35 mg, 0.0996 mmol) was added to a solution of 1-(4-fluorophenyl)-2-oxo-pyridine-3-carboxylic acid (Intermediate B)(23 mg, 0.0996 mmol), HATU (0.057 g, 0.149 mmol) and DIPEA (0.052 mL, 0.299 mmol) in DMF (2 mL) and the mixture was stirred for 96 h at room temperature. Water (15 mL) was added to the mixture and the resulting solid collected by filtration and washed with water (3×5 mL). The crude solid was purified by low pH reverse phase chromatography (eluting with a gradient of 10-100% MeCN (0.1% formic acid) in water (0.1% formic acid)). The pure fractions were loaded onto an SCX cartridge (2 g column) and the column was washed sequentially with MeOH (2×5 mL) and 7M NH₃ in MeOH (2×5 mL). The methanolic ammonia fraction was concentrated in vacuo 1-(4-fluorophenyl)-N-[5-[[6-[(1-methyl-4-piperidyl)oxy]-1,7-naphthyridin-4-yl]oxy]-2-pyridyl]-2-oxo-pyridine-3-carboxamide (19.7 mg) as a white solid.

LC-MS (Method 7A): 1.98 min; MS m/z=567.2 [M+H]+

¹H NMR (400 MHz, DMSO) δ 12.44 (s, 1H), 9.16 (d, J=0.7 Hz, 1H), 8.73-8.58 (m, 2H), 8.44 (d, J=9.2 Hz, 1H), 8.39 (d, J=2.6 Hz, 1H), 8.15 (dd, J=6.6, 2.2 Hz, 1H), 7.89 (dd, J=9.0, 2.9 Hz, 1H), 7.71-7.56 (m, 2H), 7.53-7.35 (m, 3H), 6.82 (d, J=5.0 Hz, 1H), 6.80-6.67 (m, 1H), 5.20-5.03 (m, 1H), 2.67-2.60 (m, 2H), 2.26-2.15 (m, 5H), 2.08-1.92 (m, 2H), 1.82-1.68 (m, 2H).

### Example 25: 4-[[6-[[1-(4-fluorophenyl)-2-oxo-pyridine-3-carbonyl]amino]-4-methyl-3-pyridyl]oxy]-N-(1-methyl-4-piperidyl)-1,7-naphthyridine-6-carboxamide

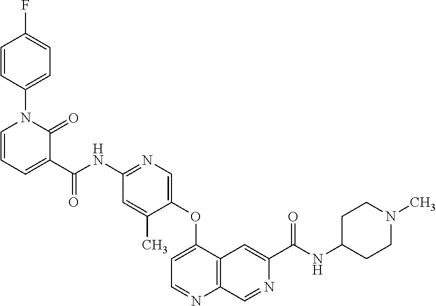 [SMILES: Cc1cc(NC(=O)c2cccn(-c3ccc(F)cc3)c2=O)ncc1Oc1ccnc2cnc(C(=O)NC3CCN(C)CC3)cc12]

### Step 1: 2-chloro-5-(methoxymethoxy)-4-methyl-pyridine

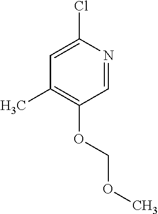

To a suspension of 6-chloro-4-methyl-pyridin-3-ol (250 mg, 1.74 mmol) and potassium carbonate (481 mg, 3.48 mmol) in dry DMF (10 mL) at 0° C. was added chloro(methoxy)methane (168 mg, 2.09 mmol). The mixture was warmed to room temperature and stirred for 7 h. The mixture was partitioned between water (10 mL) and DCM (10 mL), the organic fraction separated and the aqueous layer further extracted with DCM (2×10 mL). The combined organic fractions were passed through a hydrophobic frit before being concentrated in vacuo. The crude product was purified by silica chromatography (eluting with a gradient of 0-100% EtOAc in heptane) to afford 2-chloro-5-(methoxymethoxy)-4-methyl-pyridine (175 mg) as a colourless oil.

LC-MS (Method 2A): 1.11 min; MS m/z=188.1 [M+H]+

¹H NMR (500 MHz, Chloroform-d) δ 8.11 (s, 1H), 7.11 (s, 1H), 5.21 (s, 2H), 3.49 (s, 3H), 2.24 (s, 3H).

### Step 2: 5-(methoxymethoxy)-4-methyl-pyridin-2-amine formate salt

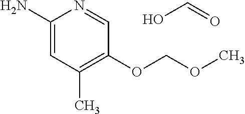

1M Lithium bis(trimethylsilyl)amide in THF (4.7 mL, 4.68 mmol) was added to a solution of 2-chloro-5-(methoxymethoxy)-4-methyl-pyridine (Example 25, step 1)(585 mg, 3.12 mmol) in dry THF (15 mL) and the mixture was flushed with nitrogen. Pd₂(dba)₃.CHCl₃ (161 mg, 0.156 mmol) and CyJohnPhos (109 mg, 0.312 mmol) were added and the mixture was heated to 60° C. for 18 h. After cooling to room temperature, the mixture was partitioned between DCM (20 mL) and water (20 mL), the organic fraction separated and the aqueous layer further extracted with DCM (2×10 mL). The combined organic fractions were passed through a hydrophobic frit before being concentrated in vacuo. The crude product was purified by low pH reverse phase chromatography (eluting with a gradient of 0-100% MeCN (0.1% formic acid) in water (0.1% formic acid) to afford 5-(methoxymethoxy)-4-methyl-pyridin-2-amine formate salt (35 mg) as a brown oil.

LC-MS (Method 2A): 0.43 min; MS m/z=169.2 [M+H]+

¹H NMR (500 MHz, DMSO-d6) δ 8.17 (s, 1H), 7.61 (s, 1H), 6.28 (s, 1H), 5.45 (s, 2H), 5.00 (s, 2H), 3.39 (s, 3H), 2.07 (s, 3H).

### Step 3: 1-(4-fluorophenyl)-N-[5-(methoxymethoxy)-4-methyl-2-pyridyl]-2-oxo-pyridine-3-carboxamide

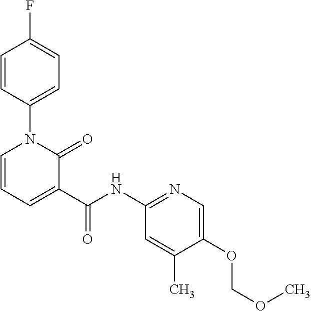 [SMILES: COCOc1cnc(NC(=O)c2cccn(-c3ccc(F)cc3)c2=O)cc1C]

5-(methoxymethoxy)-4-methyl-pyridin-2-amine (Example 25, step 2)(50 mg, 0.297 mmol) was added to a solution of DIPEA (0.72 mL, 4.16 mmol), 1-(4-fluorophenyl)-2-oxo-pyridine-3-carboxylic acid (Intermediate B)(104 mg, 0.446 mmol) and HATU (226 mg, 0.595 mmol) in DMF (5 mL) and the solution was stirred for 2 h at room temperature. Water (5 mL) was added to the mixture and the resulting solid was collected by filtration, washed with water (3×2 mL) and dried to afford 1-(4-fluorophenyl)-N-[5-(methoxymethoxy)-4-methyl-2-pyridyl]-2-oxo-pyridine-3-carboxamide (80 mg) as a brown solid.

LC-MS (Method 2A): 0.80 min; MS m/z=384.2 [M+H]+

¹H NMR (500 MHz, DMSO-d6) δ 12.10 (s, 1H), 8.58 (dd, J=7.3, 2.2 Hz, 1H), 8.16 (s, 1H), 8.11 (dd, J=6.6, 2.2 Hz, 1H), 8.02 (s, 1H), 7.62-7.57 (m, 2H), 7.44-7.39 (m, 2H), 6.71 (dd, J=7.2, 6.7 Hz, 1H), 5.23 (s, 2H), 3.41 (s, 3H), 2.25 (s, 3H).

### Step 4: 1-(4-fluorophenyl)-N-(5-hydroxy-4-methyl-2-pyridyl)-2-oxo-pyridine-3-carboxamide

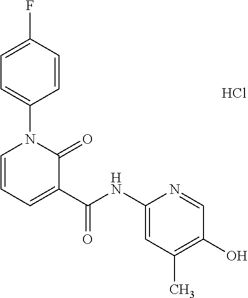 [SMILES: Cc1cc(NC(=O)c2cccn(-c3ccc(F)cc3)c2=O)ncc1O ; Cl]

1-(4-fluorophenyl)-N-[5-(methoxymethoxy)-4-methyl-2-pyridyl]-2-oxo-pyridine-3-carboxamide (Example 25, step 3) (80 mg, 0.209 mmol) was suspended in 1,4-dioxane (1 mL) and 4M hydrochloric acid in dioxane (0.45 mL, 1.82 mmol) was added. The reaction mixture was stirred at room temperature for 1 h and the formed solid was collected by filtration, washed with diethyl ether (3×2 mL) and dried to afford 1-(4-fluorophenyl)-N-(5-hydroxy-4-methyl-2-pyridyl)-2-oxo-pyridine-3-carboxamide hydrochloride (65 mg) as an off-white solid.

LC-MS (Method 2A): 0.98 min; MS m/z=339.9 [M+H]+

1H NMR (500 MHz, DMSO-d6) δ 12.14 (s, 1H), 9.85 (s, 1H), 8.58 (dd, J=7.3, 2.2 Hz, 1H), 8.12 (dd, J=6.6, 2.1 Hz, 1H), 8.06 (s, 1H), 7.80 (s, 1H), 7.67-7.54 (m, 2H), 7.46-7.36 (m, 2H), 6.72 (t, J=6.9 Hz, 1H), 2.21 (s, 3H).

### Step 5: N-[5-[(6-chloro-1,7-naphthyridin-4-yl)oxy]-4-methyl-2-pyridyl]-1-(4-fluorophenyl)-2-oxo-pyridine-3-carboxamide

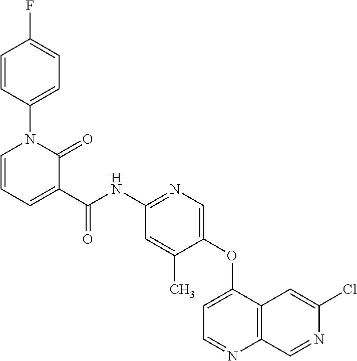 [SMILES: Cc1cc(NC(=O)c2cccn(-c3ccc(F)cc3)c2=O)ncc1Oc1ccnc2cnc(Cl)cc12]

A suspension of 1-(4-fluorophenyl)-N-(5-hydroxy-4-methyl-2-pyridyl)-2-oxo-pyridine-3-carboxamide (Example 25, step 4) (45 mg, 0.133 mmol), 4,6-dichloro-1,7-naphthyridine (40 mg, 0.199 mmol) and potassium hydroxide (15 mg, 0.265 mmol) in IPA (5 mL) was stirred at 115° C. for 5 h. The reaction mixture was cooled to room temperature, water (5 mL) was added and the resulting solid was collected by filtration and washed with MeOH (2×3 mL). The solid was purified by low pH reverse phase chromatography (eluting with a gradient of 10-100% MeCN (0.1% formic acid) in water (0.1% formic acid)) to afford N-[5-[(6-chloro-1,7-naphthyridin-4-yl)oxy]-4-methyl-2-pyridyl]-1-(4-fluorophenyl)-2-oxo-pyridine-3-carboxamide (30 mg) as a brown solid.

LC-MS (Method 2A): 1.29 min; MS m/z=502.2 [M+H]+

¹H NMR (400 MHz, DMSO-d6) δ 12.38 (s, 1H), 9.31 (s, 1H), 8.84 (d, J=5.2 Hz, 1H), 8.63 (dd, J=7.2, 1.8 Hz, 1H), 8.40 (s, 1H), 8.36-8.26 (m, 2H), 8.16 (d, J=4.5 Hz, 1H), 7.62 (m, 2H), 7.48-7.34 (m, 2H), 6.86 (d, J=5.2 Hz, 1H), 6.79-6.70 (m, 1H), 2.22 (s, 3H).

### Step 6: 4-[[6-[[1-(4-fluorophenyl)-2-oxo-pyridine-3-carbonyl]amino]-4-methyl-3-pyridyl]oxy]-N-(1-methyl-4-piperidyl)-1,7-naphthyridine-6-carboxamide formate salt

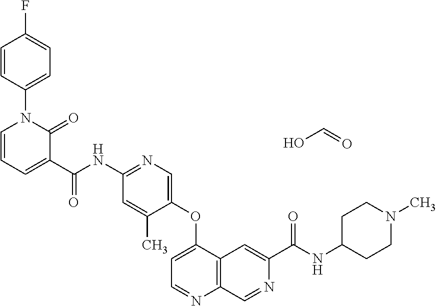 [SMILES: Cc1cc(NC(=O)c2cccn(-c3ccc(F)cc3)c2=O)ncc1Oc1ccnc2cnc(C(=O)NC3CCN(C)CC3)cc12 ; O=CO]

A suspension of N-[5-[(6-chloro-1,7-naphthyridin-4-yl)oxy]-4-methyl-2-pyridyl]-1-(4-fluorophenyl)-2-oxo-pyridine-3-carboxamide (Example 25, step 5) (30 mg, 0.0598 mmol), sodium carbonate (19 mg, 0.179 mmol), XantPhos-Pd-G3 (5.7 mg, 0.00598 mmol) and 1-methylpiperidin-4-amine (20 mg, 0.179 mmol) in DMA (2 mL) was added to Chamber A of a COware apparatus and the vessel was flushed with nitrogen. Methanesulfonyl chloride (0.014 mL, 0.179 mmol) and formic acid (0.0068 mL, 0.179 mmol) in nitrogen flushed anhydrous toluene (2 mL) was added to Chamber B followed by triethylamine (0.050 mL, 0.359 mmol). The reaction mixture was warmed to 100° C. and was stirred for 18 h. The cooled reaction mixture was partitioned between water (10 mL) and DCM (10 mL), the organic fraction separated and the aqueous layer further extracted with DCM (2×5 mL). The combined organic fractions were passed through a hydrophobic frit before being concentrated in vacuo. The crude product was purified by low pH reverse phase chromatography (eluting with a gradient of 10-100% MeCN (0.1% formic acid) in water (0.1% formic acid)) to afford 4-[[6-[[1-(4-fluorophenyl)-2-oxo-pyridine-3-carbonyl]amino]-4-methyl-3-pyridyl]oxy]-N-(1-methyl-4-piperidyl)-1,7-naphthyridine-6-carboxamide formate salt (16.4 mg) as an orange solid.

LC-MS (Method 7A): 1.87 min; MS m/z=608.3 [M+H]+

¹H NMR (400 MHz, DMSO-d6) δ 12.39 (s, 1H), 9.45 (s, 1H), 8.90 (d, J=5.2 Hz, 1H), 8.85 (s, 1H), 8.79 (d, J=8.4 Hz, 1H), 8.63 (dd, J=7.3, 2.2 Hz, 1H), 8.41 (s, 1H), 8.32 (s, 1H), 8.18 (s, 1H), 8.15 (dd, J=6.6, 2.2 Hz, 1H), 7.68-7.56 (m, 2H), 7.51-7.37 (m, 2H), 6.88 (d, J=5.2 Hz, 1H), 6.79-6.69 (m, 1H), 3.92-3.81 (m, 1H), 2.87-2.77 (m, 2H), 2.26-2.19 (m, 6H), 2.13-2.01 (m, 2H), 1.85-1.67 (m, 4H).

### Example 26: 4-[[6-[[1-(4-fluorophenyl)-2-oxo-pyridine-3-carbonyl]amino]-2-methyl-3-pyridyl]oxy]-N-(1-methyl-4-piperidyl)-1,7-naphthyridine-6-carboxamide

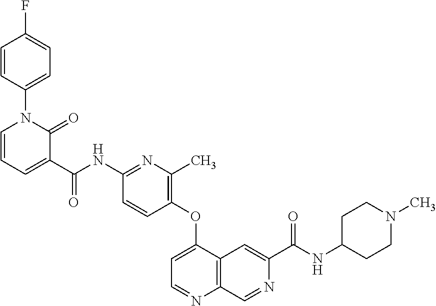 [SMILES: Cc1nc(NC(=O)c2cccn(-c3ccc(F)cc3)c2=O)ccc1Oc1ccnc2cnc(C(=O)NC3CCN(C)CC3)cc12]

### Step 1: 6-chloro-4-[(2-methyl-6-nitro-3-pyridyl)oxy]-1,7-naphthyridine

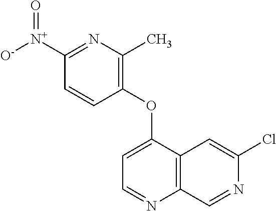 [SMILES: Cc1nc([N+](=O)[O-])ccc1Oc1ccnc2cnc(Cl)cc12]

A solution of 4,6-dichloro-1,7-naphthyridine (0.50 g, 2.51 mmol), 2-methyl-6-nitro-pyridin-3-ol (465 mg, 3.01 mmol) and DIPEA (0.88 mL, 5.02 mmol) in chlorobenzene (10 mL) was heated to 150° C. for 24 h. Water (20 mL) was added to the cooled reaction mixture and the resulting suspension was extracted with DCM (3×20 mL). The combined organic fractions were passed through a hydrophobic frit and concentrated in vacuo to afford 6-chloro-4-[(2-methyl-6-nitro-3-pyridyl)oxy]-1,7-naphthyridine (729 mg) as a brown solid.

LC-MS (Method 2A): 1.13 min; MS m/z=317.0 [M+H]+

¹H NMR (500 MHz, DMSO-d6) δ 9.36 (d, J=0.7 Hz, 1H), 8.94 (d, J=5.1 Hz, 1H), 8.34 (d, J=0.7 Hz, 1H), 8.32 (d, J=8.7 Hz, 1H), 8.07 (d, J=8.7 Hz, 1H), 7.19 (d, J=5.1 Hz, 1H), 2.55 (s, 3H).

### Step 2: 5-[(6-chloro-1,7-naphthyridin-4-yl)oxy]-6-methyl-pyridin-2-amine

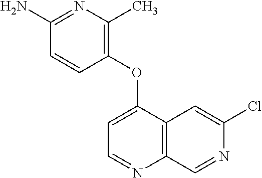 [SMILES: Cc1nc(N)ccc1Oc1ccnc2cnc(Cl)cc12]

A suspension of 6-chloro-4-[(2-methyl-6-nitro-3-pyridyl)oxy]-1,7-naphthyridine (Example 26, step 1)(365 mg, 1.15 mmol), iron powder (322 mg, 5.76 mmol) and ammonium chloride (308 mg, 5.76 mmol) in methanol (8 mL) and water (2 mL) was heated to 75° C. and stirred for 1 h. The reaction mixture was allowed to cool to room temperature before being filtered through a pad of celite. The filter bed was washed with MeOH (20 mL) and CHCl₃/IPA (2:1, 50 mL) and the combined filtrates were concentrated in vacuo. Water (30 mL) was added to the residue and the mixture was extracted with CHCl₃/IPA (2:1, 3×30 mL). The combined organic fractions were passed through a hydrophobic frit and concentrated in vacuo to afford 5-[(6-chloro-1,7-naphthyridin-4-yl)oxy]-6-methyl-pyridin-2-amine (306 mg) as a light brown solid.

LC-MS (Method 3B): 0.87 min; MS m/z=297.0 [M+H]+

¹H NMR (400 MHz, DMSO-d6) δ 9.28 (d, J=0.7 Hz, 1H), 8.82 (d, J=5.1 Hz, 1H), 8.27 (d, J=0.7 Hz, 1H), 7.34 (d, J=8.8 Hz, 1H), 6.77 (d, J=5.1 Hz, 1H), 6.44 (d, J=8.7 Hz, 1H), 6.13 (s, 2H), 2.10 (s, 3H).

### Step 3: 4-[(6-amino-2-methyl-3-pyridyl)oxy]-N-(1-methyl-4-piperidyl)-1,7-naphthyridine-6-carboxamide

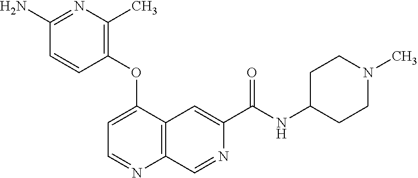 [SMILES: Cc1nc(N)ccc1Oc1ccnc2cnc(C(=O)NC3CCN(C)CC3)cc12]

1-methylpiperidin-4-amine (251 mg, 2.20 mmol), 5-[(6-chloro-1,7-naphthyridin-4-yl)oxy]pyridin-2-amine (Example 26, step 2) (200 mg, 0.733 mmol), XantPhos-Pd-G3 (70 mg, 0.0733 mmol) and sodium carbonate (233 mg, 2.20 mmol) in DMA (10 mL) were added to chamber A of a 100 mL COware apparatus and the suspension was flushed with nitrogen. Formic acid (0.083 mL, 2.20 mmol) and methanesulfonyl chloride (0.17 mL, 2.20 mmol) in nitrogen flushed toluene (5 mL) was added to chamber B. Triethylamine (0.61 mL, 4.40 mmol) was added to Chamber B and the reaction mixture was heated to 100° C. for 18 h. Water (10 mL) was added to the cooled reaction mixture and the resulting suspension was extracted with DCM (3×10 mL). The combined organic fractions were passed through a hydrophobic frit and concentrated in vacuo. The residue was loaded onto a 5 g SCX cartridge which was sequentially washed with MeOH (2×10 mL) and 7M ammonia in MeOH (2×10 mL) and the methanolic ammonia fraction was concentrated in vacuo. The crude product was purified by acidic reverse phase chromatography (eluting with 10-100% MeCN (0.1% formic acid) in water (0.1% formic acid)) to afford 4-[(6-amino-2-methyl-3-pyridyl)oxy]-N-(1-methyl-4-piperidyl)-1,7-naphthyridine-6-carboxamide (81 mg) as a pale yellow solid.

LC-MS (Method 2.5B): 1.26 min; MS m/z=393.3 [M+H]+

¹H NMR (400 MHz, DMSO-d6) δ 9.41 (d, J=0.8 Hz, 1H), 8.88 (d, J=5.2 Hz, 1H), 8.80 (d, J=0.8 Hz, 1H), 8.74 (d, J=8.4 Hz, 1H), 7.33 (d, J=8.7 Hz, 1H), 6.78 (d, J=5.2 Hz, 1H), 6.41 (d, J=8.7 Hz, 1H), 6.05 (s, 2H), 3.83 (dq, J=10.4, 4.9 Hz, 1H), 2.75 (d, J=11.7 Hz, 2H), 2.17 (s, 3H), 2.08 (s, 3H), 2.04-1.93 (m, 2H), 1.83-1.64 (m, 4H).

### Step 4: 4-[[6-[[1-(4-fluorophenyl)-2-oxo-pyridine-3-carbonyl]amino]-2-methyl-3-pyridyl]oxy]-N-(1-methyl-4-piperidyl)-1,7-naphthyridine-6-carboxamide

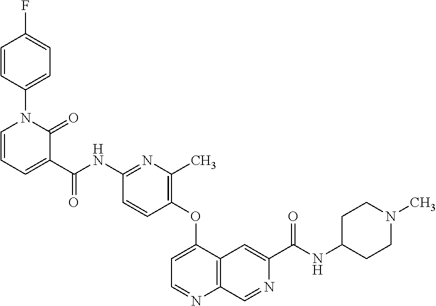 [SMILES: Cc1nc(NC(=O)c2cccn(-c3ccc(F)cc3)c2=O)ccc1Oc1ccnc2cnc(C(=O)NC3CCN(C)CC3)cc12]

HATU (102 mg, 0.268 mmol) was added to a solution of 4-[(6-amino-2-methyl-3-pyridyl)oxy]-N-(1-methyl-4-piperidyl)-1,7-naphthyridine-6-carboxamide (Example 26, step 3) (81 mg, 0.206 mmol), 1-(4-fluorophenyl)-2-oxo-pyridine-3-carboxylic acid (Intermediate B)(48 mg, 0.206 mmol) and DIPEA (0.090 mL, 0.516 mmol) in DMF (4 mL) and the mixture stirred at room temperature for 60 h. Water (5 mL) was added to the reaction mixture and the resultant emulsion was extracted with DCM (3×10 mL). The combined organic fractions were passed through a hydrophobic frit and concentrated in vacuo. The residue was purified by acidic reverse phase chromatography (eluting with 10-100% MeCN (0.1% formic acid) in water (0.1% formic acid)). The product containing fractions were concentrated in vacuo to afford a pale yellow solid which was loaded on to a 5 g SCX column which was washed sequentially with MeOH (2×10 mL) and 7M NH₃ in MeOH (2×10 mL). The methanolic ammonia fraction was concentrated in vacuo to afford 4-[[6-[[1-(4-fluorophenyl)-2-oxo-pyridine-3-carbonyl]amino]-2-methyl-3-pyridyl]oxy]-N-(1-methyl-4-piperidyl)-1,7-naphthyridine-6-carboxamide (47.5 mg) as a pale yellow solid.

LC-MS (Method 7A): 1.90 min; MS m/z=608.3 [M+H]+

¹H NMR (500 MHz, DMSO-d6) δ 12.40 (s, 1H), 9.44 (d, J=0.8 Hz, 1H), 8.90 (d, J=5.2 Hz, 1H), 8.83 (d, J=0.8 Hz, 1H), 8.78 (d, J=8.4 Hz, 1H), 8.63 (dd, J=7.3, 2.2 Hz, 1H), 8.28 (d, J=8.8 Hz, 1H), 8.15 (dd, J=6.6, 2.2 Hz, 1H), 7.84 (d, J=8.8 Hz, 1H), 7.66-7.57 (m, 2H), 7.46-7.38 (m, 2H), 6.88 (d, J=5.2 Hz, 1H), 6.77-6.70 (m, 1H), 3.91-3.78 (m, 1H), 2.83-2.73 (m, 2H), 2.27 (s, 3H), 2.19 (s, 3H), 2.09-1.97 (m, 2H), 1.83-1.66 (m, 4H).

### Example 27: 4-[[6-[[3-(4-fluorophenyl)-1-isopropyl-2,4-dioxo-pyrimidine-5-carbonyl]amino]-3-pyridyl]oxy]-N-(4-piperidyl)-1,7-naphthyridine-6-carboxamide

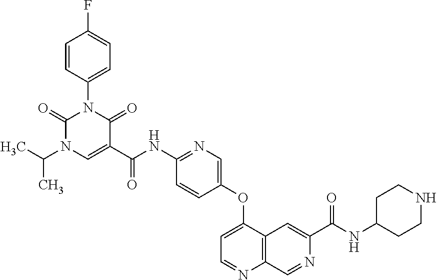 [SMILES: CC(C)n1cc(C(=O)Nc2ccc(Oc3ccnc4cnc(C(=O)NC5CCNCC5)cc34)cn2)c(=O)n(-c2ccc(F)cc2)c1=O]

### Step 1: N-[5-[(6-chloro-1,7-naphthyridin-4-yl)oxy]-2-pyridyl]-3-(4-fluorophenyl)-1-isopropyl-2,4-dioxo-pyrimidine-5-carboxamide

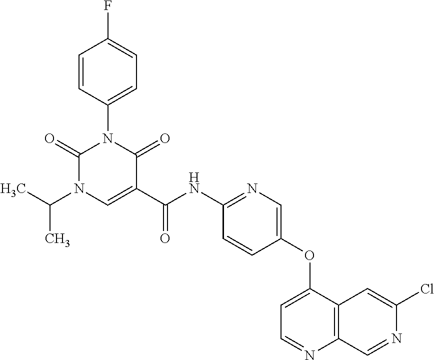 [SMILES: CC(C)n1cc(C(=O)Nc2ccc(Oc3ccnc4cnc(Cl)cc34)cn2)c(=O)n(-c2ccc(F)cc2)c1=O]

A solution of 5-[(6-chloro-1,7-naphthyridin-4-yl)oxy]pyridin-2-amine (Example 10, step 2)(2.30 g, 8.43 mmol), 3-(4-fluorophenyl)-1-isopropyl-2,4-dioxo-pyrimidine-5-carboxylic acid (Intermediate C)(2.24 g, 7.67 mmol), HATU (3.79 g, 9.97 mmol) and DIPEA (3.3 mL, 19.2 mmol) in DMF (20 mL) was stirred at 40° C. for 40 h. Water (20 mL) was added to the reaction mixture and the resultant emulsion was extracted with DCM (3×20 mL). The combined organic fractions were passed through a hydrophobic frit and concentrated in vacuo. The residue was partitioned between EtOAc (50 mL) and water (50 mL) and the organic layer was washed with water (2×50 mL),brine (3×50 mL), dried over Na₂SO₄ and concentrated in vacuo to afford N-[5-[(6-chloro-1,7-naphthyridin-4-yl)oxy]-2-pyridyl]-3-(4-fluorophenyl)-1-isopropyl-2,4-dioxo-pyrimidine-5-carboxamide (1.47 g) as a brown foam.

LC-MS (Method 2A): 1.21 min; MS m/z=547.6 [M+H]+

¹H NMR (500 MHz, DMSO) δ 11.44 (s, 1H), 9.32 (d, J=0.7 Hz, 1H), 8.87 (d, J=5.2 Hz, 1H), 8.74 (s, 1H), 8.43 (d, J=2.9 Hz, 1H), 8.40 (d, J=9.0 Hz, 1H), 8.28 (d, J=0.7 Hz, 1H), 7.95 (dd, J=9.0, 2.9 Hz, 1H), 7.47-7.41 (m, 2H), 7.40-7.34 (m, 2H), 7.01 (d, J=5.1 Hz, 1H), 4.79 (hept, J=6.8 Hz, 1H), 1.44 (d, J=6.8 Hz, 6H).

### Step 2: tert-butyl 4-[[4-[[6-[[3-(4-fluorophenyl)-1-isopropyl-2,4-dioxo-pyrimidine-5-carbonyl]amino]-3-pyridyl]oxy]-1,7-naphthyridine-6-carbonyl]amino]piperidine-1-carboxylate (Example 27.1)

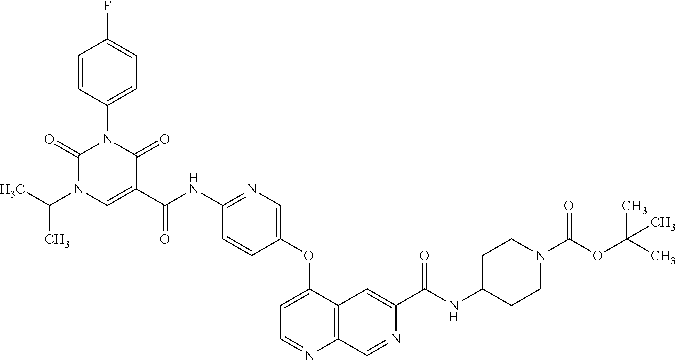 [SMILES: CC(C)n1cc(C(=O)Nc2ccc(Oc3ccnc4cnc(C(=O)NC5CCN(C(=O)OC(C)(C)C)CC5)cc34)cn2)c(=O)n(-c2ccc(F)cc2)c1=O]

tert-butyl 4-aminopiperidine-1-carboxylate (88 mg, 0.439 mmol), N-[5-[(6-chloro-1,7-naphthyridin-4-yl)oxy]-2-pyridyl]-3-(4-fluorophenyl)-1-isopropyl-2,4-dioxo-pyrimidine-5-carboxamide (Example 27, step 1)(100 mg, 0.146 mmol), XantPhos-Pd-G3 (14 mg, 0.0146 mmol) and sodium carbonate (47 mg, 0.439 mmol) in DMA (5 mL) were added to chamber A of a 100 mL COware apparatus and the suspension was flushed with nitrogen. Methanesulfonyl chloride (68 uL, 0.878 mmol) and formic acid (33 uL, 0.878 mmol) in nitrogen flushed toluene (5 mL) were added to chamber B before triethylamine (245 uL, 1.76 mmol) was added to Chamber B and the mixture was heated to 100° C. for 18 h. The mixture was cooled to room temperature, filtered and the collected solid washed through with DCM (2×5 mL). The filtrate was concentrated in vacuo and purified by high pH preparative HPLC (early elute method) to afford tert-butyl 4-[[4-[[6-[[3-(4-fluorophenyl)-1-isopropyl-2,4-dioxo-pyrimidine-5-carbonyl]amino]-3-pyridyl]oxy]-1,7-naphthyridine-6-carbonyl]amino]piperidine-1-carboxylate (Example 27.1-22.9 mg) as a white solid.

LC-MS (Method 7A): 4.00 min; MS m/z=739.3 [M+H]+

¹H NMR (400 MHz, DMSO) δ 11.44 (s, 1H), 9.45 (d, J=0.8 Hz, 1H), 8.93 (d, J=5.2 Hz, 1H), 8.89 (d, J=8.5 Hz, 1H), 8.81 (d, J=0.8 Hz, 1H), 8.73 (s, 1H), 8.45 (d, J=3.1 Hz, 1H), 8.41 (d, J=9.0 Hz, 1H), 7.97 (dd, J=9.0, 2.9 Hz, 1H), 7.48-7.41 (m, 2H), 7.40-7.32 (m, 2H), 7.04 (d, J=5.2 Hz, 1H), 4.83-4.74 (m, 1H), 4.14-4.02 (m, 1H), 4.02-3.89 (m, 2H), 3.00-2.74 (m, 2H), 1.85-1.75 (m, 2H), 1.67-1.53 (m, 2H), 1.44 (d, J=6.8 Hz, 6H), 1.42 (s, 9H).

### Step 3: 4-[[6-[[3-(4-fluorophenyl)-1-isopropyl-2,4-dioxo-pyrimidine-5-carbonyl]amino]-3-pyridyl]oxy]-N-(4-piperidyl)-1,7-naphthyridine-6-carboxamide

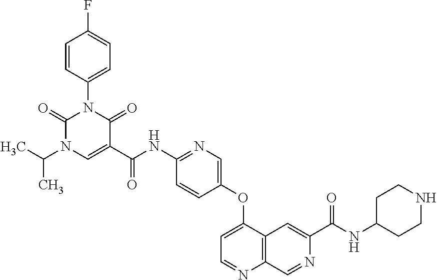 [SMILES: CC(C)n1cc(C(=O)Nc2ccc(Oc3ccnc4cnc(C(=O)NC5CCNCC5)cc34)cn2)c(=O)n(-c2ccc(F)cc2)c1=O]

tert-butyl 4-[[4-[[6-[[3-(4-fluorophenyl)-1-isopropyl-2,4-dioxo-pyrimidine-5-carbonyl]amino]-3-pyridyl]oxy]-1,7-naphthyridine-6-carbonyl]amino]piperidine-1-carboxylate (Example 27, step 2)(20 mg, 0.0271 mmol) was suspended in 1,4-dioxane (3 mL) and 4M hydrochloric acid in 1,4-dioxane (0.80 mL, 3.20 mmol) was added. The reaction mixture was stirred at room temperature for 5 h. The mixture was concentrated in vacuo and the residue purified by low PH reverse phase chromatography (eluting with a gradient of 10-100% MeCN (0.1% formic acid) in water (0.1% formic acid)) to afford 4-[[6-[[3-(4-fluorophenyl)-1-isopropyl-2,4-dioxo-pyrimidine-5-carbonyl]amino]-3-pyridyl]oxy]-N-(4-piperidyl)-1,7-naphthyridine-6-carboxamide (8.2 mg) as a white solid.

LC-MS (Method 7A): 2.04 min; MS m/z=639.3 [M+H]+

¹H NMR (500 MHz, DMSO) δ 11.45 (s, 1H), 9.50-9.42 (m, 1H), 9.00-8.90 (m, 2H), 8.85-8.80 (m, 1H), 8.74 (s, 1H), 8.46 (d, J=2.9 Hz, 1H), 8.42 (d, J=9.0 Hz, 1H), 8.31 (s, 1H), 7.98 (dd, J=9.0, 2.9 Hz, 1H), 7.47-7.41 (m, 2H), 7.41-7.34 (m, 2H), 7.06 (d, J=5.2 Hz, 1H), 4.86-4.71 (m, 1H), 4.12-4.01 (m, 1H), 3.16 (d, J=12.6 Hz, 2H), 2.86-2.75 (m, 2H), 1.96-1.85 (m, 2H), 1.83-1.65 (m, 2H), 1.44 (d, J=6.8 Hz, 6H).

The compounds of the following tabulated examples (Table 16) were prepared analogously to Example 27, step 2.

TABLE 16

| Ex. | Structure and Name | Retention Time, [M + H]+, 1H NMR |
|---|---|---|
| 27.2 | 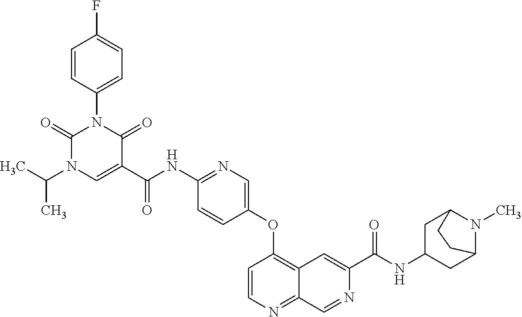 [SMILES: CC(C)n1cc(C(=O)Nc2ccc(Oc3ccnc4cnc(C(=O)NC5CC6CCC(C5)N6C)cc34)cn2)c(=O)n(-c2ccc(F)cc2)c1=O] | LC-MS (Method 7A): 2.13 min; MS m/z = 679.3 [M + H]+ ¹H NMR (400 MHz, DMSO) δ 11.44 (s, 1H), 9.48 (d, J = 0.8 Hz, 1H), 8.93 (d, J = 5.2 Hz, 1H), 8.79 (d, J = 0.8 Hz, 1H), 8.73 (s, 1H), 8.69 (d, J = 7.7 Hz, 1H), 8.45 (d, J = 2.9 Hz, 1H), 8.40 (d, J = 9.1 Hz, 1H), 7.96 (dd, J = 9.0, 2.9 Hz, 1H), 7.48-7.40 (m, 2H), 7.40-7.31 (m, 2H), 7.05 (d, J = 5.2 Hz, 1H), 4.85- 4.72 (m, 1H), 4.18-4.06 (m, 1H), 3.11 (s, 2H), 2.20 (s, 3H), 2.17-2.03 (m, 4H), 1.86 (t, J = 7.2 Hz, 2H), 1.73 (d, J = 14.0 Hz, 2H), 1.44 (d, J = 6.8 Hz, 6H). |
| | 4-[[6-[[3-(4-fluorophenyl)-1-isopropyl-2,4-dioxo- | |
| | pyrimidine-5-carbonyl]amino]-3-pyridyl]oxy]-N- | |
| | (8-methyl-8-azabicyclo[3.2.1]octan- | |
| | 3-yl)-1,7-naphthyridine-6-carboxamide | |
| 27.3 | 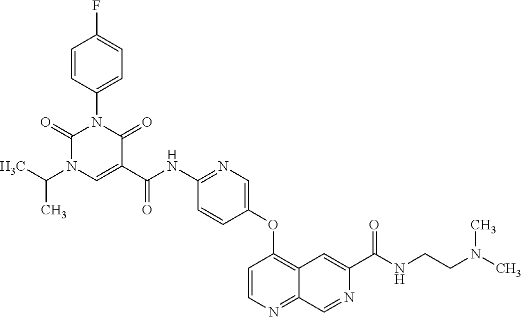 [SMILES: CC(C)n1cc(C(=O)Nc2ccc(Oc3ccnc4cnc(C(=O)NCCN(C)C)cc34)cn2)c(=O)n(-c2ccc(F)cc2)c1=O] | LC-MS (Method 7A): 2.03 min; MS m/z = 627.3 [M + H]+ ¹H NMR (400 MHz, DMSO) δ 11.43 (s, 1H), 9.45 (d, J = 0.8 Hz, 1H), 8.93 (d, J = 5.2 Hz, 1H), 8.88-8.82 (m, 1H), 8.80 (d, J = 0.7 Hz, 1H), 8.73 (s, 1H), 8.45 (d, J = 2.9 Hz, 1H), 8.40 (d, J = 9.0 Hz, 1H), 7.97 (dd, J = 9.0, 2.9 Hz, 1H), 7.49-7.40 (m, 2H), 7.40-7.30 (m, 2H), 7.04 (d, J = 5.2 Hz, 1H), 4.87-4.72 (m, 1H), 3.54-3.43 (m, 2H), 2.56-2.42 (m, 2H), 2.22 (s, 6H), 1.44 (d, J = 6.8 Hz, 6H) |
| | N-[2-(dimethylamino)ethyl]-4-[[6-[[3-(4-fluorophenyl)-1- | |
| | isopropyl-2,4-dioxo-pyrimidine-5-carbonyl]amino]- | |
| | 3-pyridyl]oxy]-1,7-naphthyridine-6-carboxamide | |
| 27.4 | 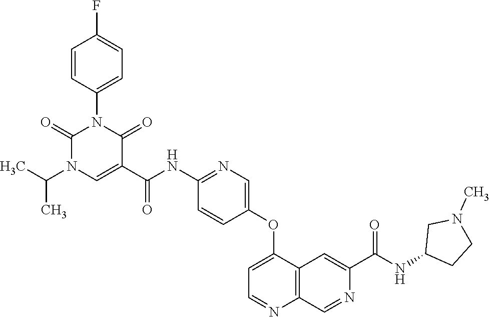 [SMILES: CC(C)n1cc(C(=O)Nc2ccc(Oc3ccnc4cnc(C(=O)N[C@H]5CCN(C)C5)cc34)cn2)c(=O)n(-c2ccc(F)cc2)c1=O] | LC-MS (Method 7A): 2.14 min; MS m/z = 639.3 [M + H]+ ¹H NMR (400 MHz, DMSO) δ 11.44 (s, 1H), 9.47-9.43 (m, 1H), 8.93 (d, J = 5.2 Hz, 1H), 8.83-8.75 (m, 2H), 8.73 (s, 1H), 8.45 (d, J = 2.9 Hz, 1H), 8.40 (d, J = 9.0 Hz, 1H), 7.97 (dd, J = 9.0, 2.9 Hz, 1H), 7.48-7.40 (m, 2H), 7.40- 7.33 (m, 2H), 7.05 (d, J = 5.2 Hz, 1H), 4.85-4.72 (m, 1H), 4.56-4.45 (m, 1H), 2.78-2.65 (m, 2H), 2.60-2.54 (m, 1H), 2.43-2.35 (m, 1H), 2.30 (s, 3H), 2.27-2.17 (m, 1H), 1.89-1.77 (m, 1H), 1.44 (d, J = 6.8 Hz, 6H). |
| | 4-[[6-[[3-(4-fluorophenyl)-1-isopropyl-2,4-dioxo- | |
| | pyrimidine-5-carbonyl]amino]-3-pyridyl]oxy]-N- | |
| | [(3S)-1-methylpyrrolidin-3-yl]-1,7-naphthyridine-6-carboxamide | |
| 27.5 | 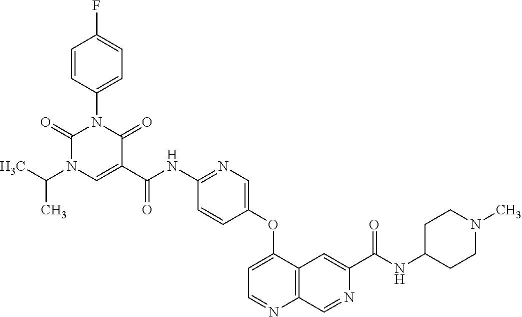 [SMILES: CC(C)n1cc(C(=O)Nc2ccc(Oc3ccnc4cnc(C(=O)NC5CCN(C)CC5)cc34)cn2)c(=O)n(-c2ccc(F)cc2)c1=O] | LC-MS (Method 7A): 2.10 min; MS m/z = 653.3 [M + H]+ ¹H NMR (500 MHz, DMSO-d₆) δ 11.46 (s, 1H), 9.48 (s, 1H), 9.19-9.06 (m, 1H), 8.95 (d, J = 5.2 Hz, 1H), 8.85- 8.79 (m, 1H), 8.77-8.70 (m, 1H), 8.48- 8.44 (m, 1H), 8.44-8.39 (m, 1H), 7.98 (dd, J = 9.0, 2.9 Hz, 1H), 7.47- 7.41 (m, 2H), 7.41-7.34 (m, 2H), 7.07 (d, J = 5.2 Hz, 1H), 4.85-4.74 (m, 1H), 4.22-4.06 (m, 1H), 3.54-3.37 (m, 2H), 3.19-3.00 (m, 2H), 2.84-2.69 (m, 3H), 2.09-1.87 (m, 4H), 1.45 (d, J = 6.8 Hz, 6H). |
| | 4-[[6-[[3-(4-fluorophenyl)-1-isopropyl-2,4- | |
| | dioxo-pyrimidine-5-carbonyl]amino]-3-pyridyl]oxy]-N- | |
| | (1-methyl-4-piperidyl)-1,7-naphthyridine-6-carboxamide | |
| 27.6 | 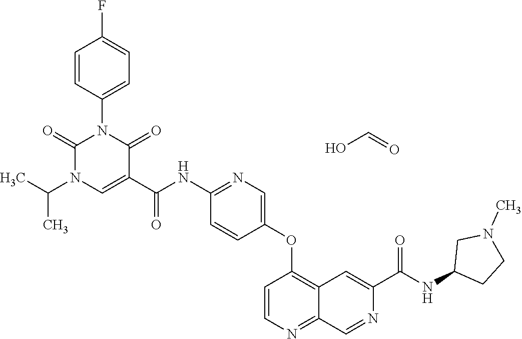 [SMILES: CC(C)n1cc(C(=O)Nc2ccc(Oc3ccnc4cnc(C(=O)N[C@@H]5CCN(C)C5)cc34)cn2)c(=O)n(-c2ccc(F)cc2)c1=O ; O=CO] | LC-MS (Method 7A): 2.06 min; MS m/z = 639.3 [M + H]+ ¹H NMR (500 MHz, DMSO) δ 11.44 (s, 1H), 9.45 (d, J = 0.8 Hz, 1H), 8.93 (d, J = 5.2 Hz, 1H), 8.89 (d, J = 8.0 Hz, 1H), 8.79 (d, J = 0.8 Hz, 1H), 8.73 (s, 1H), 8.48-8.43 (m, 1H), 8.40 (d, J = 9.0 Hz, 1H), 7.97 (dd, J = 9.0, 2.9 Hz, 1H), 7.47- 7.40 (m, 2H), 7.40-7.32 (m, 2H), 7.05 (d, J = 5.2 Hz, 1H), 4.78 (hept, J = 6.7 Hz, 1H), 4.61-4.50 (m, 1H), 2.90-2.78 (m, 2H), 2.75-2.65 (m, 1H), 2.56-2.51 (m, 1H), 2.39 (s, 3H), 2.33- 2.20 (m, 1H), 1.92-1.82 (m, 1H), 1.44 (d, J = 6.8 Hz, 6H). |
| | 4-[[6-[[3-(4-fluorophenyl)-1-isopropyl-2,4-dioxo- | |
| | pyrimidine-5-carbonyl]amino]-3-pyridyl]oxy]-N- | |
| | [(3R)-1-methylpyrrolidin-3-yl]-1,7- | |
| | naphthyridine-6-carboxamide formate salt | |
| 27.7 | 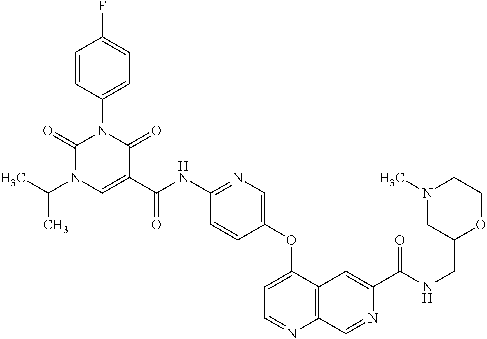 [SMILES: CC(C)n1cc(C(=O)Nc2ccc(Oc3ccnc4cnc(C(=O)NCC5CN(C)CCO5)cc34)cn2)c(=O)n(-c2ccc(F)cc2)c1=O] | LC-MS (Method 7A): 2.10 min; MS m/z = 669.3 [M + H]+ ¹H NMR (400 MHz, DMSO) δ 11.44 (s, 1H), 9.46 (d, J = 0.8 Hz, 1H), 8.97- 8.90 (m, 2H), 8.81 (d, J = 0.8 Hz, 1H), 8.73 (s, 1H), 8.48-8.44 (m, 1H), 8.43- 8.38 (m, 1H), 7.97 (dd, J = 9.0, 2.9 Hz, 1H), 7.47-7.41 (m, 2H), 7.40- 7.33 (m, 2H), 7.05 (d, J = 5.2 Hz, 1H), 4.79 (hept, J = 6.5 Hz, 1H), 3.88-3.80 (m, 1H), 3.73-3.65 (m, 1H), 3.55- 3.49 (m, 1H), 3.47-3.42 (m, 2H), 2.83- 2.71 (m, 1H), 2.65-2.59 (m, 1H), 2.20 (s, 3H), 2.11-1.96 (m, 1H), 1.94- 1.74 (m, 1H), 1.44 (d, J = 6.8 Hz, 6H). |
| | 4-[[6-[[3-(4-fluorophenyl)-1-isopropyl-2,4-dioxo- | |
| | pyrimidine-5-carbonyl]amino]-3-pyridyl]oxy]-N- | |
| | [(4-methylmorpholin-2-yl)methyl]-1,7-naphthyridine-6-carboxamide | |
| 27.8 | 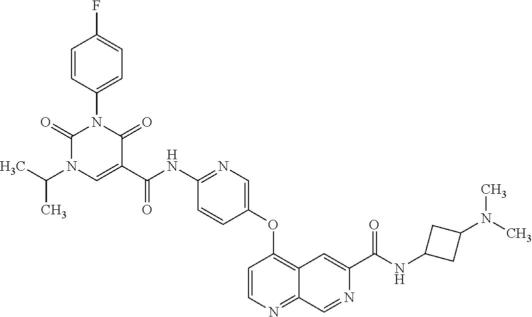 [SMILES: CC(C)n1cc(C(=O)Nc2ccc(Oc3ccnc4cnc(C(=O)NC5CC(N(C)C)C5)cc34)cn2)c(=O)n(-c2ccc(F)cc2)c1=O] | LC-MS (Method 7A): 2.09 min; MS m/z = 653.3 [M + H]+ ¹H NMR (400 MHz, DMSO) δ 11.45 (s, 1H), 9.48-9.43 (m, 1H), 9.12 (d, J = 8.1 Hz, 1H), 8.94 (d, J = 5.2 Hz, 1H), 8.82-8.76 (m, 1H), 8.74 (s, 1H), 8.46 (d, J = 2.9 Hz, 1H), 8.41 (d, J = 9.0 Hz, 1H), 7.97 (dd, J = 9.0, 2.9 Hz, 1H), 7.48-7.40 (m, 2H), 7.41-7.32 (m, 2H), 7.05 (d, J = 5.2 Hz, 1H), 4.85-4.74 (m, 1H), 4.30-4.16 (m, 1H), 2.73-2.61 (m, 1H), 2.48-2.41 (m, 2H), 2.22 (s, 6H), 2.17-2.05 (m, 2H), 1.45 (d, J = 6.8 Hz, 6H). |
| | N-[3-(dimethylamino)cyclobutyl]-4-[[6-[[3-(4-fluorophenyl)- | |
| | 1-isopropyl-2,4-dioxo-pyrimidine-5- | |
| | carbonyl]amino]-3-pyridyl]oxy]-1,7-naphthyridine-6-carboxamide | |
| 27.9 | 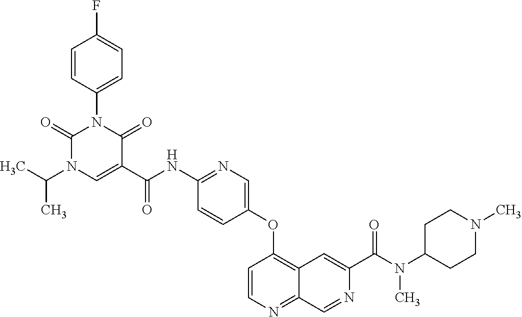 [SMILES: CC(C)n1cc(C(=O)Nc2ccc(Oc3ccnc4cnc(C(=O)N(C)C5CCN(C)CC5)cc34)cn2)c(=O)n(-c2ccc(F)cc2)c1=O] | LC-MS (Method 7A): 1.97 min; MS m/z = 667.3 [M + H]+ ¹H NMR (400 MHz, DMSO) δ 11.31 (s, 1H), 9.41 (d, J = 0.9 Hz, 1H), 8.91 (d, J = 5.2 Hz, 1H), 8.69 (s, 1H), 8.42-8.34 (m, 2H), 8.28 (d, J = 0.9 Hz, 1H), 7.89 (dd, J = 9.0, 2.9 Hz, 1H), 7.46-7.38 (m, 2H), 7.38-7.28 (m, 2H), 7.03 (d, J = 5.1 Hz, 1H), 4.80 (h, J = 6.8 Hz, 1H), 2.97-2.78 (m, 5H), 2.20 (s, 3H), 2.09-1.81 (m, 4H), 1.78-1.66 (m, 2H), 1.47 (d, J = 6.8 Hz, 6H). |
| | 4-[[6-[[3-(4-fluorophenyl)-1-isopropyl-2,4-dioxo- | |
| | pyrimidine-5-carbonyl]amino]-3-pyridyl]oxy]-N- | |
| | methyl-N-(1-methyl-4-piperidyl)-1,7-naphthyridine-6-carboxamide | |

### Example 28: 4-[[6-[[5-(4-fluorophenyl)-1-isopropyl-4-oxo-pyridine-3-carbonyl]amino]-3-pyridyl]oxy]-N-(1-methyl-4-piperidyl)-1,7-naphthyridine-6-carboxamide

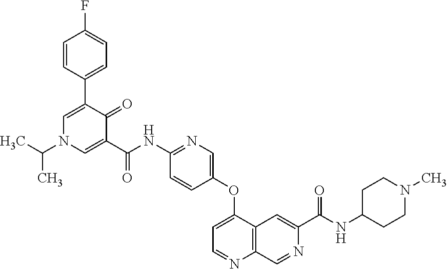 [SMILES: CC(C)n1cc(C(=O)Nc2ccc(Oc3ccnc4cnc(C(=O)NC5CCN(C)CC5)cc34)cn2)c(=O)c(-c2ccc(F)cc2)c1]

### Step 1: 4-[(6-amino-3-pyridyl)oxy]-N-(1-methyl-4-piperidyl)-1,7-naphthyridine-6-carboxamide

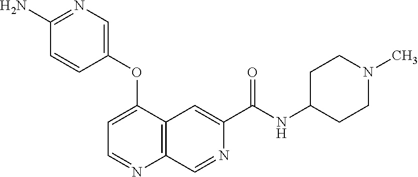 [SMILES: CN1CCC(NC(=O)c2cc3c(Oc4ccc(N)nc4)ccnc3cn2)CC1]

1-Methylpiperidin-4-amine (1.26 g, 11.0 mmol), 5-[(6-chloro-1,7-naphthyridin-4-yl)oxy]pyridin-2-amine (Example 10, step 2)(1.00 g, 3.67 mmol), xantPhos-Pd-G3 (348 mg, 0.367 mmol) and sodium carbonate (1166 mg, 11.0 mmol) in dry DMA (50 mL) were added to chamber A of a 100 mL COware apparatus and the suspension was flushed with nitrogen. Formic acid (0.42 mL, 11.0 mmol) and methanesulfonyl chloride (0.85 mL, 11.0 mmol) in nitrogen flushed dry toluene (25 mL) was added to chamber B before Triethylamine (0.61 mL, 4.40 mmol) was added to Chamber B and the reaction mixture was heated to 100° C. for 16 h. Water (100 mL) and EtOAc (100 mL) were added to the cooled mixture and the resultant suspension was filtered. The organic fraction was separated and the aqueous was further extracted with EtOAc (2×100 mL) and CHCl₃/IPA (2:1, 3×100 mL). The combined organic fractions were passed through a hydrophobic frit and concentrated in vacuo. The residue was loaded onto a 20 g SCX cartridge and the column was washed sequentially with MeOH (100 mL) and 7M NH₃ in MeOH (100 mL). The methanolic ammonia fraction was concentrated in vacuo. The crude product was purified by acidic reverse phase chromatography (eluting with 0-100% MeCN (0.1% formic acid) in water (0.1% formic acid)) to afford 4-[(6-amino-3-pyridyl)oxy]-N-(1-methyl-4-piperidyl)-1,7-naphthyridine-6-carboxamide formate salt (545 mg) as a pale yellow solid.

LC-MS (Method 11B): 0.49 min; MS m/z=379.3 [M+H]+

¹H NMR (400 MHz, DMSO) δ 9.41 (d, J=0.8 Hz, 1H), 8.90 (d, J=5.2 Hz, 1H), 8.86-8.73 (m, 2H), 8.22 (s, 2H), 7.97 (d, J=2.8 Hz, 1H), 7.45 (dd, J=8.9, 3.0 Hz, 1H), 6.91 (d, J=5.2 Hz, 1H), 6.60 (d, J=8.9 Hz, 1H), 6.14 (s, 2H), 4.00-3.85 (m, 1H), 2.98-2.86 (m, 2H), 2.35-2.20 (m, 5H), 1.88-1.76 (m, 4H).

### Step 2: 4-[[6-[[5-(4-fluorophenyl)-1-isopropyl-4-oxo-pyridine-3-carbonyl]amino]-3-pyridyl]oxy]-N-(1-methyl-4-piperidyl)-1,7-naphthyridine-6-carboxamide

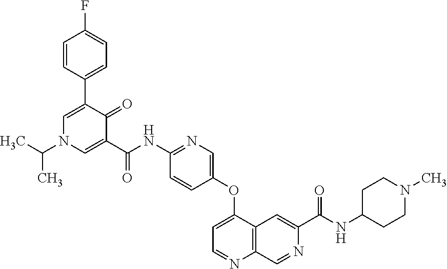 [SMILES: CC(C)n1cc(C(=O)Nc2ccc(Oc3ccnc4cnc(C(=O)NC5CCN(C)CC5)cc34)cn2)c(=O)c(-c2ccc(F)cc2)c1]

HATU (85 mg, 0.224 mmol) was added to a solution of 4-[(6-amino-3-pyridyl)oxy]-N-(1-methyl-4-piperidyl)-1,7-naphthyridine-6-carboxamide formate salt (Example 28, step 1)(73 mg, 0.172 mmol), 5-(4-fluorophenyl)-1-isopropyl-4-oxo-pyridine-3-carboxylic acid (Intermediate H)(47 mg, 0.172 mmol) and DIPEA (0.10 mL, 0.602 mmol) in DMF (2 mL) and the solution was heated to 40° C. for 19 h. The reaction mixture was cooled to room temperature and partitioned between water (5 mL) and DCM (5 mL), the organic fraction separated and the aqueous phase further extracted with DCM (2×10 mL). The combined organic fractions were passed through a hydrophobic frit and concentrated in vacuo. The residue was purified by high pH reverse phase chromatography (eluting with 10-100% MeCN (0.1% NH₄OH) in water (0.1% NH₄OH) to afford 4-[[6-[[5-(4-fluorophenyl)-1-isopropyl-4-oxo-pyridine-3-carbonyl]amino]-3-pyridyl]oxy]-N-(1-methyl-4-piperidyl)-1,7-naphthyridine-6-carboxamide (5 mg) as an off-white solid.

LC-MS (Method 7A): 2.15 min; MS m/z=636.4 [M+H]+

1H NMR (400 MHz, MeOD) δ 9.42-9.35 (m, 1H), 8.98-8.91 (m, 1H), 8.87 (d, J=5.3 Hz, 1H), 8.80 (d, J=2.4 Hz, 1H), 8.50 (d, J=9.0 Hz, 1H), 8.36 (d, J=2.8 Hz, 1H), 8.08 (d, J=2.4 Hz, 1H), 7.82 (dd, J=9.0, 2.9 Hz, 1H), 7.70-7.61 (m, 2H), 7.22-7.13 (m, 2H), 6.98 (d, J=5.3 Hz, 1H), 4.62-4.49 (m, 1H), 4.10-3.97 (m, 1H), 3.10-2.96 (m, 2H), 2.51-2.37 (m, 5H), 2.13-2.01 (m, 2H), 1.91-1.76 (m, 2H), 1.60 (d, J=6.7 Hz, 6H).

The compounds of the following tabulated example (Table 17) was prepared analogously to Example 28, step 2 with 4-[(6-amino-3-pyridyl)oxy]-N-(1-methyl-4-piperidyl)-1,7-naphthyridine-6-carboxamide formate salt (Example 28, step 1) and the appropriate carboxylic acids.

TABLE 17

| Ex. | Structure and Name | Retention Time, [M + H]+, 1H NMR |
|---|---|---|
| 28.1 | 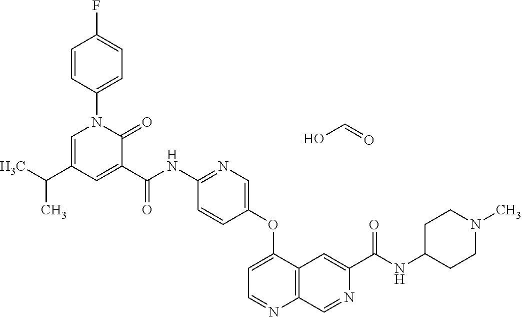 [SMILES: CC(C)c1cc(C(=O)Nc2ccc(Oc3ccnc4cnc(C(=O)NC5CCN(C)CC5)cc34)cn2)c(=O)n(-c2ccc(F)cc2)c1 ; O=CO] | LC-MS (Method 7A): 2.36 min; MS m/z = 636.3 [M + H]+ ¹H NMR (500 MHz, DMSO) δ 12.57 (s, 1H), 9.48-9.40 (m, 1H), 8.92 (d, J = 5.2 Hz, 1H), 8.82-8.74 (m, 2H), 8.59 (d, J = 2.7 Hz, 1H), 8.50-8.42 (m, 2H), 8.21 (s, 1H), 8.01-7.93 (m, 2H), 7.67- 7.58 (m, 2H), 7.47-7.37 (m, 2H), 7.04 (d, J = 5.2 Hz, 1H), 3.91-3.81 (m, 1H), 2.92 (hept, J = 6.9 Hz, 1H), 2.85- 2.74 (m, 2H), 2.21 (s, 3H), 2.13-2.02 (m, 2H), 1.84-1.69 (m, 4H), 1.23 (d, J = 6.9 Hz, 6H). |
| | 4-[[6-[[1-(4-fluorophenyl)-5-isopropyl-2-oxo-pyridine-3- | |
| | carbonyl]amino]-3-pyridyl]oxy]-N-(1-methyl-4-piperidyl)-1,7- | |
| | naphthyridine-6-carboxamide formate salt | |
| 28.2 | 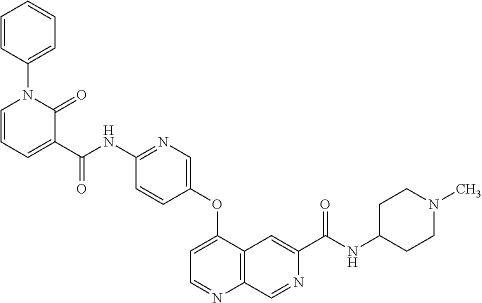 [SMILES: CN1CCC(NC(=O)c2cc3c(Oc4ccc(NC(=O)c5cccn(-c6ccccc6)c5=O)nc4)ccnc3cn2)CC1] | LC-MS (Method 7A): 1.87 min; MS m/z = 576.3 [M + H]+ ¹H NMR (500 MHz, DMSO-d6) δ 12.49 (s, 1H), 9.44 (d, J = 0.8 Hz, 1H), 8.92 (d, J = 5.2 Hz, 1H), 8.80 (d, J = 0.8 Hz, 1H), 8.76 (d, J = 8.4 Hz, 1H), 8.64 (dd, J = 7.3, 2.2 Hz, 1H), 8.49- 8.42 (m, 2H), 8.16 (dd, J = 6.6, 2.2 Hz, 1H), 7.96 (dd, J = 9.0, 2.9 Hz, 1H), 7.63-7.50 (m, 5H), 7.04 (d, J = 5.2 Hz, 1H), 6.77- 6.70 (m, 1H), 3.90-3.77 (m, 1H), 2.81-2.68 (m, 2H), 2.17 (s, 3H), 2.05- 1.93 (m, 2H), 1.83-1.65 (m, 4H). |
| | N-(1-methyl-4-piperidyl)-4-[[6-[(2-oxo-1-phenyl-pyridine-3- | |
| | carbonyl)amino]-3-pyridyl]oxy]-1,7-naphthyridine-6-carboxamide | |

### Example 29: 4-[[6-[[5-(5-fluoro-2-pyridyl)-1-isopropyl-4-oxo-pyridine-3-carbonyl]amino]-3-pyridyl]oxy]-N-(1-methyl-4-piperidyl)-1,7-naphthyridine-6-carboxamide

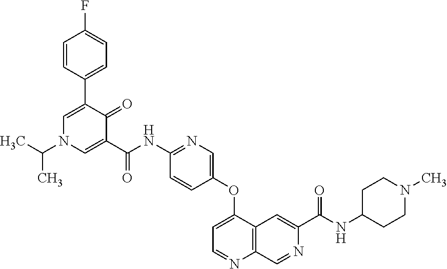 [SMILES: CC(C)n1cc(C(=O)Nc2ccc(Oc3ccnc4cnc(C(=O)NC5CCN(C)CC5)cc34)cn2)c(=O)c(-c2ccc(F)cc2)c1]

### Step 1: 4-[[6-[[5-(5-fluoro-2-pyridyl)-1-isopropyl-4-oxo-pyridine-3-carbonyl]amino]-3-pyridyl]oxy]-N-(1-methyl-4-piperidyl)-1,7-naphthyridine-6-carboxamide

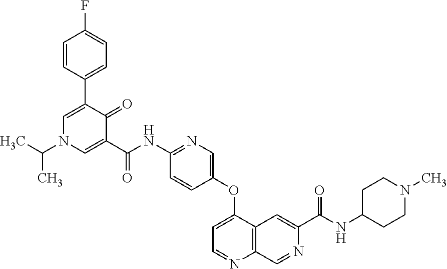 [SMILES: CC(C)n1cc(C(=O)Nc2ccc(Oc3ccnc4cnc(C(=O)NC5CCN(C)CC5)cc34)cn2)c(=O)c(-c2ccc(F)cc2)c1]

5-(5-fluoro-2-pyridyl)-1-isopropyl-4-oxo-pyridine-3-carboxylic acid, sodium salt (Intermediate M)(60 mg, 0.217 mmol) was freebased by dissolving in MeOH (20 mL). 4 M HCl in dioxane (5 mL) was added and the solution was stirred for 5 min at room temperature. The solution was then concentrated in vacuo to afford a white solid. The solid was purified by normal phase column chromatography (eluted with 0-100% EtOAc in heptane followed by 0-100% MeOH in EtOAc to afford the free base as a white solid. The free based acid and HATU (90 mg, 0.236 mmol) were dissolved in anhydrous DMF (1 mL) and DIPEA (0.10 mL, 0.589 mmol) was added. The mixture was stirred at room temperature for 5 min before 4-[(6-amino-3-pyridyl)oxy]-N-(1-methyl-4-piperidyl)-1,7-naphthyridine-6-carboxamide (Example 28, step 1)(50 mg, 0.118 mmol) in DMF (1 mL) was added and the reaction mixture was stirred at room temperature for 3 d. The reaction mixture was partitioned between water (5 mL) and DCM (5 mL), the aqueous was extracted with DCM (2×10 mL) and the combined organics were passed through a hydrophobic frit and concentrated in vacuo. The residue was purified by high pH preparative HPLC (standard method), and product containing fractions were combined and concentrated in vacuo to afford an off-white solid, (16.6 mg).

LC-MS (Method 7A): 1.98 min; MS m/z=637.3 [M+H]+

¹H NMR (500 MHz, DMSO) δ 13.42 (s, 1H), 9.48-9.39 (m, 1H), 8.93 (d, J=5.2 Hz, 1H), 8.84 (d, J=2.4 Hz, 1H), 8.82-8.79 (m, 1H), 8.77 (d, J=8.4 Hz, 1H), 8.71 (d, J=2.4 Hz, 1H), 8.68 (d, J=3.0 Hz, 1H), 8.62-8.56 (m, 1H), 8.50-8.44 (m, 2H), 8.02-7.92 (m, 1H), 7.87-7.78 (m, 1H), 7.05 (d, J=5.2 Hz, 1H), 4.74 (hept, J=6.7 Hz, 1H), 3.92-3.77 (m, 1H), 2.83-2.71 (m, 2H), 2.18 (s, 3H), 2.08-1.96 (m, 2H), 1.84-1.67 (m, 4H), 1.53 (d, J=6.7 Hz, 6H).

### Example 30: N-[3-methyl-4-[(6-piperazin-1-yl-1,7-naphthyridin-4-yl)oxy]phenyl]-2-oxo-1-phenyl-pyridine-3-carboxamide

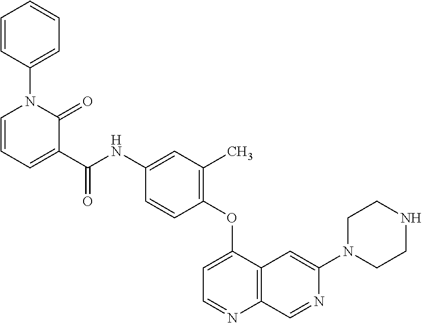 [SMILES: Cc1cc(NC(=O)c2cccn(-c3ccccc3)c2=O)ccc1Oc1ccnc2cnc(N3CCNCC3)cc12]

### Step 1: 6-Chloro-4-(2-methyl-4-nitro-phenoxy)-1,7-naphthyridine

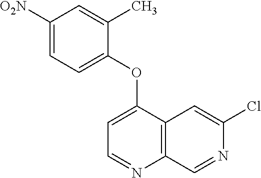 [SMILES: Cc1cc([N+](=O)[O-])ccc1Oc1ccnc2cnc(Cl)cc12]

A solution of 4,6-dichloro-1,7-naphthyridine (200 mg, 1.00 mmol), DIPEA (350 μL, 2.01 mmol) and 2-methyl-4-nitrophenol (185 mg, 1.21 mmol) in chlorobenzene (10 mL) was stirred at 150° C. for 3 days. The reaction was cooled to room temperature and water (15 mL) was added, the mixture was then extracted with DCM (3×15 mL), the combined organic layers were passed through a hydrophobic frit and concentrated in vacuo to afford a brown solid. The crude material was purified by column chromatography using KP-NH silica and eluting with 0-100% EtOAc in heptane. The product containing fractions were combined and concentrated in vacuo to afford 6-Chloro-4-(2-methyl-4-nitro-phenoxy)-1,7-naphthyridine (245 mg) as a yellow solid.

LC-MS (Method 3A): 1.47 min; MS m/z 316.0=[M+H]+

¹H NMR (500 MHz, DMSO-d6) δ 9.34 (d, J=0.7 Hz, 1H), 8.90 (d, J=5.1 Hz, 1H), 8.41 (d, J=2.8 Hz, 1H), 8.31 (d, J=0.6 Hz, 1H), 8.22 (dd, J=8.9, 2.9 Hz, 1H), 7.51 (d, J=8.9 Hz, 1H), 6.98 (d, J=5.1 Hz, 1H), 2.33 (s, 3H).

### Step 2: 4-[(6-Chloro-1,7-naphthyridin-4-yl)oxy]-3-methyl-aniline

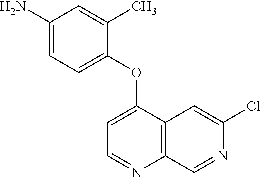 [SMILES: Cc1cc(N)ccc1Oc1ccnc2cnc(Cl)cc12]

A suspension of 6-chloro-4-(2-methyl-4-nitro-phenoxy)-1,7-naphthyridine (Example 30, step 1)(245 mg, 0.636 mmol), ammonium chloride (170 mg, 3.18 mmol) and iron (178 mg, 3.18 mmol) in water (1.4 mL) and MeOH (5.6 mL) were stirred at 75° C. for 1 h. The reaction mixture was cooled to room temperature and filtered through celite, washing with methanol (3×15 mL). The filtrate was concentrated in vacuo to afford a brown solid which was partitioned between 2:1 CHCl₃/isopropanol (20 mL) and water (20 mL). The biphasic mixture was filtered, the filtrate was separated and the aqueous phase was extracted with DCM (20 mL). The combined organic extracts were passed through a hydrophobic frit and concentrated in vacuo to afford 4-[(6-Chloro-1,7-naphthyridin-4-yl)oxy]-3-methyl-aniline (132 mg) as a brown solid.

LC-MS (Method 3A): 1.11 min; MS m/z 286.0=[M+H]+

¹H NMR (500 MHz, DMSO-d6) δ 9.26 (d, J=0.7 Hz, 1H), 8.80 (d, J=5.1 Hz, 1H), 8.26 (d, J=0.7 Hz, 1H), 6.89 (d, J=8.5 Hz, 1H), 6.66 (d, J=5.1 Hz, 1H), 6.56 (d, J=2.5 Hz, 1H), 6.51 (dd, J=8.5, 2.7 Hz, 1H), 5.14 (s, 2H), 1.96 (s, 3H).

### Step 3: N-[4-[(6-chloro-1,7-naphthyridin-4-yl)oxy]-3-methyl-phenyl]-2-oxo-1-phenyl-pyridine-3-carboxamide

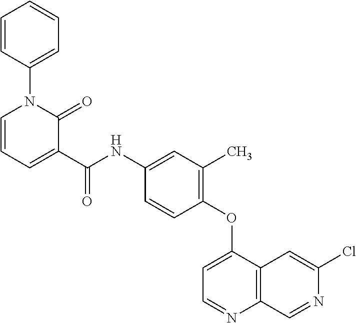 [SMILES: Cc1cc(NC(=O)c2cccn(-c3ccccc3)c2=O)ccc1Oc1ccnc2cnc(Cl)cc12]

A solution of 4-[(6-chloro-1,7-naphthyridin-4-yl)oxy]-3-methyl-aniline (Example 30, step 2)(132 mg, 0.462 mmol), 2-oxo-1-phenyl-pyridine-3-carboxylic acid (Intermediate A)(99 mg, 0.462 mmol), HATU (281 mg, 0.739 mmol) and DIPEA (0.20 mL, 1.15 mmol) in DMF (3 mL) was stirred at room temperature for 16 h. The reaction mixture was filtered and the resulting solid washed with water (2×15 mL) before being dried in vacuo at 40° C., to afford N-[4-[(6-chloro-1,7-naphthyridin-4-yl)oxy]-3-methyl-phenyl]-2-oxo-1-phenyl-pyridine-3-carboxamide (75 mg) as an off-white solid.

LC-MS (Method 3A): 1.60 min; MS m/z 483.0=[M+H]+

¹H NMR (400 MHz, DMSO-d6) δ 12.08 (s, 1H), 9.30 (d, J=0.7 Hz, 1H), 8.83 (d, J=5.1 Hz, 1H), 8.61 (dd, J=7.3, 2.2 Hz, 1H), 8.32 (d, J=0.7 Hz, 1H), 8.14 (dd, J=6.6, 2.2 Hz, 1H), 7.80-7.72 (m, 2H), 7.65-7.50 (m, 5H), 7.30-7.23 (m, 1H), 6.79-6.70 (m, 2H), 2.13 (s, 3H).

### Step 4: Tert-butyl 4-[4-[2-methyl-4-[(2-oxo-1-phenyl-pyridine-3-carbonyl)amino]phenoxy]-1,7-naphthyridin-6-yl]piperazine-1-carboxylate

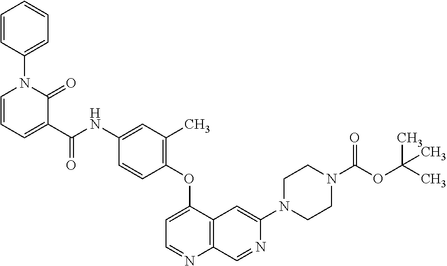 [SMILES: Cc1cc(NC(=O)c2cccn(-c3ccccc3)c2=O)ccc1Oc1ccnc2cnc(N3CCN(C(=O)OC(C)(C)C)CC3)cc12]

N-[4-[(6-chloro-1,7-naphthyridin-4-yl)oxy]-3-methyl-phenyl]-2-oxo-1-phenyl-pyridine-3-carboxamide (Example 30, step 3) (75 mg, 0.155 mmol), tert-butyl piperazine-1-carboxylate (44 mg, 0.233 mmol), cesium carbonate (152 mg, 0.466 mmol) and RuPhos Pd G3 (13 mg, 0.0155 mmol) were dissolved in DMA (7 mL), degassed with nitrogen and stirred in a sealed vial at 95° C. for 2 h. The reaction mixture was cooled to room temperature, partitioned between ethyl acetate (20 mL) and water (20 mL). The organic layer was washed with water (15 mL), brine (2×15 mL), dried over MgSO₄ and concentrated in vacuo. The crude product was purified by acidic reverse phase column chromatography (eluting with 10-100% MeCN (0.1% formic acid) in water (0.1% formic acid)) to afford tert-butyl 4-[4-[2-methyl-4-[(2-oxo-1-phenyl-pyridine-3-carbonyl)amino]phenoxy]-1,7-naphthyridin-6-yl]piperazine-1-carboxylate (36 mg) as a yellow solid.

LC-MS (Method 7A): 4.12 min; MS m/z 633.4=[M+H]+

¹H NMR (500 MHz, DMSO-d6) δ 12.05 (s, 1H), 9.08 (d, J=0.7 Hz, 1H), 8.60 (dd, J=7.3, 2.2 Hz, 1H), 8.44 (d, J=5.0 Hz, 1H), 8.13 (dd, J=6.6, 2.2 Hz, 1H), 7.78-7.69 (m, 2H), 7.62-7.49 (m, 5H), 7.25-7.16 (m, 2H), 6.77-6.68 (m, 1H), 6.46 (d, J=5.0 Hz, 1H), 3.67-3.57 (m, 4H), 3.53-3.44 (m, 4H), 2.10 (s, 3H), 1.43 (s, 9H).

### Step 5: N-[3-methyl-4-[(6-piperazin-1-yl-1,7-naphthyridin-4-yl)oxy]phenyl]-2-oxo-1-phenyl-pyridine-3-carboxamide

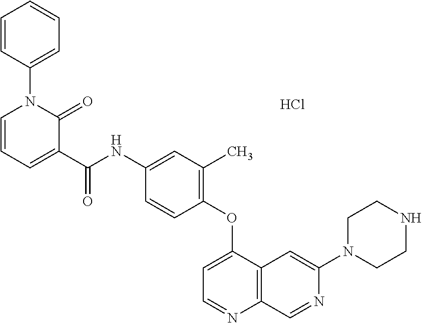 [SMILES: Cc1cc(NC(=O)c2cccn(-c3ccccc3)c2=O)ccc1Oc1ccnc2cnc(N3CCNCC3)cc12 ; Cl]

4 M hydrogen chloride in dioxane (3.0 mL, 12.0 mmol) was added to solution of tert-butyl 4-[4-[2-methyl-4-[(2-oxo-1-phenyl-pyridine-3-carbonyl)amino]phenoxy]-1,7-naphthyridin-6-yl]piperazine-1-carboxylate (Example 30, step 4) (36 mg, 0.0569 mmol) in 1,4-dioxane (2 mL) and the solution stirred for 3 h at room temperature. The reaction mixture was concentrated in vacuo to afford N-[3-methyl-4-[(6-piperazin-1-yl-1,7-naphthyridin-4-yl)oxy]phenyl]-2-oxo-1-phenyl-pyridine-3-carboxamide hydrochloride (32 mg) as a yellow solid.

LC-MS (Method 7A): 1.95 min; MS m/z 533.3=[M+H]+

¹H NMR (500 MHz, DMSO-d6) δ 12.08 (s, 1H), 9.16 (s, 1H), 8.98 (s, 2H), 8.60 (dd, J=7.3, 2.2 Hz, 1H), 8.53 (d, J=5.1 Hz, 1H), 8.14 (dd, J=6.6, 2.2 Hz, 1H), 7.80-7.72 (m, 2H), 7.63-7.50 (m, 5H), 7.38 (s, 1H), 7.23 (d, J=9.1 Hz, 1H), 6.77-6.73 (m, 1H), 6.54 (d, J=5.1 Hz, 1H), 3.92-3.85 (m, 4H), 3.33-3.24 (m, 4H), 2.12 (s, 3H).

### Example 31: N-[3-fluoro-4-[(6-piperazin-1-yl-1,7-naphthyridin-4-yl)oxy]phenyl]-2-oxo-1-phenyl-pyridine-3-carboxamide

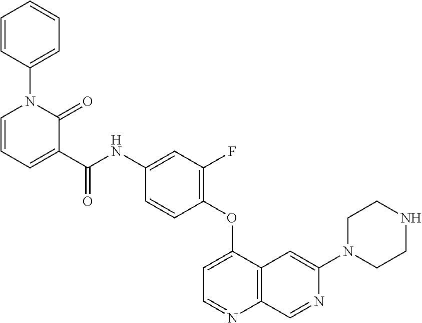 [SMILES: O=C(Nc1ccc(Oc2ccnc3cnc(N4CCNCC4)cc23)c(F)c1)c1cccn(-c2ccccc2)c1=O]

### Step 1: 6-chloro-4-(2-fluoro-4-nitro-phenoxy)-1,7-naphthyridine

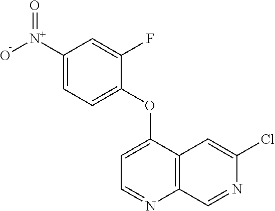

A solution of 4,6-dichloro-1,7-naphthyridine (160 mg, 0.804 mmol), DIPEA (280 uL, 1.61 mmol) and 2-fluoro-4-nitrophenol (152 mg, 0.965 mmol) in chlorobenzene (8 mL) was stirred at 140° C. for 20 h. The reaction was cooled to room temperature and water (10 mL) was added, the resulting suspension was extracted with DCM (10 mL) and 2:1 CHCl₃/IPA (2×10 mL), the combined organic layers were passed through a hydrophobic frit and concentrated in vacuo to afford 6-chloro-4-(2-fluoro-4-nitro-phenoxy)-1,7-naphthyridine (276 mg) as a brown solid.

LC-MS (Method 3A): 1.42 min; MS m/z 320.0=[M+H]+

¹H NMR (400 MHz, DMSO-d6) δ 9.37 (d, J=0.8 Hz, 1H), 8.95 (d, J=5.1 Hz, 1H), 8.52 (dd, J=10.4, 2.7 Hz, 1H), 8.30 (d, J=0.8 Hz, 1H), 8.27 (ddd, J=9.0, 2.7, 1.4 Hz, 1H), 7.80 (dd, J=8.9, 8.1 Hz, 1H), 7.22 (dd, J=5.1, 0.9 Hz, 1H).

### Step 2: 4-[(6-chloro-1,7-naphthyridin-4-yl)oxy]-3-fluoro-aniline

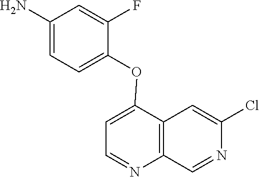 [SMILES: Nc1ccc(Oc2ccnc3cnc(Cl)cc23)c(F)c1]

A suspension of 6-chloro-4-(2-fluoro-4-nitro-phenoxy)-1,7-naphthyridine (Example 31, step 1)(276 mg, 0.717 mmol), ammonium chloride (192 mg, 3.58 mmol) and iron powder (200 mg, 3.58 mmol) in methanol (8 mL) and water (2 mL) were heated to 75° C. and were stirred for 1 h. The reaction mixture was cooled to room temperature and filtered through a pad of celite, washing the filter pad with methanol (3×15 mL). The combined filtrates were concentrated in vacuo. Water (20 mL) was added to the residue and the resulting suspension was extracted with 2:1 CHCl₃/IPA (2×20 mL). The organic layers were combined, passed through a hydrophobic frit and concentrated in vacuo to afford 4-[(6-chloro-1,7-naphthyridin-4-yl)oxy]-3-fluoro-aniline (229 mg) as a brown solid.

LC-MS (Method 3A): 1.27 min; MS m/z 290.0=[M+H]+

¹H NMR (500 MHz, DMSO-d6) δ 9.29 (d, J=0.6 Hz, 1H), 8.86 (d, J=5.1 Hz, 1H), 8.28-8.22 (m, 1H), 7.13 (dd, J=9.0 Hz, 1H), 6.88-6.81 (m, 1H), 6.58 (dd, J=13.2, 2.5 Hz, 1H), 6.49 (dd, J=8.7, 2.0 Hz, 1H), 5.57 (s, 2H).

### Step 3: N-[4-[(6-chloro-1,7-naphthyridin-4-yl)oxy]-3-fluoro-phenyl]-2-oxo-1-phenyl-pyridine-3-carboxamide

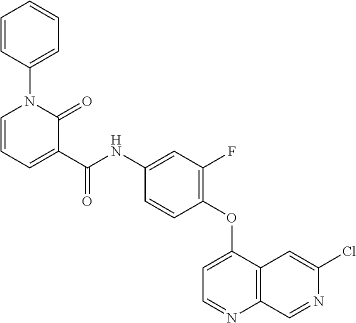 [SMILES: O=C(Nc1ccc(Oc2ccnc3cnc(Cl)cc23)c(F)c1)c1cccn(-c2ccccc2)c1=O]

A solution of 4-[(6-chloro-1,7-naphthyridin-4-yl)oxy]-3-fluoro-aniline (Example 31, step 2)(229 mg, 0.704 mmol), 2-oxo-1-phenyl-pyridine-3-carboxylic acid (Intermediate A)(151 mg, 0.704 mmol), HATU (428 mg, 1.13 mmol) and DIPEA (0.31 mL, 1.76 mmol) in DMF (5 mL) was stirred at room temperature for 3.5 h. The formed solid was collected by filtration, washed with water (2×10 mL) and dried in vacuo to afford N-[4-[(6-chloro-1,7-naphthyridin-4-yl)oxy]-3-fluoro-phenyl]-2-oxo-1-phenyl-pyridine-3-carboxamide (290 mg) as a light brown solid.

LC-MS (Method 3A): 1.57 min; MS m/z 487.2=[M+H]+

¹H NMR (500 MHz, DMSO-d6) δ 12.20 (s, 1H), 9.32 (s, 1H), 8.88 (d, J=5.1 Hz, 1H), 8.61 (dd, J=7.3, 2.1 Hz, 1H), 8.30 (s, 1H), 8.16 (dd, J=6.6, 2.2 Hz, 1H), 8.10 (dd, J=12.9, 2.3 Hz, 1H), 7.63-7.49 (m, 7H), 6.95 (d, J=4.7 Hz, 1H), 6.77-6.72 (m, 1H).

### Step 4: tert-butyl 4-[4-[2-fluoro-4-[(2-oxo-1-phenyl-pyridine-3-carbonyl)amino]phenoxy]-1,7-naphthyridin-6-yl]piperazine-1-carboxylate

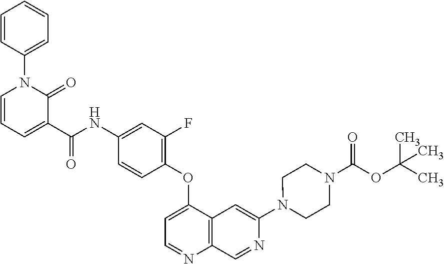 [SMILES: CC(C)(C)OC(=O)N1CCN(c2cc3c(Oc4ccc(NC(=O)c5cccn(-c6ccccc6)c5=O)cc4F)ccnc3cn2)CC1]

N-[4-[(6-chloro-1,7-naphthyridin-4-yl)oxy]-3-fluoro-phenyl]-2-oxo-1-phenyl-pyridine-3-carboxamide (Example 31, step 3)(150 mg, 0.308 mmol), tert-butyl piperazine-1-carboxylate (88 mg, 0.462 mmol), cesium carbonate (301 mg, 0.924 mmol) and RuPhos PdG3 (26 mg, 0.0308 mmol) were dissolved in dry DMA (12 mL), degassed with nitrogen and stirred in a sealed vial at 95° C. for 2 h. The reaction mixture was cooled to room temperature and partitioned between ethyl acetate (20 mL) and water (20 mL). The organic layer was washed with water (15 mL) and brine (2×15 mL), dried over MgSO₄ and concentrated in vacuo. The crude residue was purified by silica column chromatography (on KP-NH silica eluting with a gradient of 0-30% ethyl acetate in heptane to afford a yellow solid. The solid was purified by acidic reverse phase chromatography (eluting with a gradient of 10-100% MeCN (0.1% formic acid) in water (0.1% formic acid)) to afford tert-butyl 4-[4-[2-fluoro-4-[(2-oxo-1-phenyl-pyridine-3-carbonyl)amino]phenoxy]-1,7-naphthyridin-6-yl]piperazine-1-carboxylate (100 mg) as a yellow solid.

LC-MS (Method 2A): 1.43 min; MS m/z 637.3=[M+H]+

¹H NMR (500 MHz, DMSO-d6) δ 12.18 (s, 1H), 9.13-9.06 (m, 1H), 8.60 (dd, J=7.3, 2.2 Hz, 1H), 8.47 (d, J=5.0 Hz, 1H), 8.15 (dd, J=6.6, 2.2 Hz, 1H), 8.07 (dd, J=12.9, 2.4 Hz, 1H), 7.63-7.51 (m, 6H), 7.46 (t, J=8.9 Hz, 1H), 7.18 (s, 1H), 6.74 (dd, 1H), 6.65 (d, J=4.3 Hz, 1H), 3.67-3.58 (m, 4H), 3.56-3.42 (m, 4H), 1.43 (s, 9H).

### Step 5: N-[3-fluoro-4-[(6-piperazin-1-yl-1,7-naphthyridin-4-yl)oxy]phenyl]-2-oxo-1-phenyl-pyridine-3-carboxamide hydrochloride

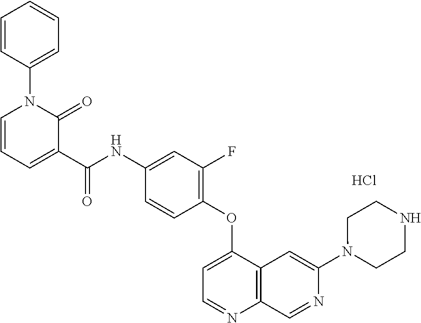 [SMILES: O=C(Nc1ccc(Oc2ccnc3cnc(N4CCNCC4)cc23)c(F)c1)c1cccn(-c2ccccc2)c1=O ; Cl]

4 M hydrogen chloride in dioxane (5.0 mL, 20.0 mmol) was added to suspension of tert-butyl 4-[4-[2-fluoro-4-[(2-oxo-1-phenyl-pyridine-3-carbonyl)amino]phenoxy]-1,7-naphthyridin-6-yl]piperazine-1-carboxylate (Example 31, step 3) (100 mg, 0.157 mmol) in 1,4-dioxane (5 mL). The solution was stirred at room temperature for 18 h. The reaction mixture was concentrated in vacuo to afford N-[3-fluoro-4-[(6-piperazin-1-yl-1,7-naphthyridin-4-yl)oxy]phenyl]-2-oxo-1-phenyl-pyridine-3-carboxamide hydrochloride (89.8 mg) as a yellow solid.

LC-MS (Method 7A): 2.03 min; MS m/z 537.2=[M+H]+

¹H NMR (500 MHz, DMSO-d6) δ 12.19 (s, 1H), 9.23-9.06 (m, 3H), 8.60 (dd, J=7.3, 2.2 Hz, 1H), 8.57 (d, J=5.2 Hz, 1H), 8.15 (dd, J=6.6, 2.2 Hz, 1H), 8.09 (dd, J=12.9, 2.4 Hz, 1H), 7.63-7.51 (m, 6H), 7.48 (dd, J=8.9 Hz, 1H), 7.34 (s, 1H), 6.77-6.72 (m, 2H), 3.93-3.86 (m, 4H), 3.29-3.20 (m, 4H).

### Example 32: 2-oxo-1-phenyl-N-[5-[(6-piperazin-1-yl-1,7-naphthyridin-4-yl)oxy]-2-pyridyl]pyridine-3-carboxamide

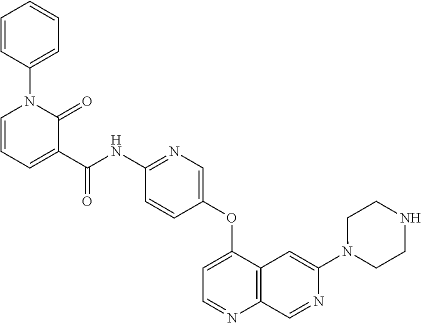 [SMILES: O=C(Nc1ccc(Oc2ccnc3cnc(N4CCNCC4)cc23)cn1)c1cccn(-c2ccccc2)c1=O]

### Step 1: N-[5-[(6-chloro-1,7-naphthyridin-4-yl)oxy]-2-pyridyl]-2-oxo-1-phenyl-pyridine-3-carboxamide

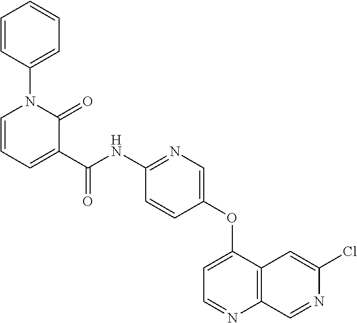 [SMILES: O=C(Nc1ccc(Oc2ccnc3cnc(Cl)cc23)cn1)c1cccn(-c2ccccc2)c1=O]

A solution of 5-[(6-chloro-1,7-naphthyridin-4-yl)oxy]pyridin-2-amine (Example 10, step 2)(160 mg, 0.587 mmol), 2-oxo-1-phenyl-pyridine-3-carboxylic acid (Intermediate A)(126 mg, 0.587 mmol), HATU (357 mg, 0.939 mmol) and DIPEA (0.26 mL, 1.47 mmol) in DMF (4 mL) was stirred at 60° C. for 18 h. The reaction was cooled to room temperature and water (10 mL) was added. The resulting solid was collected by filtration, washed with water (2×15 mL) and dried to afford N-[5-[(6-chloro-1,7-naphthyridin-4-yl)oxy]-2-pyridyl]-2-oxo-1-phenyl-pyridine-3-carboxamide (317 mg) as a brown solid.

LC-MS (Method 3A): 1.43 min; MS m/z 470.2=[M+H]+

¹H NMR (500 MHz, DMSO-d6) δ 12.49 (s, 1H), 9.31 (s, 1H), 8.87 (d, J=5.1 Hz, 1H), 8.64 (dd, J=7.2, 1.9 Hz, 1H), 8.49-8.39 (m, 2H), 8.29 (s, 1H), 8.16 (dd, J=6.6, 2.0 Hz, 1H), 7.94 (dd, J=9.0, 2.7 Hz, 1H), 7.65-7.48 (m, 5H), 7.01 (d, J=5.1 Hz, 1H), 6.75 (dd, 1H).

### Step 2: tert-butyl 4-[4-[[6-[(2-oxo-1-phenyl-pyridine-3-carbonyl)amino]-3-pyridyl]oxy]-1,7-naphthyridin-6-yl]piperazine-1-carboxylate

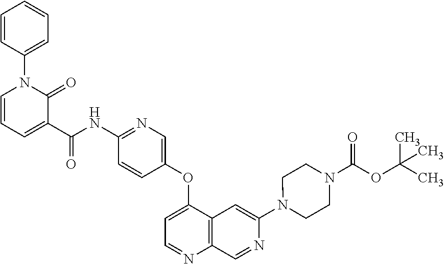 [SMILES: CC(C)(C)OC(=O)N1CCN(c2cc3c(Oc4ccc(NC(=O)c5cccn(-c6ccccc6)c5=O)nc4)ccnc3cn2)CC1]

N-[5-[(6-chloro-1,7-naphthyridin-4-yl)oxy]-2-pyridyl]-2-oxo-1-phenyl-pyridine-3-carboxamide (Example 32, step 1)(155 mg, 0.330 mmol), tert-butyl piperazine-1-carboxylate (94 mg, 0.495 mmol), cesium carbonate (322 mg, 0.990 mmol) and RuPhos PdG3 (28 mg, 0.0330 mmol) were dissolved in dry DMA (15 mL), degassed with nitrogen and stirred in a sealed vial at 95° C. for 2 h. The reaction mixture was cooled to room temperature and partitioned between ethyl acetate (20 mL) and water (20 mL). The separated organic fraction was washed with water (15 mL), brine (2×15 mL) dried over MgSO4 and concentrated in vacuo. The crude product was purified by acidic reverse phase chromatography (eluting with a gradient of 10-100% MeCN (0.1% formic acid) in water (0.1% formic acid)) to afford tert-butyl 4-[4-[[6-[(2-oxo-1-phenyl-pyridine-3-carbonyl)amino]-3-pyridyl]oxy]-1,7-naphthyridin-6-yl]piperazine-1-carboxylate (70 mg) as a yellow solid.

LC-MS (Method 7A): 3.89 min; MS m/z 620.3=[M+H]+

¹H NMR (500 MHz, DMSO) δ 12.47 (s, 1H), 9.10 (s, 1H), 8.64 (dd, J=7.3, 2.2 Hz, 1H), 8.48 (d, J=5.0 Hz, 1H), 8.44 (d, J=9.0 Hz, 1H), 8.38 (d, J=2.9 Hz, 1H), 8.16 (dd, J=6.6, 2.2 Hz, 1H), 7.88 (dd, J=9.0, 2.9 Hz, 1H), 7.62-7.52 (m, 5H), 7.19 (s, 1H), 6.77-6.70 (m, 2H), 3.66-3.58 (m, 4H), 3.53-3.45 (m, 4H), 1.43 (s, 9H).

### Step 3: 2-oxo-1-phenyl-N-[5-[(6-piperazin-1-yl-1,7-naphthyridin-4-yl)oxy]-2-pyridyl]pyridine-3-carboxamide

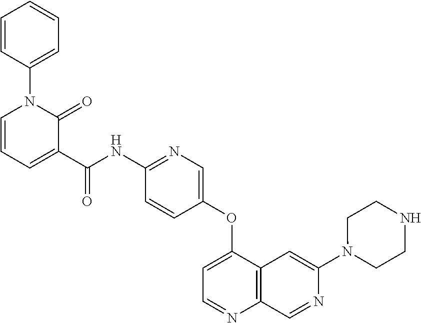 [SMILES: O=C(Nc1ccc(Oc2ccnc3cnc(N4CCNCC4)cc23)cn1)c1cccn(-c2ccccc2)c1=O]

4 M hydrogen chloride in dioxane (3.0 mL, 12.0 mmol) was added to suspension of tert-butyl 4-[4-[[6-[(2-oxo-1-phenyl-pyridine-3-carbonyl)amino]-3-pyridyl]oxy]-1,7-naphthyridin-6-yl]piperazine-1-carboxylate (Example 31, step 2) (70 mg, 0.113 mmol) in 1,4-dioxane (3 mL) and the mixture stirred at room temperature for 18 h. The mixture was concentrated in vacuo to afford a yellow solid which was loaded onto a 5 g SCX cartridge.

The column was sequentially washed with MeOH/DCM (1:1, 3×10 mL) and 7M NH₃ in MeOH (2×10 mL). The methanolic ammonia fraction was concentrated in vacuo to afford 2-oxo-1-phenyl-N-[5-[(6-piperazin-1-yl-1,7-naphthyridin-4-yl)oxy]-2-pyridyl]pyridine-3-carboxamide (45.6 mg) as a yellow solid.

LC-MS (Method 7A): 1.77 min; MS m/z 520.3=[M+H]+

¹H NMR (500 MHz, DMSO-d6) δ 12.46 (s, 1H), 9.13-8.98 (m, 1H), 8.64 (dd, J=7.3, 2.2 Hz, 1H), 8.50-8.41 (m, 2H), 8.37 (d, J=2.9 Hz, 1H), 8.16 (dd, J=6.6, 2.2 Hz, 1H), 7.88 (dd, J=9.0, 2.9 Hz, 1H), 7.64-7.56 (m, 2H), 7.56-7.50 (m, 3H), 7.12 (s, 1H), 6.74 (dd, 1H), 6.71 (d, J=5.0 Hz, 1H), 3.63-3.49 (m, 4H), 2.97-2.76 (m, 4H).

### Example 33: 4-[2-fluoro-4-[(2-oxo-1-phenyl-pyridine-3-carbonyl)amino]phenoxy]-N-(1-methyl-4-piperidyl)-1,7-naphthyridine-6-carboxamide, bis formate

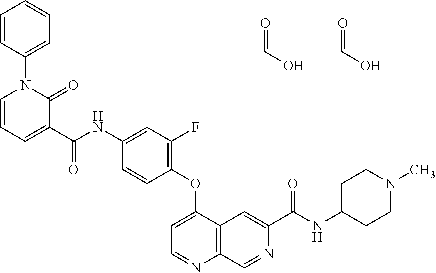 [SMILES: CN1CCC(NC(=O)c2cc3c(Oc4ccc(NC(=O)c5cccn(-c6ccccc6)c5=O)cc4F)ccnc3cn2)CC1 ; O=CO ; O=CO]

A mixture of N-[4-[(6-chloro-1,7-naphthyridin-4-yl)oxy]-3-fluoro-phenyl]-2-oxo-1-phenyl-pyridine-3-carboxamide (Example 31, step 3)(52 mg, 0.107 mmol), XantPhos Pd G3 (20 mg, 0.0214 mmol) and 1-methylpiperidin-4-amine (49 mg, 0.427 mmol) in dry DMA (5 mL) was added to chamber A of a 20 mL COware apparatus and the apparatus was flushed with nitrogen. A solution of methanesulfonyl chloride (0.025 mL, 0.320 mmol) and formic acid (0.012 mL, 0.320 mmol) in nitrogen flushed toluene (1 mL) was added to Chamber B before the addition of DIPEA (0.11 mL, 0.641 mmol) to chamber B. The reaction mixture was heated to 100° C. and was stirred for 17 h. The reaction mixture was allowed to cool to RT before being degassed with nitrogen for 5 minutes. The reaction mixture was partitioned between DCM (10 mL) and water (10 mL), the organic fraction separated and the aqueous phase further extracted with DCM (2×10 mL). The combined organic fractions were passed through a hydrophobic frit and concentrated in vacuo. The crude product was purified by acidic reverse phase chromatography (eluting with 10-100% MeCN (0.1% formic acid) in water (0.1% formic acid)) to afford 4-[2-fluoro-4-[(2-oxo-1-phenyl-pyridine-3-carbonyl)amino]phenoxy]-N-(1-methyl-4-piperidyl)-1,7-naphthyridine-6-carboxamide bis formate salt (11.9 mg).

LC-MS (Method 7B): 3.40 min; MS m/z 593.4=[M+H]+

¹H NMR (500 MHz, DMSO-d6) δ 12.21 (s, 1H), 9.46 (d, J=0.8 Hz, 1H), 8.94 (d, J=5.2 Hz, 1H), 8.83-8.76 (m, 2H), 8.62 (dd, J=7.3, 2.2 Hz, 1H), 8.23 (s, 2H), 8.16 (dd, J=6.6, 2.2 Hz, 1H), 8.14-8.08 (m, 1H), 7.62-7.52 (m, 7H), 7.00 (dd, J=4.3 Hz, 1H), 6.75 (dd, 1H), 3.91-3.82 (m, 1H), 2.80 (d, J=11.3 Hz, 2H), 2.21 (s, 3H), 2.09-2.01 (m, 2H), 1.83-1.72 (m, 4H).

### Example 34: 4-[2-Methyl-4-[(2-oxo-1-phenyl-pyridine-3-carbonyl)amino]phenoxy]-N-(1-methyl-4-piperidyl)-1,7-naphthyridine-6-carboxamide

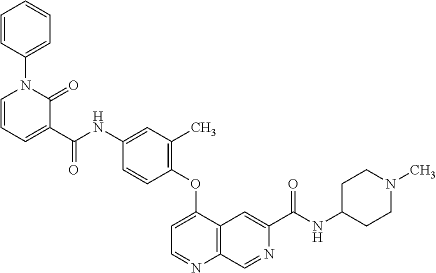 [SMILES: Cc1cc(NC(=O)c2cccn(-c3ccccc3)c2=O)ccc1Oc1ccnc2cnc(C(=O)NC3CCN(C)CC3)cc12]

A suspension of 1-methylpiperidin-4-amine (47 mg, 0.414 mmol), sodium carbonate (33 mg, 0.311 mmol), XantPhos Pd-G3 (10 mg, 0.0104 mmol) and N-[4-[(6-chloro-1,7-naphthyridin-4-yl)oxy]-3-methyl-phenyl]-2-oxo-1-phenyl-pyridine-3-carboxamide (Example 30, step 3)(50 mg, 0.104 mmol) in dry DMA (5 mL) was added to chamber A of a 20 ml COware apparatus. The apparatus was flushed with nitrogen before the addition of formic acid (0.012 mL, 0.311 mmol) and methanesulfonyl chloride (0.024 mL, 0.311 mmol) in nitrogen flushed dry toluene (2 mL) into Chamber B. Triethylamine (0.085 mL, 0.621 mmol) was added to Chamber B and the reaction mixture was heated to 100° C. and stirred for 18 h. The reaction mixture was cooled to room temperature and partitioned between water (20 mL) and DCM (20 mL). The organic fraction was separated and the aqueous layer further extracted with DCM (2×10 mL). The combined organics were passed through a hydrophobic frit before being concentrated in vacuo. The residue was purified by acidic reverse phase chromatography (eluting with a gradient of 10-100% MeCN (0.1% formic acid) in water (0.1% formic acid)). The pure fractions were combined and loaded onto a 5 g SCX cartridge and the column washed sequentially with MeOH (3×10 mL) 7M NH₃ in MeOH (3×10 mL). The methanolic ammonia fraction was concentrated in vacuo to afford 4-[2-Methyl-4-[(2-oxo-1-phenyl-pyridine-3-carbonyl)amino]phenoxy]-N-(1-methyl-4-piperidyl)-1,7-naphthyridine-6-carboxamide (38.5 mg) as a brown solid.

LC-MS (Method 7A): 2.12 min; MS m/z 589.3=[M+H]+

¹H NMR (500 MHz, DMSO-d6) δ 12.08 (s, 1H), 9.43 (d, J=0.8 Hz, 1H), 8.89 (d, J=5.2 Hz, 1H), 8.83 (d, J=0.8 Hz, 1H), 8.77 (d, J=8.4 Hz, 1H), 8.60 (dd, J=7.3, 2.2 Hz, 1H), 8.13 (dd, J=6.6, 2.2 Hz, 1H), 7.80-7.74 (m, 2H), 7.63-7.56 (m, 2H), 7.56-7.51 (m, 3H), 7.30-7.26 (m, 1H), 6.77 (d, J=5.2 Hz, 1H), 6.74 (dd, 1H), 3.91-3.80 (m, 1H), 2.82-2.72 (m, 2H), 2.19 (s, 3H), 2.12 (s, 3H), 2.08-1.97 (m, 2H), 1.84-1.69 (m, 4H).

### Example 35: 1-(4-fluorophenyl)-N-[4-[[6-(4-methylpiperazin-1-yl)-1,7-naphthyridin-4-yl]oxy]phenyl]-2-oxo-pyridine-3-carboxamide

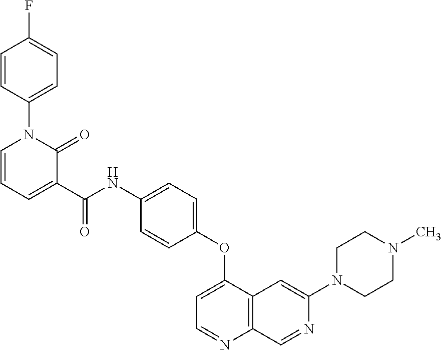 [SMILES: CN1CCN(c2cc3c(Oc4ccc(NC(=O)c5cccn(-c6ccc(F)cc6)c5=O)cc4)ccnc3cn2)CC1]

### Step 1: 6-(4-methylpiperazin-1-yl)-4-(4-nitrophenoxy)-1,7-naphthyridine

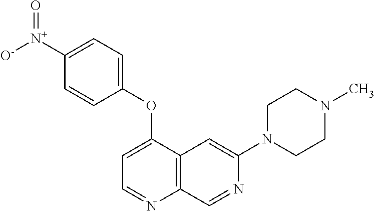 [SMILES: CN1CCN(c2cc3c(Oc4ccc([N+](=O)[O-])cc4)ccnc3cn2)CC1]

6-chloro-4-(4-nitrophenoxy)-1,7-naphthyridine (Example 10, step 1)(200 mg, 0.663 mmol), 1-methylpiperazine (100 mg, 0.998 mmol), BINAP (42 mg, 0.0675 mmol) and cesium carbonate (651 mg, 2.00 mmol) were suspended in toluene (4 mL). The reaction mixture was degassed for 10 min before addition of Pd₂dba₃ (61 mg, 0.0666 mmol) before being heated at 100° C. for 36 h. The reaction was cooled to room temperature, water (10 mL) was added and the mixture extracted with DCM (3×15 mL). The combined organic fractions were dried over MgSO4 and concentrated in vacuo. The crude material was purified by silica chromatography (eluting with a gradient of 0-10% DCM/MeOH) to afford 6-(4-methylpiperazin-1-yl)-4-(4-nitrophenoxy)-1,7-naphthyridine (55 mg) as an orange oil.

LC-MS (Method 2A): 0.85 min; MS m/z=366.0 [M+H]+

¹H NMR (400 MHz, Chloroform-d) δ 9.18-9.12 (m, 1H), 8.47 (d, J=4.9 Hz, 1H), 8.32-8.23 (m, 2H), 7.24-7.17 (m, 2H), 6.88 (s, 1H), 6.66 (d, J=4.9 Hz, 1H), 3.64-3.57 (m, 4H), 2.55-2.48 (m, 4H), 2.30 (s, 3H).

### Step 2: 4-[[6-(4-methylpiperazin-1-yl)-1,7-naphthyridin-4-yl]oxy]aniline

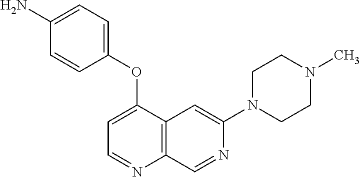 [SMILES: CN1CCN(c2cc3c(Oc4ccc(N)cc4)ccnc3cn2)CC1]

6-(4-methylpiperazin-1-yl)-4-(4-nitrophenoxy)-1,7-naphthyridine (Example 35, step 1)(55 mg, 0.151 mmol), iron powder (42 mg, 0.752 mmol) and ammonium chloride (40 mg, 0.748 mmol) were suspended in a mixture of methanol (3 mL) and water (0.5 mL) and the mixture was heated at 75° C. for 30 min. The mixture was cooled to room temperature, water (10 mL) was added and the resultant emulsion was extracted with CHCl₃/IPA (2:1. 3×10 mL). The combined organic extracts were filtered through a hydrophobic frit and concentrated in vacuo. The crude material was purified by high pH reverse phase chromatography (eluting with a gradient of 0-100% MeCN (0.1% ammonium hydroxide) in water (0.1% ammonium hydroxide)) to afford 4-[[6-(4-methylpiperazin-1-yl)-1,7-naphthyridin-4-yl]oxy]aniline (29 mg) as a yellow solid.

LC-MS (Method 2B): 1.36 min; MS m/z=336.0 [M+H]+

¹H NMR (400 MHz, DMSO-d6) δ 9.04 (s, 1H), 8.43 (d, J=5.0 Hz, 1H), 7.14 (s, 1H), 6.97-6.90 (m, 2H), 6.72-6.64 (m, 2H), 6.51 (d, J=5.0 Hz, 1H), 5.18 (s, 2H), 3.60 (s, 4H), 2.58-2.52 (m, 4H), 2.26 (s, 3H).

### Step 3: 1-(4-fluorophenyl)-N-[4-[[6-(4-methylpiperazin-1-yl)-1,7-naphthyridin-4-yl]oxy]phenyl]-2-oxo-pyridine-3-carboxamide

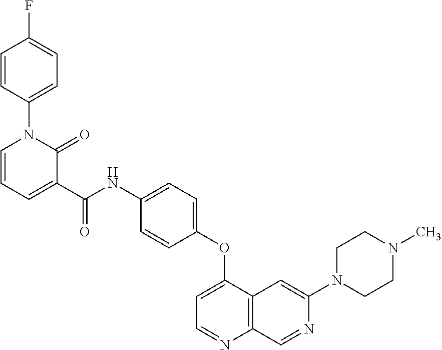 [SMILES: CN1CCN(c2cc3c(Oc4ccc(NC(=O)c5cccn(-c6ccc(F)cc6)c5=O)cc4)ccnc3cn2)CC1]

1-(4-fluorophenyl)-2-oxo-pyridine-3-carboxylic acid (Intermediate B)(1.49 eq, 30 mg, 0.129 mmol) and HATU (2.01 eq, 66 mg, 0.174 mmol) were suspended in DMF (0.75 mL). To this suspension was added 4-[[6-(4-methylpiperazin-1-yl)-1,7-naphthyridin-4-yl]oxy]aniline (Example 35, step 2)(29 mg, 0.0865 mmol) and N-methyl morpholine (29 uL, 0.264 mmol) in DMF (0.75 mL) and the mixture was stirred at room temperature for 1 h. Water (6 mL) was added and a solid formed which was collected by filtration and washed with diethyl ether (2×5 mL). The solid was purified by low pH reverse phase chromatography (eluting with 5-100% MeCN (0.1% formic acid) in water (0.1% formic acid)). The product was loaded onto a 1 g SCX cartridge and the column washed sequentially with MeOH (3×5 mL) and 7M NH₃ in MeOH (3×5 mL). The methanolic ammonia fraction was concentrated in vacuo to afford 1-(4-fluorophenyl)-N-[4-[[6-(4-methylpiperazin-1-yl)-1,7-naphthyridin-4-yl]oxy]phenyl]-2-oxo-pyridine-3-carboxamide (13.2 mg) as a yellow solid.

LC-MS (Method 7A): 1.92 min; MS m/z=551.3 [M+H]+

¹H NMR (500 MHz, DMSO-d6) δ 12.03 (s, 1H), 9.07 (d, J=0.7 Hz, 1H), 8.60 (dd, J=7.3, 2.2 Hz, 1H), 8.46 (d, J=5.0 Hz, 1H), 8.13 (dd, J=6.6, 2.2 Hz, 1H), 7.90-7.81 (m, 2H), 7.62 (dd, 2H), 7.49-7.39 (m, 2H), 7.32-7.25 (m, 2H), 7.14 (s, 1H), 6.78-6.69 (m, 1H), 6.63 (d, J=5.0 Hz, 1H), 3.60 (s, 4H), 2.50 (s, 4H), 2.26 (s, 3H).

### Example 36: 1-cyclopropyl-3-(4-fluorophenyl)-2,4-dioxo-N-[4-[(6-piperazin-1-yl-1,7-naphthyridin-4-yl)oxy]phenyl]pyrimidine-5-carboxamide hydrochloride

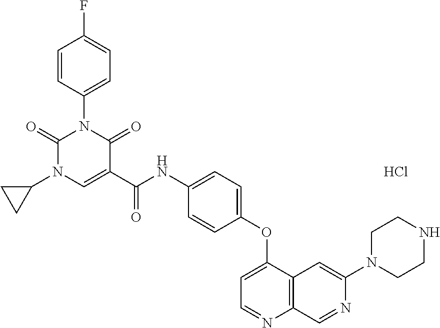 [SMILES: O=C(Nc1ccc(Oc2ccnc3cnc(N4CCNCC4)cc23)cc1)c1cn(C2CC2)c(=O)n(-c2ccc(F)cc2)c1=O ; Cl]

A solution of HATU (59 mg, 0.154 mmol), 1-cyclopropyl-3-(4-fluorophenyl)-2,4-dioxo-pyrimidine-5-carboxylic acid (Intermediate J)(38 mg, 0.130 mmol) and DIPEA (52 uL, 0.297 mmol) in DMF (2 mL) was stirred at room temperature for 15 minutes before the addition of tert-butyl 4-[4-(4-aminophenoxy)-1,7-naphthyridin-6-yl]piperazine-1-carboxylate (Example 12, step 3) (50 mg, 0.119 mmol). The reaction mixture was stirred for 1 h. Water (10 mL) was added to the reaction mixture and the resulting solid was collected by filtration, and washed with water (3×5 mL) to afford the protected intermediate as a yellow solid. The solid was suspended in 1,4-dioxane (2 mL), 4M hydrochloric acid in dioxane (4 mL, 16.00 mmol) was added and the reaction mixture was stirred for 24 h. The solid was collected by filtration, washed with diethyl ether (3×3 mL) and dried to afford 1-cyclopropyl-3-(4-fluorophenyl)-2,4-dioxo-N-[4-[(6-piperazin-1-yl-1,7-naphthyridin-4-yl)oxy]phenyl]pyrimidine-5-carboxamide hydrochloride (28.1 mg) as a yellow solid.

LC-MS (Method 7A): 1.95 min; MS m/z=594.3 [M+H]+

¹H NMR (400 MHz, DMSO-d6) δ 10.91 (s, 1H), 9.19 (s, 3H), 8.59 (d, J=5.3 Hz, 1H), 8.50 (s, 1H), 7.96-7.73 (m, 2H), 7.53-7.23 (m, 7H), 6.74 (d, J=5.3 Hz, 1H), 3.93-3.85 (m, 4H), 3.33-3.16 (m, 5H), 1.08-0.93 (m, 4H).

### Example 37: 4-[4-[[5-(4-fluorophenyl)-1-methyl-4-oxo-pyridine-3-carbonyl]amino]phenoxy]-N-(1-methyl-4-piperidyl)-1,7-naphthyridine-6-carboxamide

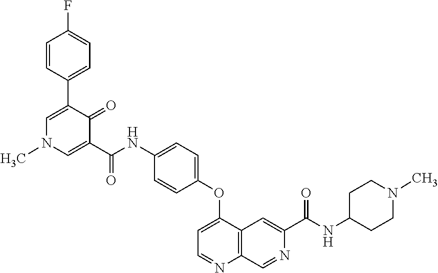

4-(4-aminophenoxy)-N-(1-methyl-4-piperidyl)-1,7-naphthyridine-6-carboxamide (Example 8, step 2)(50 mg, 0.128 mmol) was added to a solution of 5-(4-fluorophenyl)-1-methyl-4-oxo-pyridine-3-carboxylic acid (35 mg, 0.141 mmol), HATU (64 mg, 0.167 mmol) and DIPEA (56 uL, 0.321 mmol) in DMF (2 mL) and the solution stirred for 18 h. The mixture was partitioned between DCM (10 mL) and water (10 m), the organic fraction separated and aqueous phase further extracted with DCM (2×10 mL). The combined organic fractions were passed through a hydrophobic frit and concentrated in vacuo. The crude product was purified by high pH preparative HPLC (early elute method) to afford 4-[4-[[5-(4-fluorophenyl)-1-methyl-4-oxo-pyridine-3-carbonyl]amino]phenoxy]-N-(1-methyl-4-piperidyl)-1,7-naphthyridine-6-carboxamide (28.2 mg) as an off-white solid.

LC-MS (Method 7A): 2.05 min; MS m/z=607.3 [M+H]+

1H NMR (500 MHz, DMSO-d6) δ 13.13 (s, 1H), 9.43 (d, J=0.8 Hz, 1H), 8.91 (d, J=5.2 Hz, 1H), 8.79 (d, J=0.8 Hz, 1H), 8.76 (d, J=8.4 Hz, 1H), 8.70 (d, J=2.3 Hz, 1H), 8.16 (d, J=2.3 Hz, 1H), 7.93-7.85 (m, 2H), 7.77-7.70 (m, 2H), 7.39-7.34 (m, 2H), 7.33-7.25 (m, 2H), 6.94 (d, J=5.2 Hz, 1H), 3.93 (s, 3H), 3.90-3.75 (m, 1H), 2.82-2.70 (m, 2H), 2.18 (s, 3H), 2.05-1.94 (m, 2H), 1.83-1.67 (m, 4H).

The compound of the following tabulated example (Table 18) was prepared analogously to Example 37.

TABLE 18

| Ex. | Structure and Name | Retention Time, [M + H]+, 1H NMR |
|---|---|---|
| 37.1 | 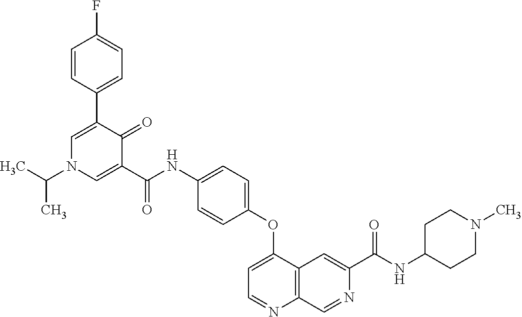 [SMILES: CC(C)n1cc(C(=O)Nc2ccc(Oc3ccnc4cnc(C(=O)NC5CCN(C)CC5)cc34)cc2)c(=O)c(-c2ccc(F)cc2)c1] | LC-MS (Method 7B): 3.73 min; MS m/z = 635.3 [M + H]+ ¹H NMR (500 MHz, DMSO-d₆) δ 13.12 (s, 1H), 9.43 (s, 1H), 8.91 (d, J = 5.2 Hz, 1H), 8.89-8.83 (m, 1H), 8.79 (s, 1H), 8.76 (d, J = 2.3 Hz, 1H), 8.27 (d, J = 2.3 Hz, 1H), 7.92-7.85 (m, 2H), 7.77- 7.69 (m, 2H), 7.37 (d, J = 8.9 Hz, 2H), 7.32-7.24 (m, 2H), 6.95 (d, J = 5.2 Hz, 1H), 4.70-4.58 (m, 1H), 3.99-3.87 (m, 1H), 3.05-2.91 (m, 2H), 2.42- 2.24 (m, 5H), 1.90-1.74 (m, 4H), 1.51 (d, J = 6.7 Hz, 6H). |
| | 4-[4-[[5-(4-fluorophenyl)-1-isopropyl-4-oxo-pyridine-3- | |
| | carbonyl]amino]phenoxy]-N-(1- | |
| | methyl-4-piperidyl)-1,7-naphthyridine-6-carboxamide | |

### Intermediates

### Intermediate A

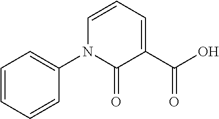 [SMILES: O=C(O)c1cccn(-c2ccccc2)c1=O]

### Step 1: methyl 2-oxo-1-phenyl-pyridine-3-carboxylate

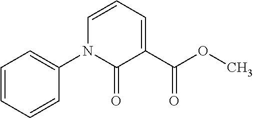 [SMILES: COC(=O)c1cccn(-c2ccccc2)c1=O]

To a stirred mixture of methyl 2-oxo-1H-pyridine-3-carboxylate (3.00 g, 19.6 mmol), phenylboronic acid (7.17 g, 58.8 mmol), Copper (II) acetate (7.12 g, 39.2 mmol) and 5.6 g of 4A molecular sieves in DCM (120 mL) was added pyridine (4.7 mL, 58.8 mmol). The resulting mixture was stirred at room temperature for 24 h. The reaction mixture was filtered through a pad of Celite and the filter bed was washed with DCM (150 mL). The filtrate was concentrated in vacuo and the residue was purified by silica chromatography (eluting with 0-100% EtOAc in heptane) to afford methyl 2-oxo-1-phenyl-pyridine-3-carboxylate (1.52 g) as an off white solid.

LC-MS (Method 2A): Rt 0.90 mins; MS m/z 230.1=[M+H]+

¹H NMR (400 MHz, DMSO-d6) δ 8.12 (dd, J=7.2, 2.2 Hz, 1H), 7.95 (dd, J=6.7, 2.2 Hz, 1H), 7.57-7.45 (m, 3H), 7.44-7.38 (m, 2H), 6.41 (dd, J=7.0 Hz, 1H), 3.75 (s, 3H)

### Step 2: 2-oxo-1-phenyl-pyridine-3-carboxylic acid

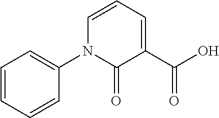 [SMILES: O=C(O)c1cccn(-c2ccccc2)c1=O]

To a stirred solution of methyl 2-oxo-1-phenyl-pyridine-3-carboxylate (Intermediate A, step 1)(1.50 g, 6.54 mmol) in THF (15 mL) and Methanol (15 mL) was added 1 M Sodium hydroxide solution (33 mL, 32.7 mmol). The resulting mixture was stirred at room temperature for 2 h. The reaction mixture was partially concentrated in vacuo to remove the organic solvent. The resulting aqueous mixture was acidified to pH 3 using aqueous 2M HCl.

The resulting solid was collected by filtration and dried in a vacuum oven at 40° C. to afford 2-oxo-1-phenyl-pyridine-3-carboxylic acid (1.32 g) as a pale yellow solid.

LC-MS (Method 2A): Rt 0.93 mins; MS m/z 216.1=[M+H]+

¹H NMR (400 MHz, DMSO-d6) δ 14.28 (s, 1H), 8.49 (dd, J=7.3, 2.1 Hz, 1H), 8.21 (dd, J=6.6, 2.1 Hz, 1H), 7.65-7.42 (m, 5H), 6.80 (dd, J=7.2, 6.7 Hz, 1H)

The compound of the following tabulated Example (Table 19) was prepared analogously to Intermediate A steps 1 & 2 from 4-fluorophenylboronic acid.

TABLE 19

| | Retention Time, [M + H]+, |
|---|---|
| Structure and Name | 1H NMR |
| 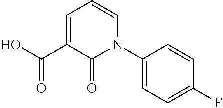 [SMILES: O=C(O)c1cccn(-c2ccc(F)cc2)c1=O] | LC-MS (Method 2A): Rt 0.96 mins; MS m/z 234.0 = [M + H]+ ¹H NMR (500 MHz, DMSO-d6) δ 14.23 (s, 1H), 8.49 (dd, J = 7.3, 2.1 Hz, 1H), 8.21 (dd, J = 6.6, 2.1 Hz, 1H), 7.66-7.54 (m, 2H), 7.49-7.34 |
| 1-(4-fluorophenyl)-2-oxo-pyridine- | (m, 2H), 6.79 (dd, J = 6.8 Hz, 1H). |
| 3-carboxylic acid (intermediate B) | |

### Intermediate C

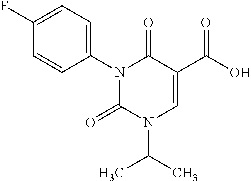 [SMILES: CC(C)n1cc(C(=O)O)c(=O)n(-c2ccc(F)cc2)c1=O]

### Step 1: Isopropyl 3-(4-fluorophenyl)-1-isopropyl-2,4-dioxo-pyrimidine-5-carboxylate

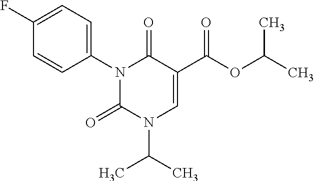 [SMILES: CC(C)OC(=O)c1cn(C(C)C)c(=O)n(-c2ccc(F)cc2)c1=O]

3-(4-fluorophenyl)-2,4-dioxo-1H-pyrimidine-5-carboxylic acid (1.00 g, 4.00 mmol), 2-iodopropane (1.2 mL, 12.0 mmol), and potassium carbonate (1.66 g, 12.0 mmol) were dissolved in DMF (6 mL) and the reaction was stirred in a sealed tube at 80° C. for 20 h. The reaction was cooled to room temperature. Water (10 mL) and EtOAc (10 mL) were added, the aqueous layer was extracted with EtOAc (2×5 mL) and the combined organic layers were washed with water (2×5 mL), brine (2 mL), passed through a hydrophobic frit and concentrated in vacuo to afford isopropyl 3-(4-fluorophenyl)-1-isopropyl-2,4-dioxo-pyrimidine-5-carboxylate (1.02 g) as a pale yellow solid.

LC-MS (Method 2A): Rt 1.21 mins; MS m/z 335.5=[M+H]+

¹H NMR (500 MHz, DMSO-d6) δ 8.40 (s, 1H), 7.37-7.23 (m, 4H), 5.05 (hept, J=6.2 Hz, 1H), 4.70 (hept, J=6.8 Hz, 1H), 1.37 (d, J=6.8 Hz, 6H), 1.26 (d, J=6.2 Hz, 6H)

### Step 2: 3-(4-fluorophenyl)-1-isopropyl-2,4-dioxo-pyrimidine-5-carboxylic acid

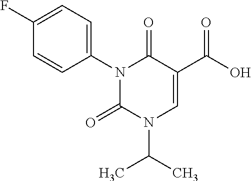 [SMILES: CC(C)n1cc(C(=O)O)c(=O)n(-c2ccc(F)cc2)c1=O]

Isopropyl 3-(4-fluorophenyl)-1-isopropyl-2,4-dioxo-pyrimidine-5-carboxylate (Intermediate C, step 1)(1.02 g, 3.05 mmol) was dissolved in 4M HCl in dioxane (9.3 mL, 37.4 mmol). Water (1.2 mL) was added and the reaction was stirred at 60° C. for 16 hours. The reaction was concentrated before being dried in a vacuum oven at 40° C. for 16 h to afford 3-(4-fluorophenyl)-1-isopropyl-2,4-dioxo-pyrimidine-5-carboxylic acid (884 mg) as a white solid.

LC-MS (Method 2A): Rt 1.05 mins; MS m/z 293.1=[M+H]+

¹H NMR (500 MHz, DMSO-d6) δ 12.64 (s, 1H), 8.58 (s, 1H), 7.44-7.25 (m, 4H), 4.73 (hept, J=6.8 Hz, 1H), 1.39 (d, J=6.8 Hz, 6H)

The compound of the following tabulated Example (Table 20) was prepared analogously to Intermediate C steps 1 & 2 from 2,4-dioxo-3-phenyl-1H-pyrimidine-5-carboxylic acid.

TABLE 20

| Structure and Name | Retention Time, [M + H]+, 1H NMR |
|---|---|
| 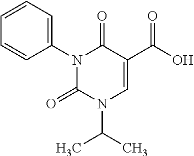 [SMILES: CC(C)n1cc(C(=O)O)c(=O)n(-c2ccccc2)c1=O] | LC-MS (Method 2A): Rt 1.01 mins; MS m/z 275.0 = [M + H]+ ¹H NMR (400 MHz, DMSO-d6) δ 12.69 (s, 1H), 8.59 (s, 1H), 7.57- 7.36 (m, 3H), 7.32-7.23 (m, 2H), 4.73 (hept, J = 6.8 Hz, 1H), 1.39 (d, J = 6.8 Hz, 6H) |
| 1-isopropyl-2,4-dioxo-3-phenyl- | |
| pyrimidine-5-carboxylic acid | |
| (intermediate D) | |

### Intermediate E

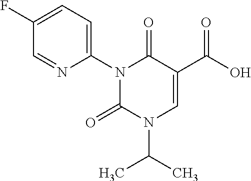 [SMILES: CC(C)n1cc(C(=O)O)c(=O)n(-c2ccc(F)cn2)c1=O]

### Step 1: Diethyl 2-[[(5-fluoro-2-pyridyl)carbamoylamino]methylene]propanedioate

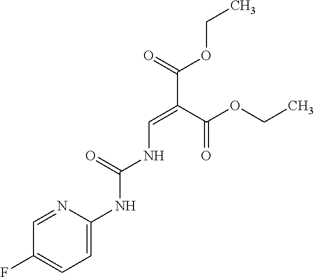 [SMILES: CCOC(=O)C(=CNC(=O)Nc1ccc(F)cn1)C(=O)OCC]

To a stirred solution of 5-fluoropyridin-2-amine (500 mg, 4.46 mmol) in anhydrous DMSO (4.5 mL) was added 1,1′-carbonyldiimidazole (795 mg, 4.91 mmol) and the reaction was stirred at room temperature for 4 h. Diethyl 2-(aminomethylene)propanedioate (835 mg, 4.46 mmol) was added and the reaction was stirred for a further 3 h at room temperature before heating to 80° C. for 18 h.

The reaction was cooled to room temperature, water (10 mL) and EtOAc (15 mL) were added, the organic phase was washed with water (5 mL), dried over Na₂SO₄ and concentrated in vacuo. The crude product was purified by silica chromatography (eluting with a 10-80% EtOAc in heptane gradient) to afford diethyl 2-[[(5-fluoro-2-pyridyl)carbamoylamino]methylene]propanedioate (583 mg) as an off-white solid.

LC-MS (Method 2.5B): 1.60 min; MS m/z 326.2=[M+H]+

¹H NMR (400 MHz, Chloroform-d) δ 9.02 (s, 1H), 8.68 (d, J=12.1 Hz, 1H), 8.23 (dd, J=14.4, 2.8 Hz, 1H), 7.52-7.40 (m, 1H), 4.34 (q, J=7.1 Hz, 2H), 4.27 (q, J=7.1 Hz, 2H), 1.37 (t, J=7.1 Hz, 3H), 1.33 (t, J=7.1 Hz, 3H).

### Step 2: Ethyl 3-(5-fluoro-2-pyridyl)-2,4-dioxo-1H-pyrimidine-5-carboxylate

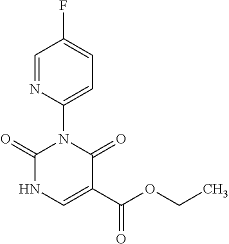 [SMILES: CCOC(=O)c1c[nH]c(=O)n(-c2ccc(F)cn2)c1=O]

To a solution of diethyl 2-[[(5-fluoro-2-pyridyl)carbamoylamino]methylene]propanedioate (Example 9, step 1) (583 mg, 1.79 mmol) in ethanol (10 mL) at room temperature was added sodium ethoxide (21%, 1.2 mL, 3.21 mmol) and the reaction mixture was stirred at room temperature for 1.5 h. The resulting mixture was diluted with EtOAc (30 mL) and washed with 10% citric acid solution (30 mL). The organic layer was separated and the aqueous layer was further extracted with 3:1 CHCl₃/IPA (3×30 mL). The combined organic layers were dried over Na₂SO₄ and concentrated in vacuo to afford crude ethyl 3-(5-fluoro-2-pyridyl)-2,4-dioxo-1H-pyrimidine-5-carboxylate (500 mg) as a yellow gum.

LC-MS (Method 2A): 0.78 min; MS m/z 280.0=[M+H]+

¹H NMR (400 MHz, DMSO-d6) δ 10.23 (s, 1H), 8.60 (d, J=3.1 Hz, 1H), 8.29 (s, 1H), 7.94 (td, J=8.4, 3.1 Hz, 1H), 7.56 (dd, J=8.7, 4.1 Hz, 1H), 4.18 (q, J=7.1 Hz, 2H), 1.23 (t, J=7.1 Hz, 3H).

### Step 3: Ethyl 3-(5-fluoro-2-pyridyl)-1-isopropyl-2,4-dioxo-pyrimidine-5-carboxylate

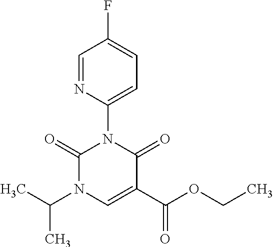 [SMILES: CCOC(=O)c1cn(C(C)C)c(=O)n(-c2ccc(F)cn2)c1=O]

To a suspension of ethyl 3-(5-fluoro-2-pyridyl)-2,4-dioxo-1H-pyrimidine-5-carboxylate (Example 9, step 2)(500 mg, 1.79 mmol) and cesium carbonate (1.75 g, 5.37 mmol) in DMF (10 mL) was added 2-iodopropane (358 μL, 3.58 mmol) and the reaction was heated to 70° C. in a sealed vial for 7 h. The reaction mixture was cooled to room temperature, filtered through Celite and the filter bed washed with MeCN (2×3 mL). Water (15 mL) was added to the filtrate and an off-white suspension formed. The suspension was aged for 30 min before the resultant solid was collected by filtration and washed with water (2×5 mL). The filtrate was diluted with EtOAc (30 mL), the organic fraction separated and the aqueous phase was extracted with EtOAc (2×10 mL). The combined organic fractions were washed with water (10 mL), brine (10 mL), dried over Na₂SO₄ and concentrated in vacuo. The crude product was purified by silica chromatography (eluting with a 10-100% EtOAc in heptane gradient) to afford ethyl 3-(5-fluoro-2-pyridyl)-1-isopropyl-2,4-dioxo-pyrimidine-5-carboxylate (231 mg) as an off-white solid.

LC-MS (Method 2A): 0.99 min; MS m/z 322.1=[M+H]+

¹H NMR (400 MHz, Chloroform-d) δ 8.49 (d, J=3.0 Hz, 1H), 8.36 (s, 1H), 7.58 (ddd, J=8.7, 7.5, 3.0 Hz, 1H), 7.30 (dd, J=8.7, 3.9 Hz, 1H), 4.92 (hept, J=6.8 Hz, 1H), 4.35 (q, J=7.1 Hz, 2H), 1.44 (d, J=6.8 Hz, 6H), 1.35 (t, J=7.1 Hz, 3H).

### Step 4: 3-(5-Fluoro-2-pyridyl)-1-isopropyl-2,4-dioxo-pyrimidine-5-carboxylic acid

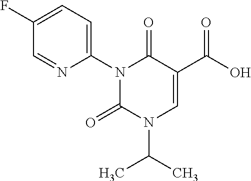 [SMILES: CC(C)n1cc(C(=O)O)c(=O)n(-c2ccc(F)cn2)c1=O]

Isopropyl 3-(5-fluoro-2-pyridyl)-1-isopropyl-2,4-dioxo-pyrimidine-5-carboxylate (Example 9, step 3)(230 mg, 0.686 mmol) was dissolved in 4 M HCl in dioxane (3.0 mL, 12.0 mmol). Water (0.3 mL) was added and the reaction was stirred at 60° C. for 22 h. The reaction was cooled to room temperature and concentrated in vacuo. 10% K₂CO₃ solution (6 mL) and diethyl ether (5 mL) were added to the residue, the aqueous phase was washed with diethyl ether (5 mL) and then acidified to pH 2 with 2 M HCl and extracted with EtOAc (3×5 mL). The combined EtOAc extracts were dried over Na₂SO₄ and concentrated in vacuo to afford 3-(5-Fluoro-2-pyridyl)-1-isopropyl-2,4-dioxo-pyrimidine-5-carboxylic acid (188 mg) as a colourless oil.

LC-MS (Method 2A): 0.92 min; MS m/z 293.8=[M+H]+

¹H NMR (500 MHz, Chloroform-d) δ 12.10 (s, 1H), 8.58 (s, 1H), 8.53 (d, J=2.9 Hz, 1H), 7.66 (ddd, J=8.7, 7.3, 3.0 Hz, 1H), 7.36 (dd, J=8.7, 3.8 Hz, 1H), 4.94 (hept, J=6.8 Hz, 1H), 1.47 (d, J=6.8 Hz, 6H).

The compounds of the following tabulated examples (Table 21) were prepared analogously to Intermediate E steps 1 to 4 from diethyl 2-(aminomethylene)propanedioate and the appropriate amine.

TABLE 21

| | Retention Time, [M + H]+, |
|---|---|
| Structure and Name | 1H NMR |
| 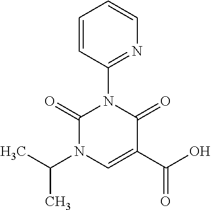 [SMILES: CC(C)n1cc(C(=O)O)c(=O)n(-c2ccccn2)c1=O] | LC-MS (Method 2A): 0.84 min; MS m/z 276.0 = [M + H]+ ¹H NMR (500 MHz, DMSO-d6) δ 8.62-8.54 (m, 2H), 8.02 (td, J = 7.7, 1.9 Hz, 1H), 7.52 (ddd, J = 7.5, 4.8, 1.0 Hz, 1H), 7.49 (d, J = 7.9 Hz, 1H), 4.71 (p, J = 6.8 Hz, 1H), 1.39 (d, J = 6.8 Hz, 6H). |
| 1-isopropyl-2,4-dioxo-3-(2- | |
| pyridyl)pyrimidine-5-carboxylic | |
| acid (Intermediate F) | |
| 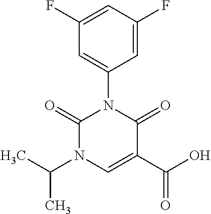 [SMILES: CC(C)n1cc(C(=O)O)c(=O)n(-c2cc(F)cc(F)c2)c1=O] | LC-MS (Method 2A): 1.08 min; MS m/z 311.0 = [M + H]+ ¹H NMR (500 MHz, DMSO-d6) δ 12.63 (s, 1H), 8.57 (s, 1H), 7.48-7.34 (m, 1H), 7.28-7.13 (m, 2H), 4.71 (hept, J = 6.7 Hz, 1H), 1.38 (d, J = 6.8 Hz, 6H). |
| 3-(3,5-difluorophenyl)-1- | |
| isopropyl-2,4-dioxo-pyrimidine- | |
| 5-carboxylic acid (Intermediate G) | |

### Intermediate H

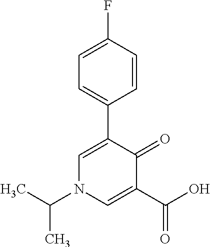 [SMILES: CC(C)n1cc(C(=O)O)c(=O)c(-c2ccc(F)cc2)c1]

### Step 1: Isopropyl 5-(4-fluorophenyl)-1-isopropyl-4-oxo-pyridine-3-carboxylate

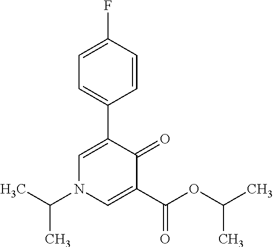 [SMILES: CC(C)OC(=O)c1cn(C(C)C)cc(-c2ccc(F)cc2)c1=O]

A suspension of 5-(4-fluorophenyl)-4-oxo-1H-pyridine-3-carboxylic acid (400 mg, 1.72 mmol), 2-iodopropane (0.51 mL, 5.15 mmol) and potassium carbonate (711 mg, 5.15 mmol) in DMF (10 mL) was stirred at 75° C. for 7 h. The reaction mixture was cooled to room temperature and partitioned between CHCl₃/IPA (2:1, 40 mL) and water (40 mL) and the organic fraction separated. The aqueous layer was extracted with CHCl₃/IPA (2×20 mL) and the combined organic fractions passed through a hydrophobic frit before being concentrated in vacuo to afford isopropyl 5-(4-fluorophenyl)-1-isopropyl-4-oxo-pyridine-3-carboxylate (760 mg) as a yellow viscous oil.

LC-MS (Method 2A): 1.10 min; MS m/z=318.1 [M+H]+

¹H NMR (400 MHz, DMSO-d6) δ 8.26 (d, J=2.5 Hz, 1H), 8.00 (d, J=2.5 Hz, 1H), 7.75-7.60 (m, 2H), 7.27-7.12 (m, 2H), 5.04 (hept, J=6.3 Hz, 1H), 4.43 (hept, J=6.7 Hz, 1H), 1.44 (d, J=6.7 Hz, 6H), 1.26 (d, J=6.3 Hz, 6H).

### Step 2: 5-(4-Fluorophenyl)-1-isopropyl-4-oxo-pyridine-3-carboxylic acid

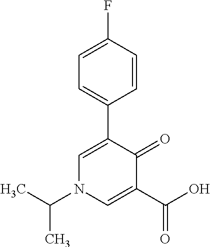 [SMILES: CC(C)n1cc(C(=O)O)c(=O)c(-c2ccc(F)cc2)c1]

A solution of isopropyl 5-(4-fluorophenyl)-1-isopropyl-4-oxo-pyridine-3-carboxylate (Intermediate H, step 1)(760 mg, 1.53 mmol) in 2 M aqueous sodium hydroxide solution (7.7 mL, 7.66 mmol) and THF (15 mL) was stirred at 50° C. for 3 h. The reaction mixture was cooled to room temperature and partitioned between water (15 mL) and diethyl ether (10 mL). The phases were separated and the organic layer was discarded. The aqueous layer was acidified to pH 3 using 1 M aqueous HCl solution before being extracted with DCM (3×10 mL). The combined organics were passed through a phase separator and concentrated in vacuo to afford 5-(4-fluorophenyl)-1-isopropyl-4-oxo-pyridine-3-carboxylic acid (370 mg) as a yellow solid

LC-MS (Method 2A): 1.06 min; MS m/z=276.1 [M+H]+

¹H NMR (500 MHz, DMSO-d6) δ 8.79 (d, J=2.2 Hz, 1H), 8.45 (d, J=2.2 Hz, 1H), 7.80-7.70 (m, 2H), 7.40-7.24 (m, 2H), 4.69 (hept, J=6.7 Hz, 1H), 1.52 (d, J=6.7 Hz, 6H).

### Intermediate I

### Step 1: Tert-butyl-[(2S)-2-iodopropoxy]-dimethyl-silane

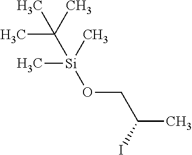

A solution of (2R)-propane-1,2-diol (1.00 g, 13.1 mmol) in DCM (10 mL) was cooled to 0° C. and imidazole (0.89 g, 13.1 mmol) followed by tert-butyl-chloro-dimethyl-silane (1.98 g, 13.1 mmol) were added. The mixture was stirred at 0° C. for 30 mins before being filtered and the filtrate concentrated in vacuo to afford the mono-silylated intermediate.

In a separate flask, imidazole (0.89 g, 13.1 mmol) and triphenylphosphane (3.45 g, 13.1 mmol) were dissolved in DCM (25 mL) at 0° C. before the addition of iodine (3.34 g, 13.1 mmol). The resulting mixture was stirred for 10 mins before the dropwise addition of the monosilylated diol over a period of 5 mins. The mixture was stirred for 30 mins at 0° C. before being washed with aqueous Na₂SO₃ solution (20 mL). The organic fraction was passed through a hydrophobic frit and concentrated in vacuo. The crude residue was purified by silica chromatography (eluting with 100% heptane) to afford tert-butyl-[(2S)-2-iodopropoxy]-dimethyl-silane (744 mg) as a colourless oil.

¹H NMR (400 MHz, Chloroform-d) δ 4.17-4.01 (m, 1H), 3.82 (dd, J=10.5, 5.5 Hz, 1H), 3.61 (dd, J=10.5, 7.8 Hz, 1H), 1.87 (d, J=6.8 Hz, 3H), 0.91 (s, 9H), 0.08 (s, 6H).

### Step 2: [(1R)-2-[tert-butyl(dimethyl)silyl]oxy-1-methyl-ethyl] 1-[(1R)-2-[tert-butyl(dimethyl)silyl]oxy-1-methyl-ethyl]-3-(4-fluorophenyl)-2,4-dioxo-pyrimidine-5-carboxylate

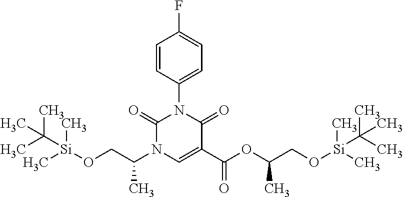 [SMILES: C[C@H](CO[Si](C)(C)C(C)(C)C)OC(=O)c1cn([C@H](C)CO[Si](C)(C)C(C)(C)C)c(=O)n(-c2ccc(F)cc2)c1=O]

A mixture of 3-(4-fluorophenyl)-2,4-dioxo-1H-pyrimidine-5-carboxylic acid (207 mg, 0.826 mmol), potassium carbonate (342 mg, 2.48 mmol) and tert-butyl-[(2S)-2-iodopropoxy]-dimethyl-silane (Intermediate I, step 1) (744 mg, 2.48 mmol) in DMF (2.5 mL) was stirred in a sealed tube at 80° C. for 17 h. The reaction was cooled to room temperature and water (10 mL) and EtOAc (10 mL) were added. The organic fraction was separated and the aqueous layer extracted with EtOAc (2×5 mL) before the combined organic layers were washed with water (2×5 mL), brine (10 mL), dried over MgSO4 and concentrated in vacuo to afford [(1R)-2-[tert-butyl(dimethyl)silyl]oxy-1-methyl-ethyl] 1-[(1R)-2-[tert-butyl(dimethyl)silyl]oxy-1-methyl-ethyl]-3-(4-fluorophenyl)-2,4-dioxo-pyrimidine-5-carboxylate (305 mg) as a yellow oil

LC-MS (Method 3A): 2.15 min; MS m/z=595.4 [M+H]+

¹H NMR (500 MHz, Chloroform-d) δ 8.41 (d, J=8.4 Hz, 1H), 7.20-7.10 (m, 4H), 3.87-3.79 (m, 1H), 3.80-3.69 (m, 2H), 3.68-3.49 (m, 2H), 2.41-2.26 (m, 1H), 1.53-1.42 (m, 3H), 1.34-1.29 (m, 3H), 0.96-0.77 (m, 18H), 0.13-−0.02 (m, 12H).

### Step 3: 3-(4-Fluorophenyl)-1-[(1R)-2-hydroxy-1-methyl-ethyl]-2,4-dioxo-pyrimidine-5-carboxylic acid

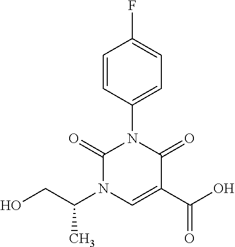 [SMILES: C[C@H](CO)n1cc(C(=O)O)c(=O)n(-c2ccc(F)cc2)c1=O]

[(1R)-2-[tert-butyl(dimethyl)silyl]oxy-1-methyl-ethyl] 1-[(1R)-2-[tert-butyl(dimethyl)silyl]oxy-1-methyl-ethyl]-3-(4-fluorophenyl)-2,4-dioxo-pyrimidine-5-carboxylate (Intermediate I, step 2)(305 mg, 0.513 mmol) was dissolved in 4 M HCl in dioxane (2.3 mL, 9.00 mmol). Water (1 mL) was added and the reaction was stirred in a pressure vial at 60° C. for 1 h. The reaction mixture was concentrated in vacuo and purified by acidic reverse phase chromatography (eluting with 10-100% MeCN (0.1% formic acid) in water (0.1% formic acid)) to afford 3-(4-fluorophenyl)-1-[(1R)-2-hydroxy-1-methyl-ethyl]-2,4-dioxo-pyrimidine-5-carboxylic acid (44 mg) as a white solid.

LC-MS (Method 3A): 0.98 min; MS m/z=308.8 [M+H]+

¹H NMR (400 MHz, DMSO-d6) δ 12.63 (s, 1H), 8.59 (s, 1H), 7.44-7.20 (m, 4H), 5.27-5.03 (m, 1H), 4.71-4.51 (m, 1H), 3.79-3.51 (m, 2H), 1.36 (d, J=7.0 Hz, 3H).

### Intermediate J

### Step 1: Diethyl 2-[(cyclopropylamino)methylene]propanedioate

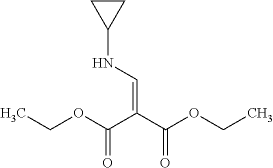 [SMILES: CCOC(=O)C(=CNC1CC1)C(=O)OCC]

Diethyl (ethoxymethylidene)propanedioate (1 g, 4.62 mmol) was added dropwise over the course of 30 mins to cyclopropylamine (0.32 mL, 4.62 mmol) at 0° C. The reaction mixture was allowed to warm to room temperature and stirred for 30 mins before being concentrated in vacuo to afford diethyl 2-[(cyclopropylamino)methylene]propanedioate (920 mg) as a colourless liquid.

LC-MS (Method 2A): 1.09 min; MS m/z 228.1=[M+H]+

¹H NMR (400 MHz, Chloroform-d) δ 9.34-9.07 (m, 1H), 8.15-8.03 (m, 1H), 4.23-4.17 (m, 4H), 2.89-2.81 (m, 1H), 1.33-1.28 (m, 6H), 0.86-0.68 (m, 4H).

### Step 2: Ethyl 1-cyclopropyl-3-(4-fluorophenyl)-2,4-dioxo-pyrimidine-5-carboxylate

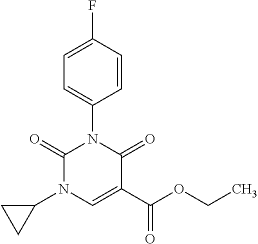 [SMILES: CCOC(=O)c1cn(C2CC2)c(=O)n(-c2ccc(F)cc2)c1=O]

Potassium 2-methylpropan-2-olate (457 mg, 4.07 mmol) was added to an ice cooled stirring solution of 1-fluoro-4-isocyanatobenzene (0.51 mL, 4.48 mmol) and diethyl 2-[(cyclopropylamino)methylene]propanedioate (Intermediate J, step 1)(925 mg, 4.07 mmol) in 1,4-dioxane (5 mL) before being warmed to room temperature and stirred for 3 h. The mixture was partitioned between 1 M aqueous citric acid solution (25 mL) and diethyl ether (25 mL), the organic fraction separated and the aqueous layer extracted with diethyl ether (2×10 mL). The combined organic fractions were concentrated in vacuo and the residue purified by silica chromatography (eluting with a gradient of 0-30% EtOAc in DCM) to afford ethyl 1-cyclopropyl-3-(4-fluorophenyl)-2,4-dioxo-pyrimidine-5-carboxylate (250 mg) as a colourless oil.

LC-MS (Method 2A): 1.03 min; MS m/z 319.1=[M+H]+

¹H NMR (400 MHz, DMSO-d₆) δ 8.29 (s, 1H), 7.33-7.23 (m, 4H), 4.20 (q, J=7.1 Hz, 2H), 3.25-3.14 (m, 1H), 1.24 (t, J=7.1 Hz, 3H), 1.03-0.89 (m, 4H).

### Step 3: 1-Cyclopropyl-3-(4-fluorophenyl)-2,4-dioxo-pyrimidine-5-carboxylic acid

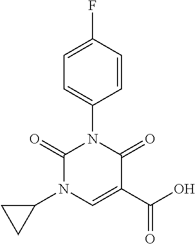 [SMILES: O=C(O)c1cn(C2CC2)c(=O)n(-c2ccc(F)cc2)c1=O]

A solution of ethyl 1-cyclopropyl-3-(4-fluorophenyl)-2,4-dioxo-pyrimidine-5-carboxylate (Intermediate J)(260 mg, 0.817 mmol) in water (1 mL) and 4 M hydrogen chloride in dioxane (3.3 mL, 13.0 mmol) was heated to 50° C. and stirred for 18 h. Additional 4 M hydrogen chloride in dioxane (3.3 mL, 13.0 mmol) was added and the reaction mixture was stirred at 60° C. for a further 1 h after which time the reaction mixture was concentrated in vacuo and the resulting solid freeze dried to afford 1-cyclopropyl-3-(4-fluorophenyl)-2,4-dioxo-pyrimidine-5-carboxylic acid (235 mg) as a beige solid.

LC-MS (Method 2A): 0.98 min; MS m/z 291.0=[M+H]+

¹H NMR (400 MHz, DMSO-d₆) δ 12.61 (s, 1H), 8.41 (s, 1H), 7.43-7.16 (m, 4H), 3.26-3.20 (m, 1H), 1.09-0.78 (m, 4H).

### Intermediate K

### Step 1: 1-Methylpyrrolidine-3-carboxamide

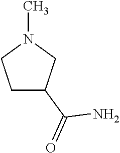

Ammonium chloride (41 mg, 0.774 mmol) was added to a solution of DIPEA (0.20 mL, 1.16 mmol), 1-methylpyrrolidine-3-carboxylic acid (50 mg, 0.387 mmol) and HATU (177 mg, 0.465 mmol) in DCM (2 mL) and the mixture stirred for 18 h at room temperature. The crude mixture was loaded onto a 5 g SCX cartridge and the column was washed sequentially with methanol (2×5 mL) and 7 M NH₃ in MeOH (2×5 mL). The methanolic ammonia fraction was concentrated in vacuo to afford 1-Methylpyrrolidine-3-carboxamide (20 mg) as an orange oil.

¹H NMR (500 MHz, Chloroform-d) δ 6.72 (s, 1H), 5.38-5.13 (m, 1H), 2.91-2.81 (m, 3H), 2.47-2.41 (m, 1H), 2.37 (s, 3H), 2.34-2.28 (m, 1H), 2.24-2.13 (m, 1H), 2.08-1.98 (m, 1H).

### Intermediate L

### Step 1: 1-(4-Fluorophenyl)-2-oxo-pyridine-3-carboxamide

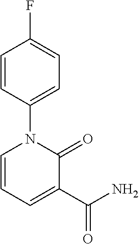 [SMILES: NC(=O)c1cccn(-c2ccc(F)cc2)c1=O]

1-(4-Fluorophenyl)-2-oxo-pyridine-3-carboxylic acid (50 mg, 0.214 mmol) was added to a solution of ammonium chloride (57 mg, 1.07 mmol), HATU (106 mg, 0.279 mmol) and DIPEA (0.28 mL, 1.61 mmol) in DMF (2 mL) and the solution was stirred for 18 h at room temperature. Water (5 mL) was added to the reaction mixture and the resulting solid was collected by filtration, washed with water (3×5 mL) and dried in vacuo at 40° C. to afford 1-(4-Fluorophenyl)-2-oxo-pyridine-3-carboxamide (32 mg) as an off-white solid.

LC-MS (Method 2A): 0.90 min; MS m/z 233.0=[M+H]+

¹H NMR (500 MHz, DMSO-d6) δ 8.85 (s, 1H), 8.43 (dd, J=7.2, 2.2 Hz, 1H), 7.98 (dd, J=6.6, 2.2 Hz, 1H), 7.62 (s, 1H), 7.57-7.49 (m, 2H), 7.43-7.33 (m, 2H), 6.61-6.53 (m, 1H).

### Intermediate M

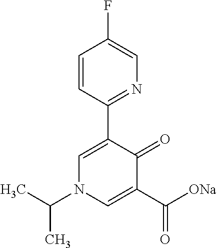

### Step 1: methyl 5-(5-fluoro-2-pyridyl)-4-oxo-1H-pyridine-3-carboxylate

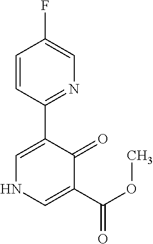 [SMILES: COC(=O)c1c[nH]cc(-c2ccc(F)cn2)c1=O]

A suspension of methyl 5-bromo-4-oxo-1H-pyridine-3-carboxylate (250 mg, 1.08 mmol), tributyl-(5-fluoro-2-pyridyl)stannane (624 mg, 1.62 mmol) and Pd(PPh₃)₄ (125 mg, 0.108 mmol) in toluene (10 mL) was purged with nitrogen for 5 minutes before being stirred at 120° C. for 18 h. The reaction mixture was cooled to room temperature and DCM (20 mL) and water (20 mL) were added. The resultant suspension was filtered and the solid was partitioned between CHCl₃/IPA (2:1, 30 mL) and 1M NaOH solution (30 mL). The phases were separated and the aqueous was extracted with CHCl₃/IPA (2:1, 5×20 mL). The aqueous layer was neutralised with 6M aq. HCl solution before being extracted further with CHCl₃/IPA (2:1, 5×20 mL). The combined CHCl₃/IPA extracts were passed through a hydrophobic frit and concentrated in vacuo to afford methyl 5-(5-fluoro-2-pyridyl)-4-oxo-1H-pyridine-3-carboxylate (310 mg) as an off-white solid.

LC-MS (Method 2A): 0.70 min; MS m/z=249.0 [M+H]+

¹H NMR (500 MHz, DMSO) δ 13.25 (s, 1H), 8.79-8.72 (m, 1H), 8.72-8.65 (m, 1H), 8.64-8.58 (m, 1H), 8.30-8.26 (m, 1H), 7.81-7.74 (m, 1H), 3.75 (s, 3H).

### Step 2: isopropyl 5-(5-fluoro-2-pyridyl)-1-isopropyl-4-oxo-pyridine-3-carboxylate

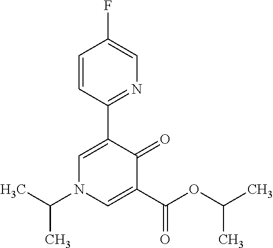 [SMILES: CC(C)OC(=O)c1cn(C(C)C)cc(-c2ccc(F)cn2)c1=O]

A suspension of methyl 5-(5-fluoro-2-pyridyl)-4-oxo-1H-pyridine-3-carboxylate (Intermediate M, step 1)(310 mg, 0.874 mmol), 2-iodopropane (0.26 mL, 2.62 mmol) and potassium carbonate (362 mg, 2.62 mmol) in DMF (10 mL) was heated to 75° C. and stirred for 18 h. The mixture was cooled to room temperature and partitioned between water (30 mL) and ethyl acetate (30 mL). The organic layer was washed with water (15 mL) and brine (2×15 mL) before being dried over MgSO₄ and concentrated in vacuo to afford isopropyl 5-(5-fluoro-2-pyridyl)-1-isopropyl-4-oxo-pyridine-3-carboxylate (120 mg) as a yellow solid.

LC-MS (Method 2A): 0.95 min; MS m/z=319.0 [M+H]+

¹H NMR (500 MHz, DMSO) δ 8.63-8.56 (m, 2H), 8.54-8.50 (m, 1H), 8.32-8.28 (m, 1H), 7.79-7.71 (m, 1H), 5.11-5.00 (m, 1H), 4.57-4.48 (m, 1H), 1.44 (d, J=6.7 Hz, 6H), 1.28 (d, J=6.3 Hz, 6H).

### Step 3: 5-(5-fluoro-2-pyridyl)-1-isopropyl-4-oxo-pyridine-3-carboxylic acid, sodium salt

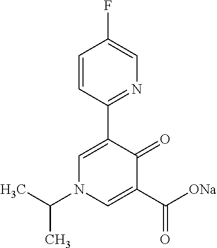

A solution of isopropyl 5-(5-fluoro-2-pyridyl)-1-isopropyl-4-oxo-pyridine-3-carboxylate (Intermediate M, step 2) (120 mg, 0.377 mmol) in 2M sodium hydroxide solution (3.8 mL, 3.76 mmol) and THF (5 mL) was stirred at room temperature for 18 h. The reaction mixture was concentrated in vacuo and the residue was purified by acidic reverse phase column chromatography (eluted with 10-100% MeCN (0.1% formic acid) in water (0.1% formic acid)) to afford 5-(5-fluoro-2-pyridyl)-1-isopropyl-4-oxo-pyridine-3-carboxylic acid, sodium salt (70 mg) as a white solid.

LC-MS (Method 2A): 0.96 min; MS m/z=277.0 [M+H]+

¹H NMR (400 MHz, DMSO) 5 (400 MHz, DMSO) δ 8.67-8.60 (m, 1H), 8.58 (d, J=3.0 Hz, 1H), 8.52 (s, 1H), 8.45 (d, J=2.4 Hz, 1H), 8.28 (d, J=2.5 Hz, 1H), 7.73-7.65 (m, 1H), 4.56-4.40 (m, 1H), 1.42 (d, J=6.7 Hz, 6H).

### Biological Evaluation

Assay 1a—MERTK HTRF KinEASE-TK Assay

Compound inhibition of human MERTK cytoplasmic domain (Carna Biosciences 08-108) enzymatic activity was tested via the HTRF KinEASE-tyrosine kinase (TK) kit (Cisbio 62TK0PEC). Experiments were performed in 20 μl kinase buffer (50 mM HEPES pH 7.5, 10 mM MgCl₂, 4 mM MnCl₂, 2 mM DTT, 1 mM EGTA, 0.01% BRIJ-35), with 2.5 nM of MERTK and the test compounds of interest. Compounds were prepared in a threefold serial dilution in DMSO and added to the assay to give the appropriate final concentration, with the assay DMSO concentration being 1%. Compounds were pre-incubated for 20 minutes with the MERTK enzyme in a 384 well white Optiplate (Perkin Elmer 6007299), followed by the addition of 30 μM ATP and 1 μM TK-Substrate-biotin (Cisbio 61TK0BLC). The enzymatic reaction was carried out for 30 min at room temperature. 10 μL TK Antibody-Cryptate/Streptavidin-XL665 (Cisbio 610SAXLG) was then added for 60 min, including EDTA containing detection buffer (Cisbio 62SDBRDF) to stop the reaction. The plate was read on a BMG Pherastar FSX using a HTRF 337/620/665 optical module to excite the terbium donor at 337 nm, and record donor emission at 620 nm and acceptor fluorophore emission at 665 nm. From this the FRET ratio (acceptor 665 nm/donor 620 nm) for each data point was calculated. Data were normalised to measurements in the absence of competing compound (0% inhibition) and in the presence of 10 μM control compound (100% inhibition). Competition curves were then fit to a 4 parameter logistic equation including a variable slope factor to determine the IC₅₀ (compound concentration that inhibits 50% of MERTK activity).

Assay 1b—AXL HTRF KinEASE-TK Assay

Compound inhibition of human AXL cytoplasmic domain (Thermo Fisher A31515) enzymatic activity was tested via the HTRF KinEASE-tyrosine kinase (TK) kit (Cisbio 62TK0PEC). Experiments were performed in 20 μl enzymatic buffer (Cisbio 62EZBFDD) supplemented with 5 mM MgCl₂, 1 mM MnCl₂, 1 mM DTT, 50 nM SEB (Cisbio, 61SEBALB), with 5 nM of AXL and the test compounds of interest. Compounds were prepared in a threefold serial dilution in DMSO and added to the assay to give the appropriate final concentration, with the assay DMSO concentration being 1%. Compounds were pre-incubated for 20 minutes with the AXL enzyme in a 384 well white Optiplate (Perkin Elmer 6007299), followed by the addition of 15 μM ATP (15× Km) and 0.13 μM TK-Substrate-biotin (Cisbio 61TK0BLC). The enzymatic reaction was carried out for 30 min at room temperature. 10 μL TK Antibody-Cryptate/Streptavidin-XL665 (Cisbio 610SAXLG) was then added for 60 min, including EDTA containing detection buffer (Cisbio 62SDBRDF) to stop the reaction. The plate was read on a BMG Pherastar FSX using a HTRF 337/620/665 optical module to excite the terbium donor at 337 nm, and record donor emission at 620 nm and acceptor fluorophore emission at 665 nm. From this the FRET ratio (acceptor 665 nm/donor 620 nm) for each data point was calculated. Data were normalised to measurements in the absence of competing compound (0% inhibition) and in the presence of 10 μM control compound (100% inhibition). Competition curves were then fit to a 4 parameter logistic equation including a variable slope factor to determine the IC₅₀ (compound concentration that inhibits 50% of AXL activity).

### Results

| | Assay 1a | Assay 1b |
|---|---|---|
| Example | Mer IC₅₀ 30 uM | Axl IC₅₀ 15 uM |
| Number | ATP (nM) | ATP (nM) |
| 1 | 68 | |
| 1.1 | 75 | |
| 1.2 | 20 | |
| 1.3 | 182 | |
| 1.4 | 33 | |
| 2 | 59 | |
| 2.1 | 196 | |
| 2.2 | 54 | |
| 3 | 55 | 200 |
| 4 | 3* | |
| 4.1 | 37 | |
| 5 | 60 | |
| 5.1 | 70 | |
| 5.2 | 17* | |
| 5.3 | 11* | |
| 5.4 | 535 | |
| 6 | 52 | |
| 7 | 392 | |
| 7.1 | 54 | |
| 8 | 154 | |
| 8.1 | 4 | 28 |
| 8.2 | 10 | 10 |
| 8.3 | 133 | |
| 8.4 | 245 | |
| 9 | 14 | 11 |
| 9.1 | 12 | 13 |
| 10 | 5 | 29 |
| 10.1 | 6 | 14 |
| 10.2 | 2 | 20 |
| 11 | 4 | 77 |
| 12 | 11 | 54 |
| 12.1 | 722 | |
| 12.2 | 20 | 16 |
| 12.3 | 56 | 19 |
| 12.4 | 8 | 6 |
| 12.5 | 3 | 5 |
| 12.6 | 3 | 31 |
| 12.7 | 16 | 36 |
| 12.8 | 134 | |
| 13 | 1 | 6 |
| 13.1 | 4 | 10 |
| 14 | 14 | 10 |
| 15 | 22 | 10 |
| 16 | 19 | 264 |
| 16.1 | 4 | 27 |
| 16.2 | 3 | 18 |
| 16.3 | 1 | 3 |
| 16.4 | 13 | 282 |
| 17 | 4 | 240 |
| 18 | 2 | 34 |
| 18.1 | 1 | 10 |
| 19 | 137 | |
| 20 | 3 | 7 |
| 20.1 | 5 | 5 |
| 20.2 | 1 | 7 |
| 21 | 57 | 247 |
| 21.1 | 9 | 46 |
| 22 | 1 | 5 |
| 23 | 3 | 12 |
| 23.1 | 13 | |
| 24 | 6 | 21 |
| 25 | 29 | 76 |
| 26 | 19 | 360 |
| 27.1 | 11 | 3^{#} |
| 27.2 | 17 | 4^{#} |
| 27.3 | 31 | 6^{#} |
| 27.4 | 14 | |
| 27.5 | 15 | 9 |
| 28.2 | 31 | 60 |
| 30 | 13 | 870 |
| 31 | 6 | 12 |
| 32 | 17 | 310 |
| 33 | 8 | 38 |
| 34 | 43 | 296 |
| 35 | 4 | 60 |
| 36 | 20 | 5 |
| 37 | 3 | 7 |
| 37.1 | 2 | 5 |

*Maximal inhibition achieved 40%
^{#}1 μM ATP used in assay

### Assay 2—MERTK Cellular Target Engagement (TE) Assay

Compound affinities for recombinant human MERTK expressed in live cells were assessed via the Promega NanoBRET TE assay technology. Human Embryonic Kidney (HEK) 293 cells were transiently transfected with the MERTK-Nanoluc fusion vector (Promega Custom Cat CS1810C272) using Fugene HD reagent (Promega E2311). 24 h later, experiments were performed in 40 μl OptiMEM (Invitrogen 15392402), dispensing 5000 cells/well into a 384 well white Optiplate (Perkin Elmer 6007299), 0.5 μM fluorescent tracer K9 (Promega CS1810C152, prepared as 20× stock in tracer dilution buffer N219B) and the test compounds of interest. Compounds were prepared in a threefold serial dilution in DMSO and added to the assay to give the appropriate final concentration, with the assay DMSO concentration being 0.75%. Incubations were conducted for 2 h at 37° C., after which 20 μl NanoGlo substrate/inhibitor mix (Promega N2161) was added to each well for 2-3 min. The plate was then read on a BMG Pherastar FSX using a NanoBRET filter to record donor emission at 450 nm and acceptor fluorophore emission at 610 nm. From this the BRET ratio (acceptor 610 nm/donor 450 nm) for each data point was calculated. Data were normalised to measurements in the absence of competing compound (0% inhibition) and in the presence of 10 μM control compound (100% inhibition). Competition curves were then fit to a 4 parameter logistic equation including a variable slope factor to determine the IC₅₀ (compound concentration that inhibits 50% tracer K9 specific binding).

### Assay 3—AXL Cellular Target Engagement (TE) Assay

Compound affinities for recombinant human AXL expressed in live cells were assessed via the Promega NanoBRET TE assay technology. Human Embryonic Kidney (HEK) cells were transiently transfected with the AXL-Nanoluc fusion vector (Promega Cat NV1071) using Fugene HD reagent (Promega E2311). 24 h later, experiments were performed in 40 μl OptiMEM (Invitrogen 15392402), dispensing 5000 cells/well into a 384 well white Optiplate (Perkin Elmer 6007299), 0.5 μM fluorescent tracer K5 (Promega NV2530, prepared as 20× stock in tracer dilution buffer N219B) and the test compounds of interest. Compounds were prepared in a threefold serial dilution in DMSO and added to the assay to give the appropriate final concentration, with the assay DMSO concentration being 0.75%. Incubations were conducted for 2 h at 37° C., after which 20 μl NanoGlo substrate/inhibitor mix (Promega N2161) was added to each well for 2-3 min. The plate was then read on a BMG Pherastar FSX using a NanoBRET filter to record donor emission at 450 nm and acceptor fluorophore emission at 610 nm. From this the BRET ratio (acceptor 610 nm/donor 450 nm) for each data point was calculated. Data were normalised to measurements in the absence of competing compound (0% inhibition) and in the presence of 10 μM control compound (100% inhibition). Competition curves were then fit to a 4 parameter logistic equation including a variable slope factor to determine the IC₅₀ (compound concentration that inhibits 50% tracer K5 specific binding).

| | Assay 2 | Assay 3 |
|---|---|---|
| Example | Cell TE MER | Cell TE Axl |
| Number | IC50 (nM) | IC50 (nM) |
| 1 | 175 | 145 |
| 1.1 | 1750 | 2.9 |
| 1.2 | 28 | 114 |
| 1.3 | 260 | 24.8 |
| 1.4 | 2187 | |
| 2 | 46.6 | 173 |
| 2.1 | 163 | 1411 |
| 2.2 | 23.7 | 189 |
| 3 | 26.3 | 7 |
| 4 | 2900 | |
| 4.1 | 131 | 133 |
| 5 | 96.1 | 97.5 |
| 5.1 | 135 | 335 |
| 5.2 | 174 | 363 |
| 5.3 | 41.9 | 46.7 |
| 6 | 110 | 110 |
| 12 | 80.4 | 114 |
| 12.2 | 18.3 | 28.8 |
| 12.3 | 244 | 80.8 |
| 12.4 | 45.4 | 12.8 |
| 13 | 6.7 | 7.1 |
| 13.1 | 12.4 | 15.9 |
| 14 | 75.9 | 31 |
| 15 | 133 | 33.2 |
| 27.5 | 30.5 | 14 |
| 28.2 | 95.6 | 83.3 |
| 30 | 244 | 339 |
| 31 | 26.1 | 34.9 |
| 32 | 80.8 | 127 |
| 33 | 74.3 | 63.8 |
| 36 | 68.1 | 25.7 |
| 37 | 41.4 | 33.5 |
| 37.1 | 8.3 | 6.1 |

### Assay 5—Ba/F3 Cell Survival Assays.

The inhibitory potencies of compounds were assessed in a Ba/F3 cell survival assay, using the Ba/F3 Pro B-cell line, recombinantly expressing a BCR-human AXL kinase domain fusion protein (pACD320/AXL, Advanced Cellular Dynamics Inc), TEL-human MERTK fusion domain (pACD1000/MERTK, Advanced Cellular Dynamics Inc), TEL-human Tyro3 fusion domain (pACD1000/TYRO3, Advanced Cellular Dynamics Inc), or control TEL-none (ACD320, Advanced Cellular Dynamics Inc) BRC-none constructs (ACD1000, Advanced Cellular Dynamics Inc). 10 ng/mL recombinant murine IL3 (R&D, 403-ML-010) was included in the media when propagating negative control TEL-none or BCR-none lines only. Cells were maintained in RMPI 1640 (Corning 15-040-CV) culture media containing 10% heat inactivated semi-synthetic FBS (RMBIO FGR-BBT), 2 mM L-Glutamine (Sigma, G7513), 100 IU/mL Penicillin-10 mg/mL Streptomycin (Sigma, P4333), and 500 ng/mL Puromycin (Sigma, P8833). Experiments were performed in 50 μl growth media, including the test compounds of interest, and dispensing 5000 cells/well in logarithmic-phase growth into a 384 well white Optiplate (Perkin Elmer 6007299). Compounds were prepared in a threefold serial dilution in DMSO and added to the assay to give the appropriate final concentration. The final assay DMSO concentration was 0.25%. Compound incubations were conducted for 48 h at 37° C./5% CO₂, after which cell plate was equilibrated at room temperature for 20 minutes prior to assessing viability. 15 μl of Promega CellTiter-Glo reagent (G7572) was added to each well for 2 min on an orbital shaker to induce cell lysis.

The plate was then equilibrated for 5 min at room temperature and luminescence read on a BMG Pherastar FSX using the LUM plus optic module. Data were normalised to measurements in the absence of competing compound (0% inhibition). Competition curves were then fit to a 4 parameter logistic equation including a variable slope factor to determine the IC₅₀ (compound concentration causing a half-maximal inhibition of cell survival).

| Example | TEL-MERTK | BCR-Axl Ba/F3 | TEL-Tyro3 |
|---|---|---|---|
| Number | Ba/F3 IC50 (nM) | IC50 (nM) | Ba/F3 IC50 (nM) |
| 3 | 314 | 45 | 956 |
| 8.1 | 17 | 7 | 48 |
| 8.2 | 30 | 3 | 120 |
| 9 | 85 | 6 | 252 |
| 9.1 | >2500 | 25 | >2500 |
| 10 | 24 | 28 | 148 |
| 10.1 | 16 | 16 | 119 |
| 10.2 | 17 | 14 | 394 |
| 11 | 52 | 32 | 251 |
| 12.2 | 56 | 48 | 238 |
| 12.3 | 431 | 64 | 1320 |
| 12.4 | 100 | 12 | 313 |
| 12.5 | 38 | 5 | 428 |
| 12.6 | 16 | 6 | 110 |
| 13 | 31 | 13 | 212 |
| 13.1 | 50 | 22 | 179 |
| 14 | 80 | 7 | 592 |
| 15 | 159 | 14 | 673 |
| 16 | 14 | 22 | 931 |
| 16.1 | 70 | 76 | 1930 |
| 16.2 | 14 | 15 | 144 |
| 16.3 | 14 | 14 | 271 |
| 16.4 | 195 | 154 | >2500 |
| 17 | 25 | 31 | 186 |
| 18 | 3 | 3 | 165 |
| 18.1 | 13 | 13 | 250 |
| 20 | 20 | 16 | 272 |
| 20.1 | 27 | 15 | 452 |
| 20.2 | 5 | 2 | 220 |
| 21 | 217 | 203 | 2390 |
| 21.1 | 35 | 46 | 861 |
| 22 | 10 | 10 | >2500 |
| 23 | 2 | 2 | 429 |
| 23.1 | 15 | 15 | 568 |
| 24 | 6 | 6 | 37 |
| 25 | 54 | 27 | 91 |
| 26 | 55 | 83 | 1420 |
| 27.1 | 67 | 15 | 881 |
| 27.2 | 49 | 13 | 376 |
| 27.3 | 44 | 21 | 631 |
| 27.4 | 44 | 14 | 377 |
| 27.5 | 9 | 2 | 107 |
| 30 | 466 | 470 | 1330 |
| 33 | 58 | 30 | 155 |
| 34 | 144 | 73 | 128 |
| 35 | 14 | 8 | 116 |
| 36 | 160 | 34 | 873 |
| 37 | 50 | 17 | 251 |
| 37.1 | 8 | 2 | 43 |

All compounds had an IC₅₀>2500 nM on the control cell lines except Example 20 (830 nM at the BCR-Ba/F3)

### Numbered Paragraphs

The following numbered paragraphs serve to define particular aspects and embodiments of the invention.
1. A compound of Formula (I) or a pharmaceutically acceptable salt thereof, as shown below:

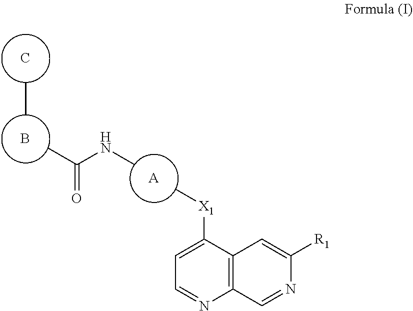

wherein:
R₁ is selected from:
(i) a cycloalkyl, aryl, heteroaryl or heterocyclyl ring,
   each of which is optionally substituted on an available carbon atom by one or more R_{100C} substituents;
   wherein each R_{100C} substituent present is independently selected from halo, hydroxy, cyano, Rₐ, NRₐR_{b}, ORₐ, C(O)Rₐ, C(O)ORₐ, OC(O)Rₐ, C(O)N(R_{b})Rₐ, C(O)N(R_{b})ORₐ, N(R_{b})C(O)Rₐ, S(O)_{y}Rₐ (wherein y is 0, 1 or 2), S(O)₂N(R_{b})Rₐ, N(R_{b})S(O)₂Rₐ, (CH₂)_{z}Rₐ or (CH₂)_{z}NRₐR_{b} (where z is 1, 2 or 3);
      wherein Rₐ is selected from:
      (a) (1-4C)alkyl which is optionally substituted by one or more substituents independently selected from halo, hydroxy, cyano, amino, (3-6C)cycloalkyl, (1-4C)alkylamino, di-(1-4C)alkylamino or (1-4C)alkoxy and wherein any alkyl or cycloalkyl moiety present in such substituent groups is optionally further substituted by halo, hydroxy and/or (1-2C)alkoxy; or
      (b) aryl, heteroaryl, cycloalkyl, heterocyclyl or heterocyclyl(1-2C)alkyl, each of which is optionally substituted on an available carbon atom by one or more substituents independently selected from halo, hydroxy, cyano, amino, (1-4C)alkyl, (1-4C)alkylamino, di-(1-4C)alkylamino, (1-4C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are each independently selected from hydrogen or (1-4C)alkyl, and wherein any alkyl moiety present in a substituent group is optionally further substituted by halo, hydroxy and/or (1-2C)alkoxy;
      and R_{b} is selected from hydrogen or (1-2C)alkyl;
      and, when R₁ or Rₐ is a heteroaryl, heterocyclyl or heterocyclyl(1-2C)alkyl, an available nitrogen atom (where valency permits) is optionally substituted by one or more R_{100N};
      wherein each R_{100N} is selected from:
         (a) (1-4C)alkyl which is optionally substituted by halo, hydroxy, cyano, amino and/or (1-4C)alkoxy (optionally substituted by halo and/or (1-2C)alkoxy); or
         (b) (3-6C)cycloalkyl, phenyl, heterocyclyl or heteroaryl, each of which is optionally further substituted on an available carbon atom by one or more substituents independently selected from halo, hydroxy, cyano, amino, (1-4C)alkyl, (1-4C)alkylamino, di-(1-4C)alkylamino, (1-4C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are as defined above, and wherein any alkyl moiety present in a substituent group is optionally further substituted by halo, hydroxy and/or (1-2C)alkoxy;
         and an available ring nitrogen atom (where valency permits) is optionally further substituted by (1-4C)alkyl optionally substituted by halo, hydroxy, cyano, amino and/or (1-4C)alkoxy (which is optionally further substituted by halo and/or (1-2C)alkoxy); or
(ii) a group:

-L-R₂;
wherein:
L is a linking group selected from: —O—; —CO—; —COO—; —OCO—; —NR₁ₐ—; —CONR₁ₐ—; —CONR₁ₐ—O—; —NR₁ₐCO—; —NR₁ₐCOO—; or —NR₁ₐCONR₁ₐ—;
R₁ₐ or R_{1b} are both independently selected from hydrogen or (1-4C)alkyl; and
R₂ is selected from:
(a) hydrogen;
(b) (1-4C)alkyl which is optionally substituted by halo, hydroxy, cyano, amino, (1-4C)alkylamino, di-(1-4C)alkylamino and/or (1-4C)alkoxy (which is optionally further substituted by halo and/or (1-4C)alkoxy); or
(c) a cycloalkyl, aryl, heteroaryl, heterocyclyl, cycloalkyl(1-2C)alkyl, aryl(1-2C)alkyl, heteroaryl(1-2C)alkyl or heterocyclyl(1-2C)alkyl ring,
   wherein each ring system is optionally substituted on an available carbon atom by one or more R_{101C} substituents;
   wherein each R_{101C} substituent is independently selected from halo, hydroxy, cyano, R_{c}, NR_{d}R_{c}, OR_{c}, C(O)R_{c}, C(O)OR_{c}, OC(O)R_{c}, C(O)N(R_{d})R_{c}, C(O)N(R_{d})OR_{c}, N(R_{d})C(O)R_{c}, S(O)_{y}R_{c} (wherein y is 0, 1 or 2), S(O)₂N(R_{d})R_{c}, N(R_{d})S(O)₂R_{c}, (CH₂)_{z}R_{c} or (CH₂)_{z}NR_{c}R_{d} (where z is 1, 2 or 3);
   and wherein R_{c} is selected from:
   1. hydrogen;
   2. (1-4C)alkyl which is optionally substituted by one or more substituents independently selected from halo, hydroxy, cyano, amino, (3-6C)cycloalkyl, (1-4C)alkylamino, di-(1-4C)alkylamino or (1-4C)alkoxy and wherein any alkyl or cycloalkyl moiety present in such substituent groups is optionally further substituted by halo, hydroxy and/or (1-2C)alkoxy; or
   3. a cycloalkyl, aryl, heteroaryl, heterocyclyl or heterocyclyl(1-2C)alkyl ring,
      wherein each ring system is optionally substituted on an available carbon atom by one or more substituents independently selected from halo, hydroxy, cyano, amino, (1-4C)alkyl, (1-4C)alkylamino, di-(1-4C)alkylamino, (1-4C)alkoxy, C(O)R_{c1}, C(O)OR_{c1}, OC(O)R_{c1}, C(O)N(R_{d1})R_{c1}, C(O)N(R_{d1})OR_{c1} N(R_{d1})C(O)R_{c1}, S(O)_{y}R_{c1} (wherein y is 0, 1 or 2), S(O)₂N(R_{b1})R_{c1} or N(R_{d1})S(O)₂R_{c1}, wherein R_{c1} and R_{d1} are each independently selected from hydrogen or (1-4C)alkyl, and wherein any alkyl moiety present in a substituent group is optionally further substituted by halo, hydroxy and/or (1-2C)alkoxy;
      and R_{d} is selected from hydrogen or (1-2C)alkyl;
   and, when R₂ or R_{c} is a heteroaryl or heterocyclyl ring, an available nitrogen atom (where valency permits) is optionally further substituted by one or more R_{101N};
   wherein each R_{101N} substituent is selected from:
   (a) (1-4C)alkyl which is optionally substituted by one or more substituents independently selected from halo, hydroxy, cyano, amino or (1-4C)alkoxy (which is optionally further substituted by halo and/or (1-2C)alkoxy);
   (b) (3-6C)cycloalkyl, phenyl, or a carbon-linked heterocyclyl or heteroaryl ring, each of which is optionally further substituted by one or more substituents independently selected from halo, hydroxy, cyano, amino, (1-4C)alkyl, (1-4C)alkylamino, di-(1-4C)alkylamino, (1-4C)alkoxy, C(O)R_{c1}, C(O)OR_{c1}, OC(O)R_{c1}, C(O)N(R_{d1})R_{c1}, C(O)N(R_{d1})OR_{c1} N(R_{d1})C(O)R_{c1}, S(O)_{y}R_{c1} (wherein y is 0, 1 or 2), S(O)₂N(R_{b1})R_{c1} or N(R_{d1})S(O)₂R_{c1}, wherein R_{c1} and R_{d1} are as defined above and wherein any alkyl moiety present in a substituent group is optionally further substituted by halo, hydroxy and/or (1-2C)alkoxy;
or, when L is a linking group selected from —NR₁ₐ—, —CONR₁ₐ—, or —NR₁ₐCO, R₁ₐ and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N};
or, when L is —NR₁ₐCONR_{1b}—, R_{1b} and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N};
X₁ is —NH— or —O—;
Ring A is selected from the following:

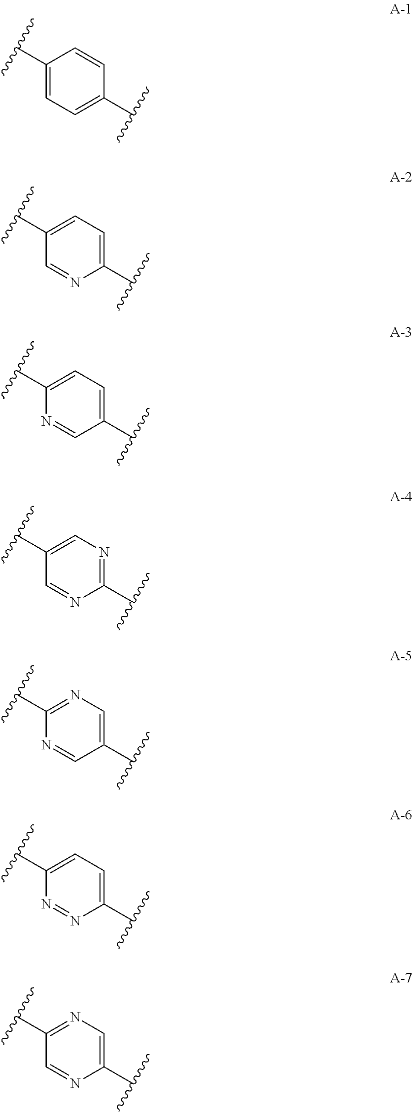

wherein [symbol] represents the respective points of attachment of Ring A to the rest of the compound of Formula (I) and wherein Ring A may be optionally substituted on an available carbon atom by one or more substituents independently selected from halo, (1-4C)alkyl and (1-4C)alkoxy;
Ring B is a 5- or 6-membered heteroaryl or a heterocyclic ring comprising one to three N atoms, linked via a carbon atom to the amide bond in the compound of Formula (I) and wherein:
   (a) a heteroaryl ring is optionally substituted on an available carbon atom by one or more substituents independently selected from hydroxy, halo, cyano, (1-6C)alkyl or (1-6C)alkoxy; or on an available nitrogen atom by (1-6C)alkyl; and wherein any alkyl moiety present in a substituent group is optionally further substituted by halo, hydroxy and/or (1-2C)alkoxy;
   and
   (b) a heterocyclyl ring is optionally substituted on an available carbon atom by one or more substituents independently selected from oxo, hydroxy, halo, cyano, (1-6C)alkyl, (1-6C)alkoxy; or on an available nitrogen atom by (1-6C)alkyl; and wherein any alkyl moiety present in a substituent group is optionally further substituted by halo, hydroxy and/or (1-2C)alkoxy;
Ring C is selected from the following:

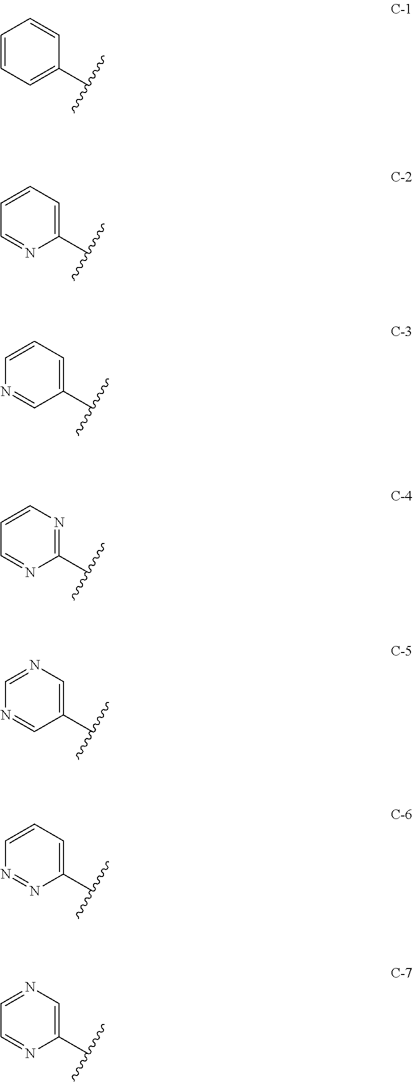

wherein [symbol] represents the point of attachment of Ring C to Ring B and wherein Ring C is optionally substituted on an available carbon atom by one or more substituents independently selected from hydroxy, halo, cyano, (1-6C)alkyl or (1-6C)alkoxy; or on an available nitrogen atom by (1-6C)alkyl; and wherein any alkyl moiety present in a substituent group is optionally further substituted by halo, hydroxy and/or (1-2C)alkoxy.
2. A compound, or a pharmaceutically acceptable salt thereof, according to paragraph 1, wherein Ring B is a 5- or 6-membered heteroaryl having the formula B-1 or B-2 shown below:

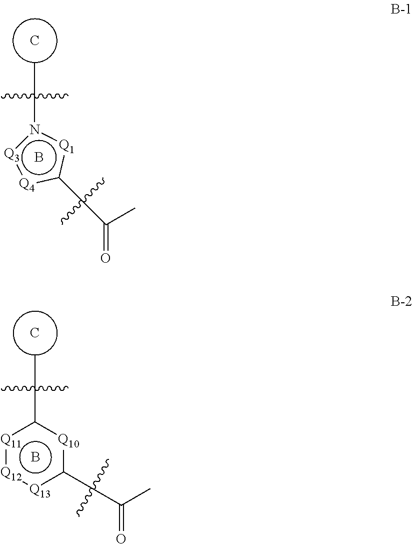

wherein
Q₁, Q₃ and Q₄ are CH or N;
wherein one of Q₁, Q₃ and Q₄ is N;
Q₁₀, Q₁₁, Q₁₂ and Q₁₃ are CR_{q2} or N;
wherein up to two of Q₁₀, Q₁₁, Q₁₂ and Q₁₃ can be N and R_{q2} is hydrogen or a substituent selected from hydroxy, halo, cyano, (1-2C)alkyl or (1-2C)alkoxy;
or Ring B is a 6-membered heterocyclic ring of the formula B-3 or B-4 shown below:

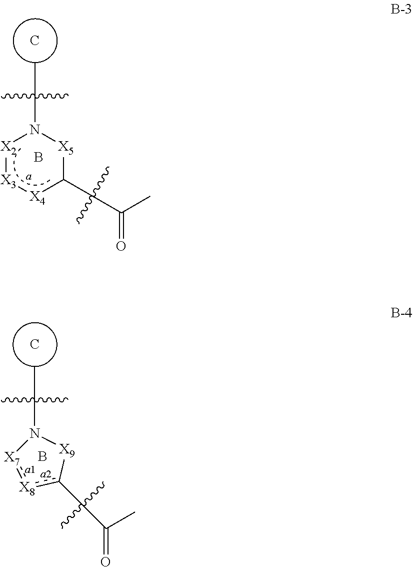

wherein
X₂, X₃ and X₄ are selected from C═O, CRₓ, CHRₓ or NR_{y};
X₅ is selected from C═O, CHRₓ or NR_{y};
wherein no more than two of X₂, X₃, X₄ and X₅ can be N and no more than two of X₂, X₃, X₄ and X₅ can be C═O;
Rₓ is hydrogen or a substituent selected from hydroxy, halo, cyano, (1-4C)alkyl, (1-4C)alkoxy;
R_{y} is hydrogen or (1-4C)alkyl;
and wherein any alkyl moiety present in a Rₓ or R_{y} substituent group is optionally further substituted by halo, hydroxy and/or (1-2C)alkoxy; and
[symbol] represents an optional double bond between X₂ and X₃ and/or between X₄ and the adjacent ring carbon atom linked to the amide bond;
X₇ and X₈ are selected from CRₓ CHRₓ or NR_{y};
X₉ is selected from C═O, CHRₓ or NR_{y};
wherein one of X₇, X₈ and X₉ can be N;
Rₓ and R_{y} are as defined above; and
both a1 and a2 are single bonds or one of a1 or a2 is a double bond.
3. A compound of Formula (Ia) or a pharmaceutically acceptable salt thereof, according to paragraph 1 or paragraph 2, as shown below:

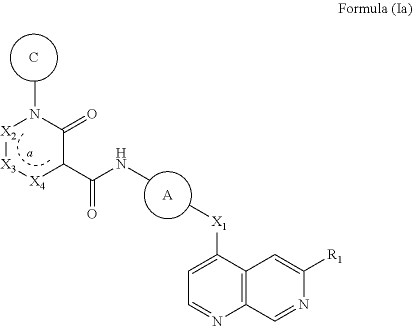

wherein R₁, X₁, Ring A and Ring C are as defined in paragraph 1 and wherein in Formula (Ia):
[symbol] represents an optional double bond between X₂ and X₃ and/or between X₄ and the adjacent ring carbon atom linked to the amide bond;
X₂ is N or CR₂ₓ wherein R₂ₓ is hydrogen, (1-4C)alkyl or (1-4C)alkoxy when there is a double bond between X₂ and X₃ or
X₂ is CO, CH₂, CHR₂ₓ or NR_{2y} wherein R_{2y} is hydrogen or (1-4C)alkyl when there is a single bond between X₂ and X₃;
X₃ is N or CR₃ wherein R₃ₓ is hydrogen, (1-4C)alkyl or (1-4C)alkoxy when there is a double bond between X₂ and X₃ or
X₃ is CO, CH₂, CHR₃ₓ or NR_{3y} wherein R_{3y} is hydrogen or (1-4C)alkyl when there is a single bond between X₂ and X₃;
X₄ is N or CR₄ₓ wherein R₄ₓ is hydrogen, (1-4C)alkyl or (1-4C)alkoxy when there is a double bond between X₄ and the adjacent ring carbon atom linked to the amide bond or
X₄ is CO, CH₂, CHR₄ₓ or NR_{4y} wherein R_{4y} is hydrogen or (1-4C)alkyl when there is a single bond between X₄ and the adjacent ring carbon atom linked to the amide bond;
wherein any alkyl moiety present in a ring substituent group is optionally further substituted by halo, hydroxy or (1-2C)alkoxy;
and provided that:
(i) a maximum of one of X₂, X₃ and X₄ is N or NR_{2y}, NR_{3y} or R_{4y} respectively;
(ii) a maximum of one of X₂, X₃ and X₄ is CO;
(iii) when X₂ is N then X₃ is CO.
4. A compound according to any one of paragraphs 1 to 3, or a pharmaceutically acceptable salt or solvate thereof, wherein R₁ is selected from:
(i) a cycloalkyl, aryl, heteroaryl or heterocyclyl ring,
   each of which is optionally substituted on an available carbon atom by one or more R_{100C} substituents;
   wherein each R_{100C} substituent is independently selected from halo, hydroxy, cyano, Rₐ, NRₐR_{b}, ORₐ, C(O)Rₐ, C(O)ORₐ, OC(O)Rₐ, C(O)N(R_{b})Rₐ, C(O)N(R_{b})ORₐ, N(R_{b})C(O)Rₐ, S(O)_{y}Rₐ (wherein y is 0, 1 or 2), S(O)₂N(R_{b})Rₐ, N(R_{b})S(O)₂Rₐ, (CH₂)_{z}Rₐ or (CH₂)_{z}NRₐR_{b} (where z is 1, 2 or 3);
   wherein Rₐ is selected from:
      (a) (1-2C)alkyl which is optionally substituted by one or more substituents independently selected from halo, hydroxy, cyano, amino, (3-4C)cycloalkyl, (1-2C)alkylamino, di-(1-2C)alkylamino or (1-2C)alkoxy and wherein any alkyl or cycloalkyl moiety present in such substituent groups is optionally further substituted by halo, hydroxy and/or (1-2C)alkoxy; or
      (b) aryl, heteroaryl, heterocyclyl or heterocyclyl(1-2C)alkyl, each of which is optionally substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ, or N(R_{b1})S(O)₂Rₐ₁, wherein Rₐ₁ and R_{b1} are each independently selected from hydrogen or (1-2C)alkyl, and wherein any alkyl moiety present in a substituent group is optionally further substituted by halo, hydroxy and/or (1-2C)alkoxy;
      and R_{b} is selected from hydrogen or (1-2C)alkyl;
      and, when R₁ or Rₐ is a heteroaryl, heterocyclyl or heterocyclyl(1-2C)alkyl, an available nitrogen atom (where valency permits) is optionally substituted by one or more R_{100N}:
      wherein R_{100N} is selected from:
      (a) (1-4C)alkyl which is optionally substituted by halo, hydroxy, cyano, amino and/or (1-1C)alkoxy (optionally substituted by halo and/or (1-2C)alkoxy); or
      (b) heterocyclyl or heteroaryl, each of which is optionally further substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are as defined above, and wherein any alkyl moiety present in a substituent group is optionally further substituted by halo, hydroxy and/or (1-2C)alkoxy;
      and an available ring nitrogen atom (where valency permits) is optionally further substituted by (1-4C)alkyl optionally substituted by halo, hydroxy, cyano, amino and/or (1-2C)alkoxy; or
(ii) a group:

-L-R₂;
wherein:
L is a linking group selected from: —O—; —CO—; —COO—; —OCO—; —NR₁ₐ—; —CONR₁ₐ—; —CONR₁ₐ—O—; —NR₁ₐCO—; —NR₁ₐCOO—; or NR₁ₐCONR_{1b}—;
R₁ₐ and R_{1b} are independently selected from hydrogen or (1-2C)alkyl; and R₂ is selected from:
(a) hydrogen;
(b) (1-4C)alkyl which is optionally substituted by halo, hydroxy, cyano, amino, (1-2C)alkylamino, di-(1-2C)alkylamino and/or (1-2C)alkoxy (which is optionally further substituted by halo and/or (1-2C)alkoxy); or
(c) a cycloalkyl, aryl, heteroaryl, heterocyclyl, or heterocyclyl(1-2C)alkyl ring,
wherein each ring system is optionally substituted on an available carbon atom by one or more R_{101C} substituents;
wherein each R_{101C} substituent is independently selected from halo, hydroxy, cyano, R_{c}, NR_{d}R_{c}, OR_{c}, C(O)R_{c}, C(O)OR_{c}, OC(O)R_{c}, C(O)N(R_{d})R_{c}, C(O)N(R_{d})OR_{c}, N(R_{d})C(O)R_{c}, S(O)_{y}R_{c} (wherein y is 0, 1 or 2), S(O)₂N(R_{d})R_{c}, N(R_{d})S(O)₂R_{c}, (CH₂)_{z}R_{c} or (CH₂)_{z}NR_{c}R_{d} (where z is 1, 2 or 3);
and wherein R_{c} is selected from:
1. hydrogen;
2. (1-4C)alkyl which is optionally substituted by one or more substituents independently selected from halo, hydroxy, cyano, amino, (3-6C)cycloalkyl, (1-2C)alkylamino, di-(1-2C)alkylamino or (1-2C)alkoxy and wherein any alkyl or cycloalkyl moiety present in such substituent groups is optionally further substituted by halo, hydroxy and/or (1-2C)alkoxy; or
3. aryl, heteroaryl, heterocyclyl or heterocyclyl(1-2C)alkyl ring,
wherein each ring system is optionally substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)R_{c1}, C(O)OR_{c1}, OC(O)R_{c1}, C(O)N(R_{d1})R_{c1}, C(O)N(R_{d1})OR_{c1} N(R_{d1})C(O)R_{c1}, S(O)_{y}R_{c1} (wherein y is 0, 1 or 2), S(O)₂N(R_{b1})R_{c1} or N(R_{d1})S(O)₂R_{c1}, wherein R_{c1} and R_{d1} are each independently selected from hydrogen or (1-2C)alkyl, and wherein any alkyl moiety present in a substituent group is optionally further substituted by halo, hydroxy and/or (1-2C)alkoxy;
and R_{d} is selected from hydrogen or (1-2C)alkyl;
and, when R₂ or R_{c} is a heteroaryl or heterocyclyl ring, an available nitrogen atom (where valency permits) is optionally further substituted by one or more R_{101N} substituents, wherein R_{101N} is a (1-4C)alkyl which is optionally substituted by one or more substituents independently selected from halo, hydroxy, cyano, amino or (1-2C)alkoxy;
or, when L is —NR₁ₐCONR_{1b}—, R_{1b} and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N}.
5. A compound, or a pharmaceutically acceptable salt thereof, according to any one of paragraphs 1 to 4, wherein R₁ is selected from:
(i) an aryl, heteroaryl or heterocyclyl ring,
   each of which is optionally substituted on an available carbon atom by one or more R_{100C}, wherein R_{100C} is hydroxy; and an available nitrogen atom (where valency permits) is optionally substituted by one or more R_{100N}, wherein R_{100N} is (1-4C)alkyl which is optionally substituted by hydroxy; or
   heterocyclyl which is optionally substituted on an available ring nitrogen atom (where valency permits) by (1-4C)alkyl; or
(ii) a group -L-R₂ wherein:
   L is a linking group selected from: —CONR₁ₐ— or —CONR₁ₐ—O— or NR₁ₐCONR_{1b}—;
   wherein R₁ₐ and R_{1b} are hydrogen; and
   R₂ is selected from: hydrogen, (1-4C)alkyl (which is optionally substituted by hydroxy), a cycloalkyl, heteroaryl or heterocyclyl ring;
   wherein each ring system is optionally substituted on an available carbon atom by one or more R_{101C} substituents independently selected from hydroxy, R_{c}, OR_{c} and C(O)N(R_{d})OR_{c}, wherein R_{c} is selected from:
   (a) (1-4C)alkyl which is optionally substituted by one or more substituents independently selected from halo, hydroxy, (3-6C)cycloalkyl or (1-4C)alkoxy; or
   (b) an aryl or heterocyclyl(1-2C)alkyl ring, wherein each ring system is optionally substituted on an available carbon atom by cyano;
   and R_{d} is selected from hydrogen or (1-2C)alkyl;
   and, when R₂ is a heterocyclyl ring, an available nitrogen atom (where valency permits) is optionally further substituted by one or more R_{101N}, wherein R_{101N} is (1-4C)alkyl;
   or, when L is —NR₁ₐCONR_{1b}—, R_{1b} and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N}.
6. A compound or a pharmaceutically acceptable salt thereof, according to any one of paragraphs 2 to 5, wherein in Formula (Ia) X₂, X₃ and X₄ define a group as depicted in (B-8) or (B-9) below:

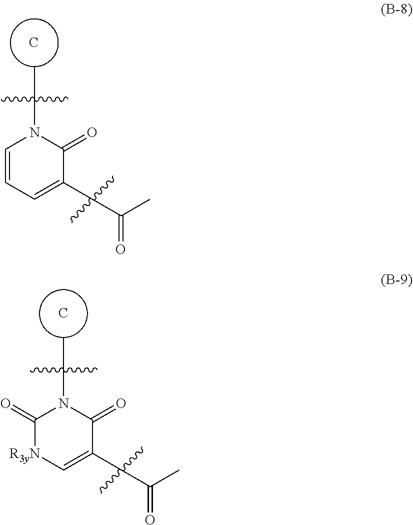

wherein R_{3y} is hydrogen or (1-4C)alkyl.
7. A compound or a pharmaceutically acceptable salt thereof, according to any one of paragraphs 1 to 6, wherein Ring A is selected from Ring A-1, i.e. phenyl, Ring A-2, Ring A-3, Ring A-4, Ring A-5 or Ring A-7, each optionally substituted by one or more substituents independently selected from halo, (1-4C)alkyl and (1-4C)alkoxy and Ring C is Ring C-1, i.e. phenyl, optionally substituted by one or more halo.
8. A compound or a pharmaceutically acceptable salt thereof, according to any one of paragraphs 1 to 7, wherein Ring A is phenyl, optionally substituted by one or more substituents independently selected from halo, (1-4C)alkyl and (1-4C)alkoxy and Ring C is phenyl, optionally substituted by halo.
9. A compound or a pharmaceutically acceptable salt thereof, according to any one of paragraphs 1 to 8, wherein:
   (i) Ring C is Ring C-1, i.e. phenyl, or Ring C-2, each optionally substituted by one or more halo; or
   (ii) Ring C is Ring C-1, i.e. phenyl, optionally substituted by one or more halo.
10. A compound of Formula (Ib), or a pharmaceutically acceptable salt thereof, according to any one of paragraphs 1 to 9, as shown below:

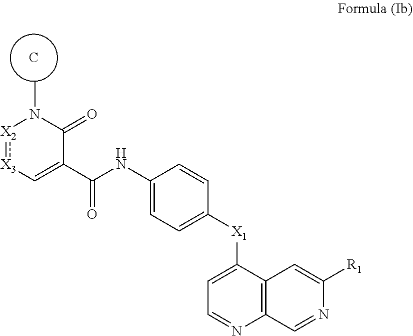

wherein
R₁ is as defined in any one of paragraphs 1, 4 or 5;
X₁ is —NH— or —O—;
X₂ is CH or CO;
X₃ is CH or NR_{3y} wherein R_{3y} is hydrogen or (1-4C)alkyl;
and wherein there is a single bond between X₂ and X₃ or a double bond when each is CH;
Ring C is as defined in paragraph 1 or paragraph 9.
11. A compound of Formula (Ic), or a pharmaceutically acceptable salt thereof, according to any one of paragraphs 1 to 9, as shown below:

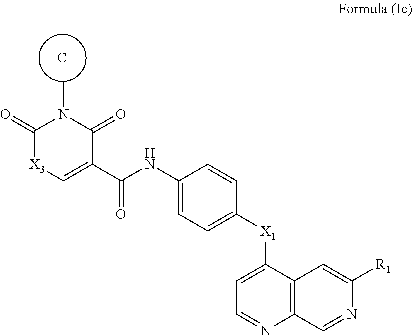

R₁ is as defined in any one of paragraphs 1, 4 or 5;
X₁ is —NH— or —O—;
X₂ is CH or CO;
X₃ is NR_{3y} wherein R_{3y} is hydrogen or (1-4C)alkyl;
and Ring C is as defined in paragraph 1 or paragraph 9.
12. A compound or a pharmaceutically acceptable salt thereof, according to any one of the preceding paragraphs, selected from one of the following:
2-oxo-1-phenyl-N-[4-[(6-piperazin-1-yl-1,7-naphthyridin-4-yl)amino]phenyl]pyridine-3-carboxamide;
1,5-dimethyl-3-oxo-2-phenyl-N-[4-[(6-piperazin-1-yl-1,7-naphthyridin-4-yl)amino]phenyl]pyrazole-4-carboxamide;
3-(4-fluorophenyl)-1-isopropyl-2,4-dioxo-N-[4-[(6-piperazin-1-yl-1,7-naphthyridin-4-yl)amino]phenyl]pyrimidine-5-carboxamide;
1-(4-fluorophenyl)-2-oxo-N-[4-[(6-piperazin-1-yl-1,7-naphthyridin-4-yl)amino]phenyl]pyridine-3-carboxamide;
1-isopropyl-2,4-dioxo-3-phenyl-N-[4-[(6-piperazin-1-yl-1,7-naphthyridin-4-yl)amino]phenyl]pyrimidine-5-carboxamide;
N-[4-[[6-(4-hydroxy-1-piperidyl)-1,7-naphthyridin-4-yl]amino]phenyl]-2-oxo-1-phenyl-pyridine-3-carboxamide;
N-[4-[(6-morpholino-1,7-naphthyridin-4-yl)amino]phenyl]-2-oxo-1-phenyl-pyridine-3-carboxamide;
N-[4-[[6-(1,4-diazepan-1-yl)-1,7-naphthyridin-4-yl]amino]phenyl]-2-oxo-1-phenyl-pyridine-3-carboxamide;
N-(1-methyl-4-piperidyl)-4-[4-[(2-oxo-1-phenyl-pyridine-3-carbonyl)amino]anilino]-1,7-naphthyridine-6-carboxamide;
N-[4-[[6-(1-methylpyrazol-4-yl)-1,7-naphthyridin-4-yl]amino]phenyl]-2-oxo-1-phenyl-pyridine-3-carboxamide;
N-[4-[[6-[1-(1-methyl-4-piperidyl)pyrazo]-4-yl]-1,7-naphthyridin-4-yl]amino]phenyl]-2-oxo-1-phenyl-pyridine-3-carboxamide;
N-(4-hydroxycyclohexyl)-4-[4-[(2-oxo-1-phenyl-pyridine-3-carbonyl)amino]anilino]-1,7-naphthyridine-6-carboxamide;
N-(2-hydroxyethoxy)-4-[4-[(2-oxo-1-phenyl-pyridine-3-carbonyl)amino]anilino]-1,7-naphthyridine-6-carboxamide;
4-[4-[(2-oxo-1-phenyl-pyridine-3-carbonyl)amino]anilino]-1,7-naphthyridine-6-carboxamide;
N-methyl-4-[4-[(2-oxo-1-phenyl-pyridine-3-carbonyl)amino]anilino]-1,7-naphthyridine-6-carboxamide;
N-(1-methyl-4-piperidyl)-4-[4-[(2-oxo-1-phenyl-pyridine-3-carbonyl)amino]phenoxy]-1,7-naphthyridine-6-carboxamide;
4-[4-[[3-(4-fluorophenyl)-1-isopropyl-2,4-dioxo-pyrimidine-5-carbonyl]amino]anilino]-N-(2-hydroxyethoxy)-1,7-naphthyridine-6-carboxamide;
N-[2-(dimethylamino)ethyl]-4-[4-[[3-(4-fluorophenyl)-1-isopropyl-2,4-dioxo-pyrimidine-5-carbonyl]amino]anilino]-1,7-naphthyridine-6-carboxamide;
4-[4-[(1,5-Dimethyl-3-oxo-2-phenyl-pyrazole-4-carbonyl)amino]phenoxy]-N-(1-methyl-4-piperidyl)-1,7-naphthyridine-6-carboxamide;
4-[4-[[1-(4-fluorophenyl)-2-oxo-pyridine-3-carbonyl]amino]phenoxy]-N-(1-methyl-4-piperidyl)-1,7-naphthyridine-6-carboxamide;
4-[4-[[3-(4-fluorophenyl)-1-isopropyl-2,4-dioxo-pyrimidine-5-carbonyl]amino]phenoxy]-N-(1-methyl-4-piperidyl)-1,7-naphthyridine-6-carboxamide;
4-[4-[[3-(5-Fluoro-2-pyridyl)-1-isopropyl-2,4-dioxo-pyrimidine-5-carbonyl]amino]phenoxy]-N-(1-methyl-4-piperidyl)-1,7-naphthyridine-6-carboxamide;
4-[4-[[3-(3,5-difluorophenyl)-1-isopropyl-2,4-dioxo-pyrimidine-5-carbonyl]amino]phenoxy]-N-(1-methyl-4-piperidyl)-1,7-naphthyridine-6-carboxamide;
N-[3-methyl-4-[(6-piperazin-1-yl-1,7-naphthyridin-4-yl)oxy]phenyl]-2-oxo-1-phenyl-pyridine-3-carboxamide;
2-oxo-1-phenyl-N-[5-[(6-piperazin-1-yl-1,7-naphthyridin-4-yl)oxy]-2-pyridyl]pyridine-3-carboxamide;
N-[3-fluoro-4-[(6-piperazin-1-yl-1,7-naphthyridin-4-yl)oxy]phenyl]-2-oxo-1-phenyl-pyridine-3-carboxamide;
1-(4-fluorophenyl)-2-oxo-N-[5-[(6-piperazin-1-yl-1,7-naphthyridin-4-yl)oxy]-2-pyridyl]pyridine-3-carboxamide;
1-(4-fluorophenyl)-N-[5-[[6-(4-methylpiperazin-1-yl)-1,7-naphthyridin-4-yl]oxy]-2-pyridyl]-2-oxo-pyridine-3-carboxamide;
1-(4-fluorophenyl)-N-[5-[[6-[4-(oxetan-3-yl)piperazin-1-yl]-1,7-naphthyridin-4-yl]oxy]-2-pyridyl]-2-oxo-pyridine-3-carboxamide;
4-[2-Methyl-4-[(2-oxo-1-phenyl-pyridine-3-carbonyl)amino]phenoxy]-N-(1-methyl-4-piperidyl)-1,7-naphthyridine-6-carboxamide;
4-[2-fluoro-4-[(2-oxo-1-phenyl-pyridine-3-carbonyl)amino]phenoxy]-N-(1-methyl-4-piperidyl)-1,7-naphthyridine-6-carboxamide;
4-[[6-[[3-(4-fluorophenyl)-1-isopropyl-2,4-dioxo-pyrimidine-5-carbonyl]amino]-3-pyridyl]oxy]-N-(1-methyl-4-piperidyl)-1,7-naphthyridine-6-carboxamide;
N-(1-methyl-4-piperidyl)-4-[[6-[(2-oxo-1-phenyl-pyridine-3-carbonyl)amino]-3-pyridyl]oxy]-1,7-naphthyridine-6-carboxamide;
4-[[6-[[1-(4-fluorophenyl)-2-oxo-pyridine-3-carbonyl]amino]-3-pyridyl]oxy]-N-(1-methyl-4-piperidyl)-1,7-naphthyridine-6-carboxamide;
2-Oxo-1-phenyl-N-[4-[(6-piperazin-1-yl-1,7-naphthyridin-4-yl)oxy]phenyl]pyridine-3-carboxamide;
2-oxo-N-[4-[(6-piperazin-1-yl-1,7-naphthyridin-4-yl)oxy]phenyl]-1-(2-pyridyl)pyridine-3-carboxamide;
1-(4-fluorophenyl)-2-oxo-N-[4-[(6-piperazin-1-yl-1,7-naphthyridin-4-yl)oxy]phenyl]pyridine-3-carboxamide;
1-isopropyl-2,4-dioxo-N-[4-[(6-piperazin-1-yl-1,7-naphthyridin-4-yl)oxy]phenyl]-3-(2-pyridyl)pyrimidine-5-carboxamide;
3-(5-fluoro-2-pyridyl)-1-isopropyl-2,4-dioxo-N-[4-[(6-piperazin-1-yl-1,7-naphthyridin-4-yl)oxy]phenyl]pyrimidine-5-carboxamide;
3-(4-fluorophenyl)-1-isopropyl-2,4-dioxo-N-[4-[(6-piperazin-1-yl-1,7-naphthyridin-4-yl)oxy]phenyl]pyrimidine-5-carboxamide;
6-(4-fluorophenyl)-1-oxido-N-[4-[(6-piperazin-1-yl-1,7-naphthyridin-4-yl)oxy]phenyl]pyridin-1-ium-2-carboxamide;
1-(4-fluorophenyl)-N-[4-[[6-(4-methylpiperazin-1-yl)-1,7-naphthyridin-4-yl]oxy]phenyl]-2-oxo-pyridine-3-carboxamide;
5-(4-fluorophenyl)-4-oxo-N-[4-[(6-piperazin-1-yl-1,7-naphthyridin-4-yl)oxy]phenyl]-1H-pyridine-3-carboxamide;
1,5-dimethyl-3-oxo-2-phenyl-N-[4-[(6-piperazin-1-yl-1,7-naphthyridin-4-yl)oxy]phenyl]pyrazole-4-carboxamide;
5-(4-Fluorophenyl)-1-isopropyl-4-oxo-N-[4-[(6-piperazin-1-yl-1,7-naphthyridin-4-yl)oxy]phenyl]pyridine-3-carboxamide;
5-(4-fluorophenyl)-1-methyl-4-oxo-N-[4-[(6-piperazin-1-yl-1,7-naphthyridin-4-yl)oxy]phenyl]pyridine-3-carboxamide;
4-[4-[[5-(4-fluorophenyl)-1-methyl-4-oxo-pyridine-3-carbonyl]amino]phenoxy]-N-(1-methyl-4-piperidyl)-1,7-naphthyridine-6-carboxamide;
4-[4-[[5-(4-fluorophenyl)-1-isopropyl-4-oxo-pyridine-3-carbonyl]amino]phenoxy]-N-(1-methyl-4-piperidyl)-1,7-naphthyridine-6-carboxamide;
4-[4-[[3-(4-Fluorophenyl)-2,4-dioxo-1-[(1R)-2-hydroxy-1-methyl-ethyl]pyrimidine-5-carbonyl]amino]phenoxy]-N-(1-methyl-4-piperidyl)-1,7-naphthyridine-6-carboxamide;
4-[4-[[1-Cyclopropyl-3-(4-fluorophenyl)-2,4-dioxo-pyrimidine-5-carbonyl]amino]phenoxy]-N-(1-methyl-4-piperidyl)-1,7-naphthyridine-6-carboxamide;
1-cyclopropyl-3-(4-fluorophenyl)-2,4-dioxo-N-[4-[(6-piperazin-1-yl-1,7-naphthyridin-4-yl)oxy]phenyl]pyrimidine-5-carboxamide;
1-(4-Fluorophenyl)-N-[5-[[6-(1-methyl-4-piperidyl)-1,7-naphthyridin-4-yl]oxy]-2-pyridyl]-2-oxo-pyridine-3-carboxamide;
1-(4-fluorophenyl)-N-[4-[[6-(1-methyl-4-piperidyl)-1,7-naphthyridin-4-yl]oxy]phenyl]-2-oxo-pyridine-3-carboxamide;
1-(4-Fluorophenyl)-N-[5-[[6-[(1-methylpyrrolidine-3-carbonyl)amino]-1,7-naphthyridin-4-yl]oxy]-2-pyridyl]-2-oxo-pyridine-3-carboxamide;
1-(4-fluorophenyl)-N-[5-[[6-[(1-methylpiperidine-4-carbonyl)amino]-1,7-naphthyridin-4-yl]oxy]-2-pyridyl]-2-oxo-pyridine-3-carboxamide;
4-[2-[[1-(4-Fluorophenyl)-2-oxo-pyridine-3-carbonyl]amino]pyrimidin-5-yl]oxy-N-(1-methyl-4-piperidyl)-1,7-naphthyridine-6-carboxamide.
13. A compound according to any one of the preceding paragraphs or a pharmaceutically acceptable salt thereof, for use in therapy.
14. A pharmaceutical composition comprising a compound according to any one of paragraphs 1 to 12 or a pharmaceutically acceptable salt thereof and a pharmaceutically acceptable carrier or excipient.
15. A compound according to any one of paragraphs 1 to 2 or a pharmaceutically acceptable salt thereof, or a pharmaceutical composition according to paragraph 13, for use in a method of inhibiting cell proliferation, such as the treatment of cancer.
16. A method for the treatment of cancer in a subject in need of such treatment, said method comprising administering a therapeutically effective amount of a compound according to any of paragraphs 1 to 12, or a pharmaceutically acceptable salt thereof, or a pharmaceutical composition according to paragraph 13.
17. A combination comprising a compound according to any of paragraphs 1 to 12, or a pharmaceutically acceptable salt thereof, or a pharmaceutical composition according to paragraph 13, and an immune checkpoint inhibitor as defined herein, or a pharmaceutically acceptable salt thereof, for use in the treatment of cancer.

1. A compound of Formula (I) or a pharmaceutically acceptable salt thereof, as shown below: 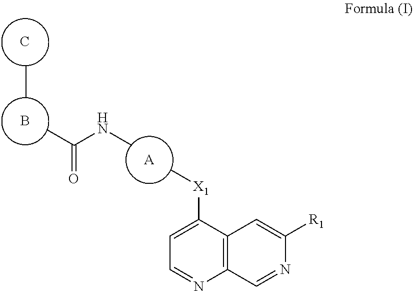
wherein:
R₁ is selected from:
(i) a cycloalkyl, aryl, heteroaryl or heterocyclyl ring,
each of which is optionally substituted on an available carbon atom by one or more R_{100C} substituents;
wherein each R_{100C} substituent present is independently selected from halo, hydroxy, cyano, Rₐ, NRₐR_{b}, ORₐ, C(O)Rₐ, C(O)ORₐ, OC(O)Rₐ, C(O)N(R_{b})Rₐ, C(O)N(R_{b})ORₐ, N(R_{b})C(O)Rₐ, S(O)_{y}Rₐ (wherein y is 0, 1 or 2), S(O)₂N(R_{b})Rₐ, N(R_{b})S(O)₂Rₐ, (CH₂)_{z}Rₐ or (CH₂)_{z}NRₐR_{b} (where z is 1, 2 or 3);
wherein Rₐ is selected from:
(a) (1-4C)alkyl which is optionally substituted by one or more substituents independently selected from halo, hydroxy, cyano, amino, (3-6C)cycloalkyl, (1-4C)alkylamino, di-(1-4C)alkylamino or (1-4C)alkoxy and wherein any alkyl or cycloalkyl moiety present in such substituent groups is optionally further substituted by halo, hydroxy and/or (1-2C)alkoxy; or
(b) aryl, heteroaryl, cycloalkyl, heterocyclyl or heterocyclyl(1-2C)alkyl, each of which is optionally substituted on an available carbon atom by one or more substituents independently selected from halo, hydroxy, cyano, amino, (1-4C)alkyl, (1-4C)alkylamino, di-(1-4C)alkylamino, (1-4C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are each independently selected from hydrogen or (1-4C)alkyl, and wherein any alkyl moiety present in a substituent group is optionally further substituted by halo, hydroxy and/or (1-2C)alkoxy;
and R_{b} is selected from hydrogen or (1-2C)alkyl;
and, when R₁ or Rₐ is a heteroaryl, heterocyclyl or heterocyclyl(1-2C)alkyl, an available nitrogen atom (where valency permits) is optionally substituted by one or more R_{100N};
wherein each R_{100N} is selected from:
(a) (1-4C)alkyl which is optionally substituted by halo, oxo, hydroxy, cyano, amino and/or (1-4C)alkoxy (optionally substituted by halo and/or (1-2C)alkoxy); or
(b) (3-6C)cycloalkyl, phenyl, heterocyclyl or heteroaryl, each of which is optionally further substituted on an available carbon atom by one or more substituents independently selected from halo, hydroxy, cyano, amino, (1-4C)alkyl, (1-4C)alkylamino, di-(1-4C)alkylamino, (1-4C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are as defined above, and wherein any alkyl moiety present in a substituent group is optionally further substituted by halo, hydroxy and/or (1-2C)alkoxy;
and an available ring nitrogen atom (where valency permits) is optionally further substituted by (1-4C)alkyl optionally substituted by halo, hydroxy, cyano, amino and/or (1-4C)alkoxy (which is optionally further substituted by halo and/or (1-2C)alkoxy);
(c) C(O)Rₐ₂, C(O)ORₐ₂, C(O)N(R_{b2})Rₐ₂ or C(O)N(R_{b2})ORₐ₂; wherein Rₐ₂ and R_{b2} are each independently selected from hydrogen or (1-4C)alkyl, and wherein any alkyl moiety present in a substituent group is optionally further substituted by halo, hydroxy and/or (1-2C)alkoxy;
or
(ii) a group:
-L-R₂;
wherein:
L is a linking group selected from: —O—; —CO—; —COO—; —OCO—; —NR₁ₐ—; —CONR₁ₐ—; —CONR₁ₐ—O—; —NR₁ₐCO—; —NR₁ₐCOO—; or —NR₁ₐCONR_{1b}—;
R₁ₐ or R_{1b} are both independently selected from hydrogen or (1-4C)alkyl; and
R₂ is selected from:
(a) hydrogen;
(b) (1-4C)alkyl which is optionally substituted by halo, hydroxy, cyano, amino, (1-4C)alkylamino, di-(1-4C)alkylamino and/or (1-4C)alkoxy (which is optionally further substituted by halo and/or (1-4C)alkoxy); or
(c) a cycloalkyl, aryl, heteroaryl, heterocyclyl, cycloalkyl(1-2C)alkyl, aryl(1-2C)alkyl, heteroaryl(1-2C)alkyl or heterocyclyl(1-2C)alkyl ring,
wherein each ring system is optionally substituted on an available carbon atom by one or more R_{101C} substituents;
wherein each R_{101C} substituent is independently selected from halo, hydroxy, cyano, R_{c}, NR_{d}R_{c}, OR_{c}, C(O)R_{c}, C(O)OR_{c}, OC(O)R_{c}, C(O)N(R_{d})R_{c}, C(O)N(R_{d})OR_{c}, N(R_{d})C(O)R_{c}, S(O)_{y}R_{c} (wherein y is 0, 1 or 2), S(O)₂N(R_{d})R_{c}, N(R_{d})S(O)₂R_{c}, (CH₂)_{z}R_{c} or (CH₂)_{z}NR_{c}R_{d} (where z is 1, 2 or 3);
and wherein R_{c} is selected from:
(i) hydrogen;
(ii) (1-4C)alkyl which is optionally substituted by one or more substituents independently selected from halo, hydroxy, cyano, amino, (3-6C)cycloalkyl, (1-4C)alkylamino, di-(1-4C)alkylamino or (1-4C)alkoxy and wherein any alkyl or cycloalkyl moiety present in such substituent groups is optionally further substituted by halo, hydroxy and/or (1-2C)alkoxy; or
(iii) a cycloalkyl, aryl, heteroaryl, heterocyclyl or heterocyclyl(1-2C)alkyl ring,
wherein each ring system is optionally substituted on an available carbon atom by one or more substituents independently selected from halo, hydroxy, cyano, amino, (1-4C)alkyl, (1-4C)alkylamino, di-(1-4C)alkylamino, (1-4C)alkoxy, C(O)R_{c1}, C(O)OR_{c1}, OC(O)R_{c1}, C(O)N(R_{d1})R_{c1}, C(O)N(R_{d1})OR_{c1} N(R_{d1})C(O)R_{c1}, S(O)_{y}R_{c1} (wherein y is 0, 1 or 2), S(O)₂N(R_{b1})R_{c1} or N(R_{d1})S(O)₂R_{c1}, wherein R_{c1} and R_{d1} are each independently selected from hydrogen or (1-4C)alkyl, and wherein any alkyl moiety present in a substituent group is optionally further substituted by halo, hydroxy and/or (1-2C)alkoxy;
and R_{d} is selected from hydrogen or (1-2C)alkyl;
and, when R₂ or R_{c} is a heteroaryl or heterocyclyl ring, an available nitrogen atom (where valency permits) is optionally further substituted by one or more R_{101N};
wherein each R_{101N} substituent is selected from:
(a) (1-4C)alkyl which is optionally substituted by one or more substituents independently selected from halo, hydroxy, cyano, amino or (1-4C)alkoxy (which is optionally further substituted by halo and/or (1-2C)alkoxy);
(b) (3-6C)cycloalkyl, phenyl, or a carbon-linked heterocyclyl or heteroaryl ring, each of which is optionally further substituted by one or more substituents independently selected from halo, hydroxy, cyano, amino, (1-4C)alkyl, (1-4C)alkylamino, di-(1-4C)alkylamino, (1-4C)alkoxy, C(O)R_{c1}, C(O)OR_{c1}, OC(O)R_{c1}, C(O)N(R_{d1})R_{c1}, C(O)N(R_{d1})OR_{c1} N(R_{d1})C(O)R_{c1}, S(O)_{y}R_{c1} (wherein y is 0, 1 or 2), S(O)₂N(R_{b1})R_{c1} or N(R_{d1})S(O)₂R_{c1}, wherein R_{c1} and R_{d1} are as defined above and wherein any alkyl moiety present in a substituent group is optionally further substituted by halo, hydroxy and/or (1-2C)alkoxy;
(c) C(O)R_{c2}, C(O)OR_{c2}, C(O)N(R_{d2})R_{c2} or C(O)N(R_{d2})OR_{C2}; wherein R_{c2} and R_{d2} are each independently selected from hydrogen or (1-4C)alkyl, and wherein any alkyl moiety present in a substituent group is optionally further substituted by halo, hydroxy and/or (1-2C)alkoxy;
or, when L is a linking group selected from —NR₁ₐ—, —CONR₁ₐ—, or —NR₁ₐCO, R₁ₐ and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N};
or, when L is —NR₁ₐCONR_{1b}—, R_{1b} and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N};
X₁ is —NH— or —O—;
Ring A is selected from the following: 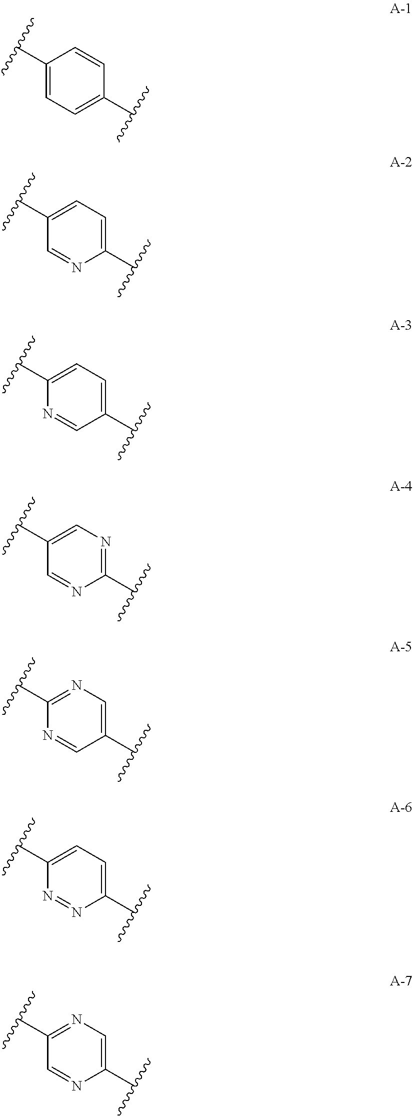
wherein [symbol] represents the respective points of attachment of Ring A to the rest of the compound of Formula (I) and wherein Ring A may be optionally substituted on an available carbon atom by one or more substituents independently selected from halo, (1-4C)alkyl and (1-4C)alkoxy;
Ring B is a 5- or 6-membered heteroaryl or a heterocyclic ring comprising one to three N atoms, linked via a carbon atom to the amide bond in the compound of Formula (I) and wherein:
(a) a heteroaryl ring is optionally substituted on an available carbon atom by one or more substituents independently selected from hydroxy, halo, cyano, (1-6C)alkyl or (1-6C)alkoxy; or on an available nitrogen atom by (1-6C)alkyl; and wherein any alkyl moiety present in a substituent group is optionally further substituted by halo, hydroxy and/or (1-2C)alkoxy;
and
(b) a heterocyclyl ring is optionally substituted on an available carbon atom by one or more substituents independently selected from oxo, hydroxy, halo, cyano, (1-6C)alkyl, (1-6C)alkoxy; or on an available nitrogen atom by (1-6C)alkyl; and wherein any alkyl moiety present in a substituent group is optionally further substituted by halo, hydroxy and/or (1-2C)alkoxy;
Ring C is selected from the following: 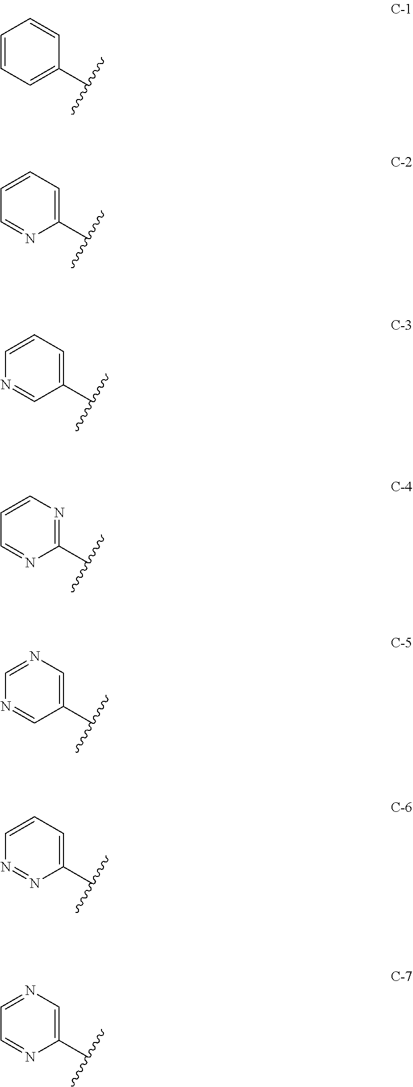
wherein [symbol] represents the point of attachment of Ring C to Ring B and wherein Ring C is optionally substituted on an available carbon atom by one or more substituents independently selected from hydroxy, halo, cyano, (1-6C)alkyl or (1-6C)alkoxy; or on an available nitrogen atom by (1-6C)alkyl; and wherein any alkyl moiety present in a substituent group is optionally further substituted by halo, hydroxy and/or (1-2C)alkoxy.

2. A compound, or a pharmaceutically acceptable salt thereof, according to claim 1, wherein Ring B is a 5- or 6-membered heteroaryl having the formula B-1 or B-2 shown below: 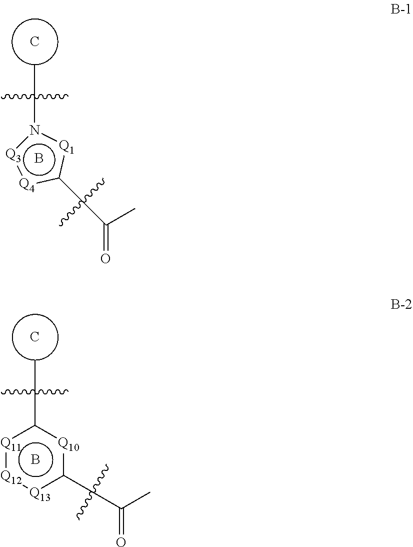
wherein
Q₁, Q₃ and Q₄ are CH or N;
wherein one of Q₁, Q₃ and Q₄ is N;
Q₁₀, Q₁₁, Q₁₂ and Q₁₃ are CR_{q2} or N;
wherein up to two of Q₁₀, Q₁₁, Q₁₂ and Q₁₃ can be N and R_{q2} is hydrogen or a substituent selected from hydroxy, halo, cyano, (1-2C)alkyl or (1-2C)alkoxy;
or Ring B is a 6-membered heterocyclic ring of the formula B-3 or B-4 shown below: 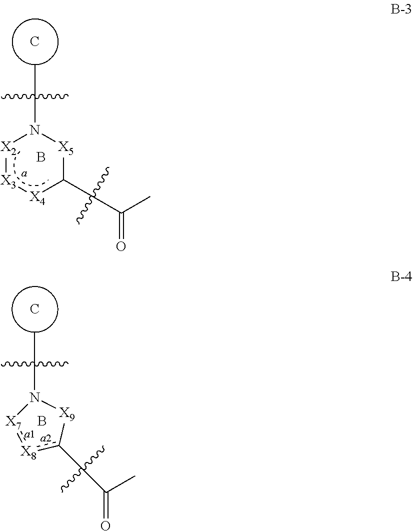
wherein
X₂, X₃ and X₄ are selected from C═O, CRₓ, CHRₓ or NR_{y};
X₅ is selected from C═O, CHRₓ or NR_{y};
wherein no more than two of X₂, X₃, X₄ and X₅ can be N and no more than two of X₂, X₃, X₄ and X₅ can be C═O;
Rₓ is hydrogen or a substituent selected from hydroxy, halo, cyano, (1-4C)alkyl, (1-4C)alkoxy;
R_{y} is hydrogen or (1-4C)alkyl;
and wherein any alkyl moiety present in a Rₓ or R_{y} substituent group is optionally further substituted by halo, hydroxy and/or (1-2C)alkoxy; and [symbol]
represents an optional double bond between X₂ and X₃ and/or between X₄ and the adjacent ring carbon atom linked to the amide bond;
X₇ and X₈ are selected from CRₓ CHRₓ or NR_{y};
X₉ is selected from C═O, CHRₓ or NR_{y};
wherein one of X₇, X₈ and X₉ can be N;
Rₓ and R_{y} are as defined above; and
both a1 and a2 are single bonds or one of a1 or a2 is a double bond;
or Ring B is a 6-membered heterocyclyl ring having the formula B-10 shown below: 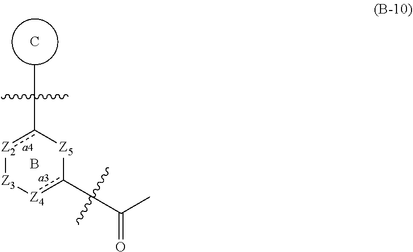
wherein
Z₂, Z₄ and Z₅ are selected from C═O, CRᵥ, CHR_{y};
Z₃ is selected from C═O, CHRᵥ or NR_{w};
wherein no more than three of Z₂, Z₃, Z₄ and Z₅ can be C═O;
Rᵥ is hydrogen or a substituent selected from hydroxy, halo, cyano, (1-6C)alkyl, (1-6C)alkoxy;
R_{w} is hydrogen or (1-6C)alkyl;
and wherein any alkyl moiety present in a Rᵥ or R_{w} substituent group is optionally further substituted by halo, hydroxy and/or (1-2C)alkoxy; and
both a3 and a4 are single bonds, one of a3 and a4 is a double bond, or both of a3 and a4 are double bonds.

3. A compound of Formula (Ia) or Formula (Id), or a pharmaceutically acceptable salt thereof, according to claim 1 or claim 2, as shown below: 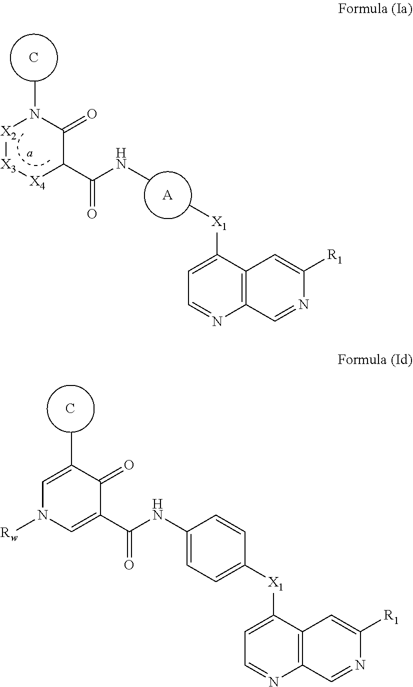
wherein R₁, X₁, Ring A and Ring C are as defined in claim 1 or claim 2, and R_{w} is as defined in claim 2; and wherein in Formula (Ia): [symbol]
represents an optional double bond between X₂ and X₃ and/or between X₄ and the adjacent ring carbon atom linked to the amide bond;
X₂ is N or CR₂ₓ wherein R₂ₓ is hydrogen, (1-4C)alkyl or (1-4C)alkoxy when there is a double bond between X₂ and X₃ or
X₂ is CO, CH₂, CHR₂ₓ or NR_{2y} wherein R_{2y} is hydrogen or (1-4C)alkyl when there is a single bond between X₂ and X₃;
X₃ is N or CR₃ wherein R₃ₓ is hydrogen, (1-4C)alkyl or (1-4C)alkoxy when there is a double bond between X₂ and X₃ or
X₃ is CO, CH₂, CHR₃ₓ or NR_{3y} wherein R_{3y} is hydrogen or (1-4C)alkyl when there is a single bond between X₂ and X₃;
X₄ is N or CR₄ₓ wherein R₄ₓ is hydrogen, (1-4C)alkyl or (1-4C)alkoxy when there is a double bond between X₄ and the adjacent ring carbon atom linked to the amide bond or
X₄ is CO, CH₂, CHR₄ₓ or NR_{4y} wherein R_{4y} is hydrogen or (1-4C)alkyl when there is a single bond between X₄ and the adjacent ring carbon atom linked to the amide bond;
wherein any alkyl moiety present in a ring substituent group is optionally further substituted by halo, hydroxy or (1-2C)alkoxy;
and provided that:
(i) a maximum of one of X₂, X₃ and X₄ is N or NR_{2y}, NR_{3y} or R_{4y} respectively;
(ii) a maximum of one of X₂, X₃ and X₄ is CO;
(iii) when X₂ is N then X₃ is CO;

4. A compound according to any one of claims 1 to 3, or a pharmaceutically acceptable salt or solvate thereof, wherein R₁ is selected from:
(i) a cycloalkyl, aryl, heteroaryl or heterocyclyl ring,
each of which is optionally substituted on an available carbon atom by one or more R_{100C} substituents;
wherein each R_{100C} substituent is independently selected from halo, hydroxy, cyano, Rₐ, NRₐR_{b}, ORₐ, C(O)Rₐ, C(O)ORₐ, OC(O)Rₐ, C(O)N(R_{b})Rₐ, C(O)N(R_{b})ORₐ, N(R_{b})C(O)Rₐ, S(O)_{y}Rₐ (wherein y is 0, 1 or 2), S(O)₂N(R_{b})Rₐ, N(R_{b})S(O)₂Rₐ, (CH₂)_{z}Rₐ or (CH₂)_{z}NRₐR_{b} (where z is 1, 2 or 3);
wherein Rₐ is selected from:
(a) (1-2C)alkyl which is optionally substituted by one or more substituents independently selected from halo, hydroxy, cyano, amino, (3-4C)cycloalkyl, (1-2C)alkylamino, di-(1-2C)alkylamino or (1-2C)alkoxy and wherein any alkyl or cycloalkyl moiety present in such substituent groups is optionally further substituted by halo, hydroxy and/or (1-2C)alkoxy; or
(b) aryl, heteroaryl, heterocyclyl or heterocyclyl(1-2C)alkyl, each of which is optionally substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are each independently selected from hydrogen or (1-2C)alkyl, and wherein any alkyl moiety present in a substituent group is optionally further substituted by halo, hydroxy and/or (1-2C)alkoxy;
and R_{b} is selected from hydrogen or (1-2C)alkyl;
and, when R₁ or Rₐ is a heteroaryl, heterocyclyl or heterocyclyl(1-2C)alkyl, an available nitrogen atom (where valency permits) is optionally substituted by one or more R_{100N}:
wherein R_{100N} is selected from:
(a) (1-4C)alkyl which is optionally substituted by halo, hydroxy, cyano, amino and/or (1-1C)alkoxy (optionally substituted by halo and/or (1-2C)alkoxy); or
(b) heterocyclyl or heteroaryl, each of which is optionally further substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)Rₐ₁, C(O)ORₐ₁, OC(O)Rₐ₁, C(O)N(R_{b1})Rₐ₁, C(O)N(R_{b1})ORₐ₁, N(R_{b1})C(O)Rₐ₁, S(O)_{y}Rₐ₁ (wherein y is 0, 1 or 2), S(O)₂N(R_{b1})Rₐ₁ or N(R_{b1})S(O)₂Rₐ₁, wherein Rₐ₁ and R_{b1} are as defined above, and wherein any alkyl moiety present in a substituent group is optionally further substituted by halo, hydroxy and/or (1-2C)alkoxy;
and an available ring nitrogen atom (where valency permits) is optionally further substituted by (1-4C)alkyl optionally substituted by halo, hydroxy, cyano, amino and/or (1-2C)alkoxy; or
(c) C(O)Rₐ₂, C(O)ORₐ₂, C(O)N(R_{b2})Rₐ₂ or C(O)N(R_{b2})ORₐ₂; wherein Rₐ₂ and R_{b2} are each independently selected from hydrogen or (1-4C)alkyl, and wherein any alkyl moiety present in a substituent group is optionally further substituted by halo, hydroxy and/or (1-2C)alkoxy; or
(ii) a group:
-L-R₂;
wherein:
L is a linking group selected from: —O—; —CO—; —COO—; —OCO—; —NR₁ₐ—; —CONR₁ₐ—; —CONR₁ₐ—O—; —NR₁ₐCO—; —NR₁ₐCOO—; or NR₁ₐCONR_{1b}—;
R₁ₐ and R_{1b} are independently selected from hydrogen or (1-2C)alkyl; and
R₂ is selected from:
(a) hydrogen;
(b) (1-4C)alkyl which is optionally substituted by halo, hydroxy, cyano, amino, (1-2C)alkylamino, di-(1-2C)alkylamino and/or (1-2C)alkoxy (which is optionally further substituted by halo and/or (1-2C)alkoxy); or
(c) a cycloalkyl, aryl, heteroaryl, heterocyclyl, or heterocyclyl(1-2C)alkyl ring,
wherein each ring system is optionally substituted on an available carbon atom by one or more R_{101C} substituents;
wherein each R_{101C} substituent is independently selected from halo, hydroxy, cyano, R_{c}, NR_{d}R_{c}, OR_{c}, C(O)R_{c}, C(O)OR_{c}, OC(O)R_{c}, C(O)N(R_{d})R_{c}, C(O)N(R_{d})OR_{c}, N(R_{d})C(O)R_{c}, S(O)_{y}R_{c} (wherein y is 0, 1 or 2), S(O)₂N(R_{d})R_{c}, N(R_{d})S(O)₂R_{c}, (CH₂)_{z}R_{c} or (CH₂)_{z}NR_{c}R_{d} (where z is 1, 2 or 3);
and wherein R_{c} is selected from:
i. hydrogen;
ii. (1-4C)alkyl which is optionally substituted by one or more substituents independently selected from halo, hydroxy, cyano, amino, (3-6C)cycloalkyl, (1-2C)alkylamino, di-(1-2C)alkylamino or (1-2C)alkoxy and wherein any alkyl or cycloalkyl moiety present in such substituent groups is optionally further substituted by halo, hydroxy and/or (1-2C)alkoxy; or
iii. aryl, heteroaryl, heterocyclyl or heterocyclyl(1-2C)alkyl ring,
wherein each ring system is optionally substituted on an available carbon atom by one or more substituents independently selected from halo, hydroxy, cyano, amino, (1-2C)alkyl, (1-2C)alkylamino, di-(1-2C)alkylamino, (1-2C)alkoxy, C(O)R_{c1}, C(O)OR_{c1}, OC(O)R_{c1}, C(O)N(R_{d1})R_{c1}, C(O)N(R_{d1})OR_{c1} N(R_{d1})C(O)R_{c1}, S(O)_{y}R_{c1} (wherein y is 0, 1 or 2), S(O)₂N(R_{b1})R_{c1} or N(R_{d1})S(O)₂R_{c1}, wherein R_{c1} and R_{d1} are each independently selected from hydrogen or (1-2C)alkyl, and wherein any alkyl moiety present in a substituent group is optionally further substituted by halo, hydroxy and/or (1-2C)alkoxy;
and R_{d} is selected from hydrogen or (1-2C)alkyl;
and, when R₂ or R_{c} is a heteroaryl or heterocyclyl ring, an available nitrogen atom (where valency permits) is optionally further substituted by one or more R_{101N} substituents, wherein R_{101N} is selected from:
a) (1-4C)alkyl which is optionally substituted by one or more substituents independently selected from halo, hydroxy, cyano, amino or (1-2C)alkoxy; or
b) C(O)R_{c2}, C(O)OR_{c2}, C(O)N(R_{d2})R_{c2} or C(O)N(R_{d2})OR_{c2}; wherein R_{c2} and R_{d2} are each independently selected from hydrogen or (1-4C)alkyl, and wherein any alkyl moiety present in a substituent group is optionally further substituted by halo, hydroxy and/or (1-2C)alkoxy;
or, when L is —NR₁ₐCONR_{1b}—, R_{1b} and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N}.

5. A compound, or a pharmaceutically acceptable salt thereof, according to any one of claims 1 to 4, wherein R₁ is selected from:
i. an aryl, heteroaryl or heterocyclyl ring,
each of which is optionally substituted on an available carbon atom by one or more R_{100C}, wherein R_{100C} is hydroxy; and an available nitrogen atom (where valency permits) is optionally substituted by one or more R_{100N}, wherein R_{100N} is
(1-4C)alkyl which is optionally substituted by hydroxy; or
heterocyclyl which is optionally substituted on an available ring nitrogen atom (where valency permits) by (1-4C)alkyl; or
C(O)ORₐ₂; wherein Rₐ₂ is (1-4C)alkyl; or
ii. a group -L-R₂ wherein:
L is a linking group selected from: —CONR₁ₐ— or —CONR₁ₐ—O— or NR₁ₐCONR_{1b}—; wherein R₁ₐ and R_{1b} are hydrogen; and
R₂ is selected from: hydrogen, (1-4C)alkyl (which is optionally substituted by hydroxy), a cycloalkyl, heteroaryl or heterocyclyl ring;
wherein each ring system is optionally substituted on an available carbon atom by one or more R_{101C} substituents independently selected from hydroxy, R_{c}, OR_{c} and C(O)N(R_{d})OR_{c}, wherein R_{c} is selected from:
a) (1-4C)alkyl which is optionally substituted by one or more substituents independently selected from halo, hydroxy, (3-6C)cycloalkyl or (1-4C)alkoxy; or
b) an aryl or heterocyclyl(1-2C)alkyl ring, wherein each ring system is optionally substituted on an available carbon atom by cyano;
and R_{d} is selected from hydrogen or (1-2C)alkyl;
and, when R₂ is a heterocyclyl ring, an available nitrogen atom (where valency permits) is optionally further substituted by one or more R_{101N}, wherein R_{101N} is (1-4C)alkyl or C(O)OR_{c2}; wherein R_{c2} is (1-4C)alkyl;
or, when L is —NR₁ₐCONR_{1b}—, R_{1b} and R₂ may be linked such that, together with the nitrogen atom to which they are attached, they form a nitrogen-linked heterocyclic ring, which is optionally substituted on any available carbon atom by one or more R_{101C} and on any available nitrogen atom (where valency permits) by one or more R_{101N}.

6. A compound, or a pharmaceutically acceptable salt thereof, according to any one of claims 1 to 4, wherein R₁ is selected from:
i) a heterocyclyl ring, which may be optionally substituted on an available carbon atom by hydroxy;
and may be optionally substituted on an available nitrogen atom (where valency permits) by (1-4C)alkyl which is optionally substituted by hydroxy.
ii) a group -L-R₂
wherein:
L is a linking group selected from: —CONH— or —CONH—O— or NHCONH—; and
R₂ is a heterocyclyl ring; which is optionally substituted on an available carbon atom by one or more substituents independently selected from hydroxy, R_{c}, OR_{c} and C(O)N(R_{d})OR_{c}; and may be optionally substituted on an available nitrogen atom (where valency permits) by (1-4C)alkyl which is optionally substituted by hydroxy;
wherein R_{c} is selected from:
a) (1-4C)alkyl which is optionally substituted by one or more substituents independently selected from halo, hydroxy, (3-6C)cycloalkyl or (1-4C)alkoxy; or
b) an aryl or heterocyclyl(1-2C)alkyl ring, wherein each ring system is optionally substituted on an available carbon atom by cyano;
and R_{d} is selected from hydrogen or (1-2C)alkyl;
and, when R₂ is a heterocyclyl ring, an available nitrogen atom (where valency permits) is optionally further substituted by (1-4C)alkyl.

7. A compound or a pharmaceutically acceptable salt thereof, according to any one of claims 1 to 6, wherein Ring B is a group as depicted in (B-8), (B-9) or (B-12) below: 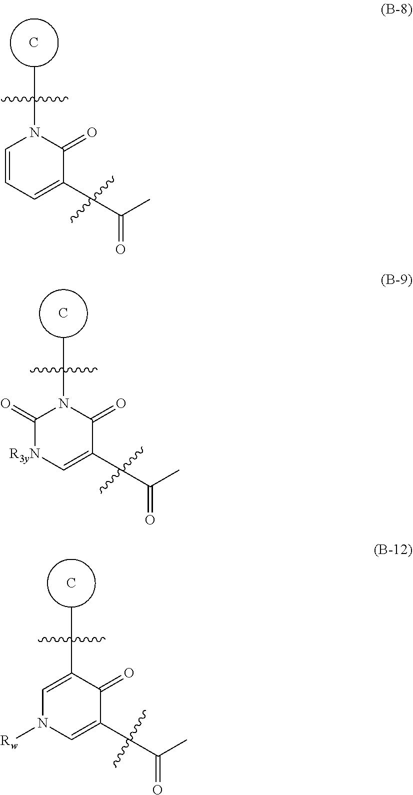
wherein:
R_{3y} is hydrogen or (1-4C)alkyl;
R_{w} is hydrogen or (1-4C)alkyl; wherein the (1-4C)alkyl is optionally further substituted by halo, hydroxy and/or (1-2C)alkoxy.

8. A compound or a pharmaceutically acceptable salt thereof, according to any one of claims 1 to 7, wherein Ring A is selected from Ring A-1, i.e. phenyl, Ring A-2, Ring A-3, Ring A-4, Ring A-5 or Ring A-7, each optionally substituted by one or more substituents independently selected from halo, (1-4C)alkyl and (1-4C)alkoxy and Ring C is Ring C-1, i.e. phenyl, optionally substituted by one or more halo.

9. A compound or a pharmaceutically acceptable salt thereof, according to any one of claims 1 to 8, wherein Ring A is phenyl, optionally substituted by one or more substituents independently selected from halo, (1-4C)alkyl and (1-4C)alkoxy and Ring C is phenyl, optionally substituted by halo.

10. A compound or a pharmaceutically acceptable salt thereof, according to any one of claims 1 to 9, wherein:
(i) Ring C is Ring C-1, i.e. phenyl, or Ring C-2, each optionally substituted by one or more halo; or
(ii) Ring C is Ring C-1, i.e. phenyl, optionally substituted by one or more halo.

11. A compound of Formula (Ib), or a pharmaceutically acceptable salt thereof, according to any one of claims 1 to 10, as shown below: 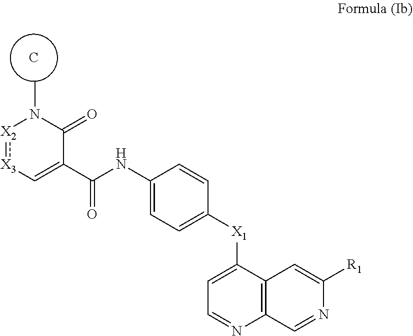
wherein
R₁ is as defined in any one of claims 1, 4 or 5;
X₁ is —NH— or —O—;
X₂ is CH or CO;
X₃ is CH or NR_{3y} wherein R_{3y} is hydrogen or (1-4C)alkyl;
and wherein there is a single bond between X₂ and X₃ or a double bond when each is CH;
Ring C is as defined in claim 1 or claim 9.

12. A compound of Formula (Ic), or a pharmaceutically acceptable salt thereof, according to any one of claims 1 to 10, as shown below: 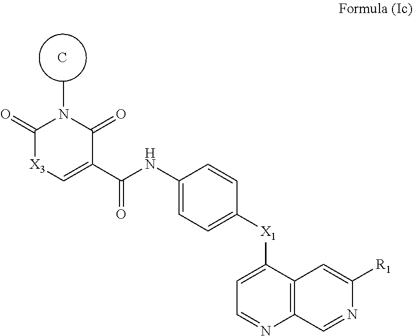
R₁ is as defined in any one of claims 1, 4, 5 or 6;
X₁ is —NH— or —O—;
X₂ is CH or CO;
X₃ is NR_{3y} wherein R_{3y} is hydrogen or (1-4C)alkyl;
and Ring C is as defined in claim 1 or claim 9.

13. A compound of Formula (Ie), or a pharmaceutically acceptable salt thereof, according to any one of claims 1 to 10, as shown below: 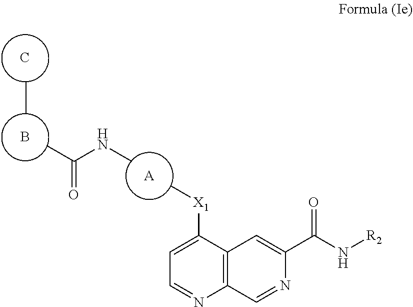
wherein R₂, Ring A, Ring B and Ring C are as defined in any one of the preceding claims.

14. A compound or a pharmaceutically acceptable salt thereof, according to any one of the preceding claims, selected from one of the following:
2-oxo-1-phenyl-N-[4-[(6-piperazin-1-yl-1,7-naphthyridin-4-yl)amino]phenyl]pyridine-3-carboxamide
3-(4-fluorophenyl)-1-isopropyl-2,4-dioxo-N-[4-[(6-piperazin-1-yl-1,7-naphthyridin-4-yl)amino]phenyl]pyrimidine-5-carboxamide
1-(4-fluorophenyl)-2-oxo-N-[4-[(6-piperazin-1-yl-1,7-naphthyridin-4-yl)amino]phenyl]pyridine-3-carboxamide
1-isopropyl-2,4-dioxo-3-phenyl-N-[4-[(6-piperazin-1-yl-1,7-naphthyridin-4-yl)amino]phenyl]pyrimidine-5-carboxamide
1,5-dimethyl-3-oxo-2-phenyl-N-[4-[(6-piperazin-1-yl-1,7-naphthyridin-4-yl)amino]phenyl]pyrazole-4-carboxamide
N-[4-[[6-(4-hydroxy-1-piperidyl)-1,7-naphthyridin-4-yl]amino]phenyl]-2-oxo-1-phenyl-pyridine-3-carboxamide
N-[4-[(6-morpholino-1,7-naphthyridin-4-yl)amino]phenyl]-2-oxo-1-phenyl-pyridine-3-carboxamide
N-[4-[[6-(1,4-diazepan-1-yl)-1,7-naphthyridin-4-yl]amino]phenyl]-2-oxo-1-phenyl-pyridine-3-carboxamide
N-(1-methyl-4-piperidyl)-4-[4-[(2-oxo-1-phenyl-pyridine-3-carbonyl)amino]anilino]-1,7-naphthyridine-6-carboxamide
N-[4-[[6-(1-methylpyrazol-4-yl)-1,7-naphthyridin-4-yl]amino]phenyl]-2-oxo-1-phenyl-pyridine-3-carboxamide
N-[4-[[6-[1-(1-methyl-4-piperidyl)pyrazo]-4-yl]-1,7-naphthyridin-4-yl]amino]phenyl]-2-oxo-1-phenyl-pyridine-3-carboxamide
N-(4-hydroxycyclohexyl)-4-[4-[(2-oxo-1-phenyl-pyridine-3-carbonyl)amino]anilino]-1,7-naphthyridine-6-carboxamide
N-(2-hydroxyethoxy)-4-[4-[(2-oxo-1-phenyl-pyridine-3-carbonyl)amino]anilino]-1,7-naphthyridine-6-carboxamide
4-[4-[(2-oxo-1-phenyl-pyridine-3-carbonyl)amino]anilino]-1,7-naphthyridine-6-carboxamide
N-methyl-4-[4-[(2-oxo-1-phenyl-pyridine-3-carbonyl)amino]anilino]-1,7-naphthyridine-6-carboxamide
N-[4-[[6-(azetidine-1-carbonyl)-1,7-naphthyridin-4-yl]amino]phenyl]-2-oxo-1-phenyl-pyridine-3-carboxamide
N-(1-methyl-4-piperidyl)-4-[4-[(2-oxo-1-phenyl-pyridine-3-carbonyl)amino]phenoxy]-1,7-naphthyridine-6-carboxamide
4-[4-[[3-(4-fluorophenyl)-1-isopropyl-2,4-dioxo-pyrimidine-5-carbonyl]amino]anilino]-N-(2-hydroxyethoxy)-1,7-naphthyridine-6-carboxamide
N-[2-(dimethylamino)ethyl]-4-[4-[[3-(4-fluorophenyl)-1-isopropyl-2,4-dioxo-pyrimidine-5-carbonyl]amino]anilino]-1,7-naphthyridine-6-carboxamide
N-(1-methyl-4-piperidyl)-4-[4-[[2-oxo-1-(2-pyridyl)pyridine-3-carbonyl]amino]phenoxy]-1,7-naphthyridine-6-carboxamide
4-[4-[[1-(4-fluorophenyl)-2-oxo-pyridine-3-carbonyl]amino]phenoxy]-N-(1-methyl-4-piperidyl)-1,7-naphthyridine-6-carboxamide
4-[4-[[3-(4-fluorophenyl)-1-isopropyl-2,4-dioxo-pyrimidine-5-carbonyl]amino]phenoxy]-N-(1-methyl-4-piperidyl)-1,7-naphthyridine-6-carboxamide
4-[4-[(1,5-dimethyl-3-oxo-2-phenyl-pyrazole-4-carbonyl)amino]phenoxy]-N-(1-methyl-4-piperidyl)-1,7-naphthyridine-6-carboxamide
4-[4-[[1-(2-hydroxy-2-methyl-propyl)-5-methyl-3-oxo-2-phenyl-pyrazole-4-carbonyl]amino]phenoxy]-N-(1-methyl-4-piperidyl)-1,7-naphthyridine-6-carboxamide
4-[4-[[3-(5-fluoro-2-pyridyl)-1-isopropyl-2,4-dioxo-pyrimidine-5-carbonyl]amino]phenoxy]-N-(1-methyl-4-piperidyl)-1,7-naphthyridine-6-carboxamide
4-[4-[[3-(3,5-difluorophenyl)-1-isopropyl-2,4-dioxo-pyrimidine-5-carbonyl]amino]phenoxy]-N-(1-methyl-4-piperidyl)-1,7-naphthyridine-6-carboxamide
1-(4-fluorophenyl)-2-oxo-N-[5-[(6-piperazin-1-yl-1,7-naphthyridin-4-yl)oxy]-2-pyridyl]pyridine-3-carboxamide
1-(4-fluorophenyl)-N-[5-[[6-(4-methylpiperazin-1-yl)-1,7-naphthyridin-4-yl]oxy]-2-pyridyl]-2-oxo-pyridine-3-carboxamide
1-(4-fluorophenyl)-N-[5-[[6-[4-(oxetan-3-yl)piperazin-1-yl]-1,7-naphthyridin-4-yl]oxy]-2-pyridyl]-2-oxo-pyridine-3-carboxamide
4-[[6-[[1-(4-fluorophenyl)-2-oxo-pyridine-3-carbonyl]amino]-3-pyridyl]oxy]-N-(1-methyl-4-piperidyl)-1,7-naphthyridine-6-carboxamide
2-oxo-1-phenyl-N-[4-[(6-piperazin-1-yl-1,7-naphthyridin-4-yl)oxy]phenyl]pyridine-3-carboxamide
2-oxo-N-[4-[(6-piperazin-1-yl-1,7-naphthyridin-4-yl)oxy]phenyl]-1-(2-pyridyl)pyridine-3-carboxamide
1-(4-fluorophenyl)-2-oxo-N-[4-[(6-piperazin-1-yl-1,7-naphthyridin-4-yl)oxy]phenyl]pyridine-3-carboxamide
1-isopropyl-2,4-dioxo-N-[4-[(6-piperazin-1-yl-1,7-naphthyridin-4-yl)oxy]phenyl]-3-(2-pyridyl)pyrimidine-5-carboxamide
3-(5-fluoro-2-pyridyl)-1-isopropyl-2,4-dioxo-N-[4-[(6-piperazin-1-yl-1,7-naphthyridin-4-yl)oxy]phenyl]pyrimidine-5-carboxamide
3-(4-fluorophenyl)-1-isopropyl-2,4-dioxo-N-[4-[(6-piperazin-1-yl-1,7-naphthyridin-4-yl)oxy]phenyl]pyrimidine-5-carboxamide
6-(4-fluorophenyl)-1-oxido-N-[4-[(6-piperazin-1-yl-1,7-naphthyridin-4-yl)oxy]phenyl]pyridin-1-ium-2-carboxamide
5-(4-fluorophenyl)-4-oxo-N-[4-[(6-piperazin-1-yl-1,7-naphthyridin-4-yl)oxy]phenyl]-1H-pyridine-3-carboxamide
1,5-dimethyl-3-oxo-2-phenyl-N-[4-[(6-piperazin-1-yl-1,7-naphthyridin-4-yl)oxy]phenyl]pyrazole-4-carboxamide
5-(4-fluorophenyl)-1-isopropyl-4-oxo-N-[4-[(6-piperazin-1-yl-1,7-naphthyridin-4-yl)oxy]phenyl]pyridine-3-carboxamide
5-(4-fluorophenyl)-1-methyl-4-oxo-N-[4-[(6-piperazin-1-yl-1,7-naphthyridin-4-yl)oxy]phenyl]pyridine-3-carboxamide
4-[4-[[3-(4-fluorophenyl)-2,4-dioxo-1-[rac-(1R)-2-hydroxy-1-methyl-ethyl]pyrimidine-5-carbonyl]amino]phenoxy]-N-(1-methyl-4-piperidyl)-1,7-naphthyridine-6-carboxamide
4-[4-[[1-cyclopropyl-3-(4-fluorophenyl)-2,4-dioxo-pyrimidine-5-carbonyl]amino]phenoxy]-N-(1-methyl-4-piperidyl)-1,7-naphthyridine-6-carboxamide
1-(4-fluorophenyl)-N-[5-[[6-(1-methyl-4-piperidyl)-1,7-naphthyridin-4-yl]oxy]-2-pyridyl]-2-oxo-pyridine-3-carboxamide
tert-butyl 3-[4-[[6-[[1-(4-fluorophenyl)-2-oxo-pyridine-3-carbonyl]amino]-3-pyridyl]oxy]-1,7-naphthyridin-6-yl]pyrrolidine-1-carboxylate
1-(4-fluorophenyl)-N-[5-[[6-[1-(oxetan-3-yl)-3,6-dihydro-2H-pyridin-4-yl]-1,7-naphthyridin-4-yl]oxy]-2-pyridyl]-2-oxo-pyridine-3-carboxamide
1-(4-fluorophenyl)-N-[5-[[6-[1-(oxetan-3-yl)-4-piperidyl]-1,7-naphthyridin-4-yl]oxy]-2-pyridyl]-2-oxo-pyridine-3-carboxamide
tert-butyl 4-[4-[[6-[[1-(4-fluorophenyl)-2-oxo-pyridine-3-carbonyl]amino]-3-pyridyl]oxy]-1,7-naphthyridin-6-yl]piperidine-1-carboxylate
1-(4-fluorophenyl)-N-[4-[[6-(1-methyl-4-piperidyl)-1,7-naphthyridin-4-yl]oxy]phenyl]-2-oxo-pyridine-3-carboxamide
1-(4-fluorophenyl)-N-[5-[[6-[(1-methylpyrrolidine-3-carbonyl)amino]-1,7-naphthyridin-4-yl]oxy]-2-pyridyl]-2-oxo-pyridine-3-carboxamide
1-(4-fluorophenyl)-N-[5-[[6-[(1-methylpiperidine-4-carbonyl)amino]-1,7-naphthyridin-4-yl]oxy]-2-pyridyl]-2-oxo-pyridine-3-carboxamide
4-[2-[[1-(4-fluorophenyl)-2-oxo-pyridine-3-carbonyl]amino]pyrimidin-5-yl]oxy-N-(1-methyl-4-piperidyl)-1,7-naphthyridine-6-carboxamide
2-(4-fluorophenyl)-N-[5-[[6-[4-(oxetan-3-yl)piperazin-1-yl]-1,7-naphthyridin-4-yl]oxy]-2-pyridyl]-3-oxo-pyridazine-4-carboxamide
3-(4-fluorophenyl)-1-isopropyl-N-[5-[[6-[4-(oxetan-3-yl)piperazin-1-yl]-1,7-naphthyridin-4-yl]oxy]-2-pyridyl]-2,4-dioxo-pyrimidine-5-carboxamide
5-(4-fluorophenyl)-1-isopropyl-N-[5-[[6-[4-(oxetan-3-yl)piperazin-1-yl]-1,7-naphthyridin-4-yl]oxy]-2-pyridyl]-4-oxo-pyridine-3-carboxamide
1-(4-fluorophenyl)-2-oxo-N-[5-[(6-pyrrolidin-3-yl-1,7-naphthyridin-4-yl)oxy]-2-pyridyl]pyridine-3-carboxamide
1-(4-fluorophenyl)-2-oxo-N-[5-[[6-(4-piperidyl)-1,7-naphthyridin-4-yl]oxy]-2-pyridyl]pyridine-3-carboxamide
1-(4-fluorophenyl)-N-[5-[[6-[1-(2-methylpropanoyl)-4-piperidyl]-1,7-naphthyridin-4-yl]oxy]-2-pyridyl]-2-oxo-pyridine-3-carboxamide
5-(4-fluorophenyl)-1-isopropyl-N-[5-[[6-(1-methyl-4-piperidyl)-1,7-naphthyridin-4-yl]oxy]-2-pyridyl]-4-oxo-pyridine-3-carboxamide
3-(4-fluorophenyl)-1-isopropyl-N-[5-[[6-(1-methyl-4-piperidyl)-1,7-naphthyridin-4-yl]oxy]-2-pyridyl]-2,4-dioxo-pyrimidine-5-carboxamide
1-(4-fluorophenyl)-N-[5-[[6-[(1-methyl-4-piperidyl)oxy]-1,7-naphthyridin-4-yl]oxy]-2-pyridyl]-2-oxo-pyridine-3-carboxamide
4-[[6-[[1-(4-fluorophenyl)-2-oxo-pyridine-3-carbonyl]amino]-4-methyl-3-pyridyl]oxy]-N-(1-methyl-4-piperidyl)-1,7-naphthyridine-6-carboxamide
4-[[6-[[1-(4-fluorophenyl)-2-oxo-pyridine-3-carbonyl]amino]-2-methyl-3-pyridyl]oxy]-N-(1-methyl-4-piperidyl)-1,7-naphthyridine-6-carboxamide
4-[[6-[[3-(4-fluorophenyl)-1-isopropyl-2,4-dioxo-pyrimidine-5-carbonyl]amino]-3-pyridyl]oxy]-N-(4-piperidyl)-1,7-naphthyridine-6-carboxamide
tert-butyl 4-[[4-[[6-[[3-(4-fluorophenyl)-1-isopropyl-2,4-dioxo-pyrimidine-5-carbonyl]amino]-3-pyridyl]oxy]-1,7-naphthyridine-6-carbonyl]amino]piperidine-1-carboxylate
4-[[6-[[3-(4-fluorophenyl)-1-isopropyl-2,4-dioxo-pyrimidine-5-carbonyl]amino]-3-pyridyl]oxy]-N-(8-methyl-8-azabicyclo[3.2.1]octan-3-yl)-1,7-naphthyridine-6-carboxamide
N-[2-(dimethylamino)ethyl]-4-[[6-[[3-(4-fluorophenyl)-1-isopropyl-2,4-dioxo-pyrimidine-5-carbonyl]amino]-3-pyridyl]oxy]-1,7-naphthyridine-6-carboxamide
4-[[6-[[3-(4-fluorophenyl)-1-isopropyl-2,4-dioxo-pyrimidine-5-carbonyl]amino]-3-pyridyl]oxy]-N-[rac-(3S)-1-methylpyrrolidin-3-yl]-1,7-naphthyridine-6-carboxamide
4-[[6-[[3-(4-fluorophenyl)-1-isopropyl-2,4-dioxo-pyrimidine-5-carbonyl]amino]-3-pyridyl]oxy]-N-(1-methyl-4-piperidyl)-1,7-naphthyridine-6-carboxamide
4-[[6-[[3-(4-fluorophenyl)-1-isopropyl-2,4-dioxo-pyrimidine-5-carbonyl]amino]-3-pyridyl]oxy]-N-[rac-(3R)-1-methylpyrrolidin-3-yl]-1,7-naphthyridine-6-carboxamide
4-[[6-[[3-(4-fluorophenyl)-1-isopropyl-2,4-dioxo-pyrimidine-5-carbonyl]amino]-3-pyridyl]oxy]-N-[(4-methylmorpholin-2-yl)methyl]-1,7-naphthyridine-6-carboxamide
N-[3-(dimethylamino)cyclobutyl]-4-[[6-[[3-(4-fluorophenyl)-1-isopropyl-2,4-dioxo-pyrimidine-5-carbonyl]amino]-3-pyridyl]oxy]-1,7-naphthyridine-6-carboxamide
4-[[6-[[3-(4-fluorophenyl)-1-isopropyl-2,4-dioxo-pyrimidine-5-carbonyl]amino]-3-pyridyl]oxy]-N-methyl-N-(1-methyl-4-piperidyl)-1,7-naphthyridine-6-carboxamide
4-[[6-[[5-(4-fluorophenyl)-1-isopropyl-4-oxo-pyridine-3-carbonyl]amino]-3-pyridyl]oxy]-N-(1-methyl-4-piperidyl)-1,7-naphthyridine-6-carboxamide
4-[[6-[[1-(4-fluorophenyl)-5-isopropyl-2-oxo-pyridine-3-carbonyl]amino]-3-pyridyl]oxy]-N-(1-methyl-4-piperidyl)-1,7-naphthyridine-6-carboxamide
N-(1-methyl-4-piperidyl)-4-[[6-[(2-oxo-1-phenyl-pyridine-3-carbonyl)amino]-3-pyridyl]oxy]-1,7-naphthyridine-6-carboxamide
4-[[6-[[5-(5-fluoro-2-pyridyl)-1-isopropyl-4-oxo-pyridine-3-carbonyl]amino]-3-pyridyl]oxy]-N-(1-methyl-4-piperidyl)-1,7-naphthyridine-6-carboxamide
N-[3-methyl-4-[(6-piperazin-1-yl-1,7-naphthyridin-4-yl)oxy]phenyl]-2-oxo-1-phenyl-pyridine-3-carboxamide
N-[3-fluoro-4-[(6-piperazin-1-yl-1,7-naphthyridin-4-yl)oxy]phenyl]-2-oxo-1-phenyl-pyridine-3-carboxamide
2-oxo-1-phenyl-N-[5-[(6-piperazin-1-yl-1,7-naphthyridin-4-yl)oxy]-2-pyridyl]pyridine-3-carboxamide
4-[2-fluoro-4-[(2-oxo-1-phenyl-pyridine-3-carbonyl)amino]phenoxy]-N-(1-methyl-4-piperidyl)-1,7-naphthyridine-6-carboxamide
4-[2-methyl-4-[(2-oxo-1-phenyl-pyridine-3-carbonyl)amino]phenoxy]-N-(1-methyl-4-piperidyl)-1,7-naphthyridine-6-carboxamide
1-(4-fluorophenyl)-N-[4-[[6-(4-methylpiperazin-1-yl)-1,7-naphthyridin-4-yl]oxy]phenyl]-2-oxo-pyridine-3-carboxamide
1-cyclopropyl-3-(4-fluorophenyl)-2,4-dioxo-N-[4-[(6-piperazin-1-yl-1,7-naphthyridin-4-yl)oxy]phenyl]pyrimidine-5-carboxamide
4-[4-[[5-(4-fluorophenyl)-1-methyl-4-oxo-pyridine-3-carbonyl]amino]phenoxy]-N-(1-methyl-4-piperidyl)-1,7-naphthyridine-6-carboxamide
4-[4-[[5-(4-fluorophenyl)-1-isopropyl-4-oxo-pyridine-3-carbonyl]amino]phenoxy]-N-(1-methyl-4-piperidyl)-1,7-naphthyridine-6-carboxamide.

15. A pharmaceutical composition comprising a compound according to any one of claims 1 to 12 or a pharmaceutically acceptable salt thereof and a pharmaceutically acceptable carrier or excipient.

16. A compound according to any one claims 1 to 14, or a pharmaceutically acceptable salt thereof, or a pharmaceutical composition according to claim 15, for use in therapy.

17. A compound according to any one of claims 1 to 14, or a pharmaceutically acceptable salt thereof, or a pharmaceutical composition according to claim 15, for use in a method of inhibiting cell proliferation, such as the treatment of cancer.

18. A method for the treatment of cancer in a subject in need of such treatment, said method comprising administering a therapeutically effective amount of a compound according to any of claims 1 to 14, or a pharmaceutically acceptable salt thereof, or a pharmaceutical composition according to claim 15.

19. A combination comprising a compound according to any of claims 1 to 14, or a pharmaceutically acceptable salt thereof, or a pharmaceutical composition according to claim 15, and an immune checkpoint inhibitor as defined herein, or a pharmaceutically acceptable salt thereof, for use in the treatment of cancer.
